(19) United States
(12) Patent Application Publication
(10) Pub. No.: US 2023/0009608 A1
(43) Pub. Date: Jan. 12, 2023

(54) GRK2 INHIBITORS AND USES THEREOF

(71) Applicant: Cygnal Therapeutics, Inc., Cambridge, MA (US)

(72) Inventors: Grazia Piizzi, Brookline, MA (US); Eugene Lvovich Piatnitski Chekler, Concord, MA (US); Jonathan Barry Hurov, Bedford, MA (US); Alexandra Lantermann, Cambridge, MA (US); Kiley Marie Couto, Quincy, MA (US); Hua Xu, Sharon, MA (US); Sourav Sarker, North Chelmsford, MA (US)

(21) Appl. No.: 17/330,796

(22) Filed: May 26, 2021

Related U.S. Application Data
(60) Provisional application No. 63/030,676, filed on May 27, 2020.

(51) Int. Cl. C07D 405/14; A61P 35/00

(52) U.S. Cl. CPC C07D 405/14; A61K 9/0019; A61P 35/00

(57) ABSTRACT

The present disclosure features useful methods to treat cancer, e.g., in a subject in need thereof. The methods described herein are useful in the treatment of disorders associated with GRK2 expression, e.g., cancer or cardiovascular disease. The present disclosure also features compounds (e.g., GRK2 inhibitors), pharmaceutically acceptable salts thereof, and pharmaceutical compositions thereof.

### RELATED APPLICATIONS

This application claims priority under 35 U.S.C. § 119(e) to U.S. Provisional Patent Application Ser. No. 63/030,676, filed May 27, 2020, the entire contents of which is incorporated herein by reference.

### BACKGROUND

G protein-coupled receptor kinases (GRKs) participate in the processes of regulation of multiple G protein-coupled receptors (GPCRs) of great physiological and pharmacological relevance. These proteins form a family of seven members that phosphorylate agonist-activated receptors in serine/threonine residues, promoting internalization, recycling and/or degradation processes of GPCRs.

GRK2, which is the most ubiquitous and best characterized isoform of the family of GRKs, has been found to regulate the activity of different GPCRs involved in cancer, along with cytosolic proteins involved in proliferative and survival signaling pathways, as well as non-GPCRs membrane proteins with oncogenic potential.

GRK2 levels and activity have also been reported to be enhanced in patients and/or in preclinical models of heart failure, cardiac hypertrophy, and hypertension.

Accordingly, there is a need to develop new compounds which decrease the level and/or activity of GRK2.

### SUMMARY OF THE DISCLOSURE

The present disclosure features useful methods to treat cancer, e.g., in a subject in need thereof. In some embodiments, the methods described herein are useful in the treatment of disorders associated with GRK2 expression, e.g., cancer or cardiovascular disease. The present disclosure also provides compounds, pharmaceutically acceptable salts thereof, and pharmaceutical compositions thereof, which are GRK2 inhibitors, e.g., GRK2-selective inhibitors.

In one aspect, the disclosure features a compound, or a pharmaceutically acceptable salt thereof, having the structure:

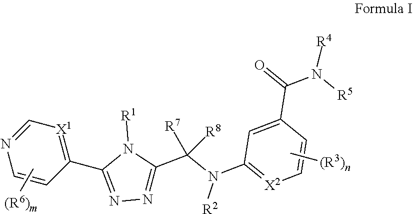

wherein m and n are, independently, 0, 1, 2, or 3;

X¹ is CR⁹ or N;

X² is CR³ or N;

R¹ is hydrogen, optionally substituted C₁-C₆ alkyl, optionally substituted C1-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;

R² and R⁴ are, independently, hydrogen or optionally substituted C₁-C₆ alkyl;

each R³ and R⁶ is, independently, halogen, optionally substituted C₁-C₆ alkyl, optionally substituted C₁-C₆ heteroalkyl, hydroxyl, thiol, or optionally substituted amino;

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl; and

R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl; and

R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl.

In some embodiments, the compound is a GRK2-selective compound.

In some embodiments, if m and n are 0, R⁷ and R⁸ are hydrogen, X² is CH, R¹ is hydrogen or optionally substituted C₁-C₆ alkyl, and R5 is optionally substituted C₁-C₆ alkyl C₆-C₁₀ aryl, then X² is N.

In some embodiments, the compound has the structure:

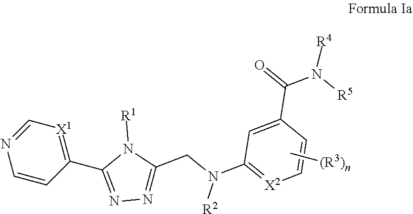

wherein n is 0, 1, 2, or 3;

X¹ and X² are, independently, CR³ or N;

R¹, R², and R⁴ are, independently, hydrogen or optionally substituted C₁-C₆ alkyl;

each R³ is, independently, hydrogen, halogen, optionally substituted C₁-C₆ alkyl, optionally substituted C₁-C₆ heteroalkyl, hydroxyl, thiol, or optionally substituted amino; and

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl, wherein if n is 0, X² is CH, and R⁵ is optionally substituted C₁-C₆ alkyl C₆-C₁₀ aryl, then X² is N.

In some embodiments, X¹ is N. In some embodiments, X¹ is CH. In some embodiments, X² is N. In some embodiments, X² is CH. In some embodiments, R² is hydrogen. In some embodiments, R⁴ is hydrogen. In some embodiments, R¹ is hydrogen. In some embodiments, R¹ is optionally substituted C₁-C₆ alkyl (e.g., methyl or ethyl). In some embodiments, R¹ is hydroxyalkyl. In some embodiments, R¹ is C₁-C₆ hydroxyalkyl. In some embodiments, R¹ is unsubstituted C₁-C₆ alkyl. In some embodiments, n is 0. In some embodiments, m is 0. In some embodiments, R⁷ is hydrogen. In some embodiments, R⁸ is hydrogen. In some embodiments, both R⁷ and R⁸ are hydrogen.

In some embodiments, R⁵ is optionally substituted C₁-C₆ alkyl C₆-C₁₀ aryl (e.g., optionally substituted C₂ alkyl C₆-C₁₀ aryl). In some embodiments, R⁵ is optionally substituted C₁ alkyl C₆-C₁₀ aryl. In some embodiments, R⁵ is optionally substituted C₁ alkyl phenyl. In some embodiments, R⁵ is:

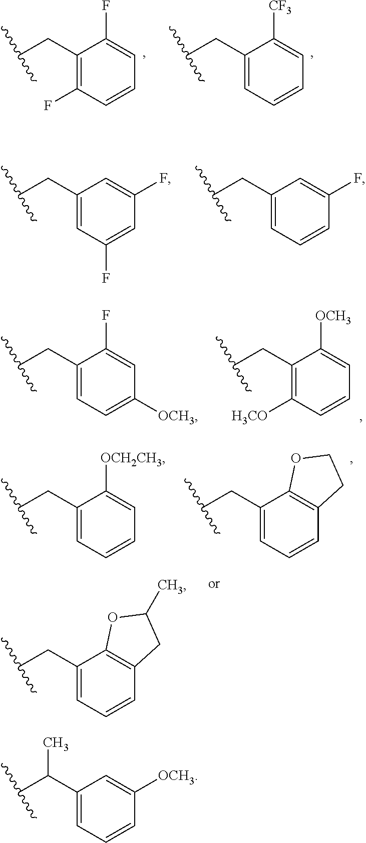

In some embodiments, R⁵ is optionally substituted C₂-C₉ heterocyclyl (e.g.,

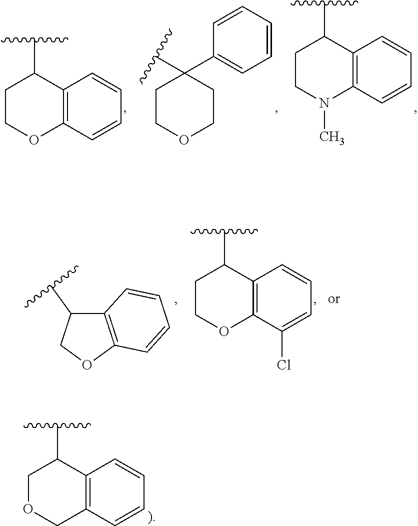

In some embodiments, R⁵ is optionally substituted C₃-C₈ cycloalkyl (e.g.,

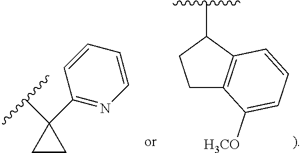 [SMILES: *C1(c2ccccn2)CC1 ; *C1CCc2c(OC)cccc21]

In some embodiments, R⁵ is optionally substituted C₁-C₆ alkyl C₂-C₉ heteroaryl (e.g.,

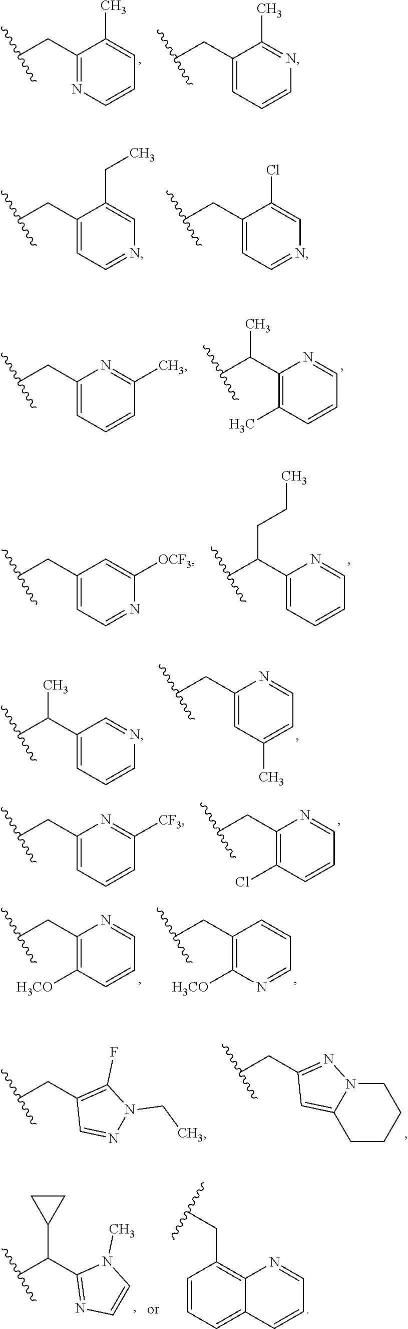

In some embodiments, R⁵ is optionally substituted C₁ alkyl C₂-C₉ heteroaryl. In some embodiments, R⁵ is optionally substituted C₁ alkyl pyridinyl.

In some embodiments, R⁵ is of the formula:

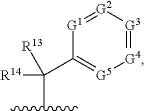

wherein:

G¹ is CR¹⁵ or N;

G², G³, G⁴, and G⁵ are each independently CR¹⁶, CH, or N;

each instance of R¹⁵ and R¹⁶ is independently hydrogen, halogen, —CN, —N₃, —NO₂, optionally substituted C₁₋C₆ alkyl, optionally substituted C₁₋C₆ heteroalkyl, optionally substituted C₂₋C₆ alkenyl, optionally substituted C₂-C₆ alkynyl, optionally substituted C₃-C₈ carbocyclyl, optionally substituted 3-8 membered heterocyclyl, optionally substituted C₆₋C₁₀ aryl, optionally substituted 5-10 membered heteroaryl, optionally substituted C₁₋C₆ acyl, optionally substituted hydroxyl, optionally substituted amino, or optionally substituted thiol;

R¹³ and R¹⁴ are independently hydrogen or optionally substituted C₁₋₆ alkyl, or R¹³ and R¹⁴ are joined together with the intervening atoms to form optionally substituted C₃₋₈ carbocyclyl or 3-8 membered heterocyclyl; and

optionally wherein R¹³ and R¹⁵ are joined together with the intervening atoms to form optionally substituted C₄₋₈ carbocyclyl or optionally substituted 4-8 membered heterocyclyl.

In some embodiments, G¹ is CR¹⁵. In some embodiments, G¹ is CH. In some embodiments, G² is CR¹⁶. In some embodiments, G² is CH. In some embodiments, G³ is CR¹⁶. In some embodiments, G³ is CH. In some embodiments, G⁴ is CR¹⁶. In some embodiments, G⁴ is CH. In some embodiments, G⁵ is CR¹⁶. In some embodiments, G⁵ is CH. In some embodiments, G², G³, G⁴, and G⁵ are independently CR¹⁶ or CH. In some embodiments, G², G³, G⁴, and G⁵ are CR¹⁶. In some embodiments, G², G³, G⁴, and G⁵ are CH.

In some embodiments, R¹³ is optionally substituted C₁₋₆ alkyl. In some embodiments, R¹³ is unsubstituted C₁₋₆ alkyl. In some embodiments, R¹³ is unsubstituted C₁₋₃ alkyl. In some embodiments, R¹³ is methyl. In some embodiments, R¹³ is hydrogen.

In some embodiments, R¹⁴ is hydrogen. In some embodiments, R¹³ and R¹⁴ are hydrogen. In some embodiments, R¹³ is optionally substituted C₁₋₆ alkyl; and R¹⁴ is hydrogen. In some embodiments, R¹³ is unsubstituted C₁₋₆ alkyl; and R¹⁴ is hydrogen. In some embodiments, R¹³ is unsubstituted C₁₋₃ alkyl; and R¹⁴ is hydrogen. In some embodiments, R¹³ is methyl; and R¹⁴ is hydrogen. In some embodiments, R¹³ and R¹⁴ are joined together with the intervening atoms to form optionally substituted C₃₋₈ carbocyclyl or 3-8 membered heterocyclyl.

In some embodiments, R¹⁵ is optionally substituted C₁₋₆ alkyl. In some embodiments, R¹⁵ is halogen. In some embodiments, R¹⁵ is —F. In some embodiments, R¹⁵ is C₁₋₆ haloalkyl. In some embodiments, R¹⁵ is trihalomethyl. In some embodiment, R¹⁵ is —CF₃. In some embodiments, R¹⁵ is optionally substituted hydroxyl. In some embodiments, R¹⁵ is —O—C₁₋₆ alkyl. In some embodiments, R¹⁵ is —OMe.

In some embodiments, R¹³ and R¹⁵ are joined together with the intervening atoms to form optionally substituted C₄₋₈ carbocyclyl or optionally substituted 4-8 membered heterocyclyl. In some embodiments, R¹³ and R¹⁵ are joined together with the intervening atoms to form optionally substituted 5-7 membered heterocyclyl comprising 1 or 2 heteroatoms independently selected from O, N, and S. In some embodiments, R¹³ and R¹⁵ are joined together with the intervening atoms to form optionally substituted 5-7 membered heterocyclyl comprising 1 heteroatom selected from O, N, and S. In some embodiments, R¹³ and R¹⁵ are joined together with the intervening atoms to form optionally substituted 6-membered heterocyclyl comprising 1 or 2 heteroatoms independently selected from O, N, and S. In some embodiments, R¹³ and R¹⁵ are joined together with the intervening atoms to form optionally substituted 6-membered heterocyclyl comprising 1 heteroatom selected from O, N, and S.

In some embodiments, at least one instance of R¹⁶ is hydrogen. In some embodiments, at least one instance of R¹⁶ is optionally substituted C₁₋₆ alkyl. In some embodiments, at least one instance of R¹⁶ is halogen. In some embodiments, at least one instance of R¹⁶ is —CN. In some embodiments, at least one instance of R¹⁶ is —N₃. In some embodiments, at least one instance of R¹⁶ is —NO₂. In some embodiments, at least one instance of R¹⁶ is optionally substituted C₁₋C₆ heteroalkyl. In some embodiments, at least one instance of R¹⁶ is optionally substituted C₂-C₆ alkenyl. In some embodiments, at least one instance of R¹⁶ is optionally substituted C₂-C₆ alkynyl. In some embodiments, at least one instance of R¹⁶ is optionally substituted C₃₋C₈ carbocyclyl. In some embodiments, at least one instance of R¹⁶ is optionally substituted 3-8 membered heterocyclyl. In some embodiments, at least one instance of R¹⁶ is optionally substituted C₆₋C₁₀ aryl. In some embodiments, at least one instance of R¹⁶ is optionally substituted 5-10 membered heteroaryl. In some embodiments, at least one instance of R¹⁶ is optionally substituted C₁₋C₆ acyl. In some embodiments, at least one instance of R¹⁶ is optionally substituted hydroxyl. In some embodiments, at least one instance of R¹⁶ is optionally substituted amino. In some embodiments, at least one instance of R¹⁶ is optionally substituted thiol.

In some embodiments, R¹⁵ and the adjacent R¹⁶ are joined together with the intervening atoms to form optionally substituted C₅₋₈ carbocyclyl, optionally substituted 5-8 membered heterocyclyl, optionally substituted C₆ aryl, or optionally substituted 5-6 membered heteroaryl.

In some embodiments, two adjacent R¹⁶ are joined together with the intervening atoms to form optionally substituted C₅₋₈ carbocyclyl, optionally substituted 5-8 membered heterocyclyl, optionally substituted C₆ aryl, or optionally substituted 5-6 membered heteroaryl.

In some embodiments, R⁵ is of the formula:

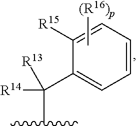

wherein p is 0, 1, 2, 3, or 4.

In some embodiments, R⁵ is of the formula:

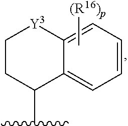

wherein Y³ is O, S, or optionally substituted N (e.g., —NH—, —N(C₁-C₆ alkyl)-). In some embodiments, Y³ is O. In some embodiments, Y³ is optionally substituted N. In some embodiments, Y³ is —NH—. In some embodiments, Y³ is —N(C₁-C₆ alkyl)- (e.g., —N(Me)-).

In some embodiments, R⁵ is of the formula:

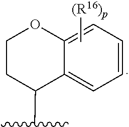

In some embodiments, R⁵ is:

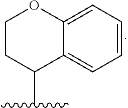 [SMILES: *C1CCOc2ccccc21]

In some embodiments, R⁵ is:

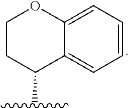 [SMILES: *[C@@H]1CCOc2ccccc21]

In some embodiments, R⁵ is:

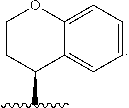 [SMILES: *[C@H]1CCOc2ccccc21]

In some embodiments, the compound or pharmaceutically acceptable salt thereof has the structure:

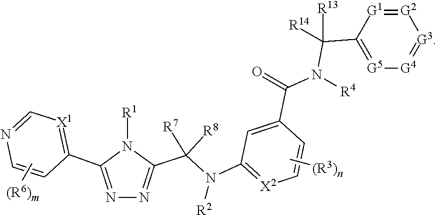

In some embodiments, the compound or pharmaceutically acceptable salt thereof has the structure:

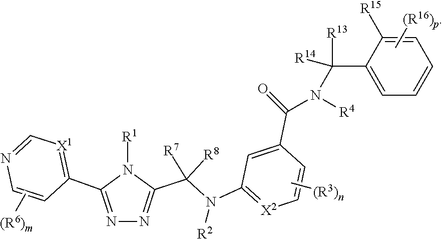

In some embodiments, the compound or pharmaceutically acceptable salt thereof has the structure:

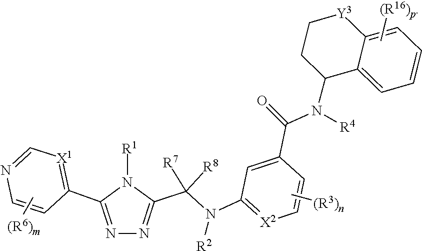

In some embodiments, the compound or pharmaceutically acceptable salt thereof has the structure:

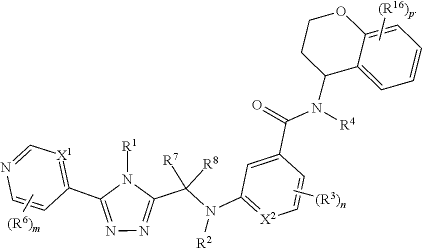

In some embodiments, the compound or pharmaceutically acceptable salt thereof has the structure:

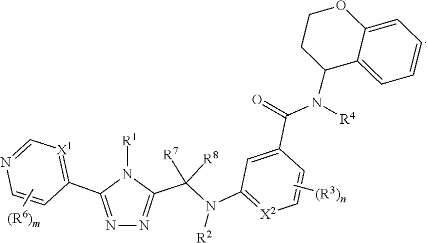

In some embodiments, the compound or pharmaceutically acceptable salt thereof has the structure:

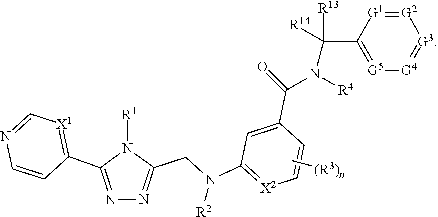

In some embodiments, the compound or pharmaceutically acceptable salt thereof has the structure:

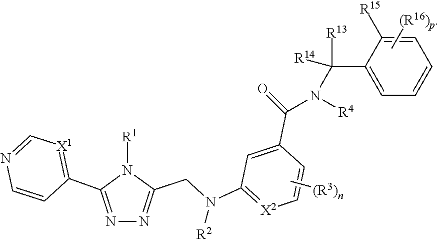

In some embodiments, the compound or pharmaceutically acceptable salt thereof has the structure:

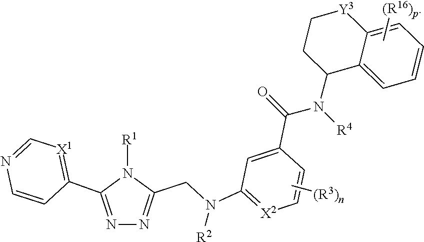

In some embodiments, the compound or pharmaceutically acceptable salt thereof has the structure:

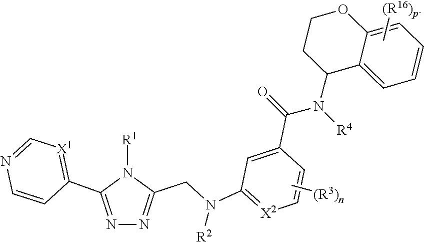

In some embodiments, the compound or pharmaceutically acceptable salt thereof has the structure:

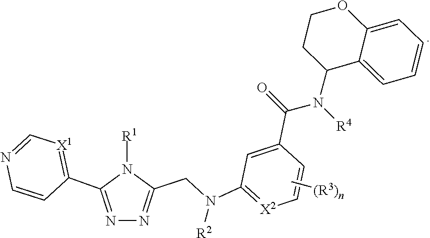

In an aspect, the disclosure features a compound, or a pharmaceutically acceptable salt thereof, having the structure of Formula II:

A-L¹-B Formula II,

wherein

L¹ is a linker;

B is a degradation moiety (e.g., a ubiquitin ligase binding moiety such as a ubiquitin ligase binding moiety including a Cereblon ligand, a IAP (Inhibitors of Apoptosis) ligand, a mouse double minute 2 homolog (MDM2), or a von Hippel-Lindau (VHL) ligand, or a derivative or an analog thereof); and

A has the structure of Formula III:

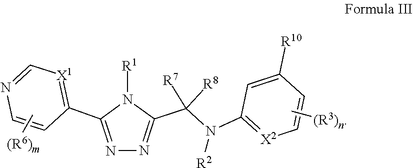

wherein m and n are, independently, 0, 1, 2, or 3;

X¹ is CR⁹ or N;

X² is CR³ or N;

R¹ is A¹, hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;

R² is hydrogen or optionally substituted C₁-C₆ alkyl;

each R³ and R⁶ is, independently, hydrogen, halogen, optionally substituted C₁-C₆ alkyl, optionally substituted C₁-C₆ heteroalkyl, hydroxyl, thiol, or optionally substituted amino;

R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl,

R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl;

R¹⁰ is —C(O)NR¹¹R¹² or —NHC(O)—R¹¹;

R¹¹ is A¹, optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl;

R¹² is hydrogen or optionally substituted C₁-C₆ alkyl; and

A¹ is a bond between A and the linker, wherein at least one, and only one of R¹ and R⁸ is A¹.

In some embodiments, A has the structure:

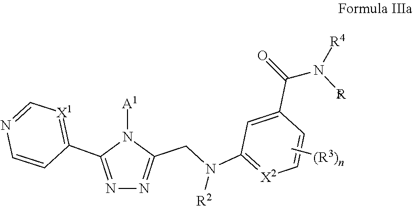

wherein n is 0, 1, 2, or 3;

X¹ and X² are, independently, CR³ or N;

R¹, R², and R⁴ are, independently, hydrogen or optionally substituted C₁-C₆ alkyl;

each R³ is, independently, hydrogen, halogen, optionally substituted C₁-C₆ alkyl, optionally substituted C₁-C₆ heteroalkyl, hydroxyl, thiol, or optionally substituted amino; and

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl; and

A¹ is a bond between A and the linker

In some embodiments, X¹ is N. In some embodiments, X¹ is CH. In some embodiments, R² is hydrogen. In some embodiments, R⁴ is hydrogen. In some embodiments, n is 0.

In some embodiments, R⁵ is optionally substituted C₁-C₆ alkyl C₆-C₁₀ aryl (e.g., optionally substituted C₂ alkyl C₆-C₁₀ aryl). In some embodiments, R⁵ is optionally substituted C₁ alkyl C₆-C₁₀ aryl. In some embodiments, R⁵ is optionally substituted C₁ alkyl phenyl. In some embodiments, R⁵ is:

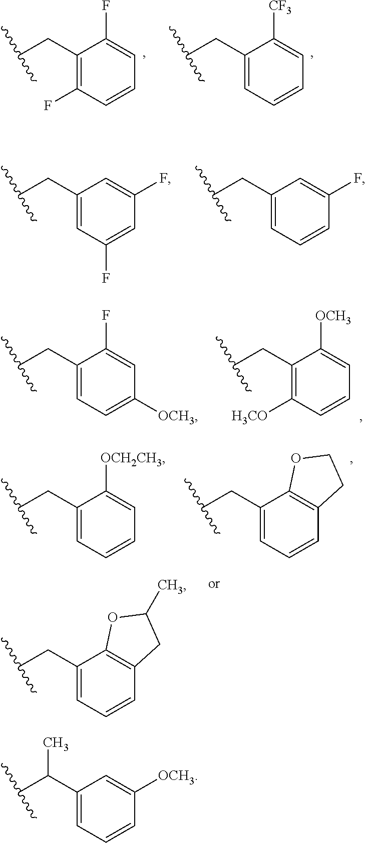

In some embodiments, R⁵ is optionally substituted C₂-C₉ heterocyclyl (e.g.,

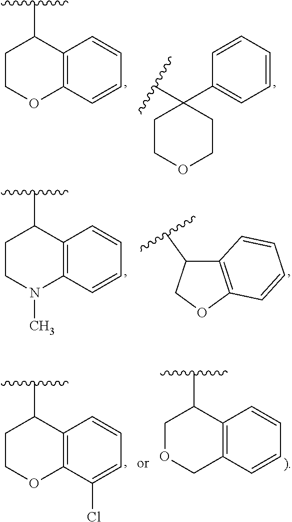

In some embodiments, R⁵ is optionally substituted C₃-C₈ cycloalkyl (e.g.,

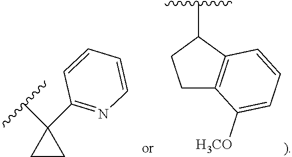 [SMILES: *C1(c2ccccn2)CC1 ; *C1CCc2c(OC)cccc21]

In some embodiments, R⁵ is optionally substituted C₁-C₆ alkyl C₂-C₉ heteroaryl (e.g.,

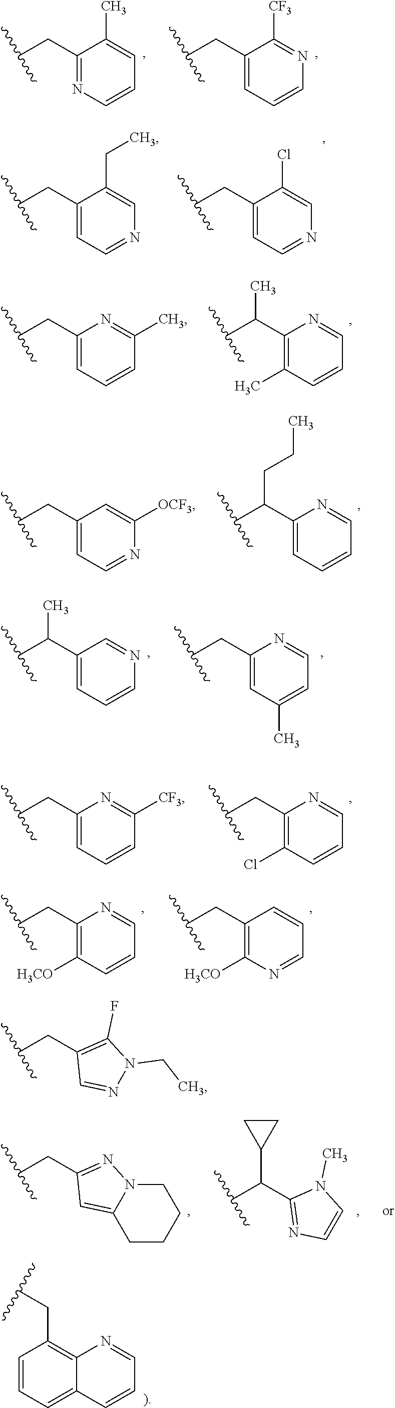

In some embodiments, R⁵ is optionally substituted C₁ alkyl C₂-C₉ heteroaryl. In some embodiments, R⁵ is optionally substituted C₁ alkyl pyridyl.

In some embodiments, the degradation moiety comprises the structure of Formula AA:

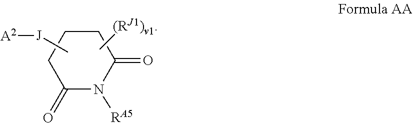

wherein

A² is a bond between the degradation moiety and the linker;

v1 is 0, 1, 2, 3, 4, or 5;

R^{5A} is H, optionally substituted C₁-C₆ alkyl, or optionally substituted C₁-C₆ heteroalkyl;

each R^{J1} is, independently, halogen, optionally substituted C₁-C₆ alkyl, or optionally substituted C₁-C₆ heteroalkyl; and

J is absent, optionally substituted C₃-C₁₀ carbocyclylene, optionally substituted C₆-C₁₀ arylene, optionally substituted C₂-C₉ heterocyclylene, or optionally substituted C₂-C₉ heteroarylene,

or a pharmaceutically acceptable salt thereof.

In some embodiments, v1 is 0. In some embodiments, R^{A5} is H or optionally substituted C₁-C₆ alkyl.

In some embodiments, the structure of Formula AA is

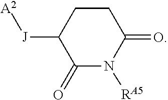

In some embodiments, J is absent.

In some embodiments, the structure of Formula AA is

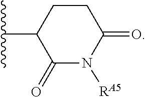

In some embodiments, J is optionally substituted C₂-C₉ heterocyclylene or optionally substituted C₂-C₉ heteroarylene.

In some embodiments, the structure of Formula AA has the structure of

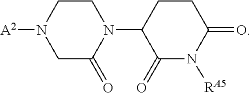

In some embodiments, the structure of Formula AA has the structure of Formula A:

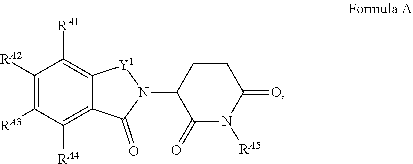

wherein in

Y¹ is

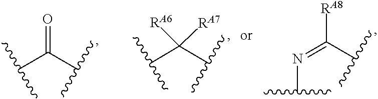

R^{A5} is H, optionally substituted C₁-C₆ alkyl, or optionally substituted C₁-C₆ heteroalkyl;

R^{A6} is H or optionally substituted C₁-C₆ alkyl; and R^{A7} is H or optionally substituted C₁-C₆ alkyl; or R^{A6} and R^{A7}, together with the carbon atom to which each is bound, form an optionally substituted C₃-C₆ carbocyclyl or optionally substituted C₂-C₅ heterocyclyl; or R^{A6} and R^{A7}, together with the carbon atom to which each is bound, form an optionally substituted C₃-C₆ carbocyclyl or optionally substituted C₂-C₅ heterocyclyl;

R^{A8} is H, optionally substituted C₁-C₆ alkyl, or optionally substituted C₁-C₆ heteroalkyl;

each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

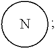

and

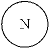

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,

wherein one of R^{A1}, R^{A2}, R^{A3} and R^{A4} is A² or

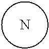

is substituted with A², or a pharmaceutically acceptable salt thereof.

In some embodiments, each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

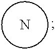

and

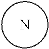

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,

where one of R^{A1}, R^{A2}, R^{A3}, and R^{A4} is A²; or

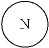

is substituted with A², or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2} and R^{A3}, or R^{A3} and R^{A4}, together with the carbon atoms to which each is attached, combine to form

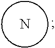

and

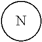

is optionally substituted C₂-C₉ heterocyclyl, which is optionally substituted with A², wherein one of R^{A1}, R^{A2}, R^{A3}, and R^{A4} is A²; or

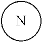

is substituted with A².

In some embodiments, each of R^{A1}, R^{A2}, R^{A3}, and R^{A4} is, independently, H, A², F,

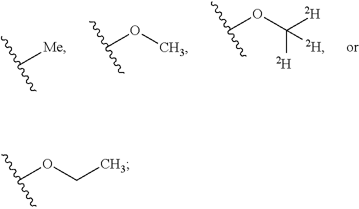

or R^{A1} and R^{A2}, R^{A2} and R^{A3}, or R^{A3} and R^{A4}, together with the carbon atoms to which each is attached, combine to form

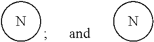

and is optionally substituted C₂-C₉ heterocyclyl, which is optionally substituted with A², wherein one of R^{A1}, R^{A2}, R^{A3}, and R^{A4} is A² or

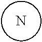

is substituted with A².

In some embodiments, Y¹ is

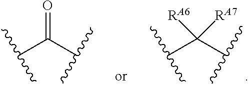

In some embodiments, Y¹ is

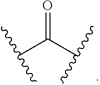 [SMILES: *C(*)=O]

In some embodiments, Y¹ is

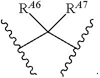

In some embodiments, Y¹ is

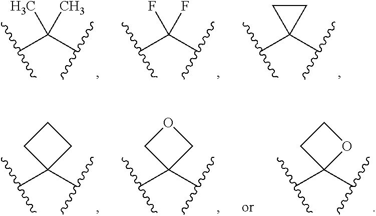

In some embodiments, the structure of Formula A has the structure of Formula A1:

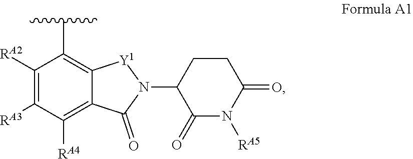

or a pharmaceutically acceptable salt thereof.
In some embodiments, the structure of Formula A has the structure of Formula A2:

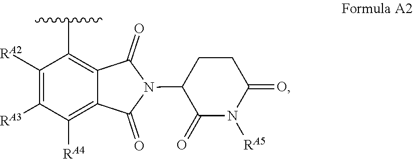

or a pharmaceutically acceptable salt thereof.
In some embodiments, the structure of Formula A has the structure of Formula A3:

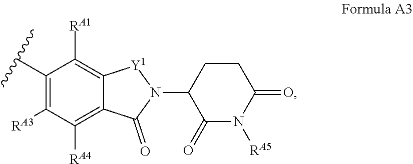

or a pharmaceutically acceptable salt thereof.
In some embodiments, the structure of Formula A has the structure of Formula A4:

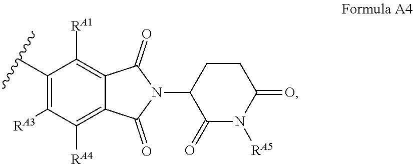

or a pharmaceutically acceptable salt thereof.

In some embodiments, the structure of Formula A has the structure of Formula A7:

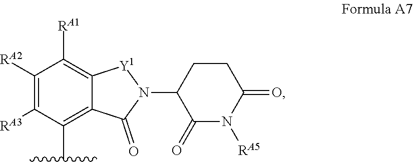

or a pharmaceutically acceptable salt thereof.
In some embodiments, the structure of Formula A has the structure of Formula A9:

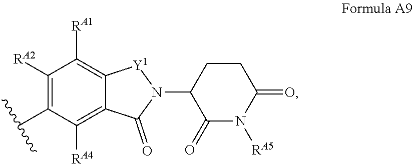

or a pharmaceutically acceptable salt thereof.
In some embodiments, the structure of Formula A has the structure of Formula A10:

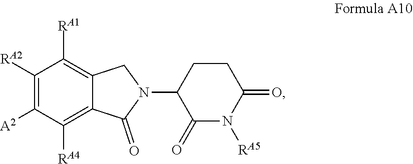

or a pharmaceutically acceptable salt thereof.
In some embodiments, the structure of Formula A is

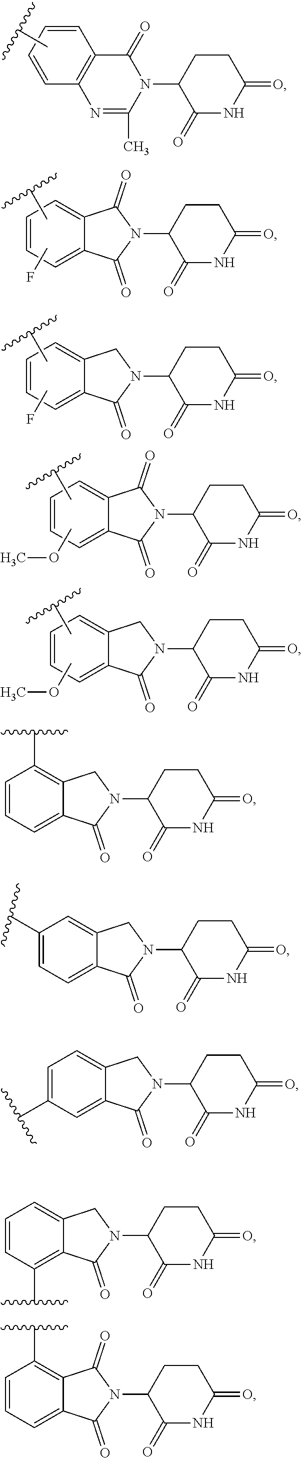

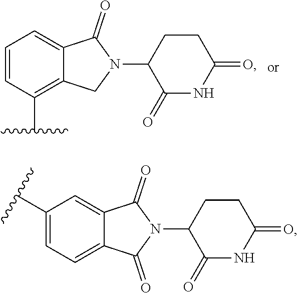 [SMILES: *c1cccc2c1CN(C1CCC(=O)NC1=O)C2=O ; *c1ccc2c(c1)C(=O)N(C1CCC(=O)NC1=O)C2=O]

or derivative or analog thereof.

In some embodiments, the structure of Formula A is

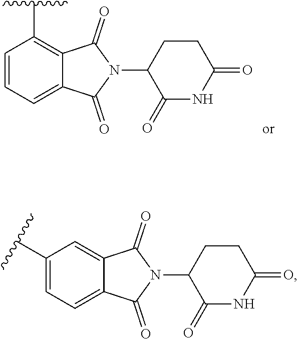 [SMILES: *c1cccc2c1C(=O)N(C1CCC(=O)NC1=O)C2=O ; *c1ccc2c(c1)C(=O)N(C1CCC(=O)NC1=O)C2=O]

or derivative or analog thereof.

In some embodiments,

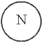

is

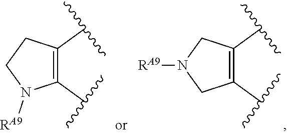

wherein R^{A9} is H, A², optionally substituted C₁-C₆ alkyl, or optionally substituted C₁-C₆ heteroalkyl.

In some embodiments, the structure of Formula A is

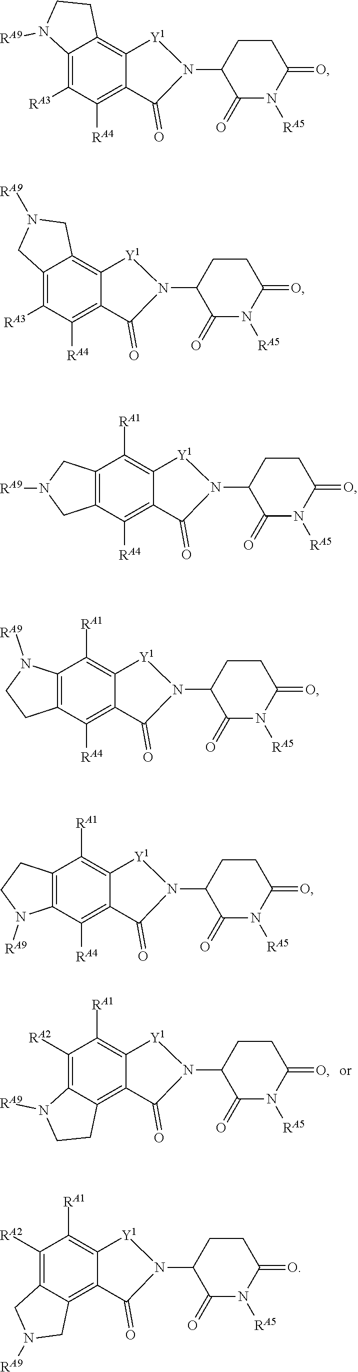

In some embodiments, R^{A9} is H. In some embodiments, R^{A9} is A².

In some embodiments, the structure of Formula A is

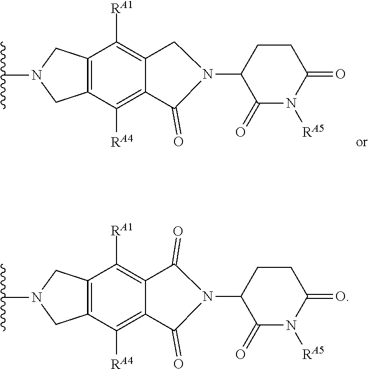

In some embodiments, the structure of Formula AA has the structure of Formula B:

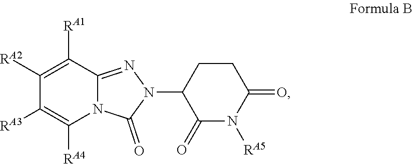

wherein

R^{A5} is H, optionally substituted C₁-C₆ alkyl, or optionally substituted C₁-C₆ heteroalkyl;

each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

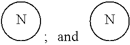

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,

wherein one of R^{A1}, R^{A2}, R^{A3}, and R^{A4} is A² or

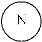

is substituted with A²,

or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2} and R^{A3}, or R^{A3} and R^{A4}, together with the carbon atoms to which each is attached, combine to form

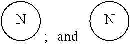

and is optionally substituted C₂-C₉ heterocyclyl, which is optionally substituted with A², wherein one of R^{A1}, R^{A2}, R^{A3}, and R^{A4} is A² or

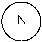

is substituted with A².

In some embodiments, each of R^{A1}, R^{A2}, R^{A3}, and R^{A4} is, independently, H, A², F,

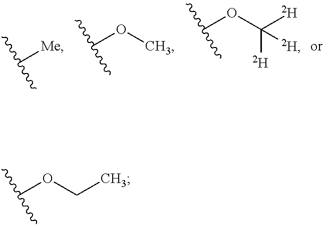

or R^{A1} and R^{A2}, R^{A2} and R^{A3}, or R^{A3} and R^{A4}, together with the carbon atoms to which each is attached, combine to form

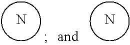

and is optionally substituted C₂-C₉ heterocyclyl, which is optionally substituted with A², wherein one of R^{A1}, R^{A2}, R^{A3}, and R^{A4} is A² or

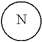

is substituted with A².

In some embodiments, the structure of Formula B has the structure of Formula B1:

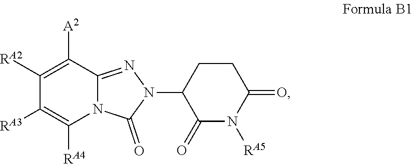

or a pharmaceutically acceptable salt thereof.

In some embodiments, the structure of Formula B has the structure of Formula B2:

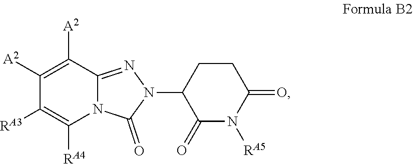

or a pharmaceutically acceptable salt thereof.

In some embodiments, the structure of Formula B has the structure of Formula B3:

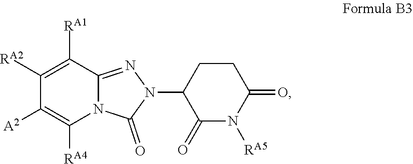

or a pharmaceutically acceptable salt thereof.

In some embodiments, the structure of Formula B has the structure of Formula B4:

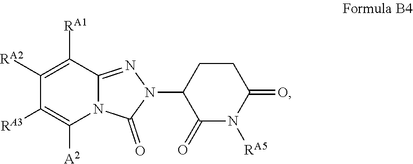

or a pharmaceutically acceptable salt thereof.

In some embodiments, the structure of Formula B is

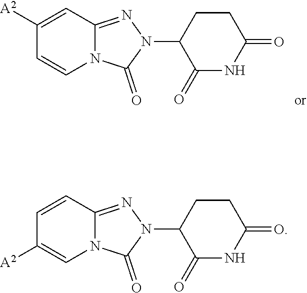

In some embodiments, the structure of Formula B is

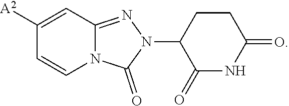

In some embodiments, the structure of Formula B is

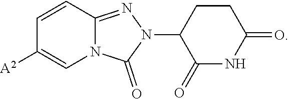

In some embodiments, the degradation moiety comprises the structure of Formula C:

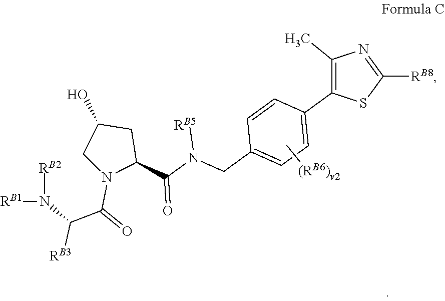

wherein

R^{B1} is H, A², optionally substituted C₁-C₆ alkyl, or optionally substituted C₁-C₆ heteroalkyl;

R^{B2} is H, optionally substituted C₁-C₆ alkyl, or optionally substituted C₁-C₆ heteroalkyl;

R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆alkyl C₆-C₁₀ aryl;

R^{B5} is H, optionally substituted C₁-C₆ alkyl, or optionally substituted C₁-C₆ heteroalkyl;

v2 is 0, 1, 2, 3, or 4;

each R^{B6} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and

each of R^{B7} and R^{B8} is, independently, H, halogen, optionally substituted C₁-C₆ alkyl, or optionally substituted C₆-C₁₀ aryl,

where one of R^{B1} and R^{B3} comprises A²,

or a pharmaceutically acceptable salt thereof.

In some embodiments, the structure of Formula C is

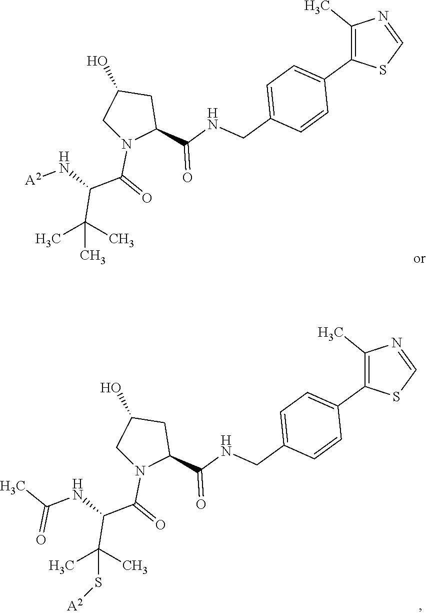

or derivative or analog thereof.

In some embodiments, the structure of Formula C is

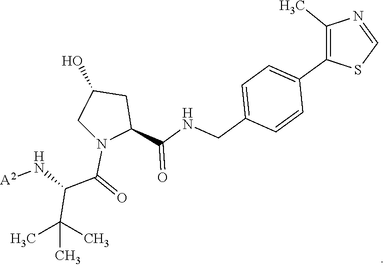

In some embodiments, the degradation moiety comprises the structure of Formula D:

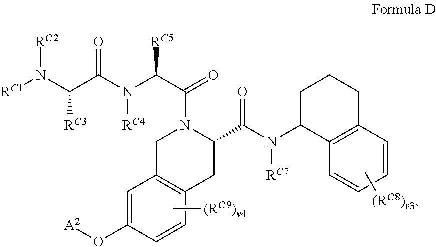

wherein

A² is a bond between B and the linker;

each of R^{C1}, R^{C2}, and R^{C7} is, independently, H, optionally substituted C₁-C₆ alkyl, or optionally substituted C₁-C₆ heteroalkyl;

R^{C3} is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

R^{C5} is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

v3 is 0, 1, 2, 3, or 4;

each R^{C8} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;

v4 is 0, 1, 2, 3, or 4; and

each R^{C9} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,

or a pharmaceutically acceptable salt thereof.

In some embodiments, the degradation moiety comprises the structure of Formula E:

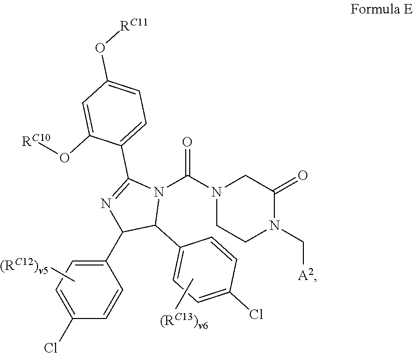

wherein

A² is a bond between B and the linker;

each of R^{C10} and R^{C11} is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;

v5 is 0, 1, 2, 3, or 4;

each R^{C12} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;

v6 is 0, 1, 2, 3, or 4; and

each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,

or a pharmaceutically acceptable salt thereof.

In some embodiments, the structure of Formula E is

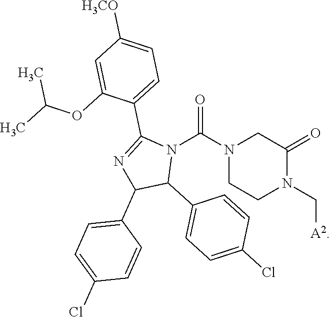

In some embodiments, the degradation moiety comprises the structure of Formula F:

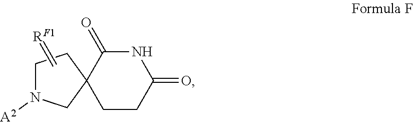

wherein

A² is a bond between the degrader and the linker; and

R^{F1} is absent or O,

or a pharmaceutically acceptable salt thereof.

In some embodiments, R^{F1} is absent.

In some embodiments, R^{F1} is O.

In some embodiments, the structure of Formula F is

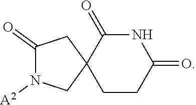

In some embodiments, the degradation moiety comprises the structure Formula G:

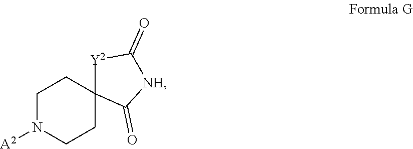

wherein

A² is a bond between the degrader and the linker; and

Y² is CH₂ or NH,

or a pharmaceutically acceptable salt thereof.

In some embodiments, Y² is CH₂.

In some embodiments, Y² is NH.

In some embodiments, the structure of Formula G is

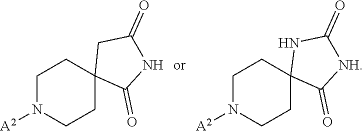

In some embodiments, the linker has the structure of Formula IV:

A¹-(B¹)_{f}—(C₁)_{g}—(B²)ₕ-(D)-(B³)ᵢ—(C²)ⱼ—(B⁴)ₖ-A² Formula IV

wherein

A¹ is a bond between A and the linker;

A² is a bond between the linker and B;

each of B¹, B², B³, and B⁴ is, independently, optionally substituted C₁-C₆ alkyl, optionally substituted C₁-C₆ heteroalkyl, O, S, S(O)₂, or NR^{N};

each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;

each of C¹ and C² is, independently, carbonyl, thiocarbonyl, sulphonyl, or phosphoryl;

each of f, g, h, i, j, and k is, independently, 0 or 1; and

D is optionally substituted C₁₋₁₂ alkyl, optionally substituted C₂₋₁₂ alkenyl, optionally substituted C₂₋₁₂ alkynyl, optionally substituted C₂-C₁₂ polyethylene glycol, or optionally substituted C₁₋₁₂ heteroalkyl, or a chemical bond linking A¹-(B¹)_{f}—(C¹)_{g}—(B²)ₕ— to —(B³)ᵢ—(C²)ⱼ—(B⁴)ₖ-A².

In an aspect, the disclosure features a compound, or a pharmaceutically acceptable salt thereof, having the structure of any one of compounds 1-65 in Table 1 or compounds D1-D51 in Table 2.

In some embodiments, the compound is any one of the compounds in Table 1. In some embodiments, the compound is any one of the compounds in Table 1, or a pharmaceutically acceptable salt thereof.

In some embodiments, the compound is any one of the compounds in Table 2. In some embodiments, the compound is any one of the compounds in Table 2, or a pharmaceutically acceptable salt thereof.

In some embodiments, the compound is any one of the compounds in Table 7. In some embodiments, the compound is any one of the compounds in Table 7, or a pharmaceutically acceptable salt thereof.

In addition to free base and pharmaceutically acceptable salt forms of the compounds described herein, the present disclosure provides stereoisomers, tautomers, isotopically labeled derivatives, solvates, hydrates, polymorphs, co-crystals, and prodrugs of the compounds described herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

TABLE 1
Compounds of the Disclosure

| # | Structure |
|---|---|
| 1 | 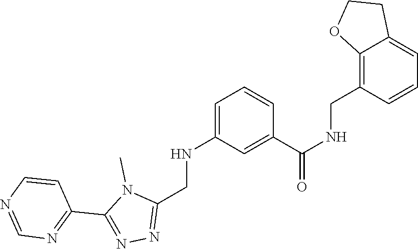 [SMILES: Cn1c(CNc2cccc(C(=O)NCc3cccc4c3OCC4)c2)nnc1-c1ccncn1] |
| 2 | 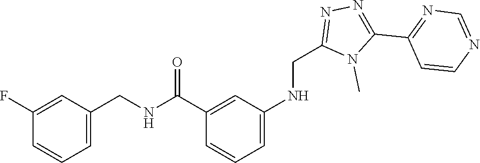 [SMILES: Cn1c(CNc2cccc(C(=O)NCc3cccc(F)c3)c2)nnc1-c1ccncn1] |
| 3 | 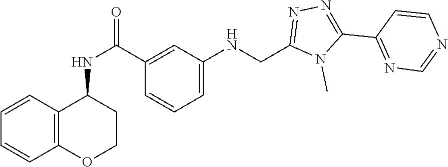 [SMILES: Cn1c(CNc2cccc(C(=O)N[C@H]3CCOc4ccccc43)c2)nnc1-c1ccncn1] |
| 4 | 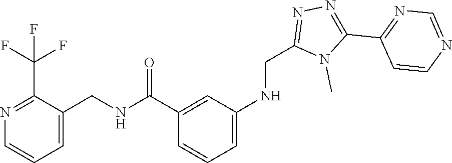 [SMILES: Cn1c(CNc2cccc(C(=O)NCc3cccnc3C(F)(F)F)c2)nnc1-c1ccncn1] |
| 5 | 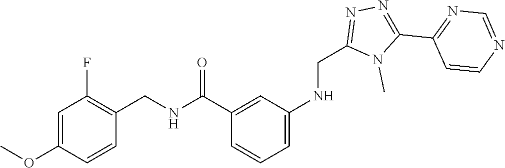 [SMILES: COc1ccc(CNC(=O)c2cccc(NCc3nnc(-c4ccncn4)n3C)c2)c(F)c1] |
| 6 | 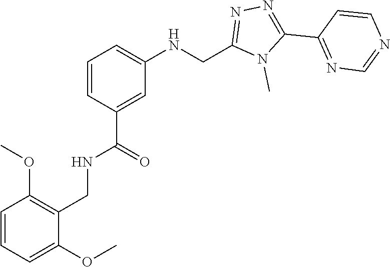 [SMILES: COc1cccc(OC)c1CNC(=O)c1cccc(NCc2nnc(-c3ccncn3)n2C)c1] |
| 7 | 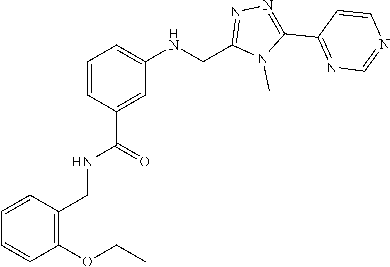 [SMILES: CCOc1ccccc1CNC(=O)c1cccc(NCc2nnc(-c3ccncn3)n2C)c1] |
| 8 | 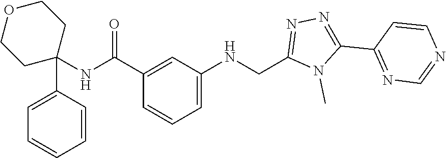 [SMILES: Cn1c(CNc2cccc(C(=O)NC3(c4ccccc4)CCOCC3)c2)nnc1-c1ccncn1] |
| 9 | 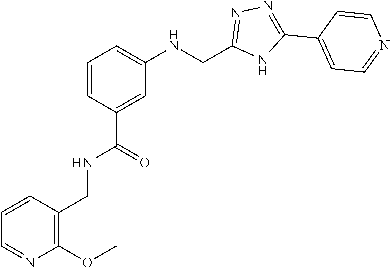 [SMILES: COc1ncccc1CNC(=O)c1cccc(NCc2nnc(-c3ccncc3)[nH]2)c1] |
| 10 | 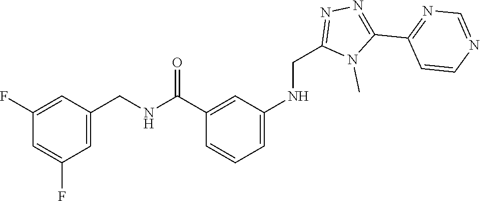 [SMILES: Cn1c(CNc2cccc(C(=O)NCc3cc(F)cc(F)c3)c2)nnc1-c1ccncn1] |
| 11 | 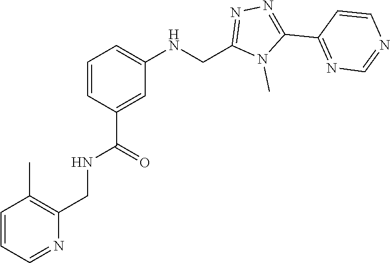 [SMILES: Cc1cccnc1CNC(=O)c1cccc(NCc2nnc(-c3ccncn3)n2C)c1] |
| 12 | 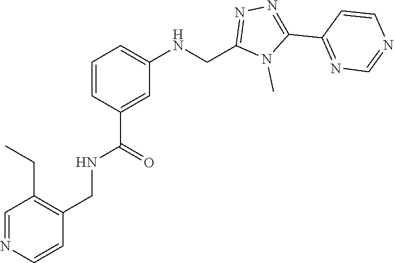 [SMILES: CCc1cnccc1CNC(=O)c1cccc(NCc2nnc(-c3ccncn3)n2C)c1] |
| 13 | 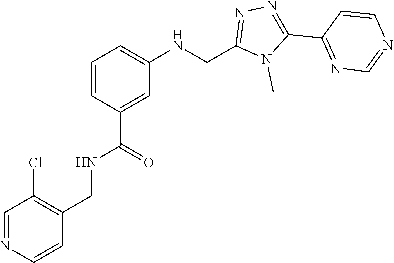 [SMILES: Cn1c(CNc2cccc(C(=O)NCc3ccncc3Cl)c2)nnc1-c1ccncn1] |
| 14 | 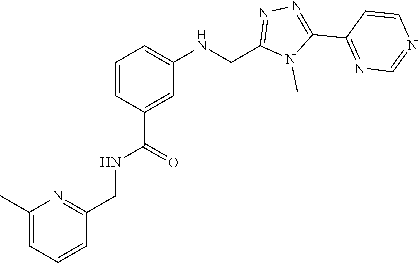 [SMILES: Cc1cccc(CNC(=O)c2cccc(NCc3nnc(-c4ccncn4)n3C)c2)n1] |
| 15 | 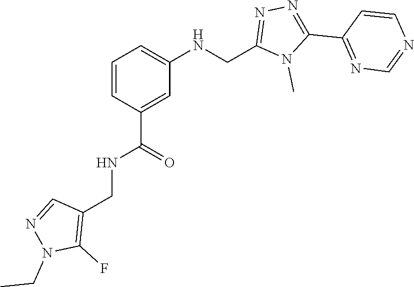 [SMILES: CCn1ncc(CNC(=O)c2cccc(NCc3nnc(-c4ccncn4)n3C)c2)c1F] |
| 16 | 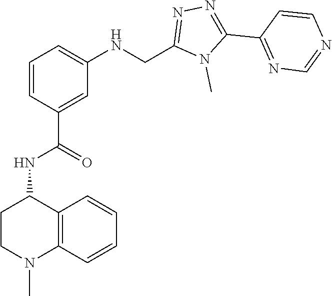 |
| 17 | 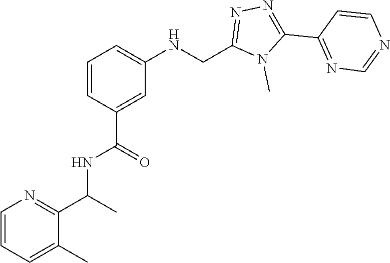 [SMILES: Cc1cccnc1C(C)NC(=O)c1cccc(NCc2nnc(-c3ccncn3)n2C)c1] |
| 18 | 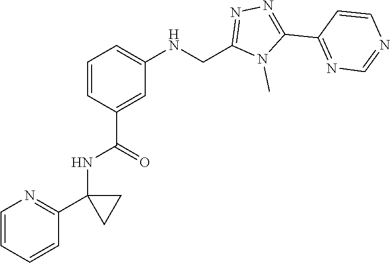 [SMILES: Cn1c(CNc2cccc(C(=O)NC3(c4ccccn4)CC3)c2)nnc1-c1ccncn1] |
| 19 | 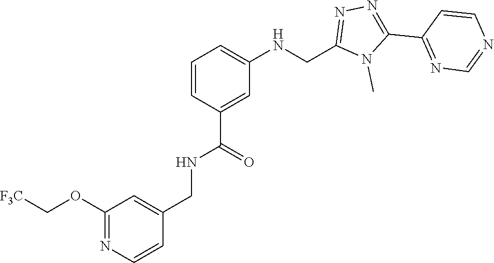 [SMILES: Cn1c(CNc2cccc(C(=O)NCc3ccnc(OCC(F)(F)F)c3)c2)nnc1-c1ccncn1] |
| 20 | 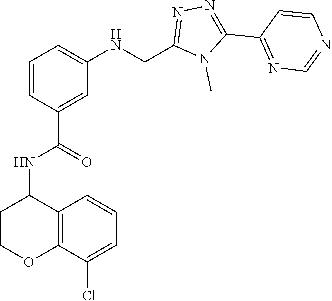 [SMILES: Cn1c(CNc2cccc(C(=O)NC3CCOc4c(Cl)cccc43)c2)nnc1-c1ccncn1] |
| 21 | 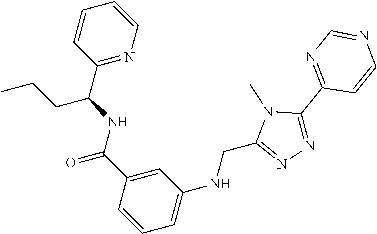 [SMILES: CCC[C@H](NC(=O)c1cccc(NCc2nnc(-c3ccncn3)n2C)c1)c1ccccn1] |
| 22 | 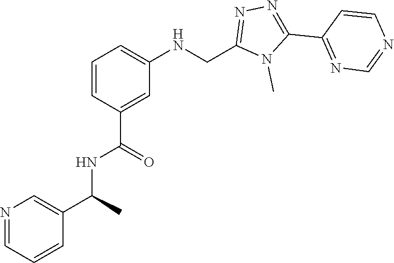 [SMILES: C[C@H](NC(=O)c1cccc(NCc2nnc(-c3ccncn3)n2C)c1)c1cccnc1] |
| 23 | 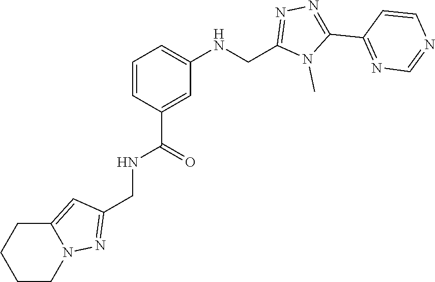 [SMILES: Cn1c(CNc2cccc(C(=O)NCc3cc4n(n3)CCCC4)c2)nnc1-c1ccncn1] |
| 24 | 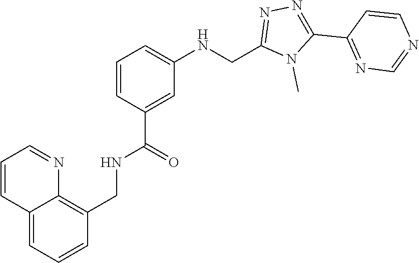 [SMILES: Cn1c(CNc2cccc(C(=O)NCc3cccc4cccnc34)c2)nnc1-c1ccncn1] |
| 25 | 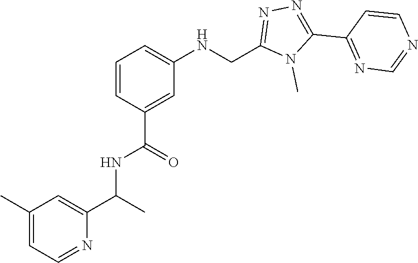 [SMILES: Cc1ccnc(C(C)NC(=O)c2cccc(NCc3nnc(-c4ccncn4)n3C)c2)c1] |
| 26 | 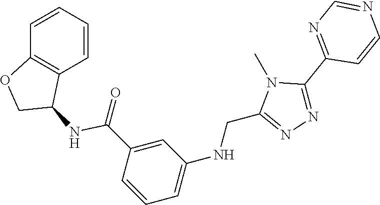 [SMILES: Cn1c(CNc2cccc(C(=O)N[C@H]3COc4ccccc43)c2)nnc1-c1ccncn1] |
| 27 | 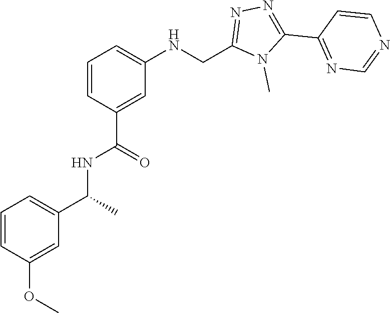 [SMILES: COc1cccc([C@@H](C)NC(=O)c2cccc(NCc3nnc(-c4ccncn4)n3C)c2)c1] |
| 28 | 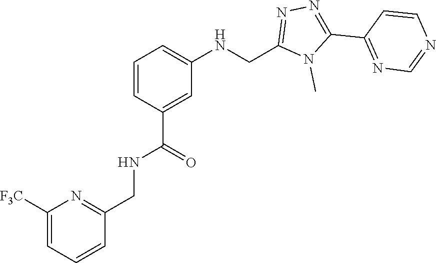 [SMILES: Cn1c(CNc2cccc(C(=O)NCc3cccc(C(F)(F)F)n3)c2)nnc1-c1ccncn1] |
| 29 | 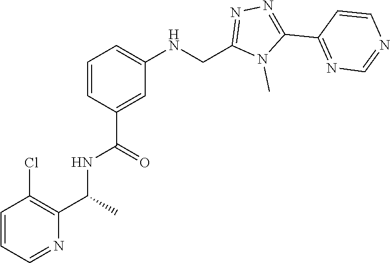 [SMILES: C[C@@H](NC(=O)c1cccc(NCc2nnc(-c3ccncn3)n2C)c1)c1ncccc1Cl] |
| 30 | 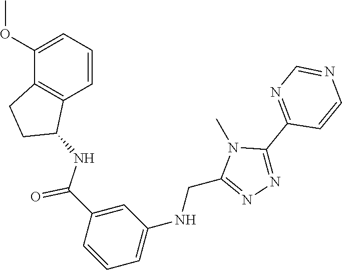 [SMILES: COc1cccc2c1CC[C@H]2NC(=O)c1cccc(NCc2nnc(-c3ccncn3)n2C)c1] |
| 31 | 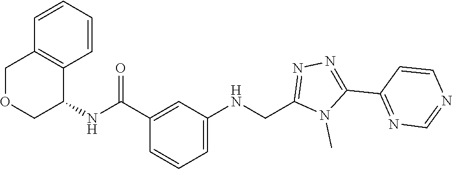 [SMILES: Cn1c(CNc2cccc(C(=O)N[C@@H]3COCc4ccccc43)c2)nnc1-c1ccncn1] |
| 32 | 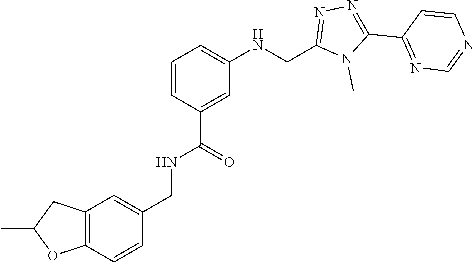 [SMILES: CC1Cc2cc(CNC(=O)c3cccc(NCc4nnc(-c5ccncn5)n4C)c3)ccc2O1] |
| 33 | 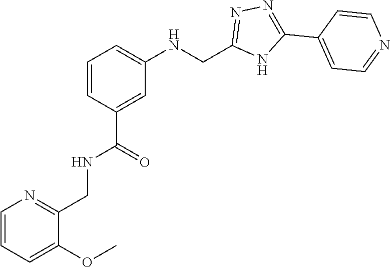 [SMILES: COc1cccnc1CNC(=O)c1cccc(NCc2nnc(-c3ccncc3)[nH]2)c1] |
| 34 | 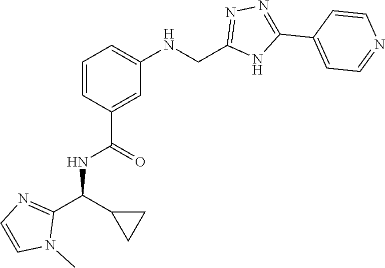 [SMILES: Cn1ccnc1[C@@H](NC(=O)c1cccc(NCc2nnc(-c3ccncc3)[nH]2)c1)C1CC1] |
| 35 | 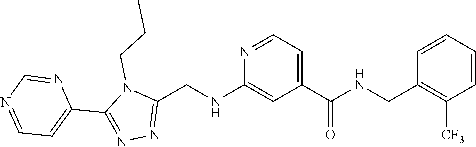 [SMILES: CCCn1c(CNc2cc(C(=O)NCc3ccccc3C(F)(F)F)ccn2)nnc1-c1ccncn1] |
| 36 | 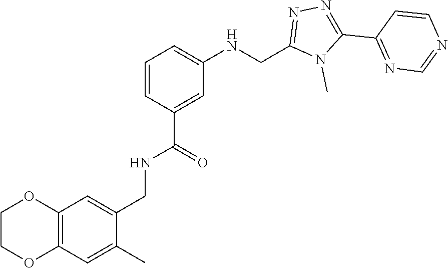 [SMILES: Cc1cc2c(cc1CNC(=O)c1cccc(NCc3nnc(-c4ccncn4)n3C)c1)OCCO2] |
| 37 | 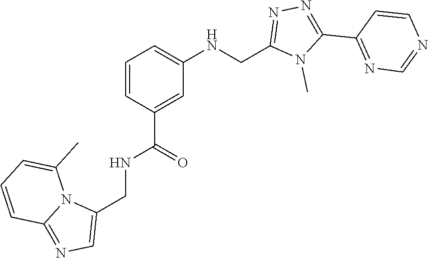 [SMILES: Cc1cccc2ncc(CNC(=O)c3cccc(NCc4nnc(-c5ccncn5)n4C)c3)n12] |
| 38 | 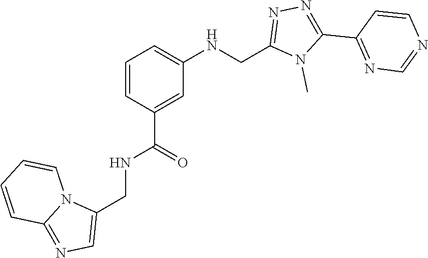 [SMILES: Cn1c(CNc2cccc(C(=O)NCc3cnc4ccccn34)c2)nnc1-c1ccncn1] |
| 39 | 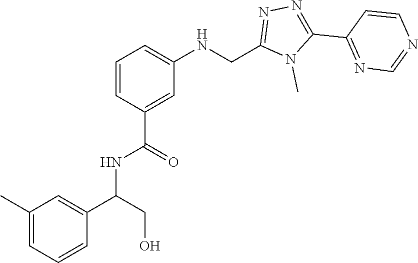 [SMILES: Cc1cccc(C(CO)NC(=O)c2cccc(NCc3nnc(-c4ccncn4)n3C)c2)c1] |
| 40 | 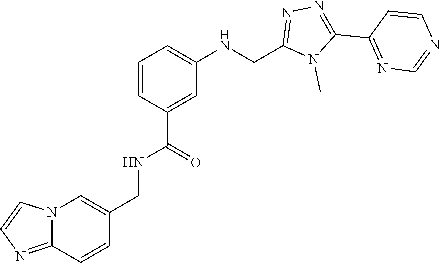 [SMILES: Cn1c(CNc2cccc(C(=O)NCc3ccc4nccn4c3)c2)nnc1-c1ccncn1] |
| 41 | 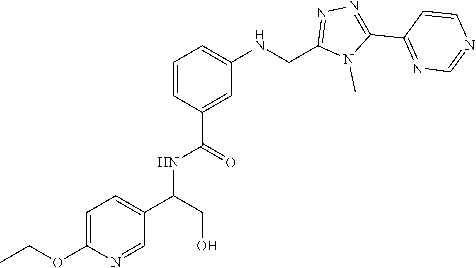 [SMILES: CCOc1ccc(C(CO)NC(=O)c2cccc(NCc3nnc(-c4ccncn4)n3C)c2)cn1] |
| 42 | 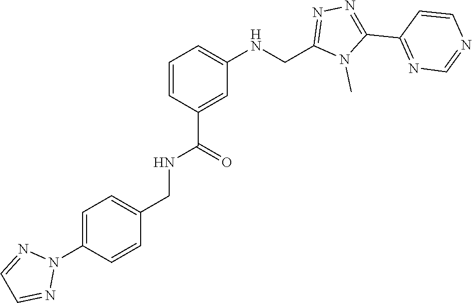 [SMILES: Cn1c(CNc2cccc(C(=O)NCc3ccc(-n4nccn4)cc3)c2)nnc1-c1ccncn1] |
| 43 | 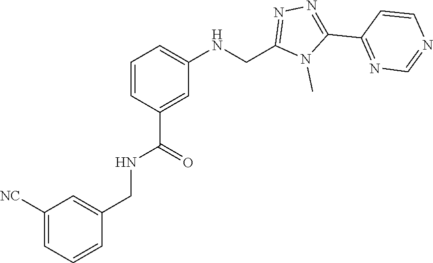 [SMILES: Cn1c(CNc2cccc(C(=O)NCc3cccc(C#N)c3)c2)nnc1-c1ccncn1] |
| 44 | 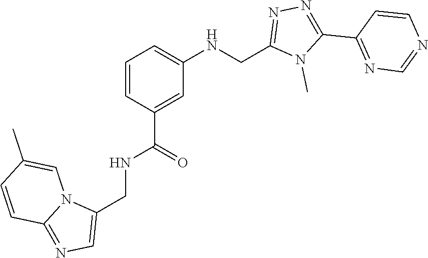 [SMILES: Cc1ccc2ncc(CNC(=O)c3cccc(NCc4nnc(-c5ccncn5)n4C)c3)n2c1] |
| 45 | 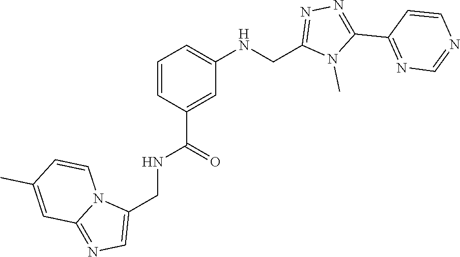 [SMILES: Cc1ccn2c(CNC(=O)c3cccc(NCc4nnc(-c5ccncn5)n4C)c3)cnc2c1] |
| 46 | 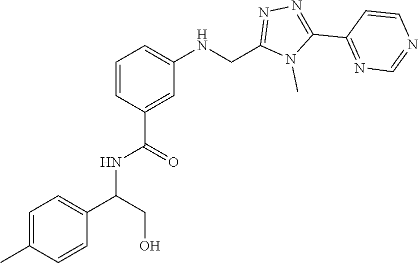 [SMILES: Cc1ccc(C(CO)NC(=O)c2cccc(NCc3nnc(-c4ccncn4)n3C)c2)cc1] |
| 47 | 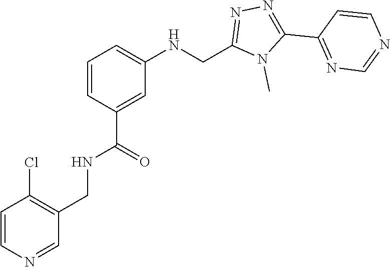 [SMILES: Cn1c(CNc2cccc(C(=O)NCc3cnccc3Cl)c2)nnc1-c1ccncn1] |
| 48 | 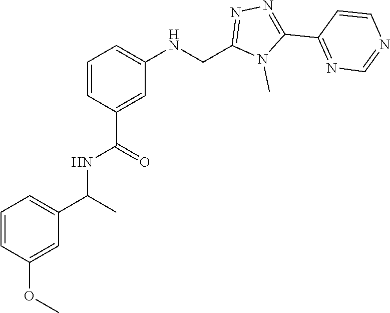 [SMILES: COc1cccc(C(C)NC(=O)c2cccc(NCc3nnc(-c4ccncn4)n3C)c2)c1] |
| 49 | 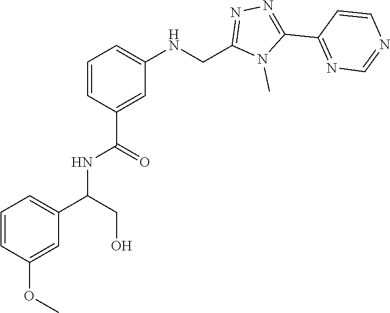 [SMILES: COc1cccc(C(CO)NC(=O)c2cccc(NCc3nnc(-c4ccncn4)n3C)c2)c1] |
| 50 | 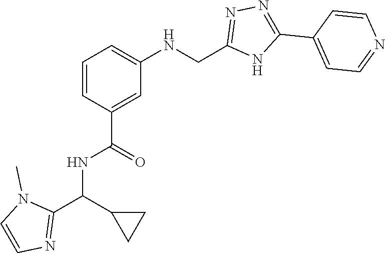 [SMILES: Cn1ccnc1C(NC(=O)c1cccc(NCc2nnc(-c3ccncc3)[nH]2)c1)C1CC1] |
| 51 | 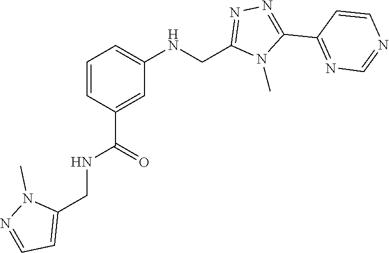 [SMILES: Cn1nccc1CNC(=O)c1cccc(NCc2nnc(-c3ccncn3)n2C)c1] |
| 52 | 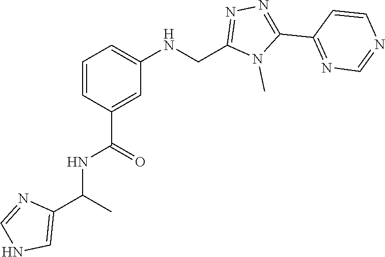 [SMILES: CC(NC(=O)c1cccc(NCc2nnc(-c3ccncn3)n2C)c1)c1c[nH]cn1] |
| 53 | 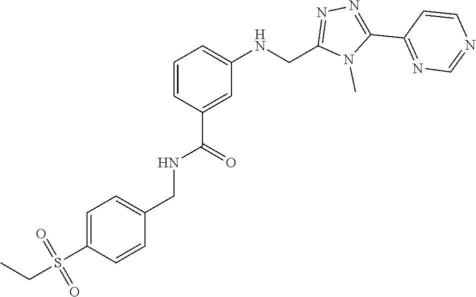 [SMILES: CCS(=O)(=O)c1ccc(CNC(=O)c2cccc(NCc3nnc(-c4ccncn4)n3C)c2)cc1] |
| 54 | 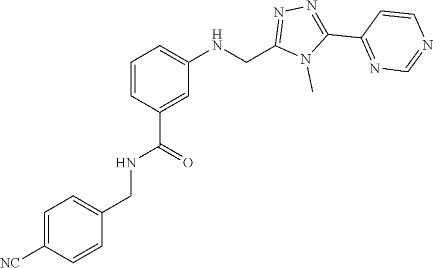 [SMILES: Cn1c(CNc2cccc(C(=O)NCc3ccc(C#N)cc3)c2)nnc1-c1ccncn1] |
| 55 | 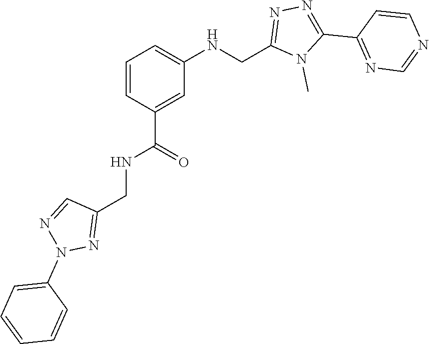 [SMILES: Cn1c(CNc2cccc(C(=O)NCc3cnn(-c4ccccc4)n3)c2)nnc1-c1ccncn1] |
| 56 | 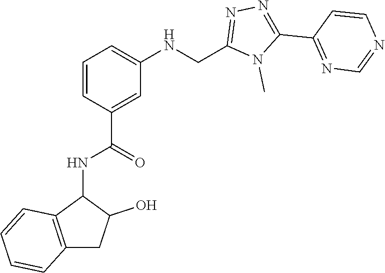 [SMILES: Cn1c(CNc2cccc(C(=O)NC3c4ccccc4CC3O)c2)nnc1-c1ccncn1] |
| 57 | 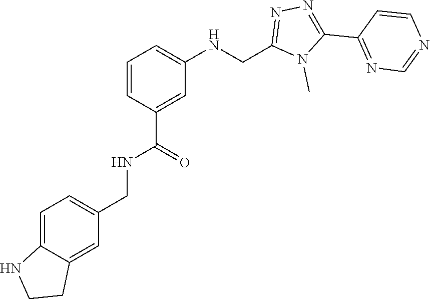 [SMILES: Cn1c(CNc2cccc(C(=O)NCc3ccc4c(c3)CCN4)c2)nnc1-c1ccncn1] |
| 58 | 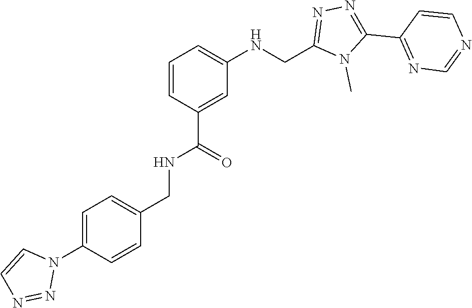 [SMILES: Cn1c(CNc2cccc(C(=O)NCc3ccc(-n4ccnn4)cc3)c2)nnc1-c1ccncn1] |
| 59 | 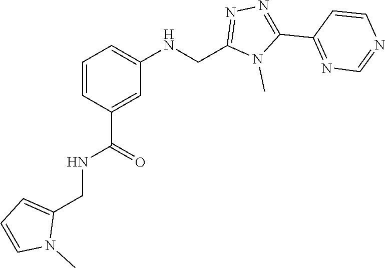 [SMILES: Cn1cccc1CNC(=O)c1cccc(NCc2nnc(-c3ccncn3)n2C)c1] |
| 60 | 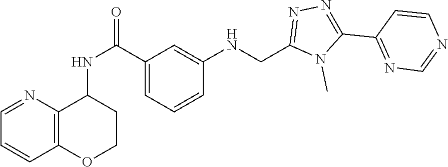 [SMILES: Cn1c(CNc2cccc(C(=O)NC3CCOc4cccnc43)c2)nnc1-c1ccncn1] |
| 61 | 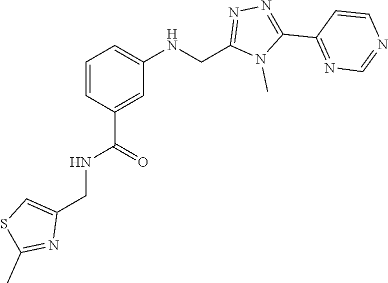 [SMILES: Cc1nc(CNC(=O)c2cccc(NCc3nnc(-c4ccncn4)n3C)c2)cs1] |
| 62 | 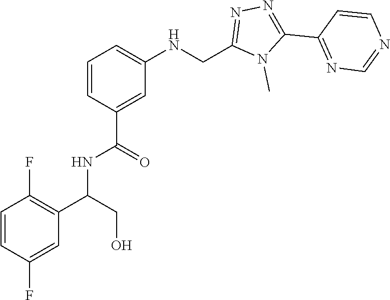 [SMILES: Cn1c(CNc2cccc(C(=O)NC(CO)c3cc(F)ccc3F)c2)nnc1-c1ccncn1] |
| 63 | 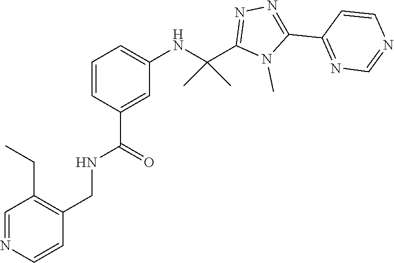 [SMILES: CCc1cnccc1CNC(=O)c1cccc(NC(C)(C)c2nnc(-c3ccncn3)n2C)c1] |
| 64 | 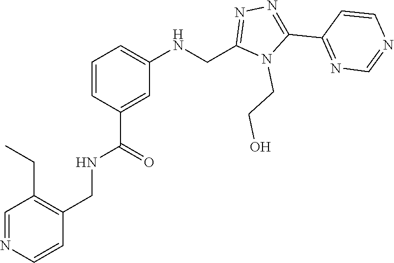 [SMILES: CCc1cnccc1CNC(=O)c1cccc(NCc2nnc(-c3ccncn3)n2CCO)c1] |
| 65 | 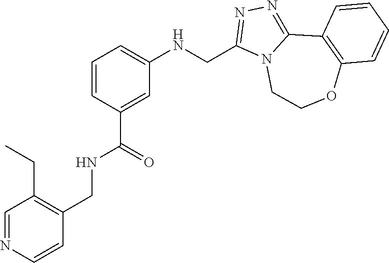 [SMILES: CCc1cnccc1CNC(=O)c1cccc(NCc2nnc3n2CCOc2ccccc2-3)c1] |

TABLE 2
Compounds of the Disclosure

| # | Structure |
|---|---|
| D1 | 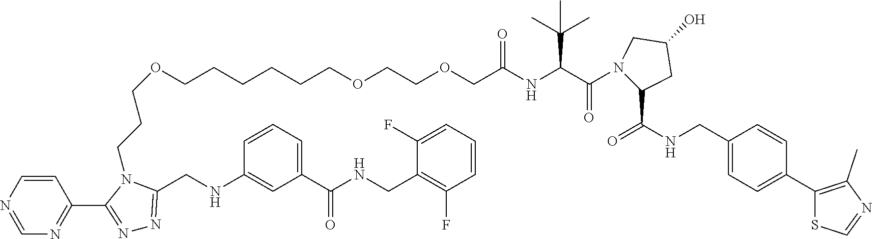 |
| D2 | 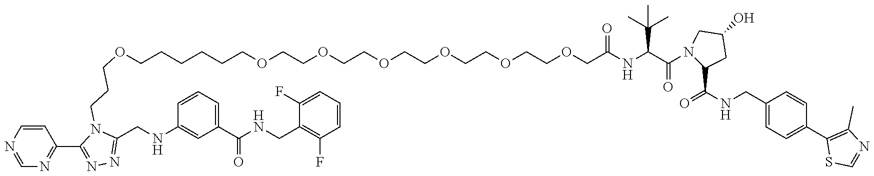 |
| D3 | 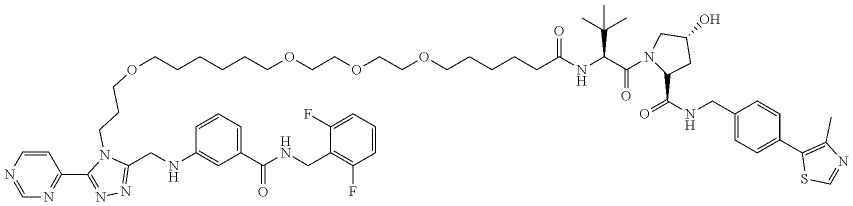 |
| D4 | 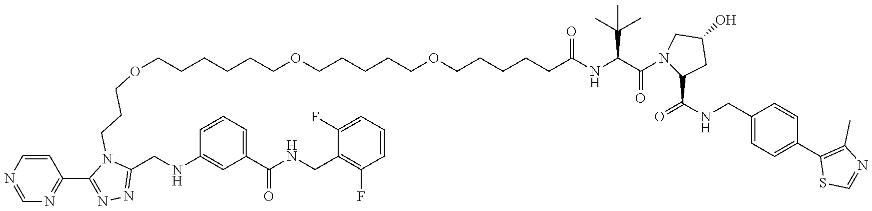 |
| D5 | 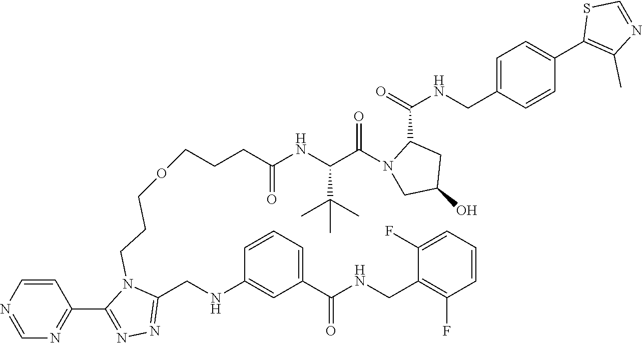 |
| D6 | 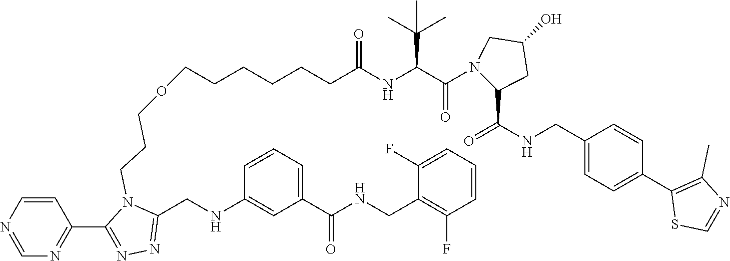 |
| D7 | 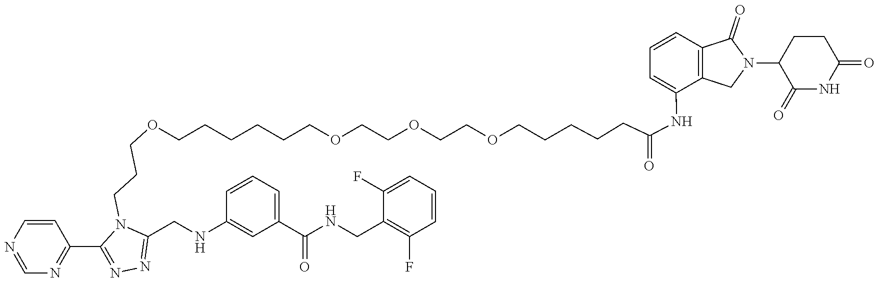 [SMILES: O=C1CCC(N2Cc3c(NC(=O)CCCCCOCCOCCOCCCCCCOCCCn4c(CNc5cccc(C(=O)NCc6c(F)cccc6F)c5)nnc4-c4ccncn4)cccc3C2=O)C(=O)N1] |
| D8 | 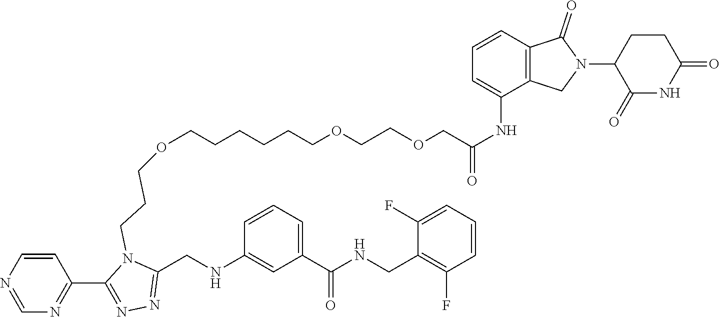 [SMILES: O=C1CCC(N2Cc3c(NC(=O)COCCOCCCCCCOCCCn4c(CNc5cccc(C(=O)NCc6c(F)cccc6F)c5)nnc4-c4ccncn4)cccc3C2=O)C(=O)N1] |
| D9 | 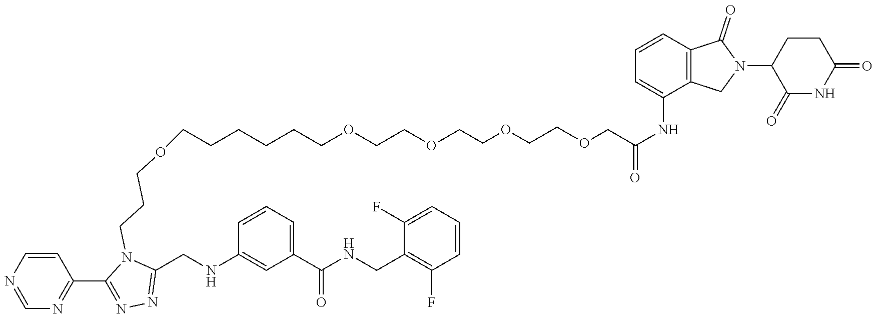 [SMILES: O=C1CCC(N2Cc3c(NC(=O)COCCOCCOCCOCCCCCCOCCCn4c(CNc5cccc(C(=O)NCc6c(F)cccc6F)c5)nnc4-c4ccncn4)cccc3C2=O)C(=O)N1] |
| D10 | 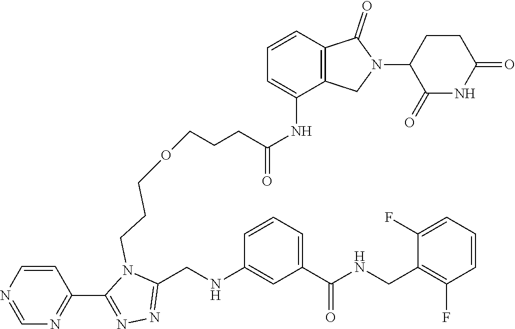 [SMILES: O=C1CCC(N2Cc3c(NC(=O)CCCOCCCn4c(CNc5cccc(C(=O)NCc6c(F)cccc6F)c5)nnc4-c4ccncn4)cccc3C2=O)C(=O)N1] |
| D11 | 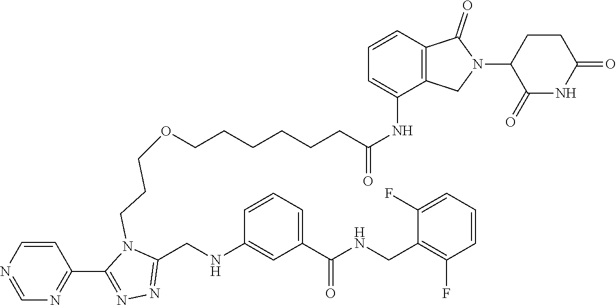 [SMILES: O=C1CCC(N2Cc3c(NC(=O)CCCCCCOCCCn4c(CNc5cccc(C(=O)NCc6c(F)cccc6F)c5)nnc4-c4ccncn4)cccc3C2=O)C(=O)N1] |
| D12 | 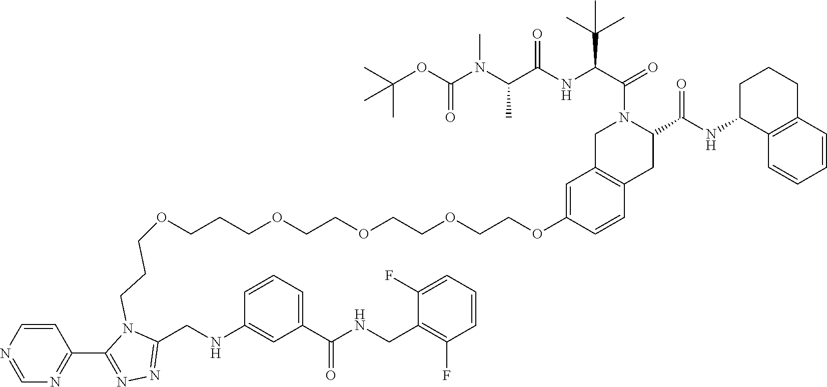 |
| D13 | 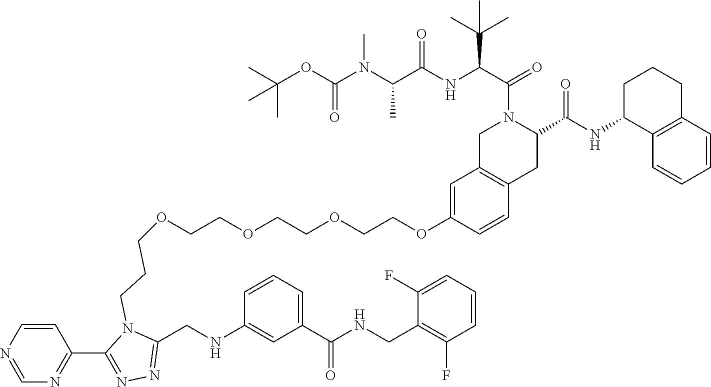 |
| D14 | 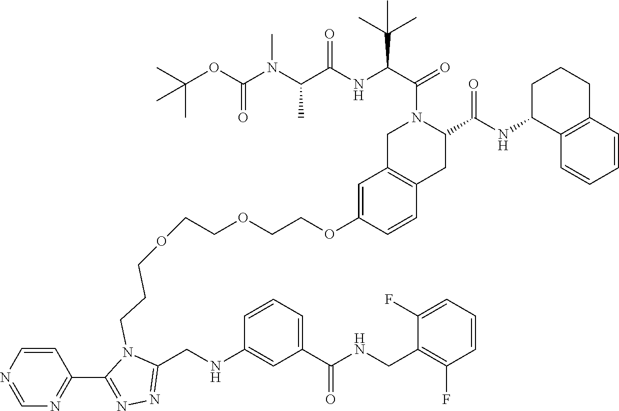 |
| D15 | 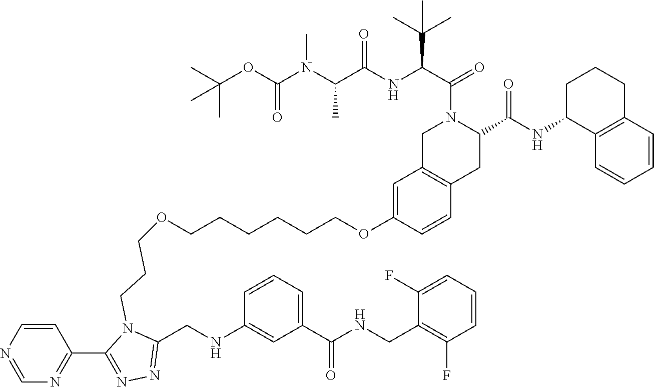 |
| D16 | 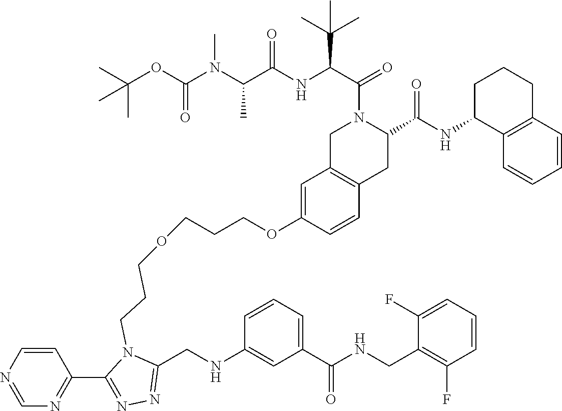 |
| D17 | 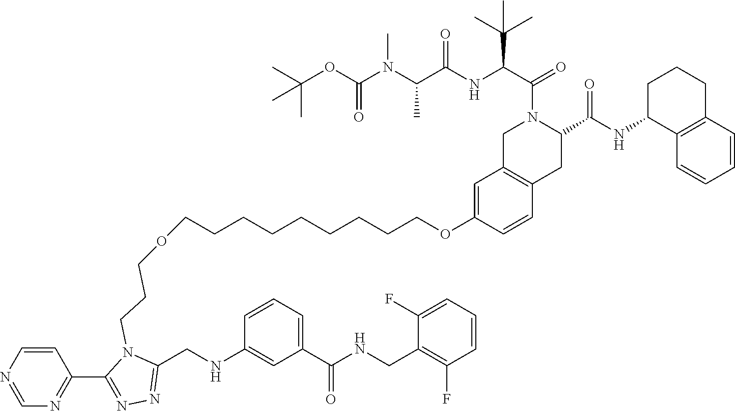 |
| D18 | 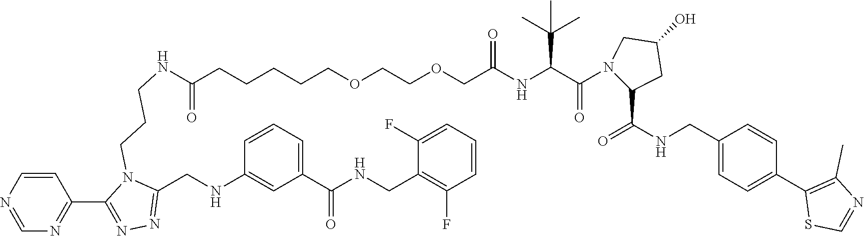 |
| D19 | 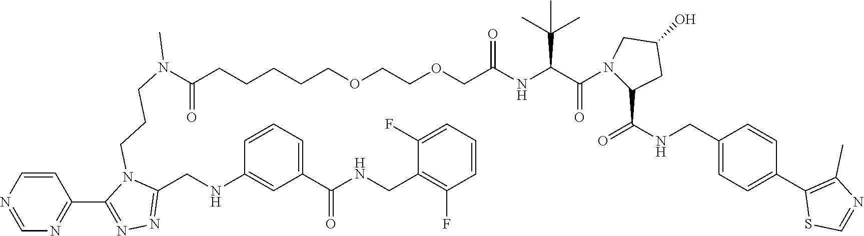 |
| D20 | 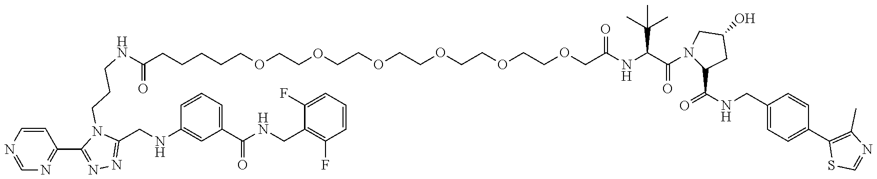 |
| D21 | 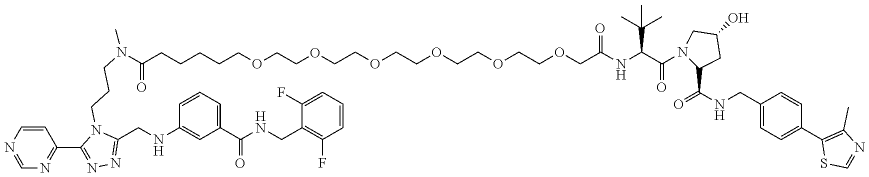 |
| D22 | 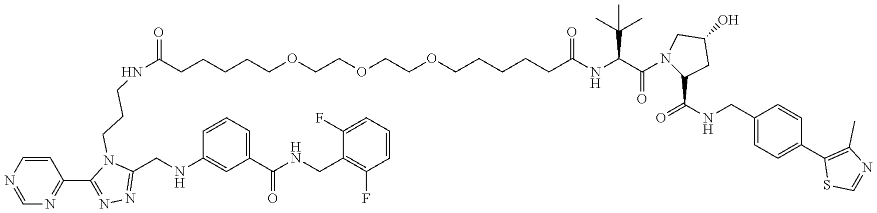 |
| D23 | 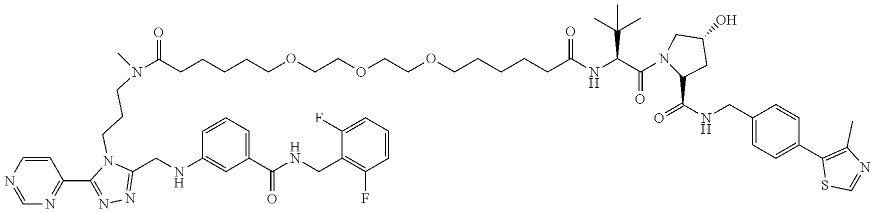 |
| D24 | 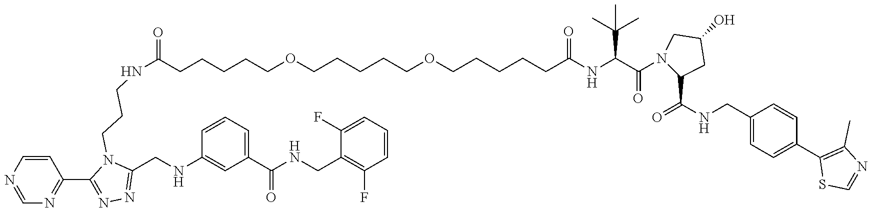 |
| D25 | 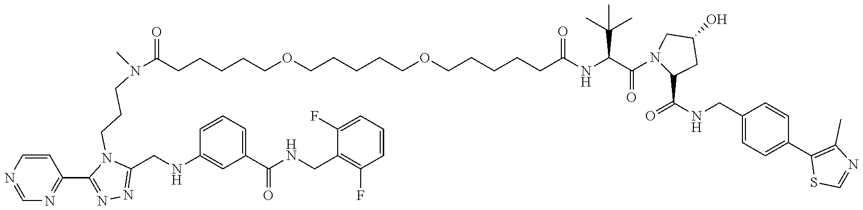 |
| D26 | 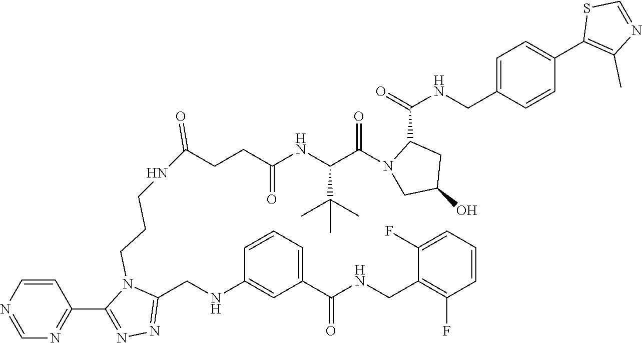 |
| D27 | 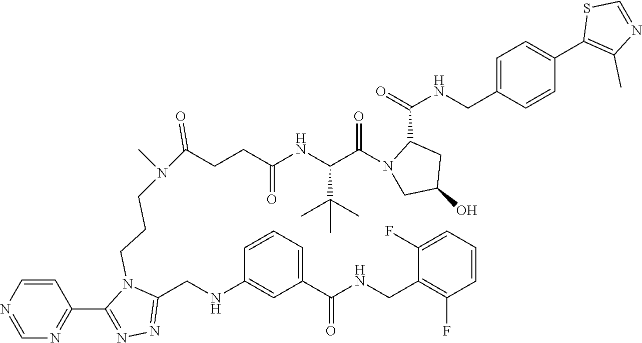 |
| D28 | 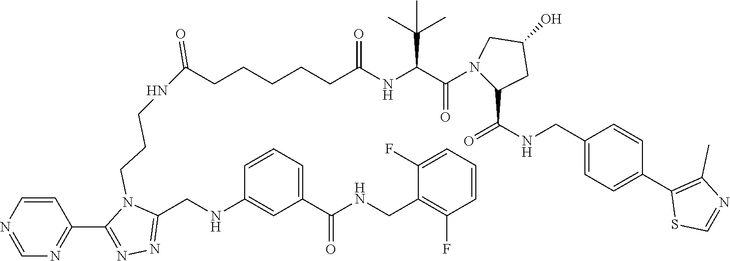 |
| D29 | 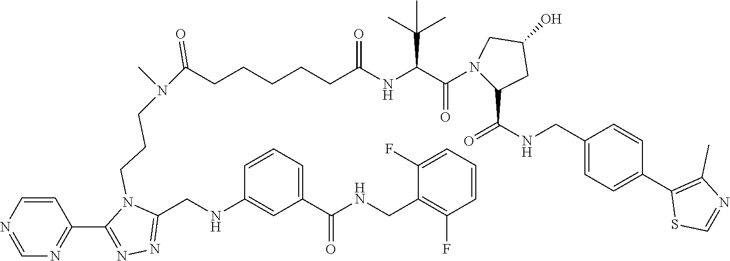 |
| D30 | 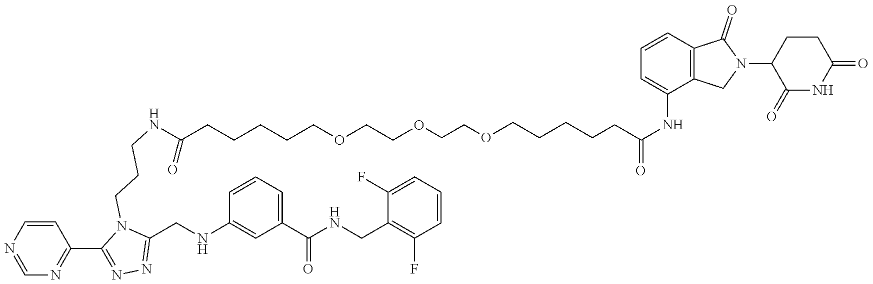 [SMILES: O=C(CCCCCOCCOCCOCCCCCC(=O)Nc1cccc2c1CN(C1CCC(=O)NC1=O)C2=O)NCCCn1c(CNc2cccc(C(=O)NCc3c(F)cccc3F)c2)nnc1-c1ccncn1] |
| D31 | 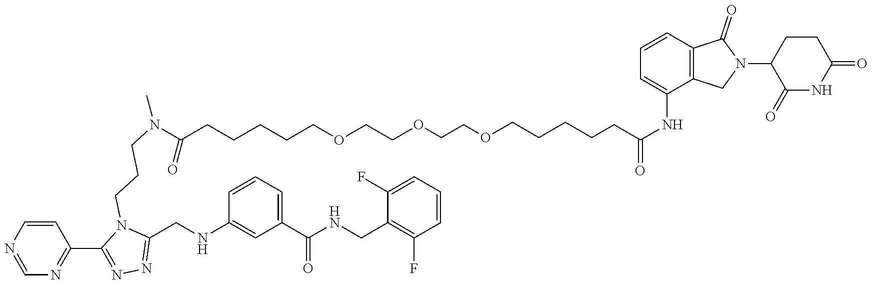 [SMILES: CN(CCCn1c(CNc2cccc(C(=O)NCc3c(F)cccc3F)c2)nnc1-c1ccncn1)C(=O)CCCCCOCCOCCOCCCCCC(=O)Nc1cccc2c1CN(C1CCC(=O)NC1=O)C2=O] |
| D32 | 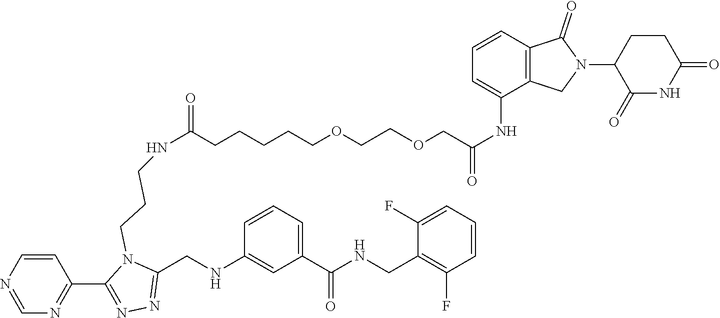 [SMILES: O=C(CCCCCOCCOCC(=O)Nc1cccc2c1CN(C1CCC(=O)NC1=O)C2=O)NCCCn1c(CNc2cccc(C(=O)NCc3c(F)cccc3F)c2)nnc1-c1ccncn1] |
| D33 | 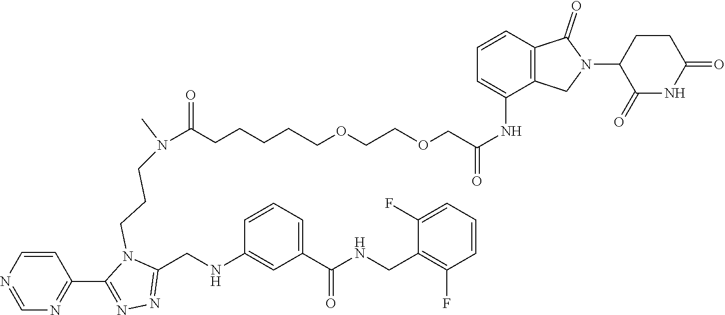 [SMILES: CN(CCCn1c(CNc2cccc(C(=O)NCc3c(F)cccc3F)c2)nnc1-c1ccncn1)C(=O)CCCCCOCCOCC(=O)Nc1cccc2c1CN(C1CCC(=O)NC1=O)C2=O] |
| D34 | 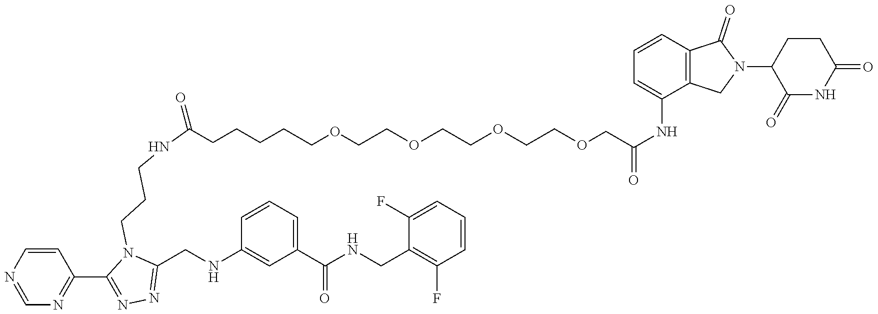 [SMILES: O=C(CCCCCOCCOCCOCCOCC(=O)Nc1cccc2c1CN(C1CCC(=O)NC1=O)C2=O)NCCCn1c(CNc2cccc(C(=O)NCc3c(F)cccc3F)c2)nnc1-c1ccncn1] |
| D35 | 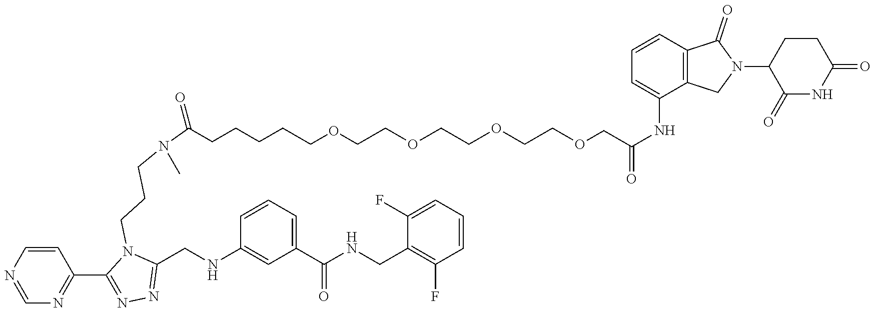 [SMILES: CN(CCCn1c(CNc2cccc(C(=O)NCc3c(F)cccc3F)c2)nnc1-c1ccncn1)C(=O)CCCCCOCCOCCOCCOCC(=O)Nc1cccc2c1CN(C1CCC(=O)NC1=O)C2=O] |
| D36 | 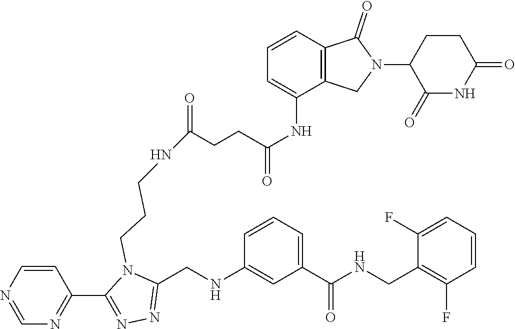 [SMILES: O=C(CCC(=O)Nc1cccc2c1CN(C1CCC(=O)NC1=O)C2=O)NCCCn1c(CNc2cccc(C(=O)NCc3c(F)cccc3F)c2)nnc1-c1ccncn1] |
| D37 | 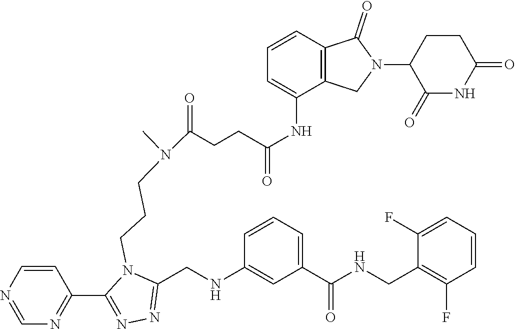 [SMILES: CN(CCCn1c(CNc2cccc(C(=O)NCc3c(F)cccc3F)c2)nnc1-c1ccncn1)C(=O)CCC(=O)Nc1cccc2c1CN(C1CCC(=O)NC1=O)C2=O] |
| D38 | 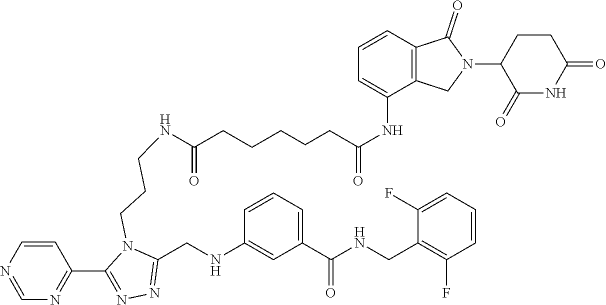 [SMILES: O=C(CCCCCC(=O)Nc1cccc2c1CN(C1CCC(=O)NC1=O)C2=O)NCCCn1c(CNc2cccc(C(=O)NCc3c(F)cccc3F)c2)nnc1-c1ccncn1] |
| D39 | 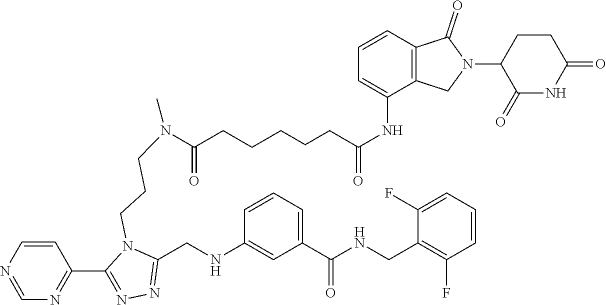 [SMILES: CN(CCCn1c(CNc2cccc(C(=O)NCc3c(F)cccc3F)c2)nnc1-c1ccncn1)C(=O)CCCCCC(=O)Nc1cccc2c1CN(C1CCC(=O)NC1=O)C2=O] |
| D40 | 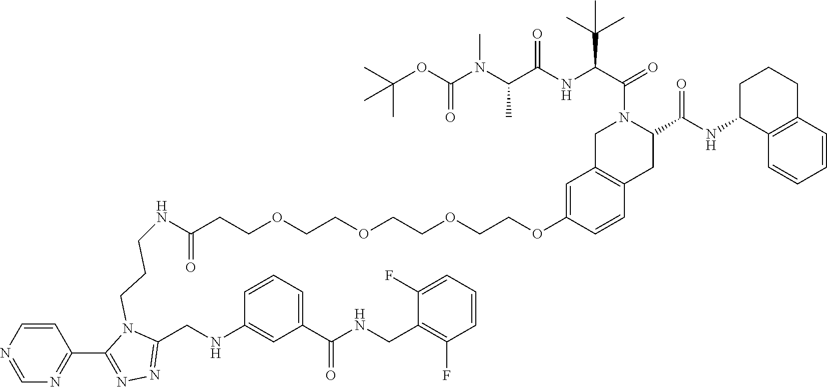 |
| D41 | 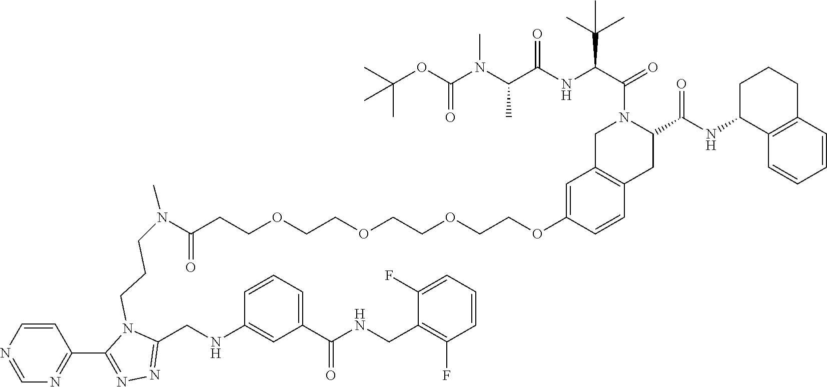 |
| D42 | 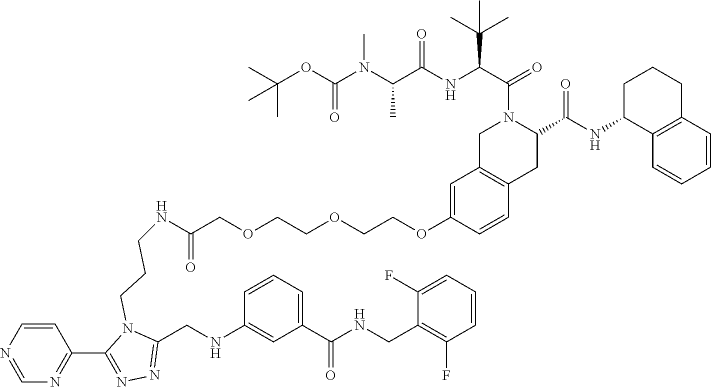 |
| D43 | 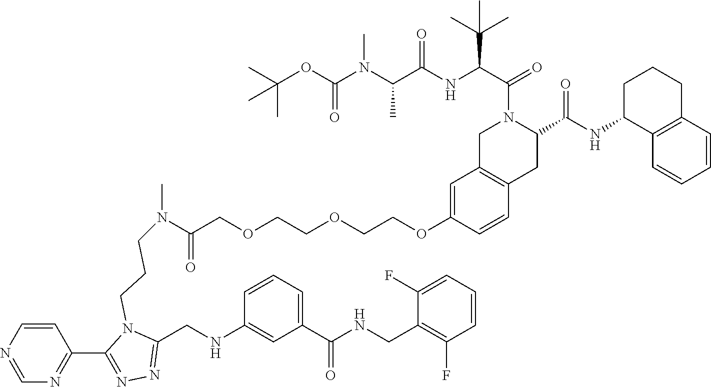 |
| D44 | 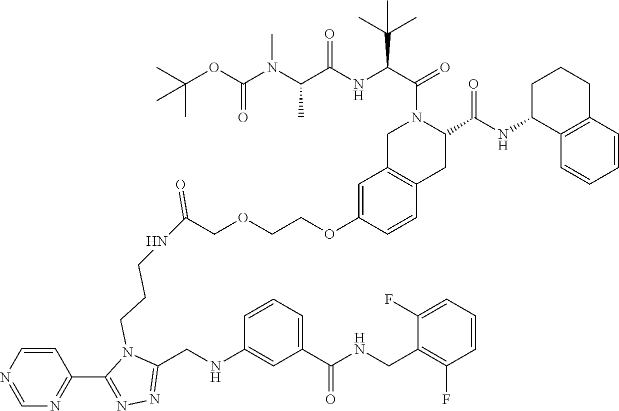 |
| D45 | 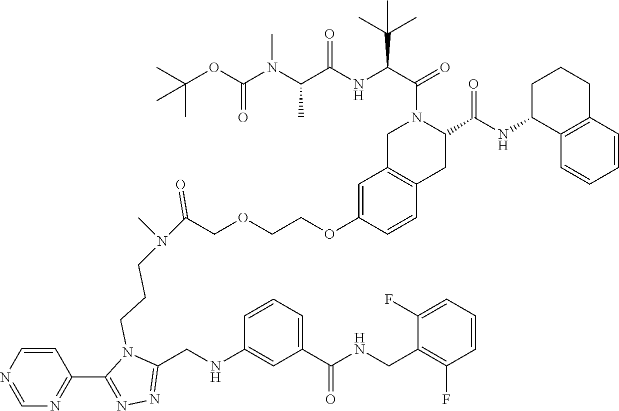 |
| D46 | 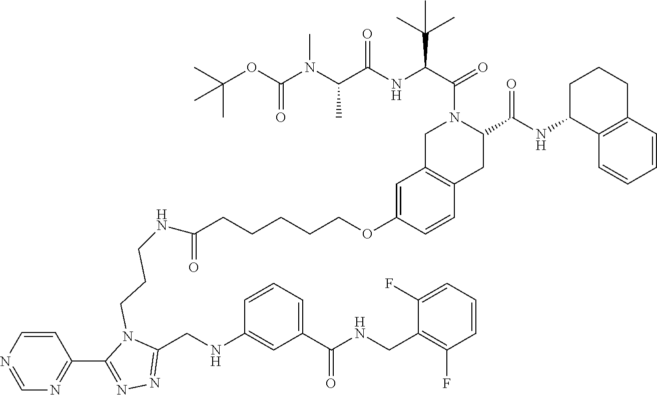 |
| D47 | 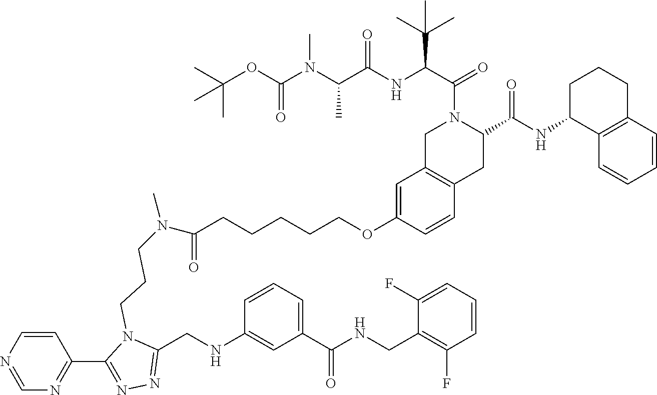 |
| D48 | 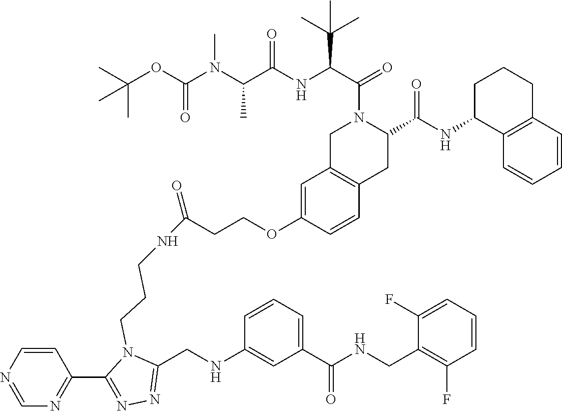 |
| D49 | 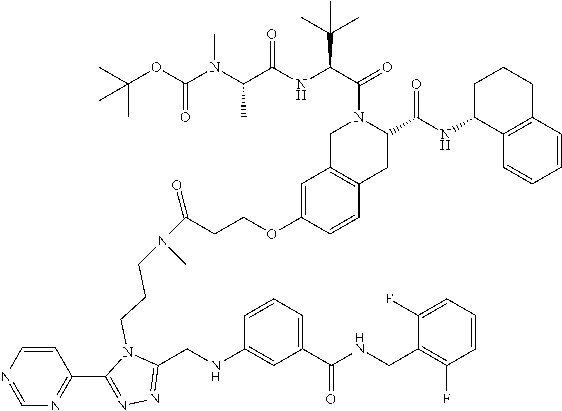 |
| D50 | 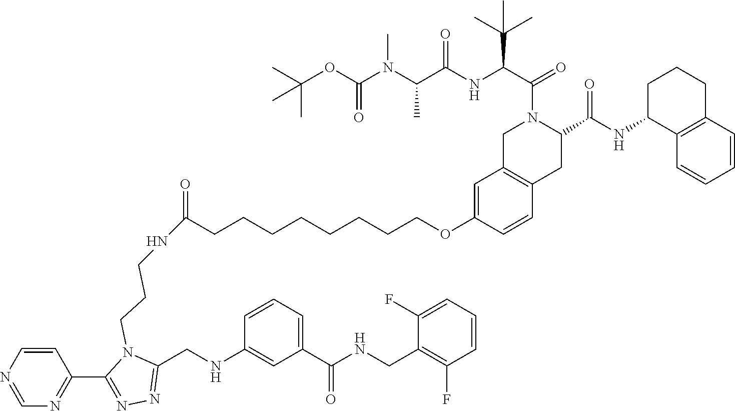 |
| D51 | 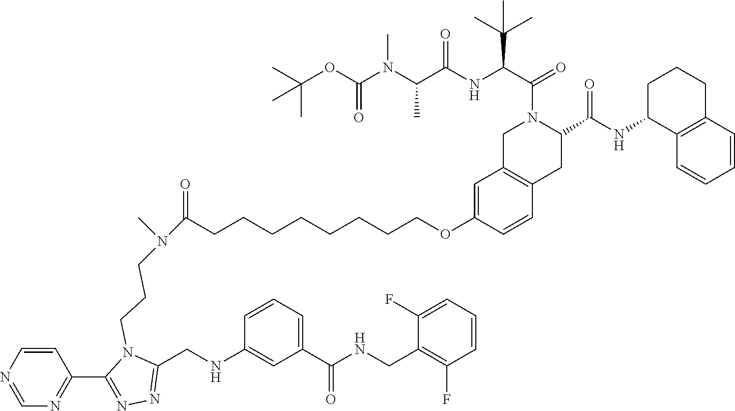 |

In an aspect, the disclosure features a pharmaceutical composition comprising any of the foregoing compounds, or pharmaceutically acceptable salt thereof, and a pharmaceutically acceptable excipient.

In an aspect, the disclosure features a method of decreasing the activity of GRK2 in a cell. This method includes contacting the cell with an effective amount of any of the foregoing compounds or pharmaceutical compositions.

In an aspect, the disclosure features a method of treating a GRK2-related disorder in a subject in need thereof. This method includes administering to the subject an effective amount of any of the foregoing compounds, or pharmaceutically acceptable salt thereof, or pharmaceutical compositions.

In some embodiments, the GRK2-related disorder is a hematological disease, an infection, a cardiovascular disease, e.g., cardiac failure, cardiac hypertrophy, and hypertension, cancer (e.g., skin cancer such as melanoma, breast cancer, ovarian cancer, prostate cancer, gliomas, thyroid cancer, pancreatic cancer, bile duct cancer, urinary tract cancer, head and neck cancer, gastric cancer, rhabdoid cancer, mesothelioma, cervical cancer, liver cancer, colorectal cancer, lymphoma, lung cancer, leukemia, and kidney cancer), an endocrinological disease, a metabolic disease, a gastroenterological disease, a respiratory disease, inflammation such as inflammatory bowel disease, a neurological disease, opioid addiction, or an urological disease.

In an aspect, the disclosure features a method of treating cancer (e.g., pancreatic cancer) in a subject in need thereof. This method includes administering to the subject an effective amount of any of the foregoing compounds, or pharmaceutically acceptable salt thereof, or s pharmaceutical composition thereof.

In some embodiments, the method further includes administering to the subject an anticancer therapy.

In an aspect, the disclosure features a method of identifying a GRK2-selective compound. This method includes contacting a first cell line that expresses GRK2 with a test compound; contacting a second cell line that has been engineered to overexpress GRK2 with the test compound; and assessing whether the proliferation of the first cell line is decreased in step a relative to the proliferation of the second cell line in step b, wherein a decrease in proliferation of the first cell line in step a of at least 2-fold (e.g., at least 3-fold, at least 4-fold, at least 5-fold, at least 10-fold, at least 15-fold, or at least 20-fold) indicates that the test compound is a GRK2-selective compound.

The details of one or more embodiments of the disclosure are set forth in the description below.

Other features, objects, and advantages of the disclosure will be apparent from the description and from the claims. Further features, objects, and advantages of the disclosure will be apparent from the examples and drawings.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIG. 1 shows in vivo tolerability of GRK2 inhibitor Compound S1 in mice with PAXF1657 tumors. All compounds were dissolved in 10% PG/50% PEG400/35% Peanut Oil/5% DMSO.

FIG. 2 shows in vivo efficacy of GRK2 inhibitor Compound S1 in a PAXF1657 pancreatic tumor model. All compounds dissolved in 10% PG/50% PEG400/35% Peanut Oil/5% DMSO. Treatment with Compound S1 led to 36% tumor growth inhibition (TGI) with 300 kg/kg QD dosing in in 10% PG/50% PEG/35% Peanoil/5% DMSO.

FIG. 3 shows the structure of Compound S1.

### DETAILED DESCRIPTION OF CERTAIN EMBODIMENTS

The present inventors have found that inhibition and/or depletion of GRK2 in cancer cells inhibits the proliferation of the cancer cells.

Accordingly, the disclosure features methods and compositions useful for the inhibition of the activity of GRK2, e.g., for the treatment of cancer such as pancreatic cancer. The disclosure further features methods and compositions useful for depletion of the GRK2 protein, e.g., for the treatment of cancer such as pancreatic cancer, e.g., in a subject in need thereof. Exemplary methods are described herein. The disclosure also provides compounds (e.g., GRK2 inhibitors) which are useful in the methods described herein.

### Compounds

In some embodiments, the compound of the disclosure, or a pharmaceutically acceptable salt thereof, has the structure:

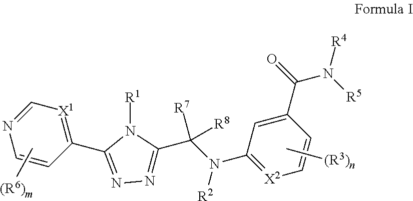

wherein m and n are, independently, 0, 1, 2, or 3;

X¹ is CR⁹ or N;

X² is CR³ or N;

R¹ is hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;

R² and R⁴ are, independently, hydrogen or optionally substituted C₁-C₆ alkyl;

each R³ and R⁶ is, independently, halogen, optionally substituted C₁-C₆ alkyl, optionally substituted C₁-C₆ heteroalkyl, hydroxyl, thiol, or optionally substituted amino;

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl; and

R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl; and

R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl.

In some embodiments, the compound is a GRK2-selective compound.

In some embodiments, if m and n are 0, R⁷ and R⁸ are hydrogen, X² is CH, R⁸ is hydrogen or optionally substituted C₁-C₆ alkyl, and R5 is optionally substituted C₁-C₆ alkyl C₆-C₁₀ aryl then X² is N.

In some embodiments, the compound of the disclosure, or a pharmaceutically acceptable salt thereof, has the structure:

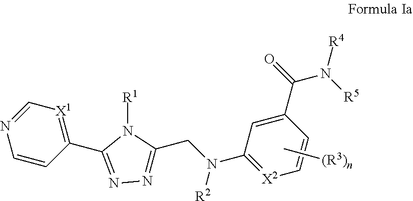

wherein n is 0, 1, 2, or 3;

X¹ and X² are, independently, CR³ or N;

R¹, R², and R⁴ are, independently, hydrogen or optionally substituted C₁-C₆ alkyl;

each R³ is, independently, hydrogen, halogen, optionally substituted C₁-C₆ alkyl, optionally substituted C₁-C₆ heteroalkyl, hydroxyl, thiol, or optionally substituted amino; and

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl, wherein if n is 0, X² is CH, and R⁵ is optionally substituted C₁-C₆ alkyl C₆-C₁₀ aryl then X² is N.

In some embodiments, the compound of the disclosure, or a pharmaceutically acceptable salt thereof, has the structure of Formula II:

A-L-B Formula II,

wherein

L is a linker;

B is a degradation moiety (e.g., a ubiquitin ligase binding moiety such as a ubiquitin ligase binding moiety including a Cereblon ligand, a IAP (Inhibitors of Apoptosis) ligand, a mouse double minute 2 homolog (MDM2), or a von Hippel-Lindau (VHL) ligand, or a derivative or an analog thereof); and

A has the structure of Formula III:

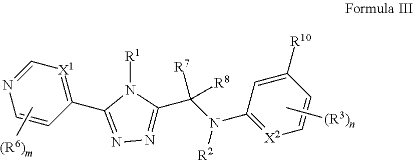

wherein m and n are, independently, 0, 1, 2, or 3;

X¹ is CR⁹ or N;

X² is CR³ or N;

R¹ is A¹, hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;

R² is hydrogen or optionally substituted C₁-C₆ alkyl;

each R³ and R⁶ is, independently, hydrogen, halogen, optionally substituted C₁-C₆ alkyl, optionally substituted C₁-C₆ heteroalkyl, hydroxyl, thiol, or optionally substituted amino;

R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl,

R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl;

R¹⁰ is —C(O)NR¹¹R¹² or —NHC(O)—R¹¹;

R¹¹ is A¹, optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl;

R¹² is hydrogen or optionally substituted C₁-C₆ alkyl; and

A¹ is a bond between A and the linker, wherein at least one, and only one of R¹ and R⁸ is A¹.

In some embodiments, the compound has the structure of any one of compounds 1-65 in Table 1 or D1-D51 in Table 2.

In some embodiments, the compound is any one of the compounds in Table 1. In some embodiments, the compound is any one of the compounds in Table 1, or a pharmaceutically acceptable salt thereof.

In some embodiments, the compound is any one of the compounds in Table 2. In some embodiments, the compound is any one of the compounds in Table 2, or a pharmaceutically acceptable salt thereof.

In some embodiments, the compound is any one of the compounds in Table 7. In some embodiments, the compound is any one of the compounds in Table 7, or a pharmaceutically acceptable salt thereof.

In addition to free base and pharmaceutically acceptable salt forms of the compounds described herein, the present disclosure provides stereoisomers, tautomers, isotopically labeled derivatives, solvates, hydrates, polymorphs, co-crystals, and prodrugs of the compounds described herein.

### Methods of Treatment

The methods described here can be used to treat cancer.

Treating cancer can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement.

Treating cancer may further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

Treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means.

An increase in average survival time of a population may be measured, for example, by calculating for a population the average length of survival following initiation of treatment with the compound of the disclosure. An increase in average survival time of a population may also be measured, for example, by calculating for a population the average length of survival following completion of a first round of treatment with the compound of the disclosure.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with the compound of the disclosure. A decrease in the mortality rate of a population may also be measured, for example, by calculating for a population the average number of disease-related deaths per unit time following completion of a first round of treatment with the compound of the disclosure.

Treating cancer can also result in an increased average progression-free survival time of a population of treated subjects in comparison to an untreated population. For example, the average progression-free survival time is increased by more than 30 days (more than 60 days, 90 days, or 120 days). An increase in average progression-free survival time of a population may be measured by any reproducible means. An increase in average progression-free survival time of a population may be measured, for example, by calculating for a population the average length of progression-free survival following initiation of treatment with the compound of the disclosure. An increase in average progression-free survival time of a population may also be measured, for example, by calculating for a population the average length of progression-free survival following completion of a first round of treatment with the compound of the disclosure.

### Combination Therapies

A method of the disclosure can be used alone or in combination with an additional anti-cancer therapy, e.g., surgery, radiation, and/or a therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In any of the combination embodiments described herein, the first and second therapeutic agents are administered simultaneously or sequentially, in either order.

### Pharmaceutical Compositions

The pharmaceutical compositions described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the disclosure, the described compounds or salts, solvates, or prodrugs thereof may be administered to a patient in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present disclosure may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response. In general, satisfactory results may be obtained when the compounds described herein are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

### Chemical Terms and Definitions

### Chemical Terms

For any of the following chemical definitions, a number following an atomic symbol indicates that total number of atoms of that element that are present in a particular chemical moiety. As will be understood, other atoms, such as hydrogen atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

When a range of values (“range”) is listed, it encompasses each value and sub-range within the range. A range is inclusive of the values at the two ends of the range unless otherwise provided. For example “C₁-C₆ alkyl” encompasses, C₁, C₂, C₃, C₄, C₅, C₆, C₁₋C₆, C₁₋C₅, C₁₋C₄, C₁₋C₃, C₁₋C₂, C₂₋C₆, C₂₋C₅, C₂₋C₄, C₂₋C₃, C₃₋C₆, C₃₋C₅, C₃₋C₄, C₄₋C₆, C₄₋C₅, and C₅₋C₆ alkyl.

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂)

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged cyclyl,” as used herein, refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

The term “cyano,” as used herein, represents a —CN group.

The term “carbocyclyl,” as used herein, refers to a non-aromatic C₃-C₁₂ monocyclic or polycyclic (e.g., bicyclic or tricyclic) structure in which the rings are formed by carbon atoms. Carbocyclyl structures include cycloalkyl groups and unsaturated carbocyclyl radicals. Polycyclic carbocyclyl includes spirocyclic carbocyclyl, bridged carbocyclyl, and fused carbocyclyl.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic monocyclic or polycyclic structure of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a monocyclic or polycyclic (e.g., bicyclic or tricyclic) structure having 3 to 12 atoms having at least one ring containing 1, 2, 3, or 4 ring atoms selected from N, O or S, wherein no ring is aromatic. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, Protective Groups in Organic Synthesis, 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. “Optionally substituted” as used herein refers to the group being either substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxyl), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Exemplary substituents (e.g., “optional substituents”) include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{b})₂)₂, —OP(═O)(N(R^{b})₂)₂, —NR^{bb}P(═O)(R^{aa})₂, —NR^{bb}P(═O)(OR^{cc})₂, —NR^{bb}P(═O)(N(R^{b})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋C₆ alkyl, C₁₋C₆ perhaloalkyl, C₂-C₆ alkenyl, C₂-C₆ alkynyl, C₁-C₆ heteroalkyl, C₂₋C₆ heteroalkenyl, C₂₋C₆ heteroalkynyl, C₃-C₈ carbocyclyl, 3-8 membered heterocyclyl, C₆₋C₁₀ aryl, and 5-10 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
each instance of R^{aa} is, independently, selected from C₁₋C₆ alkyl, C₁₋C₆ perhaloalkyl, C₂₋C₆ alkenyl, C₂₋C₆ alkynyl, C₁₋C₆ heteroalkyl, C₂₋C₆ heteroalkenyl, C₂₋C₆ heteroalkynyl, C₃₋C₈ carbocyclyl, 3-8 membered heterocyclyl, C₆₋C₁₀ aryl, and 5-10 membered heteroaryl, or two R^{aa} groups are joined to form a 3-8 membered heterocyclyl or 5-10 membered heteroaryl ring;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋C₆ alkyl, C₁₋C₆ perhaloalkyl, C₂₋C₆ alkenyl, C₂₋C₆ alkynyl, C₁₋C₆ heteroalkyl, C₂₋C₆ heteroalkenyl, C₂₋C₆ heteroalkynyl, C₃₋C₈ carbocyclyl, 3-8 membered heterocyclyl, C₆₋C₁₀ aryl, and 5-10 membered heteroaryl, or two R^{bb} groups are joined to form a 3-8 membered heterocyclyl or 5-10 membered heteroaryl ring;
each instance of R^{cc} is, independently, selected from hydrogen, C₁₋C₆ alkyl, C₁₋C₆ perhaloalkyl, C₂₋C₆ alkenyl, C₂₋C₆ alkynyl, C₁₋C₆ heteroalkyl, C₂₋C₆ heteroalkenyl, C₂₋C₆ heteroalkynyl, C₃₋C₈ carbocyclyl, 3-8 membered heterocyclyl, C₆₋C₁₀ aryl, and 5-10 membered heteroaryl, or two R^{cc} groups are joined to form a 3-8 membered heterocyclyl or 5-10 membered heteroaryl ring; and each X⁻ is a counterion.

In some embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂; wherein each R^{aa} is independently hydrogen or C₁₋C₆ alkyl; and each R^{bb} is independently hydrogen or C₁₋C₆ alkyl.

Compounds described herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained, for example, by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on 25 opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide 35 of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The disclosure embraces all of these forms.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Other Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

The term “cancer” refers to any cancer caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “degrader” refers to a small molecule compound including a degradation moiety, wherein the compound interacts with a protein (e.g., GRK2) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., GRK2. In one example, the moiety binds to a protease or a ubiquitin ligase that metabolizes the protein, e.g., GRK2.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

The term “effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “effective amount” does not in fact require successful treatment be achieved in a particular individual. Rather, an effective amount may be that amount that provides a particular desired pharmacological response in a significant number of subjects when administered to patients in need of such treatment. It is specifically understood that particular subjects may, in fact, be “refractory” to an “effective amount.” To give but one example, a refractory subject may have a low bioavailability such that clinical efficacy is not obtainable. In some embodiments, reference to an effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, an effective amount may be formulated and/or administered in a single dose. In some embodiments, an effective amount may be formulated and/or administered in a plurality of doses, for example, as part of a dosing regimen.

As used herein, the term “GRK2” refers to G-protein-coupled receptor kinase 2 and belongs to the G-protein-coupled receptor kinase subfamily of the Ser/Thr protein kinases. GRK2 is encoded by the ADRBK1 gene, the nucleic acid sequence of which is set forth in SEQ ID NO: 1.

SEQ ID NO: 1
ATGGCGGACCTGGAGGCGGTGCTGGCCGACGTGAGCTACCTGATGGCCAT
GGAGAAGAGCAAGGCCACGCCGGCCGCGCGCGCCAGCAAGAAGATCCTGC
TGCCCGAGCCCAGCATCCGCAGTGTCATGCAGAAGTACCTGGAGGACCGG
GGCGAGGTGACCTTTGAGAAGATCTTTTCCCAGAAGCTGGGGTACCTGCT
CTTCCGAGACTTCTGCCTGAACCACCTGGAGGAGGCCAGGCCCTTGGTGG
AATTCTATGAGGAGATCAAGAAGTACGAGAAGCTGGAGACGGAGGAGGAG
CGTGTGGCCCGCAGCCGGGAGATCTTCGACTCATACATCATGAAGGAGCT
GCTGGCCTGCTCGCATCCCTTCTCGAAGAGTGCCACTGAGCATGTCCAAG
GCCACCTGGGGAAGAAGCAGGTGCCTCCGGATCTCTTCCAGCCATACATC
GAAGAGATTTGTCAAAACCTCCGAGGGGACGTGTTCCAGAAATTCATTGA
GAGCGATAAGTTCACACGGTTTTGCCAGTGGAAGAATGTGGAGCTCAACA
TCCACCTGACCATGAATGACTTCAGCGTGCATCGCATCATTGGGCGCGGG
GGCTTTGGCGAGGTCTATGGGTGCCGGAAGGCTGACACAGGCAAGATGTA
CGCCATGAAGTGCCTGGACAAAAAGCGCATCAAGATGAAGCAGGGGGAGA
CCCTGGCCCTGAACGAGCGCATCATGCTCTCGCTCGTCAGCACTGGGGAC
TGCCCATTCATTGTCTGCATGTCATACGCGTTCCACACGCCAGACAAGCT
CAGCTTCATCCTGGACCTCATGAACGGTGGGGACCTGCACTACCACCTCT
CCCAGCACGGGGTCTTCTCAGAGGCTGACATGCGCTTCTATGCGGCCGAG
ATCATCCTGGGCCTGGAGCACATGCACAACCGCTTCGTGGTCTACCGGGA
CCTGAAGCCAGCCAACATCCTTCTGGACGAGCATGGCCACGTGCGGATCT
CGGACCTGGGCCTGGCCTGTGACTTCTCCAAGAAGAAGCCCCATGCCAGC
GTGGGCACCCACGGGTACATGGCTCCGGAGGTCCTGCAGAAGGGCGTGGC
CTACGACAGCAGTGCCGACTGGTTCTCTCTGGGGTGCATGCTCTTCAAGT
TGCTGCGGGGGCACAGCCCCTTCCGGCAGCACAAGACCAAAGACAAGCAT
GAGATCGACCGCATGACGCTGACGATGGCCGTGGAGCTGCCCGACTCCTT
CTCCCCTGAACTACGCTCCCTGCTGGAGGGGTTGCTGCAGAGGGATGTCA
ACCGGAGATTGGGCTGCCTGGGCCGAGGGGCTCAGGAGGTGAAAGAGAGC
CCCTTTTTCCGCTCCCTGGACTGGCAGATGGTCTTCTTGCAGAAGTACCC
TCCCCCGCTGATCCCCCCACGAGGGGAGGTGAACGCGGCCGACGCCTTCG
ACATTGGCTCCTTCGATGAGGAGGACACAAAAGGAATCAAGTTACTGGAC
AGTGATCAGGAGCTCTACCGCAACTTCCCCCTCACCATCTCGGAGCGGTG
GCAGCAGGAGGTGGCAGAGACTGTCTTCGACACCATCAACGCTGAGACAG
ACCGGCTGGAGGCTCGCAAGAAAGCCAAGAACAAGCAGCTGGGCCATGAG
GAAGACTACGCCCTGGGCAAGGACTGCATCATGCATGGCTACATGTCCAA
GATGGGCAACCCCTTCCTGACCCAGTGGCAGCGGCGGTACTTCTACCTGT
TCCCCAACCGCCTCGAGTGGCGGGGCGAGGGCGAGGCCCCGCAGAGCCTG
CTGACCATGGAGGAGATCCAGTCGGTGGAGGAGACGCAGATCAAGGAGCG
CAAGTGCCTGCTCCTCAAGATCCGCGGTGGGAAACAGTTCATTTTGCAGT
GCGATAGCGACCCTGAGCTGGTGCAGTGGAAGAAGGAGCTGCGCGACGCC
TACCGCGAGGCCCAGCAGCTGGTGCAGCGGGTGCCCAAGATGAAGAACAA
GCCGCGCTCGCCCGTGGTGGAGCTGAGCAAGGTGCCGCTGGTCCAGCGCG
GCAGTGCCAACGGCCTCTGA

The term “GRK2” also refers to natural variants of the wild-type GRK2 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type GRK2, which is set forth in SEQ ID NO: 2.

SEQ ID NO: 2
MADLEAVLADVSYLMAMEKSKATPAARASKKILLPEPSIRSVMQKYLEDR
GEVTFEKIFSQKLGYLLFRDFCLNHLEEARPLVEFYEEIKKYEKLETEEE
RVARSREIFDSYIMKELLACSHPFSKSATEHVQGHLGKKQVPPDLFQPYI
EEICQNLRGDVFQKFIESDKFTRFCQWKNVELNIHLTMNDFSVHRIIGRG
GFGEVYGCRKADTGKMYAMKCLDKKRIKMKQGETLALNERIMLSLVSTGD
CPFIVCMSYAFHTPDKLSFILDLMNGGDLHYHLSQHGVFSEADMRFYAAE
IILGLEHMHNRFVVYRDLKPANILLDEHGHVRISDLGLACDFSKKKPHAS
VGTHGYMAPEVLQKGVAYDSSADWFSLGCMLFKLLRGHSPFRQHKTKDKH
EIDRMTLTMAVELPDSFSPELRSLLEGLLQRDVNRRLGCLGRGAQEVKES
PFFRSLDWQMVFLQKYPPPLIPPRGEVNAADAFDIGSFDEEDTKGIKLLD
SDQELYRNFPLTISERWQQEVAETVFDTINAETDRLEARKKAKNKQLGHE
EDYALGKDCIMHGYMSKMGNPFLTQWQRRYFYLFPNRLEWRGEGEAPQSL
LTMEEIQSVEETQIKERKCLLLKIRGGKQFILQCDSDPELVQWKKELRDA
YREAQQLVQRVPKMKNKPRSPVVELSKVPLVQRGSANGL

As used herein, the term “GRK2-related disorder,” refers to a diseases or condition that is associated with cells that express or overexpress GRK2 (e.g., cancer cells that express or overexpress GRK2 compared to a reference). GRK2-related disorders can be identified by assessing a cell or a biopsy of a tissue sample for GRK2 expression and comparing it to GRK2 expression in a reference cell or tissue sample.

As used herein, the term “GRK2-selective compound,” refers to a compound having a ratio of greater than 2 (e.g., greater than 3, greater than 4, greater than 5, greater than 10, greater than 15, or greater than 20) of the GI₅₀ in the PAXF1657 cell line stably overexpressing GRK2 over the GI₅₀ in the PAXF1657 control empty vector cell line in the assay described in Example 9. For example, compound 1 has a ratio of 12.34, i.e., a GI₅₀ in the PAXF1657 cell line stably overexpressing GRK2 of 24.30 and a GI₅₀ in the PAXF1657 control empty vector cell line of 1.97 (24.30:1.97). Thus, compound 1 is a GRK2-selective compound.

By “reducing the activity of GRK2” or “decreasing the activity of GRK2” is meant decreasing the level of an activity related to a GRK2 protein, or a related downstream effect. In some embodiments, the level of an activity related to a GRK2 protein, or a downstream effect, is decreased in a cell. In some embodiments, the cell is a mammalian cell. In some embodiments, the cell is a cancer cell (e.g., pancreatic cancer cell).

By “level” is meant a level of a protein, or mRNA encoding the protein, as compared to a reference (i.e., control). The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

“Progression-free survival” as used herein, refers to the length of time during and after medication or treatment during which the disease being treated (e.g., cancer) does not get worse.

“Proliferation” as used in this application involves reproduction or multiplication of similar forms (cells) due to constituting (cellular) elements.

By a “reference” or “control” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material. The details of one or more embodiments of the disclosure are set forth in the description below. Other features, objects, and advantages of the disclosure will be apparent from the description and from the claims.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### EXAMPLES

### Example 1. Preparation of Key Intermediates

Schemes below illustrate the preparation of key intermediates.

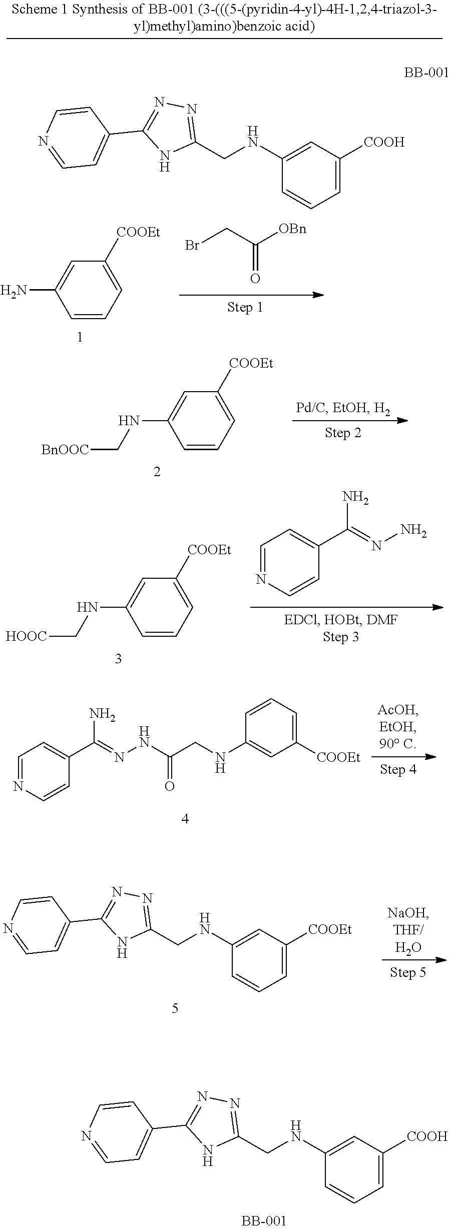

### Step 1: Ethyl 3-[[2-(benzyloxy)-2-oxoethyl]amino]benzoate

To a stirred solution of tricaine (5.0 g, 30.37 mmol, 1.0 equiv.) and benzyl 2-bromoacetate (6.9 g, 30.27 mmol, 1.0 equiv.) in DMF (40.0 mL), was added K₂CO₃ (6.3 g, 45.40 mmol, 1.5 equiv.) in portions at 0° C. under atmosphere of nitrogen. The resulting mixture was stirred for overnight at 45° C. and then quenched by the addition of water. The resulting mixture was extracted with EtOAc, washed with brine, dried over anhydrous Na₂SO₄, and concentrated under reduced pressure. The residue was purified by silica gel column chromatography, eluted with petroleum ether/EtOAc (1:25) to afford ethyl 3-[[2-(benzyloxy)-2-oxoethyl]amino]benzoate (6.3 g, 66.4%) as a yellow solid. MS-ESI: 314.1 [M+H]⁺.

### Step 2: [[3-(ethoxycarbonyl)phenyl]amino]acetic acid

To a solution of ethyl 3-[[2-(benzyloxy)-2-oxoethyl]amino]benzoate (30.0 g, 95.74 mmol, 1.0 equiv.) in 300 mL MeOH was added Pd/C (10%, 8.9 g). The solution was degassed and back filled with nitrogen for three times and then stirred for overnight under atmosphere of hydrogen. After filtration through a Celite pad, the solution was concentrated under reduced pressure to afford [[3-(ethoxycarbonyl)phenyl]amino]acetic acid (15.1 g, crude) as a yellow oil. MS-ESI: 224.1 [M+H]⁺.

### Step 3: ethyl 3-[([N-[(1Z)-amino(pyridin-4yl)methylidene]hydrazinecarbonyl]methyl)amino]benzoate

To a stirred solution of [[3-(ethoxycarbonyl)phenyl]amino]acetic acid (15.0 g, 67.20 mmol, 1.0 equiv.) and (Z)—N-aminopyridine-4-carboximidamide (9.2 g, 67.20 mmol, 1.0 equiv.) in DMF (150.0 mL), were added HOBt (13.6 g, 100.80 mmol, 1.5 equiv.) and WSC.HCl (19.3 g, 100.80 mmol, 1.5 equiv.) at room temperature. The final reaction mixture was stirred for overnight at room temperature and then quenched by the addition of water. The resulting mixture was extracted with EtOAc, washed with brine, dried over anhydrous MgSO₄, and concentrated under reduced pressure. The residue was purified by silica gel column chromatography, eluted with DCM/MeOH (10:1) to afford ethyl 3-[([N-[(1Z)-amino(pyridin-4yl)methylidene]hydrazinecarbonyl]methyl)amino]benzoate (12.1 g, 52.3%) as a yellow solid. MS-ESI: 342.2 [M+H]⁺.

### Step 4: ethyl 3-([[5-(pyridin-4-yl)-4H-1,2,4-triazol-3-yl]methyl]amino)benzoate

To a stirred solution of ethyl 3-[([N-[(1Z)-amino(pyridin-4-yl)methylidene]hydrazinecarbonyl]methyl)amino]benzoate (12.0 g, 35.15 mmol, 1.0 equiv.) in EtOH (120.0 mL), was added AcOH (12.0 mL) dropwise at room temperature. The resulting mixture was stirred for overnight at 90° C. and then concentrated under vacuum. The residue was purified by silica gel column chromatography, eluted with DCM/MeOH (10:1) to afford ethyl 3-([[5-(pyridin-4-yl)-4H-1,2,4-triazol-3-yl]methyl]amino)benzoate (7.1 g, 61.6%) as a yellow solid. MS-ESI: 324.1 [M+H]⁺.

### Step 5: 3-([[5-(pyridin-4-yl)-4H-1,2,4-triazol-3-yl]methyl]amino)benzoic acid

To a stirred solution of ethyl 3-([[5-(pyridin-4-yl)-4H-1,2,4-triazol-3-yl]methyl]amino)benzoate (7.0 g, 21.65 mmol, 1.0 equiv.) in MeOH/water (30.0 mL/30.0 mL), NaOH (3.5 g, 86.59 mmol, 4.0 equiv.) was added at 0° C. The resulting mixture was stirred for overnight at room temperature and then concentrated under vacuum to remove MeOH. The resulting mixture was acidified to pH 6 with conc. HCl. The precipitated solids were collected by filtration and dried to afford 3-([[5-(pyridin-4-yl)-4H-1,2,4-triazol-3-yl]methyl]amino)benzoic acid (4.68 g, 73.2%) as a white solid. MS-ESI: 296.1 [M+H]⁺. ¹H NMR (300 MHz, DMSO-d₆) δ 14.34 (brs, 1H), 12.68 (brs, 1H), 8.68 (d, J=4.8 Hz, 2H), 7.91-7.78 (m, 2H), 7.24-7.15 (m, 3H), 6.87-6.83 (m, 1H), 6.59 (t, J=5.7 Hz, 1H), 4.48 (d, J=5.7 Hz, 2H).

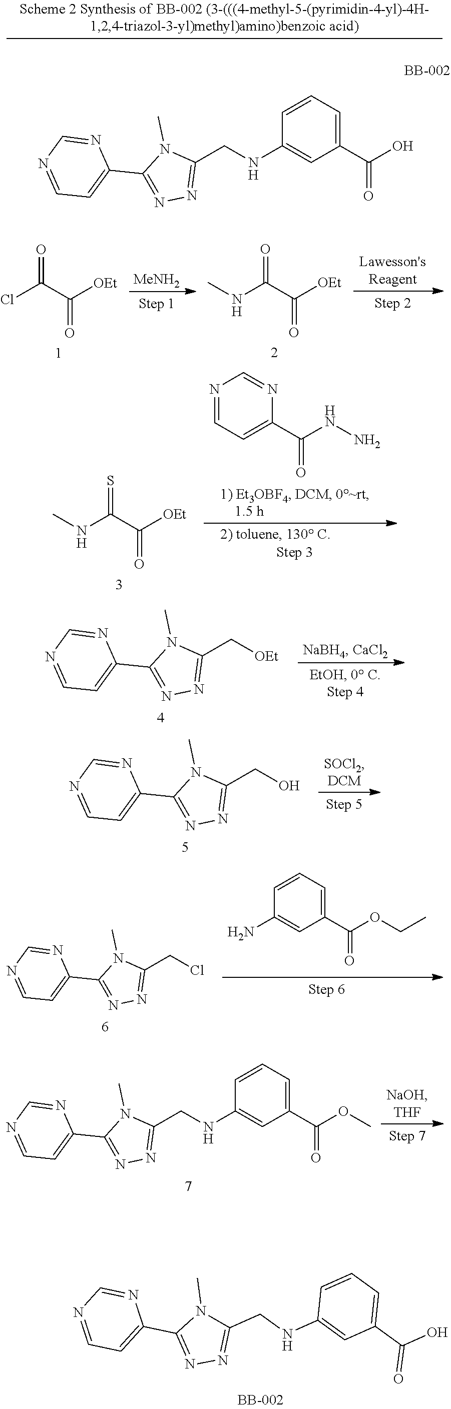

### Step 1: ethyl (methylcarbamoyl)formate

To a stirred solution of methylamine (12.5 g, 402.84 mmol, 1.1 equiv.) and triethylamine (92.7 g, 915.58 mmol, 2.5 equiv.) in DCM (1.5 L), was added ethyl chloroglyoxylate (50.0 g, 366.22 mmol, 1.0 equiv.) dropwise at 0° C. The resulting mixture was stirred for 1 h at 0° C. and then quenched by the addition of water. The mixture was extracted with DCM and concentrated under vacuum to afford ethyl (methylcarbamoyl)formate (25 g, 52.1%) as a brown yellow oil. MS-ESI: 132.1 [M+H]⁺.

### Step 2: ethyl (methylcarbamothioyl)formate

To a solution of ethyl (methylcarbamoyl)formate (25.0 g, 190.65 mmol, 1.0 equiv.) in toluene (500.0 mL), was added Lawesson reagent (38.5 g, 95.33 mmol, 0.5 equiv.). The resulting mixture was stirred for overnight at 90° C. and then concentrated under reduced pressure. The residue was purified by silica gel column chromatography, eluted with petroleum ether/EtOAc (1:1) to afford ethyl (methylcarbamothioyl)formate (12 g, 42.7%) as a yellow oil. MS-ESI: 148.0 [M+H]⁺.

### Step 3: ethyl 4-methyl-5-(pyrimidin-4-yl)-1,2,4-triazole-3-carboxylate

To a stirred solution of ethyl (methylcarbamothioyl)formate (12 g, 81.53 mmol, 1.0 equiv.) in DCM (100 mL), was added a solution of Et₃OBF₄ (23.2 g, 122.29 mmol, 1.5 equiv.) in DCM (200.0 mL) dropwise at 0° C. The resulting mixture was stirred for 1.5 h at room temperature under nitrogen atmosphere. The solution was washed with brine and concentrated under vacuum. The residue was dissolved in toluene (400.0 mL), and to the mixture was added pyrimidine-4-carbohydrazide (11.3 g, 81.53 mmol, 1.0 equiv.). The resulting mixture was stirred for additional overnight at 130° C. and concentrated under reduced pressure. The residue was purified by silica gel column chromatography, eluted with DCM/MeOH (20:1) to afford ethyl 4-methyl-5-(pyrimidin-4-yl)-1,2,4-triazole-3-carboxylate (13 g, 63.9%) as a yellow solid. MS-ESI: 234.1 [M+H]⁺.

### Step 4: [4-methyl-5-(pyrimidin-4-yl)-1,2,4-triazol-3-yl]methanol

To a stirred solution of ethyl 4-methyl-5-(pyrimidin-4-yl)-1,2,4-triazole-3-carboxylate (12.0 g, 51.45 mmol, 1.0 equiv.) in EtOH (160.0 mL), were added CaCl₂ (1.7 g, 15.44 mmol, 0.3 equiv.), and NaBH₄ (2.4 g, 61.74 mmol, 1.2 equiv.) in portions at 0° C. The final reaction mixture was stirred for 3 h at room temperature and then quenched by the addition of water. After concentration under vacuum, the residue was purified by silica gel column chromatography, eluted with DCM/MeOH (12:1) to afford [4-methyl-5-(pyrimidin-4-yl)-1,2,4-triazol-3-yl]methanol (9.0 g, 91.5%) as a white solid. MS-ESI: 192.1 [M+H]⁺.

### Step 5: 4-[5-(chloromethyl)-4-methyl-1,2,4-triazol-3-yl]pyrimidine

To a stirred solution of [4-methyl-5-(pyrimidin-4-yl)-1,2,4-triazol-3-yl]methanol (9.0 g, 47.07 mmol, 1.0 equiv.) in DCM (45.0 mL), was added SOCl₂ (45.0 mL) dropwise at 0° C. under atmosphere of nitrogen. The resulting mixture was stirred for 2 h at room temperature and then concentrated under reduced pressure to afford 4-[5-(chloromethyl)-4-methyl-1,2,4-triazol-3-yl]pyrimidine (12.6 g, crude) as a yellow crude solid. MS-ESI: 210.1 [M+H]⁺.

### Step 6: ethyl 3-([[4-methyl-5-(pyrimidin-4-yl)-1,2,4-triazol-3-yl]methyl]amino)benzoate

To a stirred solution of 4-[5-(chloromethyl)-4-methyl-1,2,4-triazol-3-yl]pyrimidine (12.6 g, 60.10 mmol, 1.0 equiv.) in DMF (300.0 mL), was added ethyl 3-aminobenzoate (29.8 g, 180.31 mmol, 3.0 equiv.). The resulting mixture was stirred for 14 h at 90° C. and then quenched by the addition of water. The aqueous layer was extracted with DCM and concentrated under reduced pressure. The residue was purified by silica gel column chromatography, eluted with DCM/MeOH (12:1) to afford ethyl 3-([[4-methyl-5-(pyrimidin-4-yl)-1,2,4-triazol-3-yl]methyl]amino)benzoate (11.0 g, 54.1%) as a brown solid. MS-ESI: 325.1 [M+H]⁺.

### Step 7: 3-([[4-methyl-5-(pyrimidin-4-yl)-1,2,4-triazol-3-yl]methyl]amino)benzoic acid

To a stirred solution of ethyl 3-([[4-methyl-5-(pyrimidin-4-yl)-1,2,4-triazol-3-yl]methyl]amino) benzoate (11.0 g, 32.51 mmol, 1.0 equiv.) in THF/water (80.0 mL/80.0 mL), was added NaOH (5.2 g, 130.04 mmol, 4.0 equiv.). The resulting mixture was stirred for overnight at room temperature and concentrated under reduced pressure to remove THF. The resulting solution was acidified to pH=6 with HCl (aq.). The resulting solids were collected by filtration, washed with DCM and dried to afford 3-([[4-methyl-5-(pyrimidin-4-yl)-1,2,4-triazol-3-yl]methyl]amino)benzoic acid (10.1 g, 99.1%) as an off-white solid. MS-ESI: 311.2 [M+H]⁺. ¹H NMR (400 MHz, DMSO-d₆) δ 9.34 (s, 1H), 8.98 (d, J=5.2 Hz, 1H), 8.19-8.17 (m, 1H), 7.35 (s, 1H), 7.24-7.17 (m, 2H), 7.001-6.98 (m, 1H), 4.58 (s, 2H), 3.35 (s, 3H).

The intermediates in Table 3 below were prepared using methods similar to those described for BB-002 above.

TABLE 3

| Building | Starting | | |
|---|---|---|---|
| Block | material | Structure | Analytical data |
| BB-005 | 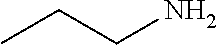 | 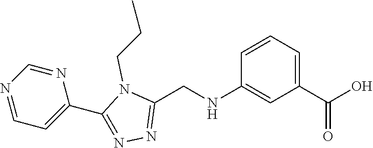 [SMILES: CCCn1c(CNc2cccc(C(=O)O)c2)nnc1-c1ccncn1] | MS-ESI: 339.1 [M + H]⁺. ¹H NMR (400 MHz, DMSO-d₆) δ 12.75 (brs, 1H), 9.34 (s, 1H), 8.97 (d, J = 5.2 Hz, 1H), 8.22-8.20 (m, 1H), 7.35 (s, 1H), 7.24-7.17 (m, 2H), 7.00-6.98 (m, 1H), 4.60 (s, 2H), 4.54 (t, J = 7.6 Hz, 2H), 1.76-1.67 (m, 2H), 0.86 (t, J = 7.6 Hz, 3H). |
| BB-007 | 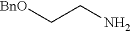 [SMILES: NCCOCc1ccccc1] | 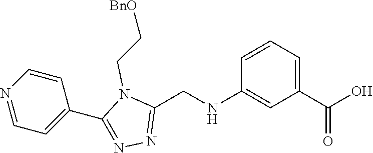 [SMILES: O=C(O)c1cccc(NCc2nnc(-c3ccncc3)n2CCOCc2ccccc2)c1] | MS-ESI: 430.2 [M + H]⁺. ¹H NMR (300 MHz, DMSO-d₆) δ 12.66 (brs, 1H), 9.34 (s, 1H), 8.67 (d, J = 4.8 Hz, 2H), 7.67 (d, J = 6.0 Hz, 2H), 7.34 (s, 1H), 7.27-7.18 (m, 5H), 7.04-7.02 (m, 2H), 6.97-6.93 (m, 1H), 6.55-6.51 (m, 1H), 4.57-4.55 (m, 2H), 4.45 (t, J = 4.8 Hz, 2H), 4.32 (s, 2H), 3.60 (t, J = 4.8 Hz, 2H), 3.60 (t, J = 4.8 Hz, 2H). |

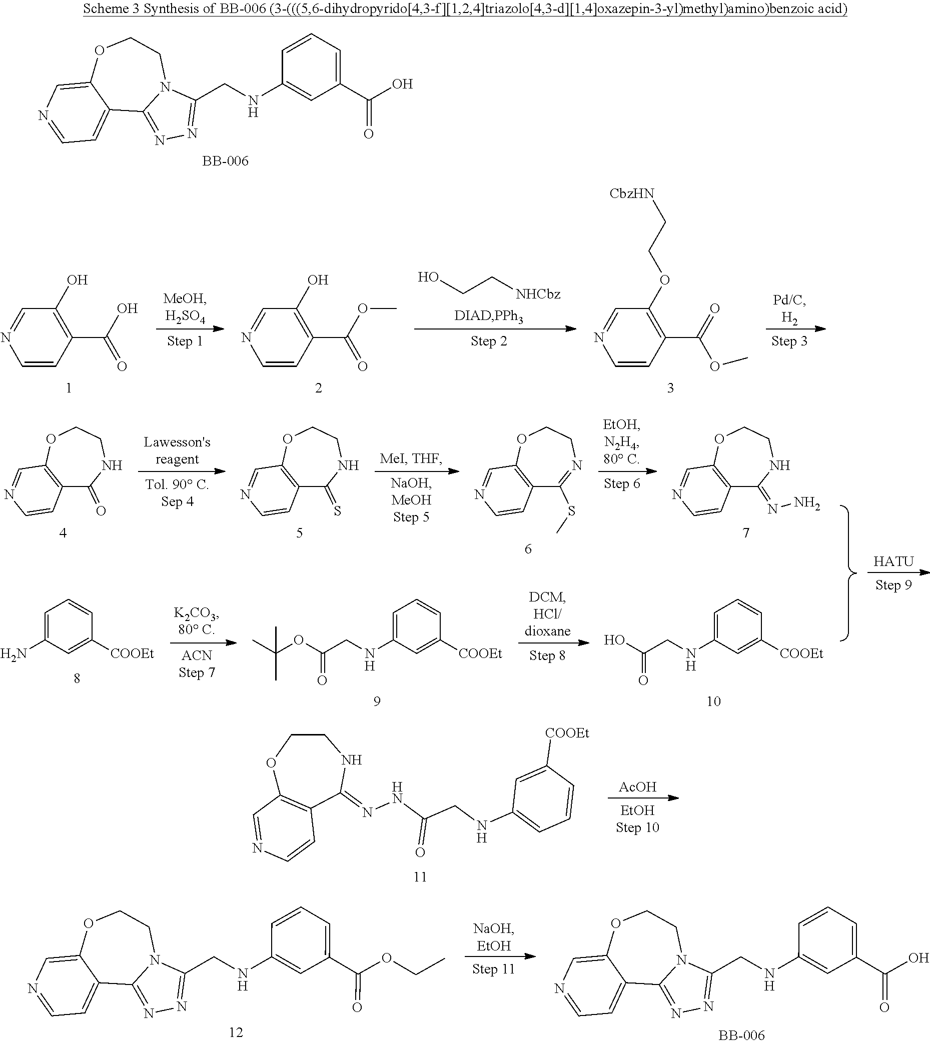

### Step 1: Methyl 2-mercaptothiazole-5-carboxylate

To a stirred solution of 3-hydroxypyridine-4-carboxylic acid (50.0 g, 359.43 mmol, 1.0 equiv.) in MeOH (500.0 mL), was added conc. H₂SO₄ (70.0 mL, 1313.24 mmol, 3.7 equiv.) dropwise at 0° C. The resulting solution was stirred for 16 h at 80° C. and then quenched by the addition of water. The pH value of the solution was adjusted to 7 with K₂CO₃ aqueous. The resulting solution was extracted DCM and concentrated under reduced pressure to afford methyl 3-hydroxypyridine-4-carboxylate (42.0 g, 76.3%) as a light yellow solid. MS-ESI: 154.0 [M+H]⁺.

### Step 2: Methyl 3-(2-[[(benzyloxy)carbonyl]amino]ethoxy)pyridine-4-carboxylate

To a solution of methyl 3-hydroxypyridine-4-carboxylate (42.0 g, 274.26 mmol, 1.0 equiv.), PPh₃ (86.3 g, 329.12 mmol, 1.2 equiv.), and benzyl (2-hydroxyethyl)carbamate (53.5 g, 274.26 mmol, 1.0 equiv.) in THF (300.0 mL), was added a solution of DIAD (66.6 g, 329.12 mmol, 1.2 equiv.) in THF (100.0 mL) dropwise with stirring at 0° C. The resulting solution was stirred for 16 h at room temperature and concentrated under reduced pressure to afford methyl 3-(2-[[(benzyloxy)carbonyl]amino]ethoxy)pyridine-4-carboxylate (200 g, crude) as yellow oil. MS-ESI: 331.1 [M+H]⁺.

### Step 3: 2H,3H,4H-pyrido[4,3-f][1,4]oxazepin-5-one

To a solution of methyl 3-(2-[[(benzyloxy)carbonyl]amino]ethoxy)pyridine-4-carboxylate (200.0 g, 605.44 mmol, 1.0 equiv.) in EtOH (700.0 mL), was added Pd/C (20 g, 10%). The solution was degassed and back filled with hydrogen for three times and the resulting solution was stirred for additional 48 h at 50° C. under atmosphere of hydrogen. The solids were filtered out and the filtrate was concentrated under reduced pressure. The residue was applied onto a silica gel column, eluted with ethyl acetate/petroleum ether (1:1 to 1:0) to afford 2H,3H,4H-pyrido[4,3-f][1,4]oxazepin-5-one (40 g) as a light yellow solid. MS-ESI: 165.1 [M+H]⁺.

### Step 4: 2H,3H,4H-pyrido[4,3-f][1,4]oxazepine-5-thione

To a solution of 2H,3H,4H-pyrido[4,3-f][1,4]oxazepin-5-one (30.0 g, 182.74 mmol, 1.0 equiv.) in toluene (600.0 mL), was added Lawesson Reagent (37.0 g, 91.37 mmol, 0.5 equiv.). The resulting solution was stirred for 3 h at 100° C. and then concentrated under reduced pressure. The residue was applied onto a silica gel column with ethyl acetate/petroleum ether (1:1 to 1:0) to afford 2H,3H,4H-pyrido[4,3-f][1,4]oxazepine-5-thione (16.0 g, 48.6%) as a yellow solid. MS-ESI: 181.0 [M+H]⁺.

### Step 5: 5-(methylsulfanyl)-2H,3H-pyrido[4,3-f][1,4]oxazepine

To a solution of 2H,3H,4H-pyrido[4,3-f][1,4]oxazepine-5-thione (16.0 g, 88.78 mmol, 1.0 equiv.) in MeOH/THF (100.0 mL/200.0 mL), was added NaOH (5.3 g, 133.26 mmol, 1.5 equiv.). After stirred for 30 min, to the solution was Mel (15.1 g, 106.53 mmol, 1.2 equiv.) dropwise with stirring at 0° C. The resulting solution was stirred for 2 h at 0° C. and the quenched by the addition of water. The resulting solution was extracted with DCM and concentrated under reduced pressure. The residue was applied onto a silica gel column with ethyl acetate/petroleum ether (1:3 to 1:1) to afford 5-(methylsulfanyl)-2H,3H-pyrido[4,3-f][1,4]oxazepine (12 g, 69.6%) as a light yellow solid., MS-ESI: 195.1 [M+H]⁺.

### Step 6: 5-(methylsulfanyl)-2H,3H-pyrido[4,3-f][1,4]oxazepine

To a solution of 5-(methylsulfanyl)-2H,3H-pyrido[4,3-f] [1,4] oxazepine (5.0 g, 25.74 mmol, 1.0 equiv.) in EtOH (100.0 mL), was added NH₂NH₂.H₂O (6.4 g, 128.65 mmol, 5.0 equiv.). The resulting solution was stirred for 14 h at 80° C. and then concentrated under reduced pressure to afford (5Z)-2H,3H,4H-pyrido[4,3-f] [1,4] oxazepin-5-ylidenehydrazine (4.0 g, 87.2%) as a yellow solid. MS-ESI: 179.1 [M+H]⁺.

### Step 7: ethyl 3-[[2-(tert-butoxy)-2-oxoethyl] amino]benzoate

To a solution of tricaine (20.0 g, 121.07 mmol, 1.0 equiv.) in ACN (200.0 mL), were added 2-bromoacetate (35.4 g, 181.61 mmol, 1.5 equiv.) and K₂CO₃ (33.5 g, 242.14 mmol, 2.0 equiv.). The resulting solution was stirred for 14 h at 60° C. and diluted with water. The resulting solution was extracted with DCM and concentrated under reduced pressure. The residue was applied onto a silica gel column with ethyl acetate/petroleum ether (1:30 to 1:15) to afford ethyl 3-[[2-(tert-butoxy)-2-oxoethyl]amino]benzoate (30 g, 88.7%) as light yellow oil. MS-ESI: 280.2 [M+H]⁺.

### Step 8: [[3-(ethoxycarbonyl)phenyl]amino]acetic acid

To a solution of ethyl 3-[[2-(tert-butoxy)-2-oxoethyl]amino]benzoate (40.0 g, 143.20 mmol, 1.0 equiv.) in DCM (150.0 mL), was added HCl/1,4-dioxane (150.0 mL). The resulting solution was stirred for 14 h at room temperature and then concentrated under reduced pressure to afford [[3-(ethoxycarbonyl)phenyl]amino]acetic acid (30 g, 93.9%) as a light yellow solid. MS-ESI: 224.1 [M+H]⁺.

### Step 9: ethyl 3-[([N′-[(5Z)-2H,3H,4H-pyrido[4,3-f][1,4]oxazepin-5-ylidene]hydrazinecarbonyl]methyl)amino]benzoate

To a solution of 2H,3H-pyrido[4,3-f][1,4]oxazepin-5-ylhydrazine (20.0 g, 112.24 mmol, 1.0 equiv.) in THF (300.0 mL), were added TEA (22.7 g, 224.47 mmol, 2.0 equiv.), [[3-(ethoxycarbonyl)phenyl]amino]acetic acid (25.1 g, 112.24 mmol, 1.0 equiv.) and HATU (51.2 g, 134.68 mmol, 1.2 equiv.). The resulting solution was stirred for 6 h at room temperature and concentrated under reduced pressure to afford ethyl 3-[([N′-[(5Z)-2H,3H,4H-pyrido[4,3-f][1,4]oxazepin-5-ylidene]hydrazinecarbonyl]methyl)amino]benzoate (40 g, crude) as yellow oil. MS-ESI: 384.2 [M+H]⁺.

### Step 10: ethyl 3-(((5,6-dihydropyrido[4,3-f][1,2,4]triazolo[4,3-d][1,4]oxazepin-3-yl)methyl)amino)benzoate

To a solution of ethyl 3-[([N′-[(5Z)-2H,3H,4H-pyrido[4,3-f][1,4]oxazepin-5-ylidene]hydrazinecarbonyl]methyl)amino]benzoate (40.0 g, 104.33 mmol, 1.0 equiv.) in EtOH (200.0 mL), was added AcOH (25.1 g, 417.12 mmol, 4.0 equiv.). The resulting solution was stirred for 5 h at 80° C. and then concentrated under reduced pressure. The residue was dissolved in DCM (200 mL), the resulting mixture was washed with brine and concentrated under reduced pressure. The residue was applied onto a silica gel column with petroleum ether/EtOAc (1:100) to afford ethyl 3-(((5,6-dihydropyrido[4,3-f][1,2,4]triazolo[4,3-d][1,4]oxazepin-3-yl)methyl)amino)benzoate (30 g, 78.7%) as yellow oil. MS-ESI: 366.2 [M+H]⁺.

### Step 11: 3-(((5,6-dihydropyrido[4,3-f][1,2,4]triazolo[4,3-d][1,4]oxazepin-3-yl)methyl)amino)benzoic acid

To a solution of ethyl 3-(((5,6-dihydropyrido[4,3-f][1,2,4]triazolo[4,3-d][1,4]oxazepin-3-yl)methyl)amino)benzoate (30.0 g, 82.10 mmol, 1.0 equiv.) in MeOH/water (200.0 mL/30.0 mL), was added NaOH (13.1 g, 328.41 mmol, 4.0 equiv.). The resulting solution was stirred for 14 h at room temperature and diluted with 200 mL of water. The pH value of the solution was adjusted to 5 with HCl aqueous. The solids were collected by filtration and dried in an oven under reduced pressure to afford 3-(((5,6-dihydropyrido[4,3-f][1,2,4]triazolo[4,3-d][1,4]oxazepin-3-yl)methyl)amino)benzoic acid (20 g, 72.2%) as a yellow solid. MS-ESI: 338.1 [M+H]⁺. ¹H NMR (400 MHz, DMSO-d₆) δ 8.51 (s, 1H), 8.36 (s, 2H), 7.35 (s, 1H), 7.24-7.17 (m, 2H), 6.99-6.97 (m, 1H), 4.70-4.56 (m, 6H).

### Example 2: N-((2,3-dihydrobenzofuran-7-yl)methyl)-3-(((4-methyl-5-(pyrimidin-4-yl)-4H-1,2,4-triazol-3-yl)methyl)amino)benzamide (Compound 1)

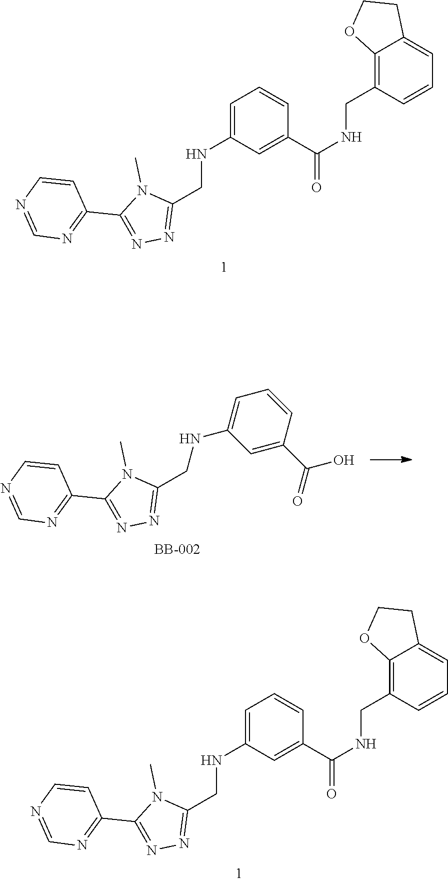 [SMILES: Cn1c(CNc2cccc(C(=O)NCc3cccc4c3OCC4)c2)nnc1-c1ccncn1 ; Cn1c(CNc2cccc(C(=O)O)c2)nnc1-c1ccncn1 ; Cn1c(CNc2cccc(C(=O)NCc3cccc4c3OCC4)c2)nnc1-c1ccncn1]

To a solution of 3-([[4-methyl-5-(pyrimidin-4-yl)-1,2,4-triazol-3-yl]methyl]amino)benzoic acid (312.0 mg, 1.0 mmol, 1.0 equiv.) and DIEA (389.8 mg, 3.0 mmol, 3.0 equiv.) in DMF (15 mL), were added EDCI (289.1 mg, 1.5 mmol, 1.5 equiv.) and HOBT (203.8 mg, 1.5 mmol, 1.5 equiv.) at room temperature. This was followed by the addition of 1-(2,3-dihydro-1-benzofuran-7-yl)methanamine (150.0 mg, 1.0 mmol, 1.0 equiv.). The resulting mixture was stirred for 3 hours at room temperature and then concentrated under vacuum. The residue was applied onto a silica gel column with DCM/MeOH (8:1) to afford the crude product which was further purified by Prep-HPLC with following conditions: Column: YMC-Actus Triart C18, 30*250, 5 um; Mobile Phase A: Water (10 MMOL/L NH₄HCO₃+0.1% NH₃.H₂O), Mobile Phase B: ACN; Flow rate: 60 mL/min; Gradient: 22 B to 42 B in 10 min; 254/210 nm; RT1:10.33. This afforded N-(2,3-dihydro-1-benzofuran-7-ylmethyl)-3-([[4-methyl-5-(pyrimidin-4-yl)-1,2,4-triazol-3-yl]methyl]amino)benzamide (186.4 mg, 42.0%) as a white solid. MS-ES: 442 [M+H]⁺. ¹H NMR (300 MHz, DMSO-d₆) δ 9.29 (s, 1H), 8.93 (d, J=5.1 Hz, 1H), 8.21-8.19 (i, 1H), 7.26-7.07 (m, 5H), 6.97-6.93 (m, 1H), 6.81-6.76 (m, 1H), 4.66 (s, 2H), 4.57 (t, J=8.7 Hz, 2H), 4.51 (s, 2H), 4.18 (s, 3H), 3.20 (t, J=8.7 Hz, 2H).

The analogs in Table 4 below were prepared using similar methods as for Compound 1 above.

TABLE 4

| | Building | |
|---|---|---|
| # | Block | Anal. data |
| 2 | BB-002 | MS-ESI: 418 [M + H]⁺. |
| | | ¹H NMR (300 MHz, DMSO-d₆) δ 9.35 (s, 1H), 8.98 (d, J = 4.5 Hz, 1H), 8.90-8.87 |
| | | (m, 1H), 8.19 (d, J = 5.4 Hz, 1H), 7.40-7.34 (m, 1H), 7.25-7.10 (m, 6H), 6.97-6.93 |
| | | (m, 1H), 6.55-6.51 (m, 1H), 4.59 (d, J =5.7 Hz, 2H), 4.47 (t, J = 6.0 Hz, 2H), 4.08 |
| | | (s, 3H). |
| 3 | BB-002 | MS-ESI: 442 [M + H]⁺. |
| | | 1H NMR (400 MHz, DMSO-d₆) δ 9.34 (s, 1H), 8.98 (d, J = 5.2 Hz, 1H), 8.68 (d, |
| | | J = 8.4 Hz, 1H), 8.19-8.18 (m, 1H), 7.27 (s, 1H), 7.17-7.12 (m, 2H), 6.80-6.78 (m, |
| | | 1H), 5.30-5.26 (m, 1H), 4.59 (s, 2H), 4.31-4.23 (m, 2H), 4.08 (s, 3H), 2.09-2.05 |
| | | (m, 2H). |
| 4 | BB-002 | MS-ESI: 469 [M + H]⁺. |
| | | 1H NMR (300 MHz, DMSO-d₆) δ 9.33 (s, 1H), 9.00-8.96 (m, 2H), 8.61 (d, J = 3.9 |
| | | Hz, 1H), 8.18-8.16 (m, 1H), 7.96-7.93 (m, 1H), 7.73-7.69 (m, 1H), 7.26-7.12 (m, |
| | | 3H), 6.94-6.92 (m, 1H), 4.65-4.63 (m, 2H), 4.58 (s, 2H), 4.07 (s, 3H). |
| 5 | BB-002 | MS-ESI: 448 [M + H]⁺. |
| | | 1H NMR (400 MHz, DMSO-d₆) δ 9.32 (s, 1H), 8.95 (d, J = 5.2 Hz, 1H), 8.22-8.21 |
| | | (m, 1H), 7.33-7.29 (m, 1H), 7.26-7.21 (m, 2H), 7.16-7.13 (m, 1H), 6.98-6.95 (m, |
| | | 1H), 6.75-6.71 (m, 1H), 6.68 (d, J = 2.8 Hz, 1H), 4.68 (s, 2H), 4.54 (s, 2H), 4.20 |
| | | (s, 3H), 3.79 (s, 3H). |
| 6 | BB-002 | MS-ESI: 460 [M + H]⁺. |
| | | 1H NMR (400 MHz, DMSO-d₆) δ 9.34 (s, 1H), 8.97 (d, J = 5.2 Hz, 1H), 8.19-8.17 |
| | | (m, 1H), 7.82 (t, J = 4.4 Hz, 1H), 7.27-7.23 (m, 1H), 7.16-7.10 (m, 2H), 7.03 (d, |
| | | J = 7.6 Hz, 1H), 6.87-6.85 (m, 1H), 6.67 (d, J = 8.4 Hz, 2H), 4.57-4.56 (m, 2H), |
| | | 4.44 (d, J = 4.4 Hz, 2H), 4.08 (s, 3H), 3.79 (s, 6H). |
| 7 | BB-002 | MS-ESI: 444 [M + H]⁺. |
| | | 1H NMR (400 MHz, DMSO-d₆) δ 9.34 (s, 1H), 8.98 (d, J = 5.2 Hz, 1H), 8.65-8.63 |
| | | (m, 1H), 8.19-8.18 (m, 1H), 7.27 (s, 1H), 7.26-7.16 (m, 4H), 6.98-6.91 (m, 3H), |
| | | 4.59 (s, 2H), 4.43 (d, J = 5.6 Hz, 2H), 4.10-4.05 (m, 2H), 4.08 (s, 3H), 1.36 (t, J = |
| | | 7.2 Hz, 3H). |
| 8 | BB-002 | MS-ESI: 470 [M + H]⁺. |
| | | 1H NMR (400 MHz, DMSO-d₆) δ 9.34 (s, 1H), 8.98 (d, J = 5.2 Hz, 1H), 8.23 (s, |
| | | 1H), 8.19-8.17 (m, 1H), 7.42-7.40 (m, 2H), 7.34-7.30 (m, 2H), 7.22-7.14 (m, 3H), |
| | | 7.08 (d, J = 7.6 Hz, 1H), 6.93 (d, J = 7.6 Hz, 1H), 4.58 (s, 2H), 4.08 (s, 3H), 3.77- |
| | | 3.72 (m, 4H), 2.34-2.32 (m, 2H), 1.99-1.93 (m, 2H). |
| 9 | BB-001 | MS-ESI: 416 [M + H]⁺. |
| | | 1H NMR (400 MHz, DMSO-d₆) δ 8.78-8.76 (m, 1H), 8.67 (d, J = 4.4 Hz, 2H), |
| | | 8.05-8.04 (m, 1H), 7.91-7.90 (m, 2H), 7.49-7.47 (m, 1H), 7.19-7.13 (m, 3H), 6.94- |
| | | 6.91 (m, 1H), 6.82-6.80 (m, 1H), 4.50 (brs, 2H), 4.36 (d, J = 6.47 Hz, 2H), 3.91 (s, |
| | | 3H). |
| 10 | BB-002 | MS-ESI: 435.18 [M + H]⁺. |
| 11 | BB-002 | MS-ESI: 414.21 [M + H]⁺. |
| 12 | BB-002 | MS-ESI: 428.23 [M + H]⁺. |
| 13 | BB-002 | MS-ESI: 434.15 [M + H]⁺. |
| 14 | BB-002 | MS-ESI: 414.21 [M + H]⁺. |
| 15 | BB-002 | MS-ESI: 435.21 [M + H]⁺. |
| 16 | BB-002 | MS-ESI: 454.25 [M + H]⁺. |
| 17 | BB-002 | MS-ESI: 428.23 [M + H]⁺. |
| 18 | BB-002 | MS-ESI: 428.23 [M + H]⁺. |
| 19 | BB-002 | MS-ESI: 498.19 [M + H]⁺. |
| 20 | BB-002 | MS-ESI: 475.17 [M + H]⁺. |
| 21 | BB-002 | MS-ESI: 442.25 [M + H]⁺. |
| 22 | BB-002 | MS-ESI: 414.21 [M + H]⁺. |
| 23 | BB-002 | MS-ESI: 443.24 [M + H]⁺. |
| 24 | BB-002 | MS-ESI: 450.21 [M + H]⁺. |
| 25 | BB-002 | MS-ESI: 428.23 [M + H]⁺. |
| 26 | BB-002 | MS-ESI: 427.19 [M + H]⁺. |
| 27 | BB-002 | MS-ESI: 443.23 [M + H]⁺. |
| 28 | BB-002 | MS-ESI: 468.18 [M + H]⁺. |
| 29 | BB-002 | MS-ESI: 448.17 [M + H]⁺. |
| 30 | BB-002 | MS-ESI: 455.23 [M + H]⁺. |
| 31 | BB-002 | MS-ESI: 441.21 [M + H]⁺. |
| 32 | BB-002 | MS-ESI: 455.23 [M + H]⁺. |
| 33 | BB-001 | MS-ESI: 415.19 [M + H]⁺. |
| 34 | BB-001 | MS-ESI: 428.23 [M + H]⁺. |
| 36 | BB-002 | MS-ESI: 472.2 [M + H]⁺. |
| 37 | BB-002 | MS-ESI: 454.2 [M + H]⁺. |
| 38 | BB-002 | MS-ESI: 454.2 [M + H]⁺. |
| 39 | BB-002 | MS-ESI: 444.2 [M + H]⁺. |
| 40 | BB-002 | MS-ESI: 440.2 [M + H]⁺. |
| 41 | BB-002 | MS-ESI: 475.2 [M + H]⁺. |
| 42 | BB-002 | MS-ESI: 467.2 [M + H]⁺. |
| 43 | BB-002 | MS-ESI: 425.2 [M + H]⁺. |
| 44 | BB-002 | MS-ESI: 454.2 [M + H]⁺. |
| 45 | BB-002 | MS-ESI: 454.2 [M + H]⁺. |
| 46 | BB-002 | MS-ESI: 444.2 [M + H]⁺. |
| 47 | BB-002 | MS-ESI: 4335.1 [M + H]⁺. |
| 49 | BB-002 | MS-ESI: 460.2 [M + H]⁺. |
| 51 | BB-002 | MS-ESI: 404.2 [M + H]⁺. |
| 52 | BB-002 | MS-ESI: 404.2 [M + H]⁺. |
| 53 | BB-002 | MS-ESI: 492.2 [M + H]⁺. |
| 54 | BB-002 | MS-ESI: 425.2 [M + H]⁺. |
| 55 | BB-002 | MS-ESI: 467..2 [M + H]⁺. |
| 56 | BB-002 | MS-ESI: 442.2 [M + H]⁺. |
| 57 | BB-002 | MS-ESI: 441.1 [M + H]⁺. |
| 58 | BB-002 | MS-ESI: 467.2 [M + H]⁺. |
| 59 | BB-002 | MS-ESI: 403.2 [M + H]⁺. |
| 60 | BB-002 | MS-ESI: 443.2 [M + H]⁺. |
| 61 | BB-002 | MS-ESI: 421.2 [M + H]⁺. |
| 62 | BB-002 | MS-ESI: 466.2 [M + H]⁺. |

### Example 3: 2-(((4-propyl-5-(pyrimidin-4-yl)-4H-1,2,4-triazol-3-yl)methyl)amino)-N-(2-(trifluoromethyl)benzyl)isonicotinamide (Compound 35)

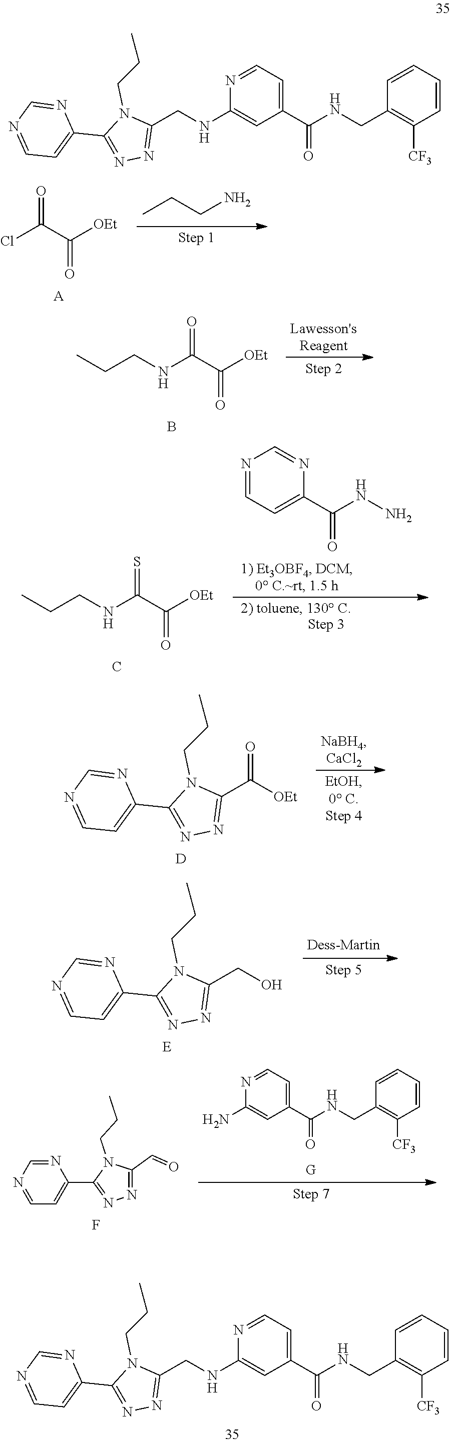

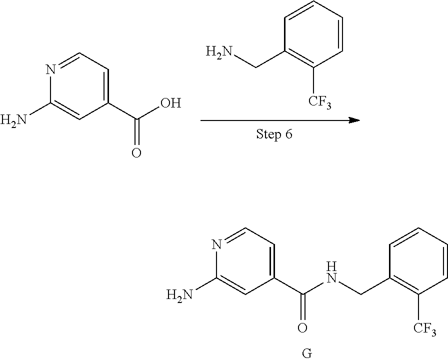 [SMILES: Nc1cc(C(=O)O)ccn1 ; NCc1ccccc1C(F)(F)F ; Nc1cc(C(=O)NCc2ccccc2C(F)(F)F)ccn1]

### Step 1: ethyl 2-oxo-2-(propylamino)acetate

To a stirred solution of propan-1-amine (25.0 g, 423.73 mmol, 1.2 equiv.) and triethylamine (92.7 g, 915.58 mmol, 2.5 equiv.) in DCM (1.5 L), was added ethyl chloroglyoxylate (50.0 g, 366.22 mmol, 1.0 equiv.) dropwise at 0° C. The resulting mixture was stirred for 1 h at 0° C. and then quenched by the addition of water. The mixture was extracted with DCM and concentrated under vacuum to afford ethyl 2-oxo-2-(propylamino)acetate (25 g) as a brown yellow oil. MS-ESI: 160.1 [M+H]⁺.

### Step 2: ethyl 2-(propylamino)-2-thioxoacetate

To a solution of ethyl 2-oxo-2-(propylamino)acetate (30.0 g, 188.68 mmol, 1.0 equiv.) in toluene (500.0 mL), was added Lawesson reagent (38.5 g, 95.33 mmol, 0.5 equiv.). The resulting mixture was stirred for overnight at 90° C. and then concentrated under reduced pressure. The residue was purified by silica gel column chromatography, eluted with petroleum ether/EtOAc (1:1) to afford ethyl 2-(propylamino)-2-thioxoacetate (12 g) as a yellow oil. MS-ESI: 176.1 [M+H]⁺.

### Step 3: ethyl 4-propyl-5-(pyrimidin-4-yl)-4H-1,2,4-triazole-3-carboxylate

To a stirred solution of ethyl 2-(propylamino)-2-thioxoacetate (14.5 g, 82.86 mmol, 1.0 equiv.) in DCM (100 mL), was added a solution of Et₃OBF₄ (23.2 g, 122.29 mmol, 1.5 equiv.) in DCM (200.0 mL) dropwise at 0° C. The resulting mixture was stirred for 1.5 h at room temperature under nitrogen atmosphere. The solution was washed with brine and concentrated under vacuum. The residue was dissolved in toluene (400.0 mL), and to the mixture was added pyrimidine-4-carbohydrazide (11.3 g, 81.53 mmol, 1.0 equiv.). The resulting mixture was stirred for additional overnight at 130° C. and concentrated under reduced pressure. The residue was purified by silica gel column chromatography, eluted with DCM/MeOH (20:1) to afford ethyl 4-propyl-5-(pyrimidin-4-yl)-4H-1,2,4-triazole-3-carboxylate (13 g) as a yellow solid. MS-ESI: 262.1 [M+H]⁺.

### Step 4: (4-propyl-5-(pyrimidin-4-yl)-4H-1,2,4-triazol-3-yl)methanol

To a stirred solution of ethyl 4-propyl-5-(pyrimidin-4-yl)-4H-1,2,4-triazole-3-carboxylate (13.5 g, 51.66 mmol, 1.0 equiv.) in EtOH (160.0 mL), were added CaCl₂ (1.7 g, 15.44 mmol, 0.3 equiv.) and NaBH₄ (2.4 g, 61.74 mmol, 1.2 equiv.) in portions at 0° C. The final reaction mixture was stirred for 3 h at room temperature and then quenched by the addition of water. After concentration under vacuum, the residue was purified by silica gel column chromatography, eluted with DCM/MeOH (12:1) to afford (4-propyl-5-(pyrimidin-4-yl)-4H-1,2,4-triazol-3-yl)methanol (9.0 g) as a white solid. MS-ESI: 220.1 [M+H]⁺.

### Step 5: 4-propyl-5-(pyrimidin-4-yl)-4H-1,2,4-triazole-3-carbaldehyde

To a stirred solution of [4-propyl-5-(pyrimidin-4-yl)-1,2,4-triazol-3-yl]methanol (400.0 mg, 1.82 mmol, 1.0 equiv.) in DCM (5.0 mL), was added Dess-Martin reagent (2321.4 mg, 5.47 mmol, 3.0 equiv.). The resulting mixture was stirred for 2 hours at room temperature under atmosphere of nitrogen and ten diluted with water. The resulting mixture was extracted with DCM, washed with brine, dried over anhydrous Na₂SO₄ and concentrated under reduced pressure. The residue was purified by silica gel column chromatography, eluted with DCM/MeOH (8:1) to afford 4-propyl-5-(pyrimidin-4-yl)-1,2,4-triazole-3-carbaldehyde (250 mg, 63.1%) as a yellow solid. MS-ESI: 218.1 [M+H]⁺.

### Step 6: 2-amino-N-(2-(trifluoromethyl)benzyl)isonicotinamide

To a stirred solution of 2-aminopyridine-4-carboxylic acid (500.0 mg, 3.62 mmol, 1.0 equiv.) and EDCI (1040.9 mg, 5.43 mmol, 1.5 equiv.) in DMF (5.0 mL), were added DIEA (1403.5 mg, 10.86 mmol, 3.0 equiv.) and HOBT (733.7 mg, 5.43 mmol, 1.5 equiv.). This was followed by the addition of 1-[2-(trifluoromethyl)phenyl]methanamine (760.8 mg, 4.34 mmol, 1.2 equiv.). The resulting mixture was stirred for overnight at room temperature and then concentrated under vacuum. The residue was purified by silica gel column chromatography, eluted with DCM/MeOH (10:1) to afford 2-amino-N-[[2-(trifluoromethyl)phenyl]methyl]pyridine-4-carboxamide (400 mg, 37.4%) as light yellow oil. MS-ESI: 296.1 [M+H]⁺.

### Step 7: 2-(((4-propyl-5-(pyrimidin-4-yl)-4H-1,2,4-triazol-3-yl)methyl)amino)-N-(2-(trifluoromethyl)benzyl)isonicotinamide

To a stirred solution of 4-propyl-5-(pyrimidin-4-yl)-1,2,4-triazole-3-carbaldehyde (270.0 mg, 1.24 mmol, 1.0 equiv.) and 2-amino-N-[[2-(trifluoromethyl)phenyl]methyl]pyridine-4-carboxamide (440.4 mg, 1.49 mmol, 1.2 equiv.) in THF (5.0 mL), was added tetraethoxytitanium (850.6 mg, 3.73 mmol, 3.0 equiv.) under nitrogen. The resulting mixture was stirred for 12 hours at 70° C. under atmosphere of nitrogen. After cooling down to room temperature, NaBH₄ (141.1 mg, 3.73 mmol, 3.0 equiv.) was added. The resulting mixture was stirred for additional 2 hours at room temperature under atmosphere of nitrogen and then quenched by the addition of water. The resulting mixture was extracted with EtOAc, washed with brine, dried over anhydrous Na₂SO₄ and concentrated under reduced pressure. The residue was purified by Prep-HPLC with following conditions: Column: YMC-Actus Triart C18, 30*250, 5 um; Mobile Phase A: Water (10 MMOL/L NH₄HCO₃+0.1% NH₃.H₂O), Mobile Phase B: ACN; Flow rate: 60 mL/min; Gradient: 32 B to 62 B in 7 min; 210/254 nm; RT1: 6.42. This resulted in 2-([[4-propyl-5-(pyrimidin-4-yl)-1,2,4-triazol-3-yl]methyl]amino)-N-[[2-(trifluoromethyl)phenyl]methyl]pyridine-4-carboxamide (12.2 mg, 2.0%) as a white solid. MS-ESI: 497 [M+H]⁺. ¹H NMR (400 MHz, DMSO-d₆) δ 9.34 (s, 1H), 9.20-9.18 (m, 1H), 8.97 (d, J=5.2 Hz, 1H), 8.22-8.20 (m, 1H), 8.15 (d, J=5.2 Hz, 1H), 7.76-7.74 (m, 1H), 7.67-7.65 (m, 1H), 7.51-7.49 (m, 3H), 7.05 (s, 1H), 7.01-6.99 (m, 1H), 4.84-4.83 (m, 2H), 4.65-4.63 (m, 2H), 4.54 (t, J=7.6 Hz, 2H), 1.74-1.70 (m, 2H), 0.80 (t, J=7.6 Hz, 3H).

### Example 5. Synthesis of BB-013

BB-013 was prepared as described in the scheme below:

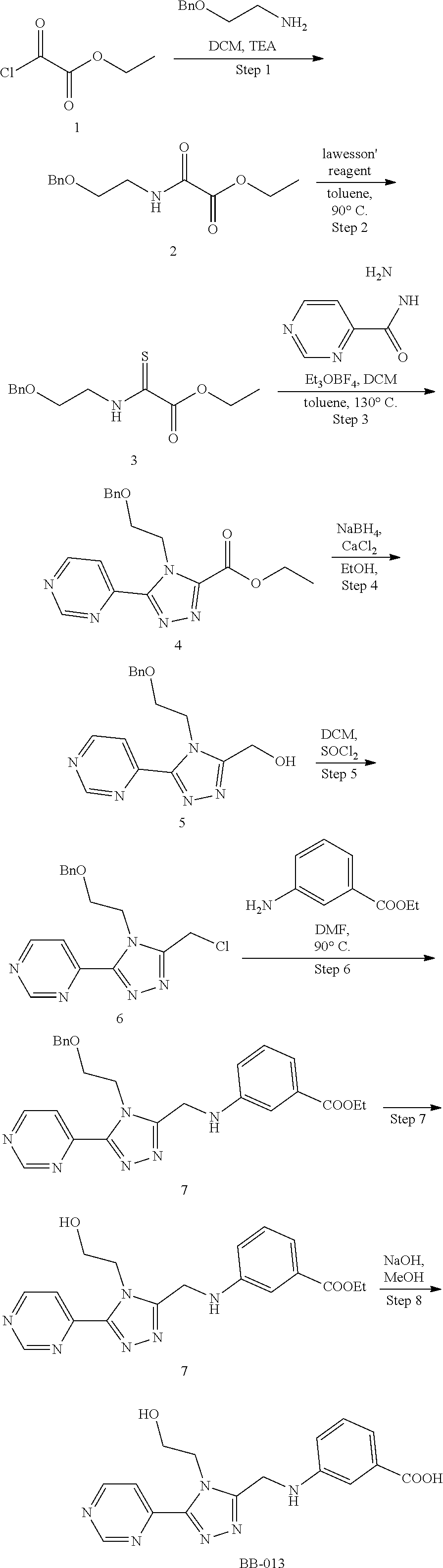

### Example 6. Synthesis of BB-011

BB-011 was prepared as described in the scheme below:

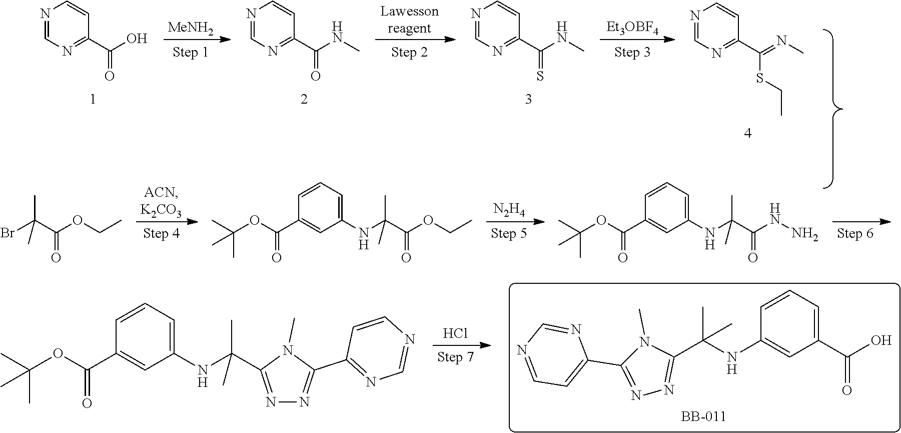

### Example 7. GRK2 Inhibition Assay

Enzyme GRK2 (1 nM final concentration) was diluted in 25 mMv HEPES, 10 mM MgCl2, 2 mM DTT, 0.01% Tween-20, and 1 mM EGTA. Then the GRK2 mixture was added into ProxiPlate-384 white plate and pre-incubated for 30 min with test compounds at room temperature. ATP (7 μM final concentration) and Ulight TopoIIα (50 nM final concentration) were added into the assay plate to initiate the reaction and the mixture was incubated at room temperature for 90 min. Then, Eu anti-TopoIIα (0.12 nM final concentration), BSA (0.01% final concentration), and EDTA (11 mMv final concentration) in LANCE assay buffer were added into each well. After 60 min incubation time at room temperature, TR-FRET signal was measured by EnVision plate reader.

Results: As shown in Table 5 below, compounds of the disclosure were found to inhibit GRK2.

### Example 8. ADPGlo GRK2 Inhibition Assay

Assay: GRK2 (7.5 nM) was incubated with ATP (10 μM) and GRKtide (0.3 mg/mL) in 5 μL of assay buffer (see above) for 180 min at room temperature. HTS was performed using 1 μM compound.

Compounds were dissolved in 100% DMSO, serially diluted tree-fold from 100 μM concentration to 46 nM and transferred (50 nL) into assay ready plate.

Materials: GRK2 was purchased from SignalChem (Cat #A14-10G, Lot #X⁶⁴⁵-3). Substrate GRKtide was from SignalChem (Cat #G46-58, Lot #R339-6). ADP-Glo Kinase Assay from Promega (Cat #V9102). Assay buffer consisted of 25 mMv HEPES (pH7.5), 10 mMv MgCl2, 0.01% Tween-20, 1 mM DDT. 384-well white plates were from Greiner Bio-Rad (Item #784075).

HTS protocol: Take 384 well plate with 50 nL of compound in columns 3-22/DMSO solution 1-2,23-24. Add 2.5 μL assay buffer to columns 23 and 24 using Thermo Scientific Multidrop Combi Dispenser. Add 2.5 μL of 2× enzyme solution (15 nM in 1× assay buffer) using Thermo Scientific Multidrop Combi Reagent Dispenser to all columns except of 23 and 24. Incubate for 15 minutes. Fill plate with 2.5 μL of 2× substrate mix (20 μM ATP and 0.6 mg/nt GRKtide in Ix assay buffer) using Thermo Scientific Multidrop Combi Reagent Dispenser. Spin 15 seconds at 1000 rpm and incubate for 180 min at room temperature. Add 5 μL ADP-Glo reagent to all the wells. Spin 15 seconds at 1000 rpm and incubate for 40 min at room temperature. Add 10 μL Detection solution to all the wells. Incubate for 30 min at room temperature. Read plate in Luminescent modeon BMG PheraStar FSX (gain=3600).

Data Analysis: Data were analyzed in GraphPad Prism 8.0.2. Each HTS plate contains compounds in columns 3-22, controls (enzyme, no compound) in columns 1 and 2, and blanks (no enzyme) in columns 23 and 24. HTS percent inhibition was calculated for each compound from the signal in luminescence units and the mean of the plate controls and the mean of the plate blanks using the following equation: % Inhibition=100*(1−((signal-blank mean)/(control mean-blank mean))). At the final stage of data processing, we obtain dose-response curves, tables and SDF-files with the results of screening for each substance.

Results: As shown in Table 5 below, compounds of the disclosure were found to inhibit GRK2.

TABLE 5

| | GRK2 IC₅₀ | GRK2 IC₅₀ |
|---|---|---|
| | (nM) | (nM) |
| Compound | (Example 7) | (Example 8) |
| 1 | 4.7 | 27 |
| 2 | 56 | 49 |
| 3 | 10 | 18 |
| 4 | 28 | 36 |
| 5 | 5.4 | 19 |
| 6 | 19 | 44 |
| 7 | 33 | 30 |
| 8 | 170 | 140 |
| 9 | 5.8 | 12 |
| 10 | | 15 |
| 11 | | 14 |
| 12 | | 51 |
| 13 | | 9.1 |
| 14 | | |
| 15 | | 18 |
| 16 | | 37 |
| 17 | | 10 |
| 18 | | |
| 19 | | 17 |
| 20 | | 10 |
| 21 | | 67 |
| 22 | | 420 |
| 23 | | |
| 24 | | 61 |
| 25 | | |
| 26 | | 9.8 |
| 27 | | 12 |
| 28 | | |
| 29 | | 12 |
| 30 | | 15 |
| 31 | | 22 |
| 32 | | 22 |
| 33 | | 7.8 |
| 34 | | 10 |
| 35 | 4.5 | |
| 36 | | 0.5 |
| 37 | | 0.79 |
| 38 | | 0.96 |
| 39 | | 1.1 |
| 40 | | 1.7 |
| 41 | | 1.8 |
| 42 | | 2.1 |
| 43 | | 2.7 |
| 44 | | 2.8 |
| 45 | | 3 |
| 46 | | 3.3 |
| 47 | | 4.3 |
| 48 | | 4.4 |
| 49 | | 5.4 |
| 50 | | 5.5 |
| 51 | | 5.7 |
| 52 | | 5.7 |
| 53 | | 6.2 |
| 54 | | 6.3 |
| 55 | | 6.5 |
| 56 | | 7.3 |
| 57 | | 9.4 |
| 58 | | 9.5 |
| 59 | | 9.5 |
| 60 | | 9.6 |
| 61 | | 9.8 |
| 62 | | 10 |

### Example 9. Proliferation Assay

In order to identify compounds specific for GRK2, we generated two isogenic cell line pairs of the pancreatic cancer cell line PAXF1657, a pair of cells stably overexpressing GRK2 cDNA or the control empty vector and a cell line pair of PAXF1657 GRK2 knockout clonal cells versus non-targeting control clonal cells. The goal was to identify compounds that effectively impair proliferation in PAXF1657 empty vector or non-targeting control cells, but not in GRK2 cDNA overexpressing or GRK2 knockout cells, thereby identifying compounds superior to compounds of Okawa et al. J. Med. Chem. 2017, 60, 6942-6990.

Proliferation Assay: Cellular anti-proliferative activity of compounds was assessed by using the pancreatic cancer cell line, PAXF1657, stably overexpressing GRK2 cDNA or the control empty vector, as well as the parental urinary bladder cancer cell line, 5637. In addition, we also took advantage of GRK2 knockout cell lines that were generated via CRISPR. Cell lines were seeded into tissue culture treated, white-walled, 96-well plates at a density of 500 cells/well in RPMI1640 media supplemented with 10% H.I. FBS and penicillin/streptomycin. Plates were incubated overnight at 37° C., 5% CO₂ to allow cells to adhere to the wells. GRK2 inhibitors were added to the cells using a 10-point dilution series with a final concentration ranging from 30 μM-0.0002 μM in 0.3% DMSO. At the time of compound addition, a set of plates, that were not treated with compounds, were collected and cell viability was measured using CellTiter-Glo (Promega). CellTiter-Glo reagent was added to the designed plates and luminescence was measuring using a Biotek Synergy plate reader. The compound treated cells were incubated for 3 days at 37° C., 5% CO₂. The media was then aspirated from each well and replaced with fresh media containing GRK2 inhibitors. The compound treated cells were then incubated for an additional 4 days at 37° C., 5% CO₂. Cell viability was assessed and at end of the 7-day compound treatment by CellTiterGlo.

Results: As shown in Table 6, the compounds of the disclosure were found to inhibit proliferation the PAXF1657 control cell line significantly more than a PAXF1657 cell line that overexpresses GRK2. This indicates the inhibition of proliferation by the compounds of the disclosure is the result of GRK2 inhibition by the compounds. In contrast, compounds 133a, 115h, and 139c described in Okawa et al. as GRK2 inhibitors has similar effects on both cell lines indicating the inhibition of proliferation by these compounds is likely not the result of GRK2 inhibition. Compounds 133a, 115h, and 139c have the structure:

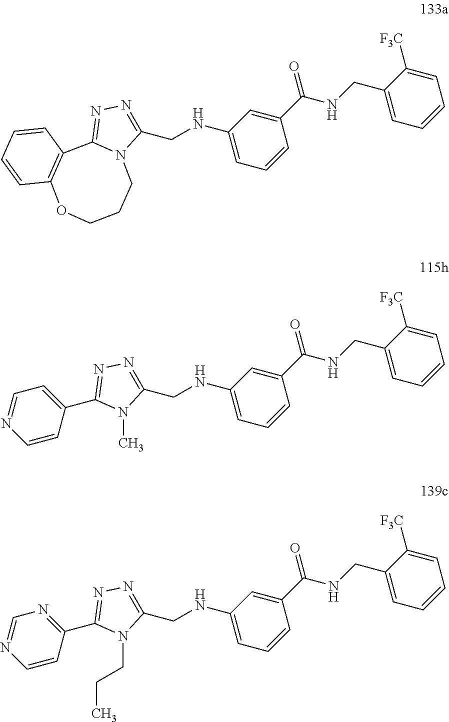 [SMILES: O=C(NCc1ccccc1C(F)(F)F)c1cccc(NCc2nnc3n2CCCOc2ccccc2-3)c1 ; Cn1c(CNc2cccc(C(=O)NCc3ccccc3C(F)(F)F)c2)nnc1-c1ccncc1 ; CCCn1c(CNc2cccc(C(=O)NCc3ccccc3C(F)(F)F)c2)nnc1-c1ccncn1]

TABLE 6

| | GI₅₀, (μM); | GI₅₀, (μM); | Ratio GRK2 |
|---|---|---|---|
| | PAXF1657 | PAXF1657 | over- |
| | control | stably | expressing |
| Com- | empty vector | overexpressing | cells/Empty |
| pound | cell line | GRK2 | vector cells |
| 133a | 0.14 | 0.24 | 1.71 |
| 115h | 8.91 | 10 | 1.12 |
| 139c | 0.84 | 1.47 | 1.75 |
| 35 | 1.30 | 3.22 | 2.49 |
| 10 | 1.58 | 13.67 | 8.65 |
| 1 | 1.97 | 24.30 | 12.34 |
| 2 | 1.79 | 20.50 | 11.46 |
| 3 | 1.16 | 8.61 | 7.42 |
| 4 | 0.79 | 4.59 | 5.79 |
| 12 | 0.97 | 8.01 | 8.24 |
| 5 | 1.51 | 10.26 | 6.79 |
| 6 | 2.08 | 12.49 | 6.01 |
| 15 | 4.01 | 17.73 | 4.42 |
| 7 | 3.32 | 14.25 | 4.29 |
| 8 | >30 | 22.66 | |
| 17 | 2.11 | 10.32 | 4.89 |
| 20 | 1.03 | 7.99 | 7.76 |
| 25 | >30 | 25.83 | |
| 26 | 0.68 | 4.18 | 6.15 |
| 29 | 1.24 | 5.01 | 4.04 |
| 33 | 1.33 | 17.71 | 13.27 |

### Example 10. Additional Compounds of the Disclosure

Additional compounds of the disclosure are provided in Table 7. The data in Table 7 also show that the compounds are effective GRK2 inhibitors and antiproliferative agents.

The enzymatic assays in Table 7 are as follows:
A: Enzymatic GRK2: IC50 (nM) (Example 8); and
B: Enzymatic GRK2: IC50 (nM) (Example 7).

The proliferation assays in Table 7 are as follows:
C: 7-day proliferation assay: IC50 GeoMean (uM) [Cell Line: PAXF1657 empty vector (EV) cell line] (Example 9); and
D: 7-day proliferation assay: % Minimum GeoMean (%) [Cell Line: PAXF1657 empty vector (EV) cell line] (Example 9).

TABLE 7
Additional Compounds and Data

| | Enzyme Assay | | Proliferation Assay | |
|---|---|---|---|---|
| Compound Structure | A | B | C | D |
| 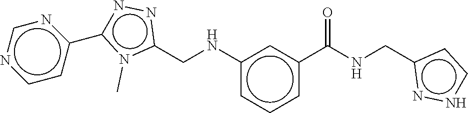 | | 39 | >10.0 | 70.6 |
| 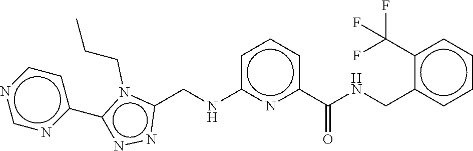 | | 200 | >10.0 | 34 |
| 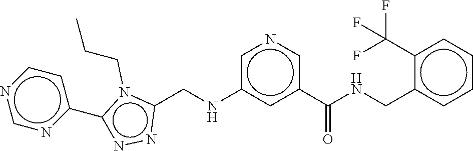 | | 30 | >30.0 | 85.6 |
| 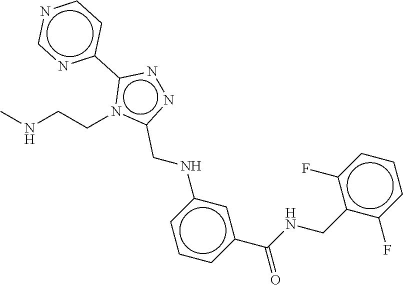 | | 1400 | | |
| 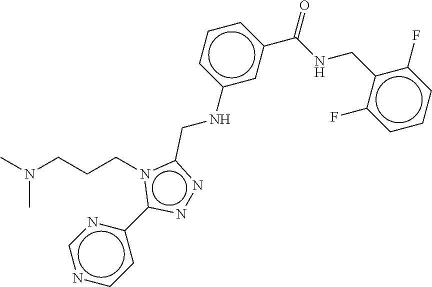 | | 170 | | |
| 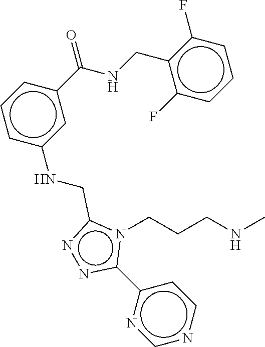 | | 660 | | |
| 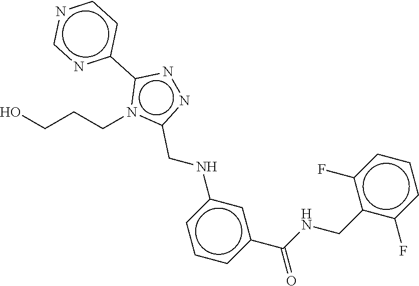 | | 47 | | |
| 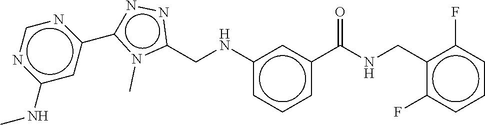 | | 4400 | | |
| 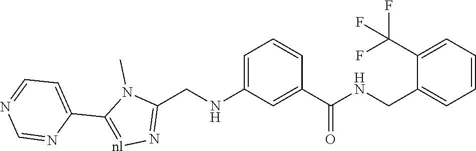 | 29 | | 1.15 | 13.1 |
| 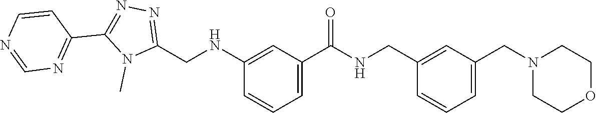 [SMILES: Cn1c(CNc2cccc(C(=O)NCc3cccc(CN4CCOCC4)c3)c2)nnc1-c1ccncn1] | | | >10.0 | 78.5 |
| 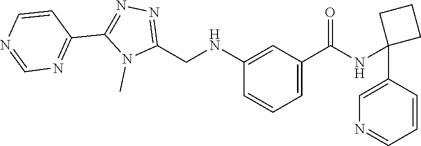 [SMILES: Cn1c(CNc2cccc(C(=O)NC3(c4cccnc4)CCC3)c2)nnc1-c1ccncn1] | | | >10.0 | 66.5 |
| 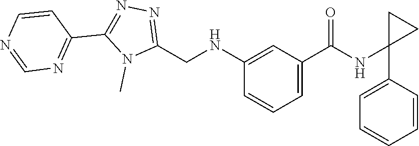 [SMILES: Cn1c(CNc2cccc(C(=O)NC3(c4ccccc4)CC3)c2)nnc1-c1ccncn1] | 34 | | >10.0 | 68.7 |
| 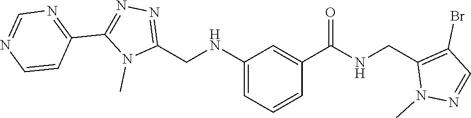 [SMILES: Cn1ncc(Br)c1CNC(=O)c1cccc(NCc2nnc(-c3ccncn3)n2C)c1] | 22 | | >10.0 | 26.5 |
| 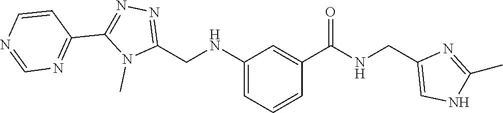 [SMILES: Cc1nc(CNC(=O)c2cccc(NCc3nnc(-c4ccncn4)n3C)c2)c[nH]1] | | | >10.0 | 71.7 |
| 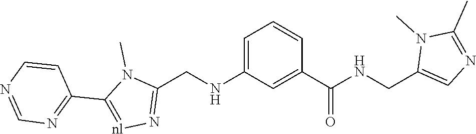 | | | >10.0 | 74.4 |
| 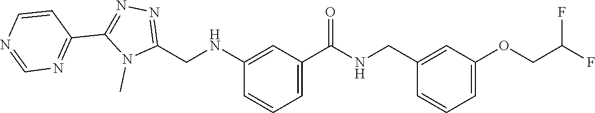 [SMILES: Cn1c(CNc2cccc(C(=O)NCc3cccc(OCC(F)F)c3)c2)nnc1-c1ccncn1] | 43 | | >10.0 | 102 |
| 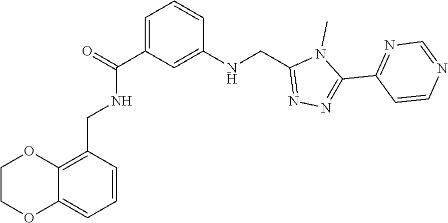 [SMILES: Cn1c(CNc2cccc(C(=O)NCc3cccc4c3OCCO4)c2)nnc1-c1ccncn1] | 15 | | 2.07 | 37.4 |
| 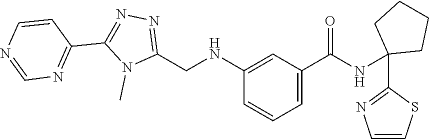 [SMILES: Cn1c(CNc2cccc(C(=O)NC3(c4nccs4)CCCC3)c2)nnc1-c1ccncn1] | | | >10.0 | 89.8 |
| 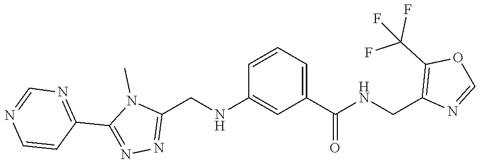 [SMILES: Cn1c(CNc2cccc(C(=O)NCc3ncoc3C(F)(F)F)c2)nnc1-c1ccncn1] | | | >10.0 | 74.2 |
| 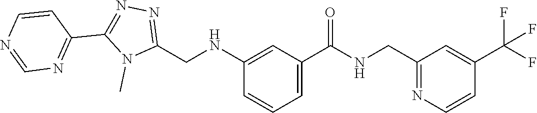 [SMILES: Cn1c(CNc2cccc(C(=O)NCc3cc(C(F)(F)F)ccn3)c2)nnc1-c1ccncn1] | | | >10.0 | 82.6 |
| 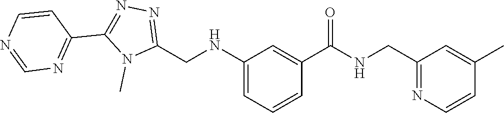 [SMILES: Cc1ccnc(CNC(=O)c2cccc(NCc3nnc(-c4ccncn4)n3C)c2)c1] | 430 | | >10.0 | 45.5 |
| 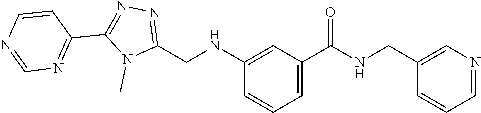 [SMILES: Cn1c(CNc2cccc(C(=O)NCc3cccnc3)c2)nnc1-c1ccncn1] | 540 | | >10.0 | 87.2 |
| 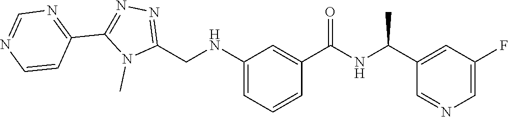 [SMILES: C[C@H](NC(=O)c1cccc(NCc2nnc(-c3ccncn3)n2C)c1)c1cncc(F)c1] | | | 2.3 | 41.4 |
| 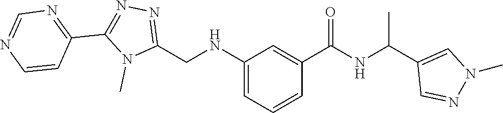 [SMILES: CC(NC(=O)c1cccc(NCc2nnc(-c3ccncn3)n2C)c1)c1cnn(C)c1] | 17 | | 0.19 | 47.6 |
| 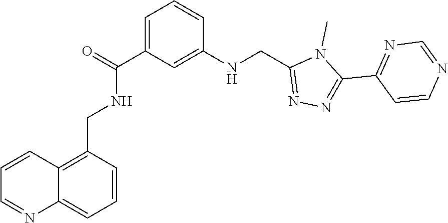 [SMILES: Cn1c(CNc2cccc(C(=O)NCc3cccc4ncccc34)c2)nnc1-c1ccncn1] | 11 | | >10.0 | 71.1 |
| 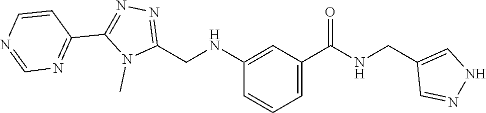 [SMILES: Cn1c(CNc2cccc(C(=O)NCc3cn[nH]c3)c2)nnc1-c1ccncn1] | | | >10.0 | 76.5 |
| 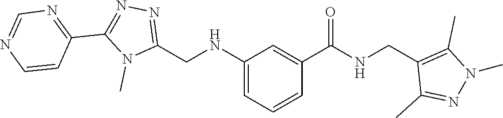 [SMILES: Cc1nn(C)c(C)c1CNC(=O)c1cccc(NCc2nnc(-c3ccncn3)n2C)c1] | 26 | | >10.0 | 54.1 |
| 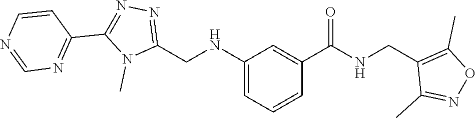 [SMILES: Cc1noc(C)c1CNC(=O)c1cccc(NCc2nnc(-c3ccncn3)n2C)c1] | 19 | | 0.173 | 62.5 |
| 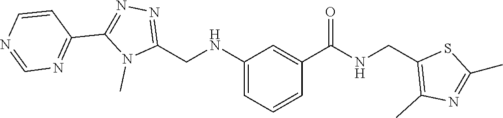 [SMILES: Cc1nc(C)c(CNC(=O)c2cccc(NCc3nnc(-c4ccncn4)n3C)c2)s1] | 26 | | >10.0 | 36.9 |
| 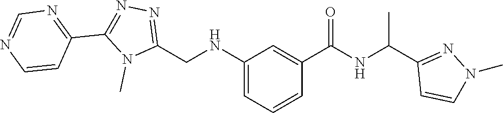 [SMILES: CC(NC(=O)c1cccc(NCc2nnc(-c3ccncn3)n2C)c1)c1ccn(C)n1] | 27 | | 2.15 | 71.3 |
| 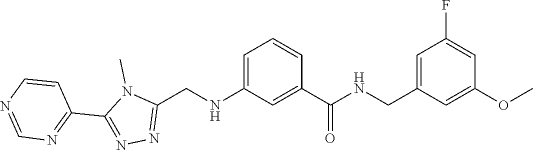 [SMILES: COc1cc(F)cc(CNC(=O)c2cccc(NCc3nnc(-c4ccncn4)n3C)c2)c1] | 54 | | >10.0 | 43.3 |
| 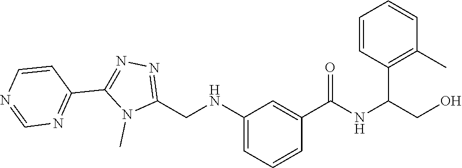 [SMILES: Cc1ccccc1C(CO)NC(=O)c1cccc(NCc2nnc(-c3ccncn3)n2C)c1] | 33 | | 8.66 | 46.8 |
| 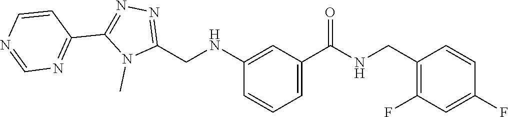 [SMILES: Cn1c(CNc2cccc(C(=O)NCc3ccc(F)cc3F)c2)nnc1-c1ccncn1] | 15 | | | 27.2 |
| 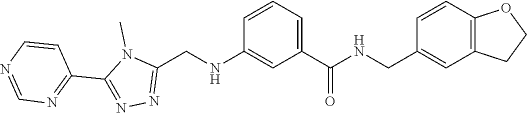 [SMILES: Cn1c(CNc2cccc(C(=O)NCc3ccc4c(c3)CCO4)c2)nnc1-c1ccncn1] | 16 | | 1.67 | 52.8 |
| 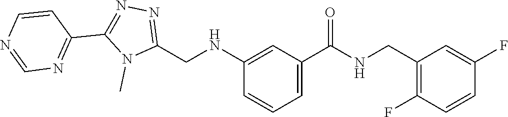 [SMILES: Cn1c(CNc2cccc(C(=O)NCc3cc(F)ccc3F)c2)nnc1-c1ccncn1] | 27 | | | 42 |
| 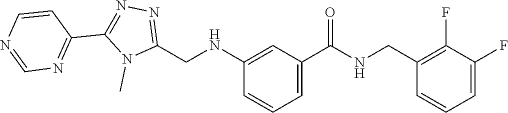 [SMILES: Cn1c(CNc2cccc(C(=O)NCc3cccc(F)c3F)c2)nnc1-c1ccncn1] | 16 | | >10.0 | 61.5 |
| 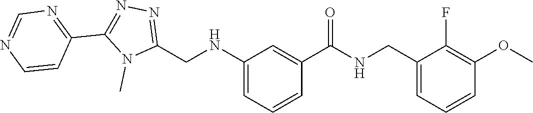 [SMILES: COc1cccc(CNC(=O)c2cccc(NCc3nnc(-c4ccncn4)n3C)c2)c1F] | 10 | | >10.0 | 64.5 |
| 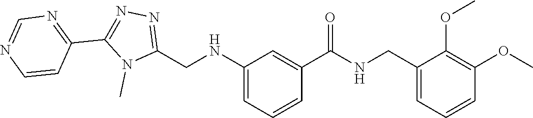 [SMILES: COc1cccc(CNC(=O)c2cccc(NCc3nnc(-c4ccncn4)n3C)c2)c1OC] | | | >10.0 | 45 |
| 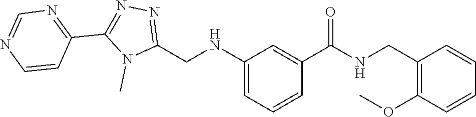 [SMILES: COc1ccccc1CNC(=O)c1cccc(NCc2nnc(-c3ccncn3)n2C)c1] | 20 | | 1.9 | 72.3 |
| 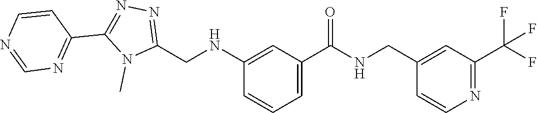 [SMILES: Cn1c(CNc2cccc(C(=O)NCc3ccnc(C(F)(F)F)c3)c2)nnc1-c1ccncn1] | | | 0.995 | 79 |
| 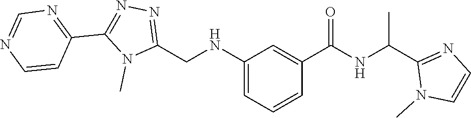 [SMILES: CC(NC(=O)c1cccc(NCc2nnc(-c3ccncn3)n2C)c1)c1nccn1C] | 24 | | 0.591 | 75.1 |
| 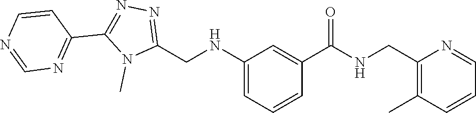 [SMILES: Cc1cccnc1CNC(=O)c1cccc(NCc2nnc(-c3ccncn3)n2C)c1] | 14 | | 1.61 | 33.5 |
| 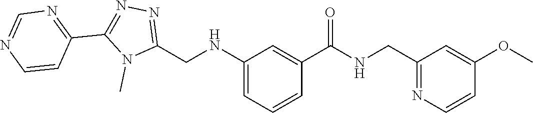 [SMILES: COc1ccnc(CNC(=O)c2cccc(NCc3nnc(-c4ccncn4)n3C)c2)c1] | | | 3.15 | 76.6 |
| 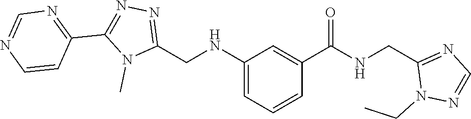 [SMILES: CCn1ncnc1CNC(=O)c1cccc(NCc2nnc(-c3ccncn3)n2C)c1] | 370 | | >10.0 | 75.1 |
| 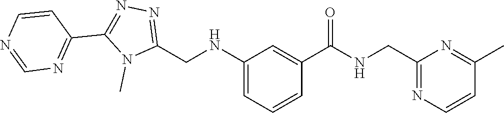 [SMILES: Cc1ccnc(CNC(=O)c2cccc(NCc3nnc(-c4ccncn4)n3C)c2)n1] | | | >10.0 | 63.4 |
| 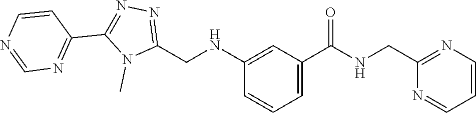 [SMILES: Cn1c(CNc2cccc(C(=O)NCc3ncccn3)c2)nnc1-c1ccncn1] | | | >10.0 | 94.9 |
| 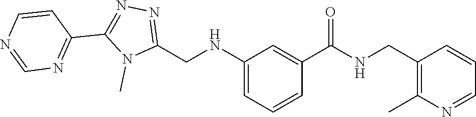 [SMILES: Cc1ncccc1CNC(=O)c1cccc(NCc2nnc(-c3ccncn3)n2C)c1] | 16 | | 1.49 | 36.2 |
| 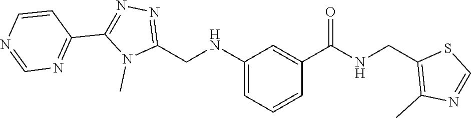 [SMILES: Cc1ncsc1CNC(=O)c1cccc(NCc2nnc(-c3ccncn3)n2C)c1] | 16 | | | 54.5 |
| 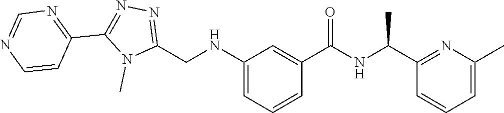 [SMILES: Cc1cccc([C@H](C)NC(=O)c2cccc(NCc3nnc(-c4ccncn4)n3C)c2)n1] | 95 | | | 52.2 |
| 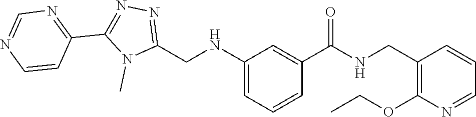 [SMILES: CCOc1ncccc1CNC(=O)c1cccc(NCc2nnc(-c3ccncn3)n2C)c1] | | | 3.61 | 61 |
| 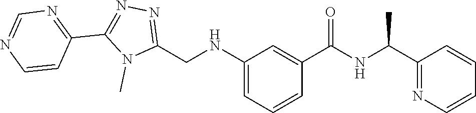 [SMILES: C[C@H](NC(=O)c1cccc(NCc2nnc(-c3ccncn3)n2C)c1)c1ccccn1] | | | 2.58 | 75.3 |
| 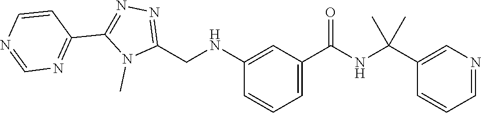 [SMILES: Cn1c(CNc2cccc(C(=O)NC(C)(C)c3cccnc3)c2)nnc1-c1ccncn1] | | | | 49.3 |
| 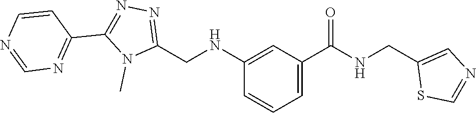 [SMILES: Cn1c(CNc2cccc(C(=O)NCc3cncs3)c2)nnc1-c1ccncn1] | | | >10.0 | 69.4 |
| 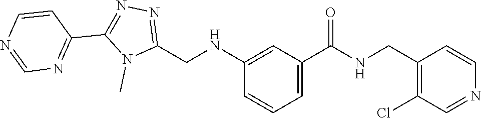 [SMILES: Cn1c(CNc2cccc(C(=O)NCc3ccncc3Cl)c2)nnc1-c1ccncn1] | 9.1 | | 4.06 | 27 |
| 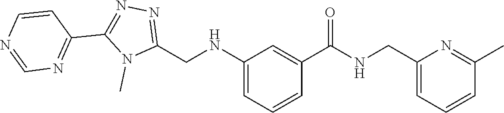 [SMILES: Cc1cccc(CNC(=O)c2cccc(NCc3nnc(-c4ccncn4)n3C)c2)n1] | | | 3.92 | 31.8 |
| 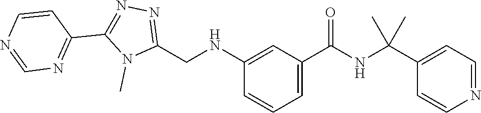 [SMILES: Cn1c(CNc2cccc(C(=O)NC(C)(C)c3ccncc3)c2)nnc1-c1ccncn1] | 49 | | >10.0 | 75.8 |
| 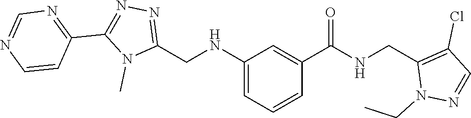 [SMILES: CCn1ncc(Cl)c1CNC(=O)c1cccc(NCc2nnc(-c3ccncn3)n2C)c1] | 16 | | >10.0 | 93.6 |
| 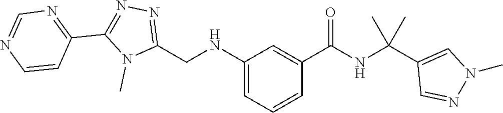 [SMILES: Cn1cc(C(C)(C)NC(=O)c2cccc(NCc3nnc(-c4ccncn4)n3C)c2)cn1] | | | >10.0 | 59.3 |
| 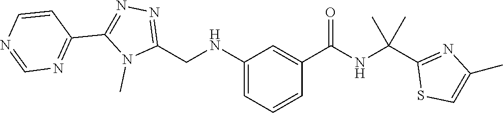 [SMILES: Cc1csc(C(C)(C)NC(=O)c2cccc(NCc3nnc(-c4ccncn4)n3C)c2)n1] | | | >10.0 | 59.7 |
| 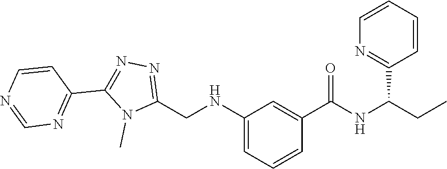 [SMILES: CC[C@H](NC(=O)c1cccc(NCc2nnc(-c3ccncn3)n2C)c1)c1ccccn1] | | | >10.0 | 94.3 |
| 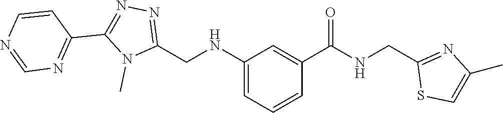 [SMILES: Cc1csc(CNC(=O)c2cccc(NCc3nnc(-c4ccncn4)n3C)c2)n1] | 100 | | | 59.7 |
| 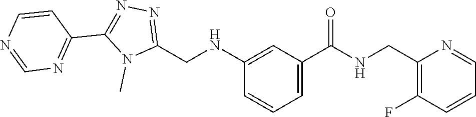 [SMILES: Cn1c(CNc2cccc(C(=O)NCc3ncccc3F)c2)nnc1-c1ccncn1] | 69 | | 0.164 | 61.7 |
| 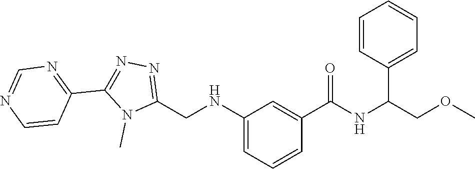 [SMILES: COCC(NC(=O)c1cccc(NCc2nnc(-c3ccncn3)n2C)c1)c1ccccc1] | 36 | | >10.0 | 84.1 |
| 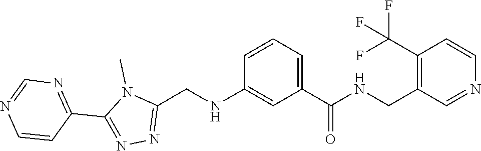 [SMILES: Cn1c(CNc2cccc(C(=O)NCc3cnccc3C(F)(F)F)c2)nnc1-c1ccncn1] | 39 | | >10.0 | 35.8 |
| 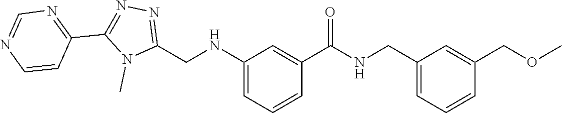 [SMILES: COCc1cccc(CNC(=O)c2cccc(NCc3nnc(-c4ccncn4)n3C)c2)c1] | | | 2.53 | 69.7 |
| 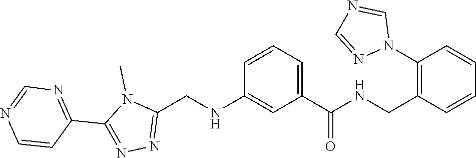 [SMILES: Cn1c(CNc2cccc(C(=O)NCc3ccccc3-n3cncn3)c2)nnc1-c1ccncn1] | | | >10.0 | 73.1 |
| 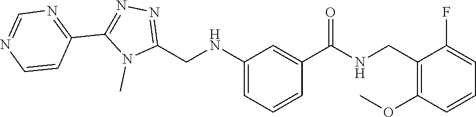 [SMILES: COc1cccc(F)c1CNC(=O)c1cccc(NCc2nnc(-c3ccncn3)n2C)c1] | 16 | | 0.149 | 55.8 |
| 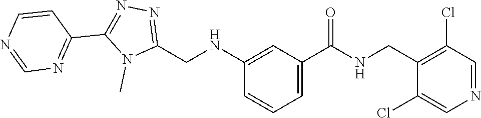 [SMILES: Cn1c(CNc2cccc(C(=O)NCc3c(Cl)cncc3Cl)c2)nnc1-c1ccncn1] | 31 | | 4.32 | 33.7 |
| 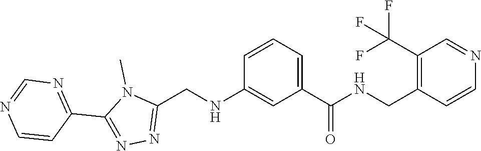 [SMILES: Cn1c(CNc2cccc(C(=O)NCc3ccncc3C(F)(F)F)c2)nnc1-c1ccncn1] | 33 | | >10.0 | 58.1 |
| 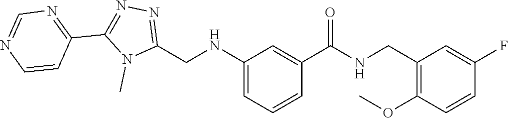 [SMILES: COc1ccc(F)cc1CNC(=O)c1cccc(NCc2nnc(-c3ccncn3)n2C)c1] | 15 | | | 54.5 |
| 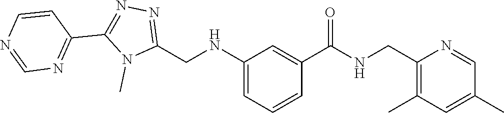 [SMILES: Cc1cnc(CNC(=O)c2cccc(NCc3nnc(-c4ccncn4)n3C)c2)c(C)c1] | 10 | | >10.0 | 47.8 |
| 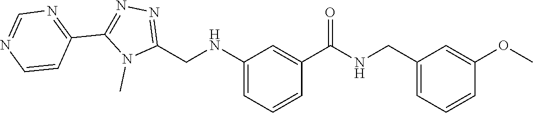 [SMILES: COc1cccc(CNC(=O)c2cccc(NCc3nnc(-c4ccncn4)n3C)c2)c1] | 22 | | >30.0 | 65.9 |
| 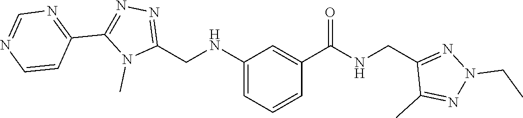 [SMILES: CCn1nc(C)c(CNC(=O)c2cccc(NCc3nnc(-c4ccncn4)n3C)c2)n1] | | | 0.555 | 34.8 |
| 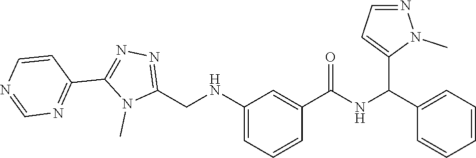 [SMILES: Cn1nccc1C(NC(=O)c1cccc(NCc2nnc(-c3ccncn3)n2C)c1)c1ccccc1] | | | >10.0 | 90.8 |
| 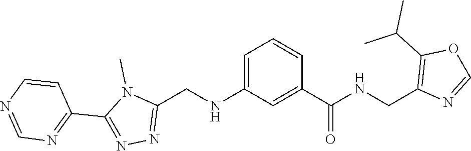 [SMILES: CC(C)c1ocnc1CNC(=O)c1cccc(NCc2nnc(-c3ccncn3)n2C)c1] | 350 | | >10.0 | 85.3 |
| 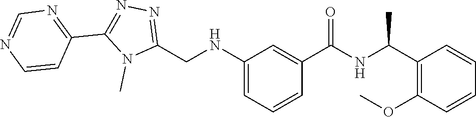 [SMILES: COc1ccccc1[C@H](C)NC(=O)c1cccc(NCc2nnc(-c3ccncn3)n2C)c1] | 94 | | >10.0 | 45.5 |
| 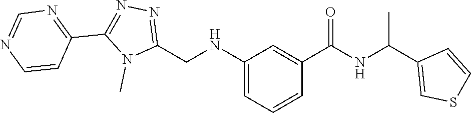 [SMILES: CC(NC(=O)c1cccc(NCc2nnc(-c3ccncn3)n2C)c1)c1ccsc1] | 4.9 | | >10.0 | 73.4 |
| 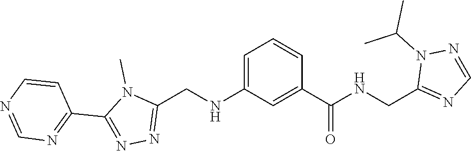 [SMILES: CC(C)n1ncnc1CNC(=O)c1cccc(NCc2nnc(-c3ccncn3)n2C)c1] | | | >10.0 | 99.8 |
| 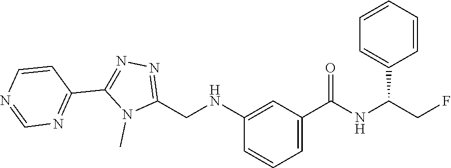 [SMILES: Cn1c(CNc2cccc(C(=O)N[C@@H](CF)c3ccccc3)c2)nnc1-c1ccncn1] | 97 | | | 47.1 |
| 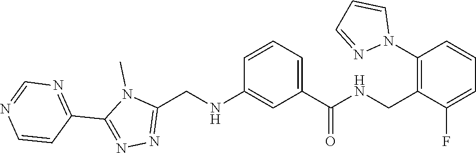 [SMILES: Cn1c(CNc2cccc(C(=O)NCc3c(F)cccc3-n3cccn3)c2)nnc1-c1ccncn1] | 24 | | >10.0 | 85.9 |
| 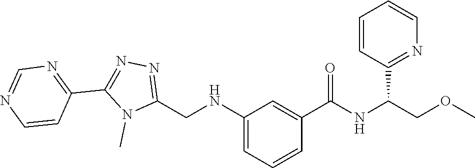 [SMILES: COC[C@H](NC(=O)c1cccc(NCc2nnc(-c3ccncn3)n2C)c1)c1ccccn1] | 54 | | >10.0 | 56.8 |
| 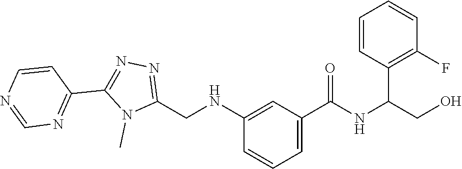 [SMILES: Cn1c(CNc2cccc(C(=O)NC(CO)c3ccccc3F)c2)nnc1-c1ccncn1] | 60 | | | 57.7 |
| 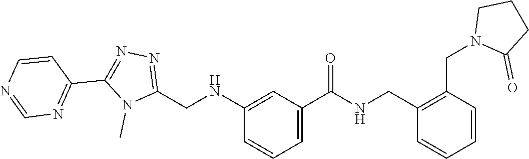 [SMILES: Cn1c(CNc2cccc(C(=O)NCc3ccccc3CN3CCCC3=O)c2)nnc1-c1ccncn1] | 49 | | >10.0 | 40 |
| 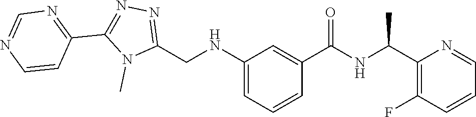 [SMILES: C[C@H](NC(=O)c1cccc(NCc2nnc(-c3ccncn3)n2C)c1)c1ncccc1F] | | | 0.738 | 63.6 |
| 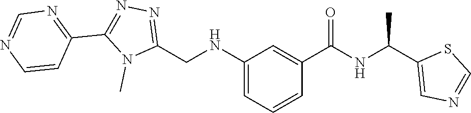 [SMILES: C[C@H](NC(=O)c1cccc(NCc2nnc(-c3ccncn3)n2C)c1)c1cncs1] | 410 | | >10.0 | 47.6 |
| 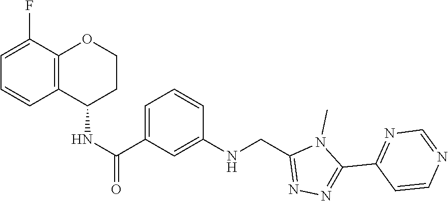 [SMILES: Cn1c(CNc2cccc(C(=O)N[C@H]3CCOc4c(F)cccc43)c2)nnc1-c1ccncn1] | 11 | | 1.23 | 39.4 |
| 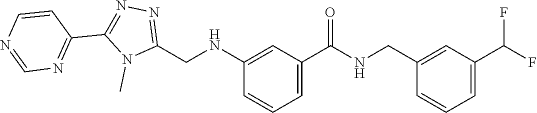 [SMILES: Cn1c(CNc2cccc(C(=O)NCc3cccc(C(F)F)c3)c2)nnc1-c1ccncn1] | 22 | | >10.0 | 38.3 |
| 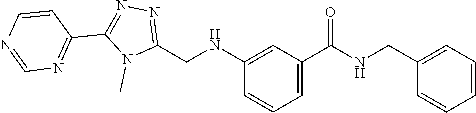 [SMILES: Cn1c(CNc2cccc(C(=O)NCc3ccccc3)c2)nnc1-c1ccncn1] | 20 | | >10.0 | 87.7 |
| 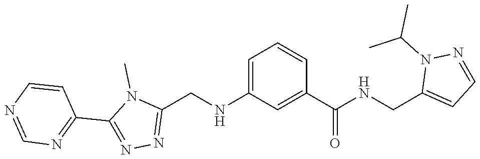 [SMILES: CC(C)n1nccc1CNC(=O)c1cccc(NCc2nnc(-c3ccncn3)n2C)c1] | | | >10.0 | 77.2 |
| 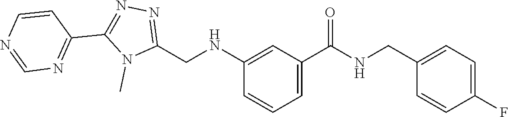 [SMILES: Cn1c(CNc2cccc(C(=O)NCc3ccc(F)cc3)c2)nnc1-c1ccncn1] | 19 | | 2.16 | 72.2 |
| 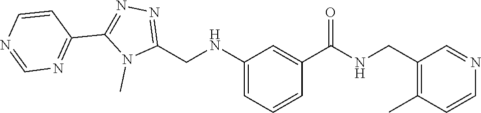 [SMILES: Cc1ccncc1CNC(=O)c1cccc(NCc2nnc(-c3ccncn3)n2C)c1] | 13 | | 0.557 | 44.4 |
| 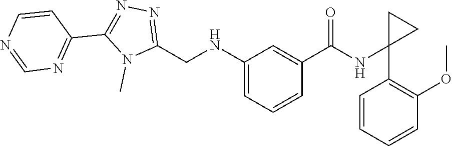 [SMILES: COc1ccccc1C1(NC(=O)c2cccc(NCc3nnc(-c4ccncn4)n3C)c2)CC1] | | | >10.0 | 76.2 |
| 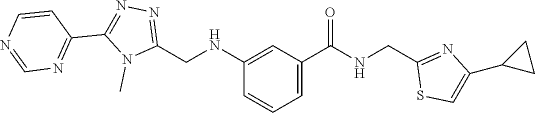 [SMILES: Cn1c(CNc2cccc(C(=O)NCc3nc(C4CC4)cs3)c2)nnc1-c1ccncn1] | | | 2.6 | 27.6 |
| 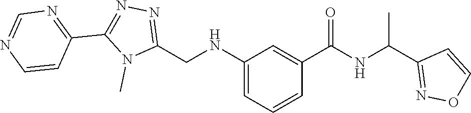 [SMILES: CC(NC(=O)c1cccc(NCc2nnc(-c3ccncn3)n2C)c1)c1ccon1] | | | >10.0 | 37.8 |
| 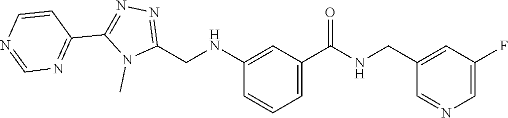 [SMILES: Cn1c(CNc2cccc(C(=O)NCc3cncc(F)c3)c2)nnc1-c1ccncn1] | | | >10.0 | 52.4 |
| 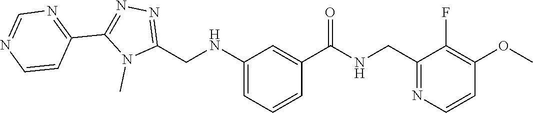 [SMILES: COc1ccnc(CNC(=O)c2cccc(NCc3nnc(-c4ccncn4)n3C)c2)c1F] | 200 | | >10.0 | 70.5 |
| 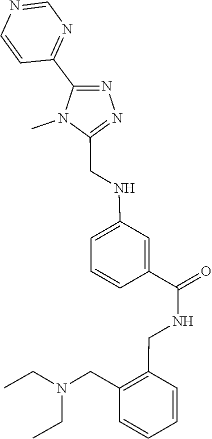 [SMILES: CCN(CC)Cc1ccccc1CNC(=O)c1cccc(NCc2nnc(-c3ccncn3)n2C)c1] | | | >10.0 | 64.4 |
| 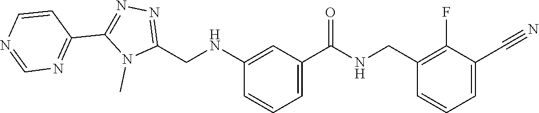 [SMILES: Cn1c(CNc2cccc(C(=O)NCc3cccc(C#N)c3F)c2)nnc1-c1ccncn1] | 30 | | >10.0 | 36.6 |
| 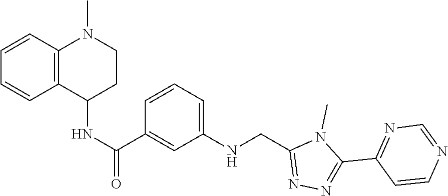 [SMILES: CN1CCC(NC(=O)c2cccc(NCc3nnc(-c4ccncn4)n3C)c2)c2ccccc21] | 37 | | 9.34 | 38.9 |
| 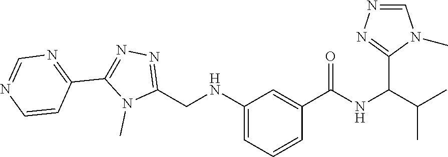 [SMILES: CC(C)C(NC(=O)c1cccc(NCc2nnc(-c3ccncn3)n2C)c1)c1nncn1C] | | | >10.0 | 101 |
| 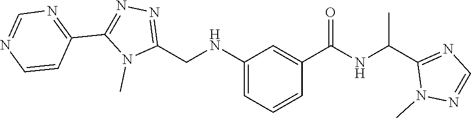 [SMILES: CC(NC(=O)c1cccc(NCc2nnc(-c3ccncn3)n2C)c1)c1ncnn1C] | 230 | | >10.0 | 75.7 |
| 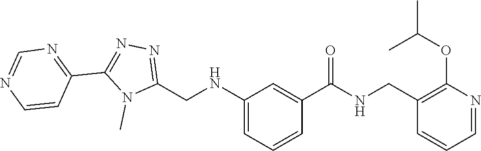 [SMILES: CC(C)Oc1ncccc1CNC(=O)c1cccc(NCc2nnc(-c3ccncn3)n2C)c1] | | | 2.36 | 48.9 |
| 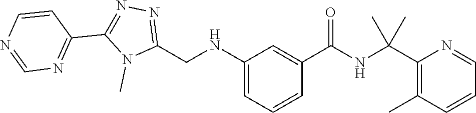 [SMILES: Cc1cccnc1C(C)(C)NC(=O)c1cccc(NCc2nnc(-c3ccncn3)n2C)c1] | 630 | | >10.0 | 62.4 |
| 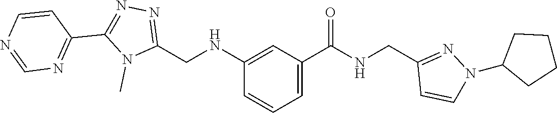 [SMILES: Cn1c(CNc2cccc(C(=O)NCc3ccn(C4CCCC4)n3)c2)nnc1-c1ccncn1] | | | >10.0 | 54.2 |
| 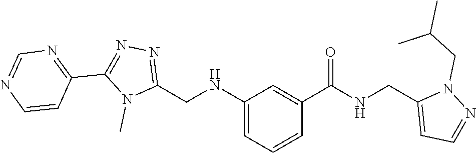 [SMILES: CC(C)Cn1nccc1CNC(=O)c1cccc(NCc2nnc(-c3ccncn3)n2C)c1] | | | 2.01 | 63.4 |
| 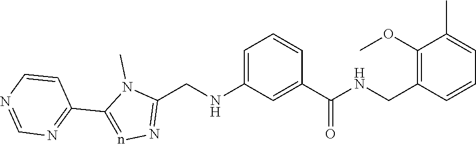 | 29 | | 2.06 | 30.4 |
| 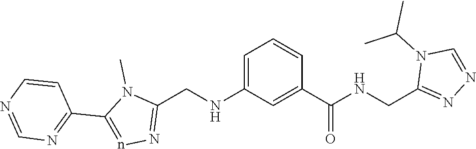 | | | >10.0 | 80 |
| 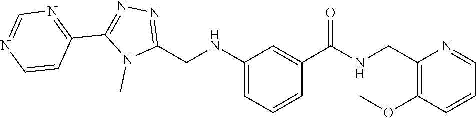 [SMILES: COc1cccnc1CNC(=O)c1cccc(NCc2nnc(-c3ccncn3)n2C)c1] | 290 | | >10.0 | 71.2 |
| 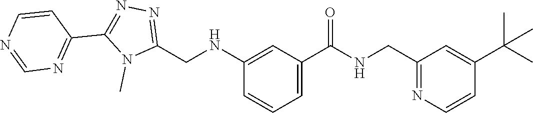 [SMILES: Cn1c(CNc2cccc(C(=O)NCc3cc(C(C)(C)C)ccn3)c2)nnc1-c1ccncn1] | | | 2.67 | 49 |
| 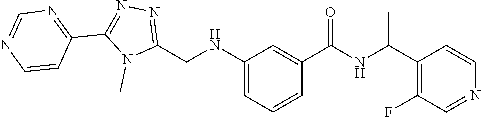 [SMILES: CC(NC(=O)c1cccc(NCc2nnc(-c3ccncn3)n2C)c1)c1ccncc1F] | 15 | | 0.103 | 49 |
| 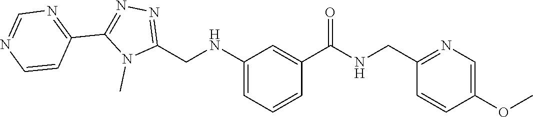 [SMILES: COc1ccc(CNC(=O)c2cccc(NCc3nnc(-c4ccncn4)n3C)c2)nc1] | | | >10.0 | 69.7 |
| 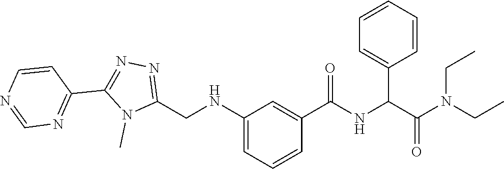 [SMILES: CCN(CC)C(=O)C(NC(=O)c1cccc(NCc2nnc(-c3ccncn3)n2C)c1)c1ccccc1] | | | >10.0 | 70.9 |
| 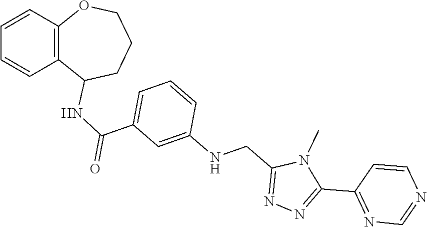 [SMILES: Cn1c(CNc2cccc(C(=O)NC3CCCOc4ccccc43)c2)nnc1-c1ccncn1] | 92 | | | 64.4 |
| 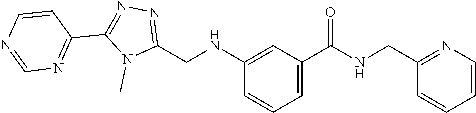 [SMILES: Cn1c(CNc2cccc(C(=O)NCc3ccccn3)c2)nnc1-c1ccncn1] | | | >10.0 | 84.3 |
| 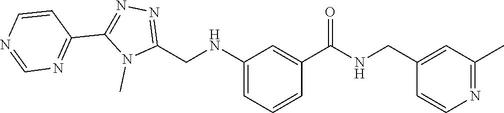 [SMILES: Cc1cc(CNC(=O)c2cccc(NCc3nnc(-c4ccncn4)n3C)c2)ccn1] | | | >10.0 | 68.7 |
| 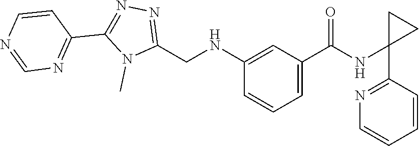 [SMILES: Cn1c(CNc2cccc(C(=O)NC3(c4ccccn4)CC3)c2)nnc1-c1ccncn1] | | | 2.03 | 16.5 |
| 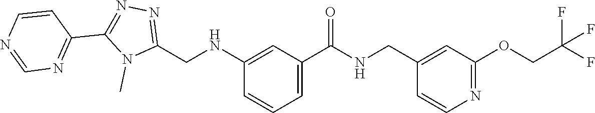 [SMILES: Cn1c(CNc2cccc(C(=O)NCc3ccnc(OCC(F)(F)F)c3)c2)nnc1-c1ccncn1] | 17 | | 3.76 | 18.7 |
| 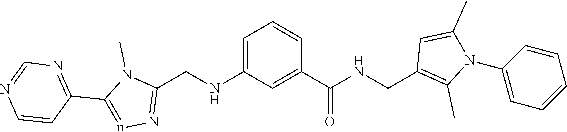 | 15 | | | 35.5 |
| 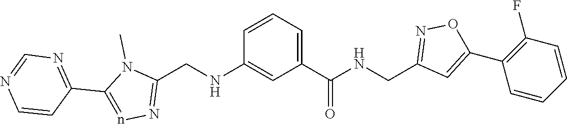 | | | 9.88 | 54.8 |
| 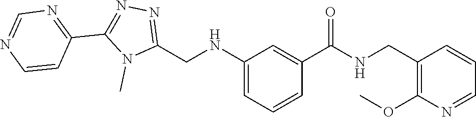 [SMILES: COc1ncccc1CNC(=O)c1cccc(NCc2nnc(-c3ccncn3)n2C)c1] | 70 | | >10.0 | 51.3 |
| 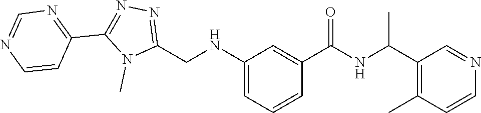 [SMILES: Cc1ccncc1C(C)NC(=O)c1cccc(NCc2nnc(-c3ccncn3)n2C)c1] | 50 | | 0.537 | 51.6 |
| 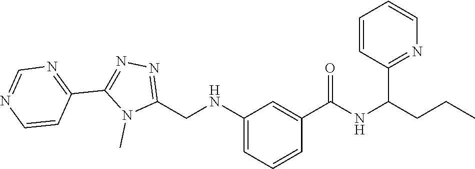 [SMILES: CCCC(NC(=O)c1cccc(NCc2nnc(-c3ccncn3)n2C)c1)c1ccccn1] | 67 | | 1.22 | 39.8 |
| 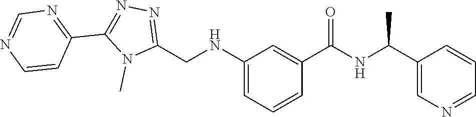 [SMILES: C[C@H](NC(=O)c1cccc(NCc2nnc(-c3ccncn3)n2C)c1)c1cccnc1] | 420 | | >8.57 | 55.1 |
| 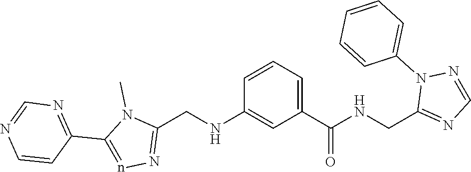 | 59 | | >10.0 | 68.1 |
| 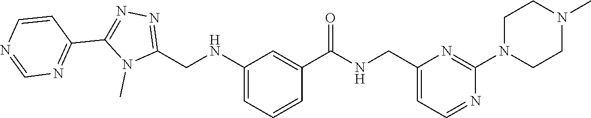 [SMILES: CN1CCN(c2nccc(CNC(=O)c3cccc(NCc4nnc(-c5ccncn5)n4C)c3)n2)CC1] | | | >10.0 | 100 |
| 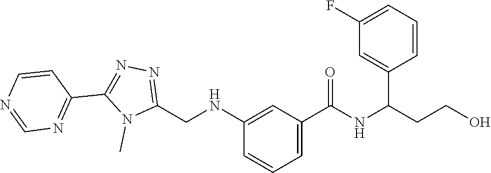 [SMILES: Cn1c(CNc2cccc(C(=O)NC(CCO)c3cccc(F)c3)c2)nnc1-c1ccncn1] | 37 | | >10.0 | 40.4 |
| 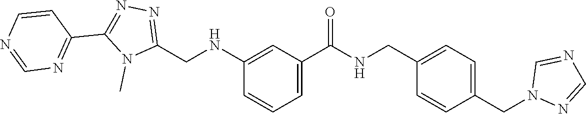 [SMILES: Cn1c(CNc2cccc(C(=O)NCc3ccc(Cn4cncn4)cc3)c2)nnc1-c1ccncn1] | | | >10.0 | 63.6 |
| 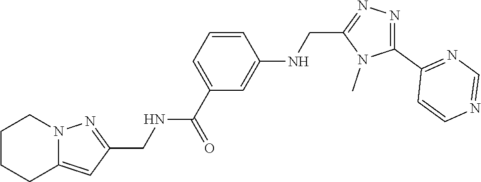 [SMILES: Cn1c(CNc2cccc(C(=O)NCc3cc4n(n3)CCCC4)c2)nnc1-c1ccncn1] | | | 2.21 | 14.1 |
| 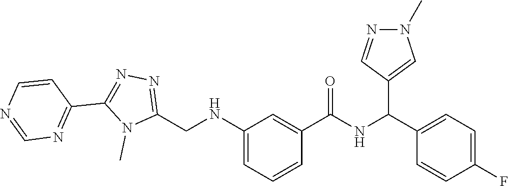 [SMILES: Cn1cc(C(NC(=O)c2cccc(NCc3nnc(-c4ccncn4)n3C)c2)c2ccc(F)cc2)cn1] | | | >10.0 | 114 |
| 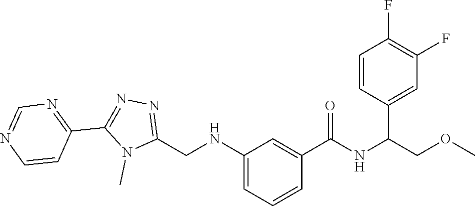 [SMILES: COCC(NC(=O)c1cccc(NCc2nnc(-c3ccncn3)n2C)c1)c1ccc(F)c(F)c1] | 46 | | 0.28 | 57.4 |
| 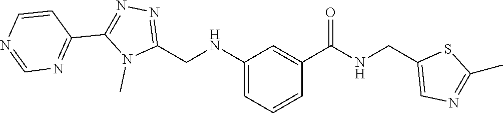 [SMILES: Cc1ncc(CNC(=O)c2cccc(NCc3nnc(-c4ccncn4)n3C)c2)s1] | | | >10.0 | 66.2 |
| 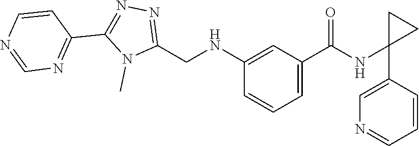 [SMILES: Cn1c(CNc2cccc(C(=O)NC3(c4cccnc4)CC3)c2)nnc1-c1ccncn1] | 200 | | 0.583 | 55.5 |
| 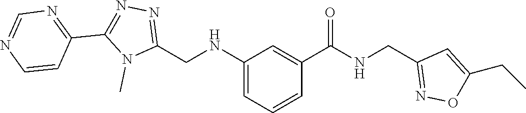 [SMILES: CCc1cc(CNC(=O)c2cccc(NCc3nnc(-c4ccncn4)n3C)c2)no1] | | | 3.55 | 40.4 |
| 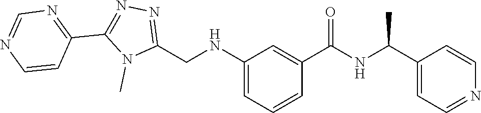 [SMILES: C[C@H](NC(=O)c1cccc(NCc2nnc(-c3ccncn3)n2C)c1)c1ccncc1] | 45 | | 0.771 | 63.3 |
| 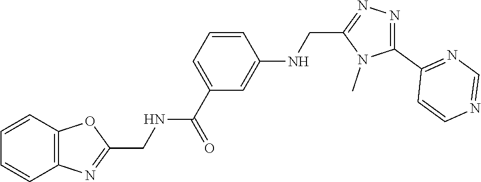 [SMILES: Cn1c(CNc2cccc(C(=O)NCc3nc4ccccc4o3)c2)nnc1-c1ccncn1] | | | >10.0 | 15.7 |
| 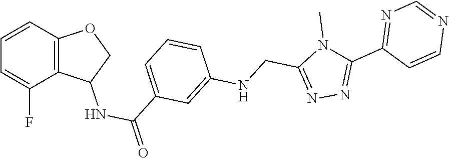 [SMILES: Cn1c(CNc2cccc(C(=O)NC3COc4cccc(F)c43)c2)nnc1-c1ccncn1] | 12 | | >10.0 | 35 |
| 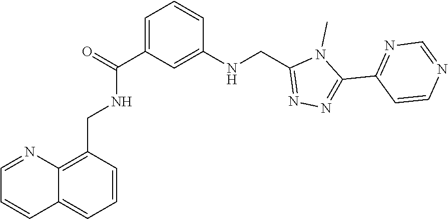 [SMILES: Cn1c(CNc2cccc(C(=O)NCc3cccc4cccnc34)c2)nnc1-c1ccncn1] | 61 | | 3.64 | 11.2 |
| 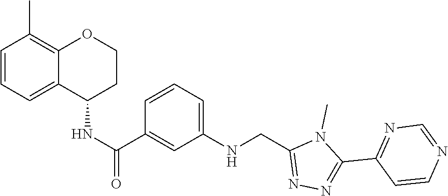 [SMILES: Cc1cccc2c1OCC[C@@H]2NC(=O)c1cccc(NCc2nnc(-c3ccncn3)n2C)c1] | 15 | | >10.0 | 36.7 |
| 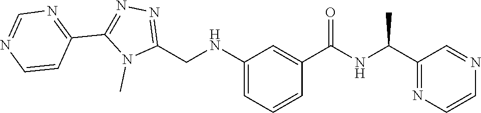 [SMILES: C[C@H](NC(=O)c1cccc(NCc2nnc(-c3ccncn3)n2C)c1)c1cnccn1] | | | 1.51 | 67.5 |
| 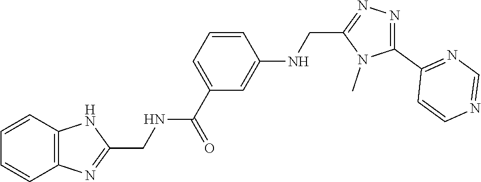 [SMILES: Cn1c(CNc2cccc(C(=O)NCc3nc4ccccc4[nH]3)c2)nnc1-c1ccncn1] | 100 | | | 58 |
| 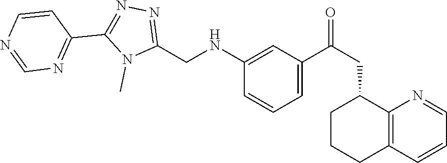 [SMILES: Cn1c(CNc2cccc(C(=O)C[C@H]3CCCc4cccnc43)c2)nnc1-c1ccncn1] | 20 | | 0.224 | 65.7 |
| 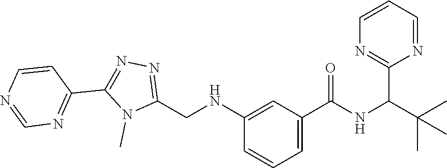 [SMILES: Cn1c(CNc2cccc(C(=O)NC(c3ncccn3)C(C)(C)C)c2)nnc1-c1ccncn1] | | | >10.0 | 65.9 |
| 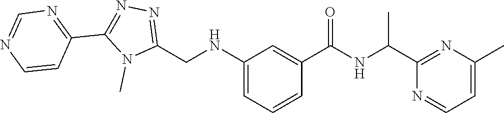 [SMILES: Cc1ccnc(C(C)NC(=O)c2cccc(NCc3nnc(-c4ccncn4)n3C)c2)n1] | | | >10.0 | 65.1 |
| 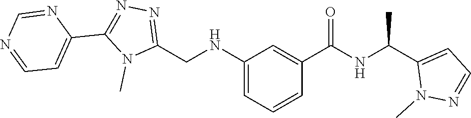 [SMILES: C[C@H](NC(=O)c1cccc(NCc2nnc(-c3ccncn3)n2C)c1)c1ccnn1C] | | | >10.0 | 63.1 |
| 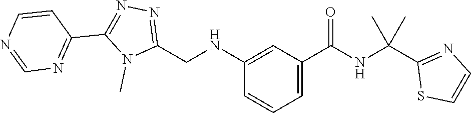 [SMILES: Cn1c(CNc2cccc(C(=O)NC(C)(C)c3nccs3)c2)nnc1-c1ccncn1] | | | | 71.6 |
| 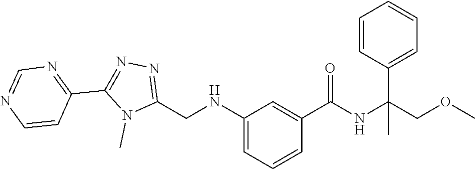 [SMILES: COCC(C)(NC(=O)c1cccc(NCc2nnc(-c3ccncn3)n2C)c1)c1ccccc1] | | | 0.88 | 54.2 |
| 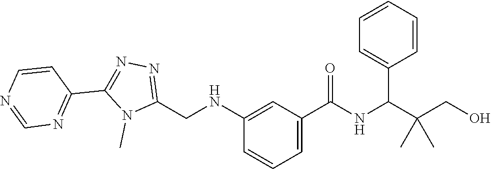 [SMILES: Cn1c(CNc2cccc(C(=O)NC(c3ccccc3)C(C)(C)CO)c2)nnc1-c1ccncn1] | 880 | | | 57.8 |
| 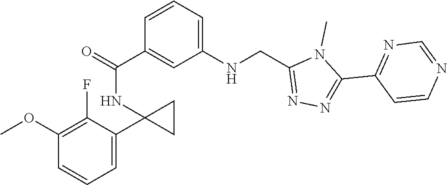 [SMILES: COc1cccc(C2(NC(=O)c3cccc(NCc4nnc(-c5ccncn5)n4C)c3)CC2)c1F] | 23 | | | 38.3 |
| 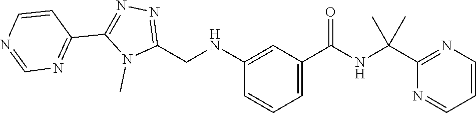 [SMILES: Cn1c(CNc2cccc(C(=O)NC(C)(C)c3ncccn3)c2)nnc1-c1ccncn1] | | | >10.0 | 76 |
| 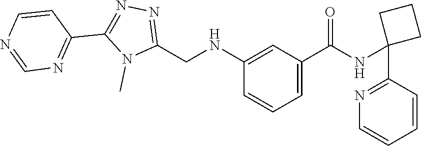 [SMILES: Cn1c(CNc2cccc(C(=O)NC3(c4ccccn4)CCC3)c2)nnc1-c1ccncn1] | | | >10.0 | 78.5 |
| 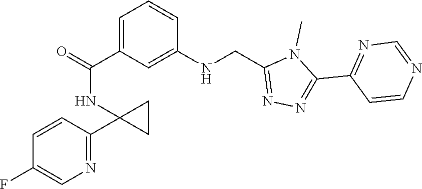 [SMILES: Cn1c(CNc2cccc(C(=O)NC3(c4ccc(F)cn4)CC3)c2)nnc1-c1ccncn1] | | | 0.291 | 23.3 |
| 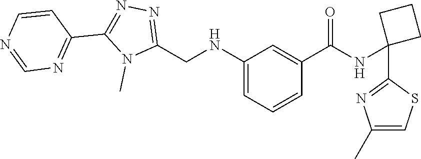 [SMILES: Cc1csc(C2(NC(=O)c3cccc(NCc4nnc(-c5ccncn5)n4C)c3)CCC2)n1] | 140 | | 0.445 | 64.5 |
| 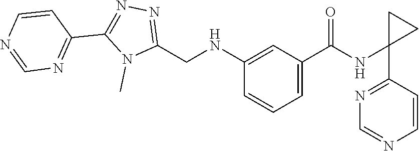 [SMILES: Cn1c(CNc2cccc(C(=O)NC3(c4ccncn4)CC3)c2)nnc1-c1ccncn1] | 100 | | 0.726 | 75.4 |
| 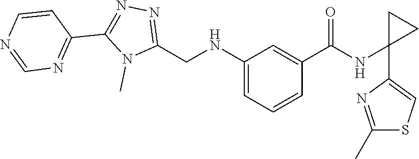 [SMILES: Cc1nc(C2(NC(=O)c3cccc(NCc4nnc(-c5ccncn5)n4C)c3)CC2)cs1] | | | 4.74 | 46.4 |
| 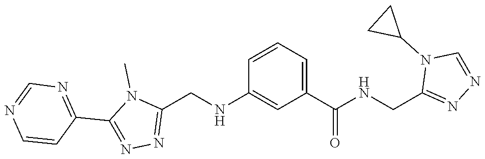 [SMILES: Cn1c(CNc2cccc(C(=O)NCc3nncn3C3CC3)c2)nnc1-c1ccncn1] | | | >10.0 | 71.3 |
| 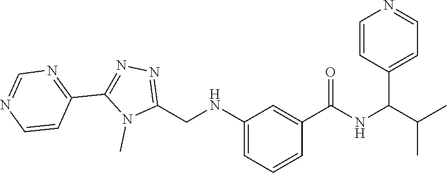 [SMILES: CC(C)C(NC(=O)c1cccc(NCc2nnc(-c3ccncn3)n2C)c1)c1ccncc1] | 32 | | 1.17 | 28.4 |
| 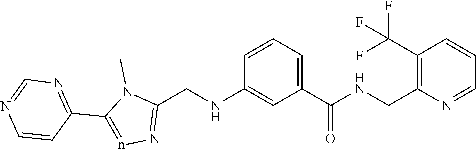 | 17 | | >10.0 | 34.9 |
| 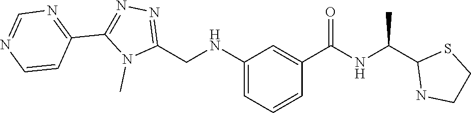 [SMILES: C[C@H](NC(=O)c1cccc(NCc2nnc(-c3ccncn3)n2C)c1)C1NCCS1] | 150 | | 0.172 | 66.7 |
| 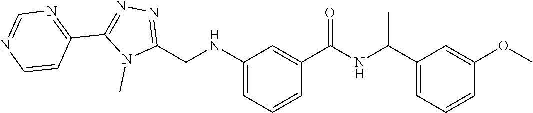 [SMILES: COc1cccc(C(C)NC(=O)c2cccc(NCc3nnc(-c4ccncn4)n3C)c2)c1] | 12 | | 0.377 | 40.7 |
| 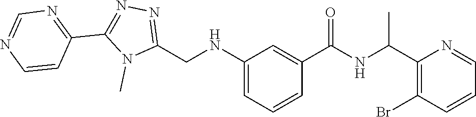 [SMILES: CC(NC(=O)c1cccc(NCc2nnc(-c3ccncn3)n2C)c1)c1ncccc1Br] | 9.4 | | >10.0 | 65.5 |
| 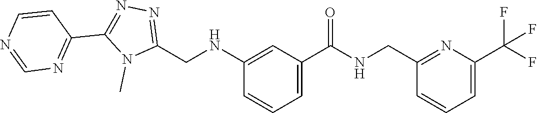 [SMILES: Cn1c(CNc2cccc(C(=O)NCc3cccc(C(F)(F)F)n3)c2)nnc1-c1ccncn1] | | | 1.86 | 10.5 |
| 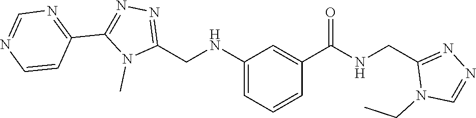 [SMILES: CCn1cnnc1CNC(=O)c1cccc(NCc2nnc(-c3ccncn3)n2C)c1] | 72 | | >10.0 | 94.4 |
| 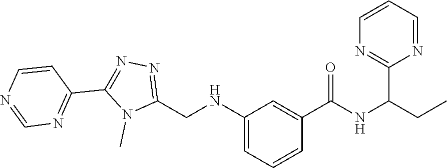 [SMILES: CCC(NC(=O)c1cccc(NCc2nnc(-c3ccncn3)n2C)c1)c1ncccn1] | | | 5.39 | 62.2 |
| 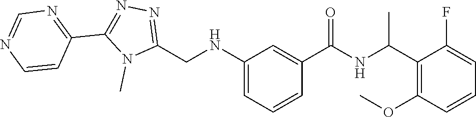 [SMILES: COc1cccc(F)c1C(C)NC(=O)c1cccc(NCc2nnc(-c3ccncn3)n2C)c1] | 27 | | >10.0 | 64.2 |
| 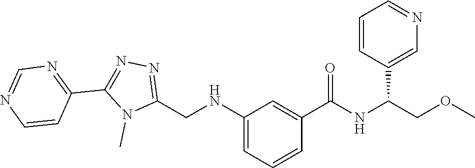 [SMILES: COC[C@H](NC(=O)c1cccc(NCc2nnc(-c3ccncn3)n2C)c1)c1cccnc1] | 46 | | 6.11 | 43.5 |
| 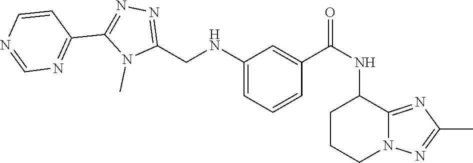 [SMILES: Cc1nc2n(n1)CCCC2NC(=O)c1cccc(NCc2nnc(-c3ccncn3)n2C)c1] | | | >10.0 | 77.8 |
| 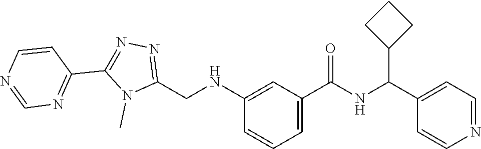 [SMILES: Cn1c(CNc2cccc(C(=O)NC(c3ccncc3)C3CCC3)c2)nnc1-c1ccncn1] | 22 | | 0.136 | 72.5 |
| 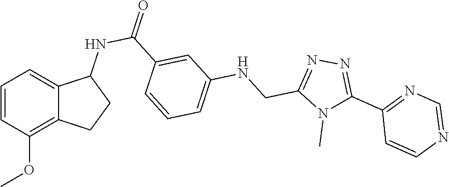 [SMILES: COc1cccc2c1CCC2NC(=O)c1cccc(NCc2nnc(-c3ccncn3)n2C)c1] | 15 | | 0.543 | 30.3 |
| 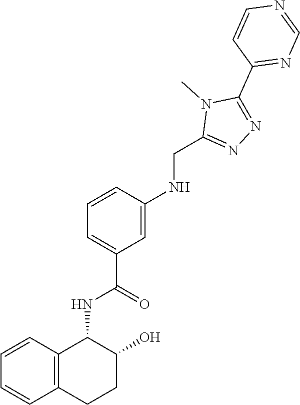 [SMILES: Cn1c(CNc2cccc(C(=O)N[C@H]3c4ccccc4CC[C@H]3O)c2)nnc1-c1ccncn1] | | | >10.0 | 90.6 |
| 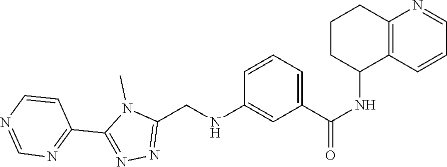 [SMILES: Cn1c(CNc2cccc(C(=O)NC3CCCc4ncccc43)c2)nnc1-c1ccncn1] | | | 2.5 | 53.7 |
| 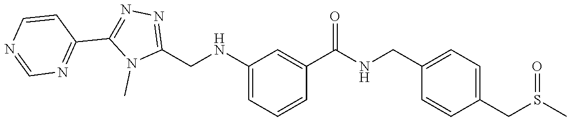 [SMILES: Cn1c(CNc2cccc(C(=O)NCc3ccc(CS(C)=O)cc3)c2)nnc1-c1ccncn1] | 170 | | >10.0 | 61.5 |
| 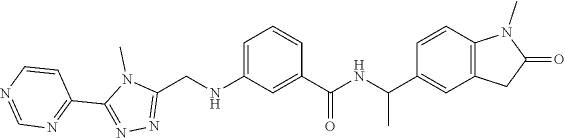 [SMILES: CC(NC(=O)c1cccc(NCc2nnc(-c3ccncn3)n2C)c1)c1ccc2c(c1)CC(=O)N2C] | 21 | | 0.158 | 46.9 |
| 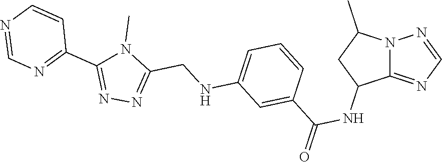 [SMILES: CC1CC(NC(=O)c2cccc(NCc3nnc(-c4ccncn4)n3C)c2)c2ncnn21] | | | >10.0 | 89.4 |
| 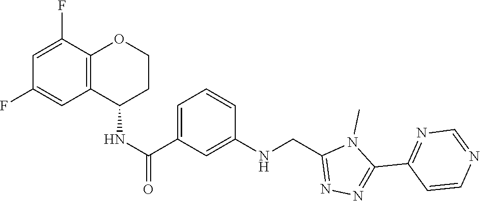 [SMILES: Cn1c(CNc2cccc(C(=O)N[C@H]3CCOc4c(F)cc(F)cc43)c2)nnc1-c1ccncn1] | | | >10.0 | 56.8 |
| 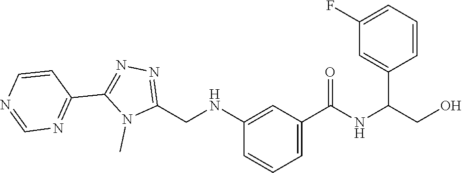 [SMILES: Cn1c(CNc2cccc(C(=O)NC(CO)c3cccc(F)c3)c2)nnc1-c1ccncn1] | | | 2.48 | 66.6 |
| 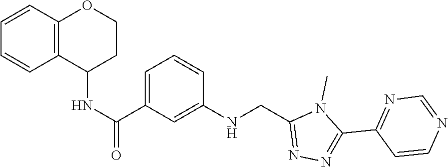 [SMILES: Cn1c(CNc2cccc(C(=O)NC3CCOc4ccccc43)c2)nnc1-c1ccncn1] | 22 | | 0.563 | 51.1 |
| 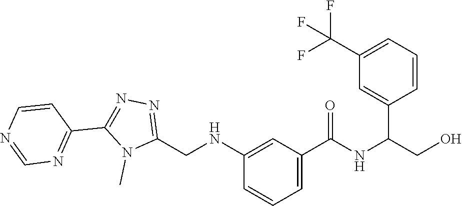 [SMILES: Cn1c(CNc2cccc(C(=O)NC(CO)c3cccc(C(F)(F)F)c3)c2)nnc1-c1ccncn1] | 34 | | >10.0 | 33.3 |
| 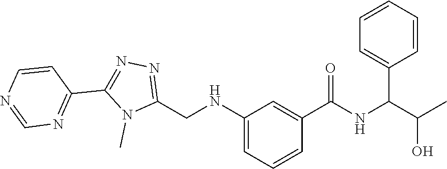 [SMILES: CC(O)C(NC(=O)c1cccc(NCc2nnc(-c3ccncn3)n2C)c1)c1ccccc1] | | | 1.76 | 69 |
| 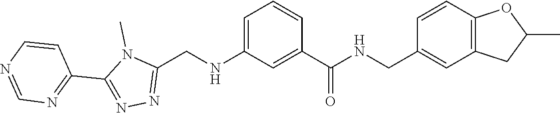 [SMILES: CC1Cc2cc(CNC(=O)c3cccc(NCc4nnc(-c5ccncn5)n4C)c3)ccc2O1] | 22 | | 3.37 | 31.1 |
| 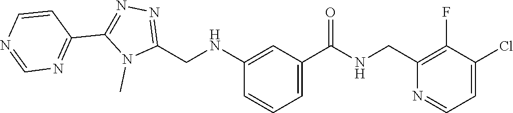 [SMILES: Cn1c(CNc2cccc(C(=O)NCc3nccc(Cl)c3F)c2)nnc1-c1ccncn1] | 220 | | <0.0390 | 61.7 |
| 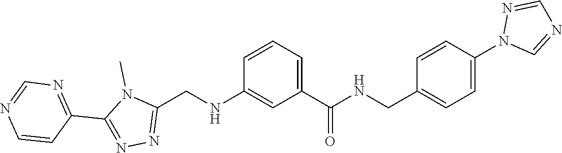 [SMILES: Cn1c(CNc2cccc(C(=O)NCc3ccc(-n4cncn4)cc3)c2)nnc1-c1ccncn1] | | | >10.0 | 67.7 |
| 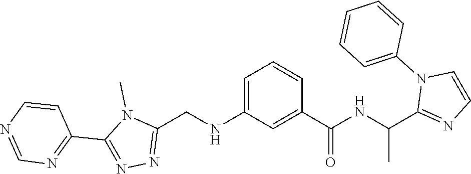 [SMILES: CC(NC(=O)c1cccc(NCc2nnc(-c3ccncn3)n2C)c1)c1nccn1-c1ccccc1] | | | >10.0 | 78.2 |
| 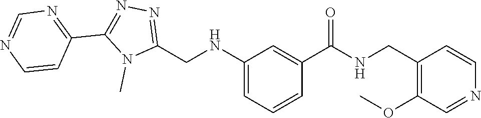 [SMILES: COc1cnccc1CNC(=O)c1cccc(NCc2nnc(-c3ccncn3)n2C)c1] | | | >10.0 | 88.9 |
| 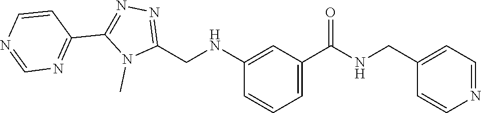 [SMILES: Cn1c(CNc2cccc(C(=O)NCc3ccncc3)c2)nnc1-c1ccncn1] | | | >10.0 | 81.6 |
| 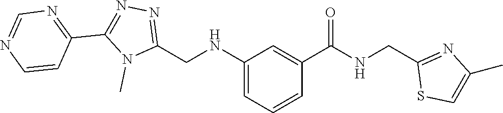 [SMILES: Cc1csc(CNC(=O)c2cccc(NCc3nnc(-c4ccncn4)n3C)c2)n1] | | | 0.862 | 46.6 |
| 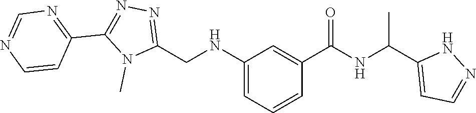 [SMILES: CC(NC(=O)c1cccc(NCc2nnc(-c3ccncn3)n2C)c1)c1ccn[nH]1] | | | >10.0 | 84.4 |
| 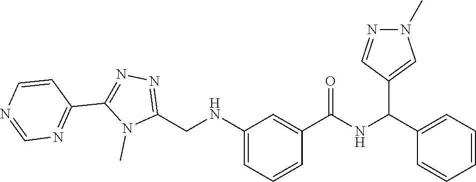 [SMILES: Cn1cc(C(NC(=O)c2cccc(NCc3nnc(-c4ccncn4)n3C)c2)c2ccccc2)cn1] | 41 | | 0.0664 | 63 |
| 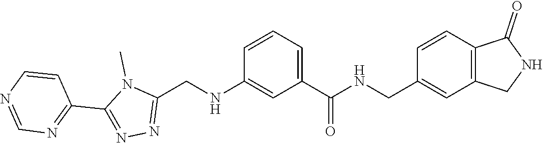 [SMILES: Cn1c(CNc2cccc(C(=O)NCc3ccc4c(c3)CNC4=O)c2)nnc1-c1ccncn1] | | | >10.0 | 57.2 |
| 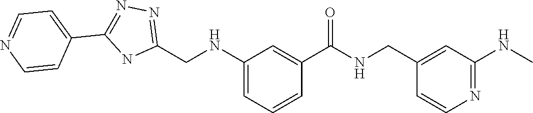 [SMILES: CNc1cc(CNC(=O)c2cccc(NCc3nnc(-c4ccncc4)[nH]3)c2)ccn1] | | | >10.0 | 83.7 |
| 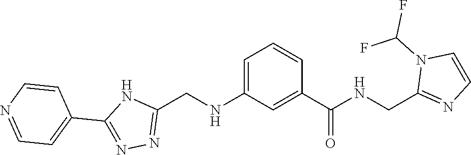 [SMILES: O=C(NCc1nccn1C(F)F)c1cccc(NCc2nnc(-c3ccncc3)[nH]2)c1] | 14 | | 2.88 | 45.1 |
| 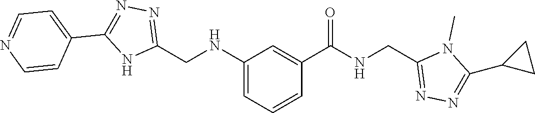 [SMILES: Cn1c(CNC(=O)c2cccc(NCc3nnc(-c4ccncc4)[nH]3)c2)nnc1C1CC1] | | | >10.0 | 70.7 |
| 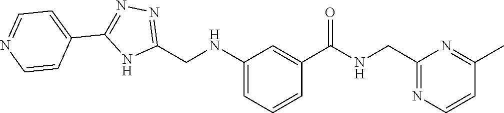 [SMILES: Cc1ccnc(CNC(=O)c2cccc(NCc3nnc(-c4ccncc4)[nH]3)c2)n1] | 15 | | >10.0 | 100 |
| 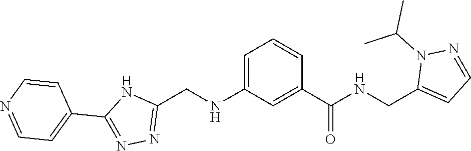 [SMILES: CC(C)n1nccc1CNC(=O)c1cccc(NCc2nnc(-c3ccncc3)[nH]2)c1] | 20 | | >10.0 | 37.7 |
| 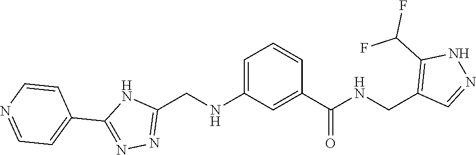 [SMILES: O=C(NCc1cn[nH]c1C(F)F)c1cccc(NCc2nnc(-c3ccncc3)[nH]2)c1] | 19 | | >10.0 | 59.8 |
| 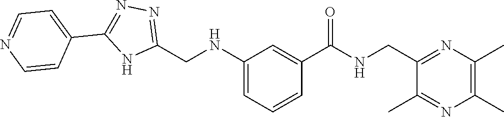 [SMILES: Cc1nc(C)c(CNC(=O)c2cccc(NCc3nnc(-c4ccncc4)[nH]3)c2)nc1C] | 28 | | >10.0 | 46.4 |
| 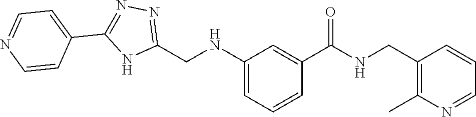 [SMILES: Cc1ncccc1CNC(=O)c1cccc(NCc2nnc(-c3ccncc3)[nH]2)c1] | 19 | | >10.0 | 83.4 |
| 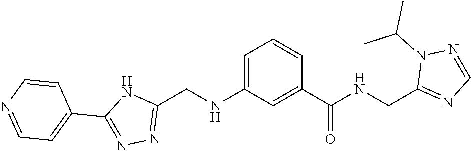 [SMILES: CC(C)n1ncnc1CNC(=O)c1cccc(NCc2nnc(-c3ccncc3)[nH]2)c1] | | | >10.0 | 92.7 |
| 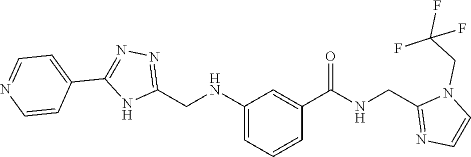 [SMILES: O=C(NCc1nccn1CC(F)(F)F)c1cccc(NCc2nnc(-c3ccncc3)[nH]2)c1] | | | >10.0 | 20.1 |
| 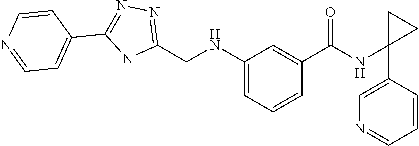 [SMILES: O=C(NC1(c2cccnc2)CC1)c1cccc(NCc2nnc(-c3ccncc3)[nH]2)c1] | | | >10.0 | 61.9 |
| 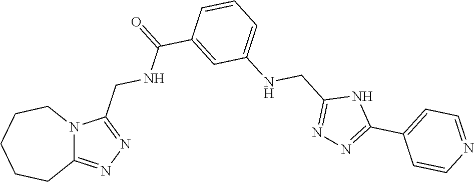 [SMILES: O=C(NCc1nnc2n1CCCCC2)c1cccc(NCc2nnc(-c3ccncc3)[nH]2)c1] | | | >10.0 | 94.4 |
| 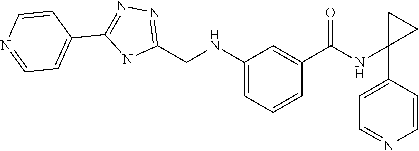 [SMILES: O=C(NC1(c2ccncc2)CC1)c1cccc(NCc2nnc(-c3ccncc3)[nH]2)c1] | | | >10.0 | 136 |
| 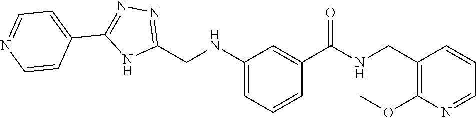 [SMILES: COc1ncccc1CNC(=O)c1cccc(NCc2nnc(-c3ccncc3)[nH]2)c1] | 12 | 5.8 | >11.0 | 29.8 |
| 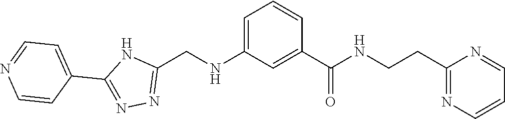 [SMILES: O=C(NCCc1ncccn1)c1cccc(NCc2nnc(-c3ccncc3)[nH]2)c1] | | | >10.0 | 96.6 |
| 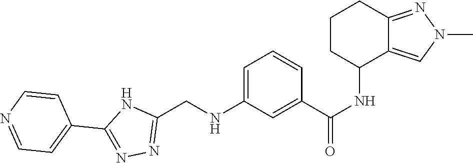 [SMILES: Cn1cc2c(n1)CCCC2NC(=O)c1cccc(NCc2nnc(-c3ccncc3)[nH]2)c1] | | | >10.0 | 64.7 |
| 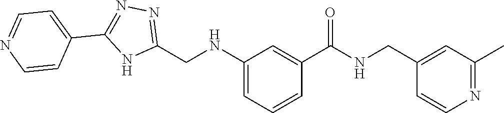 [SMILES: Cc1cc(CNC(=O)c2cccc(NCc3nnc(-c4ccncc4)[nH]3)c2)ccn1] | | | >10.0 | 56.8 |
| 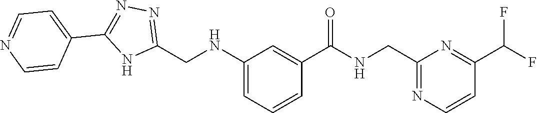 [SMILES: O=C(NCc1nccc(C(F)F)n1)c1cccc(NCc2nnc(-c3ccncc3)[nH]2)c1] | 27 | | >10.0 | 89.8 |
| 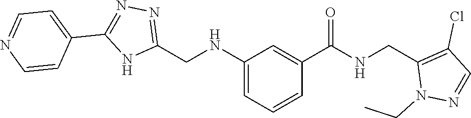 [SMILES: CCn1ncc(Cl)c1CNC(=O)c1cccc(NCc2nnc(-c3ccncc3)[nH]2)c1] | 12 | | 0.56 | 45.8 |
| 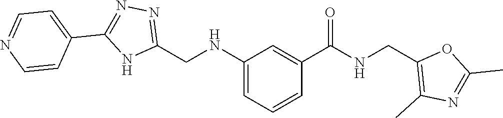 [SMILES: Cc1nc(C)c(CNC(=O)c2cccc(NCc3nnc(-c4ccncc4)[nH]3)c2)o1] | | | >10.0 | 66.7 |
| 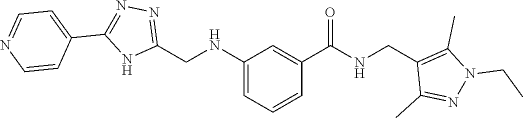 [SMILES: CCn1nc(C)c(CNC(=O)c2cccc(NCc3nnc(-c4ccncc4)[nH]3)c2)c1C] | | | >10.0 | 26 |
| 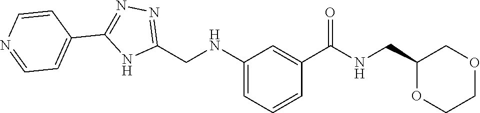 [SMILES: O=C(NC[C@H]1COCCO1)c1cccc(NCc2nnc(-c3ccncc3)[nH]2)c1] | | | 1.12 | 76 |
| 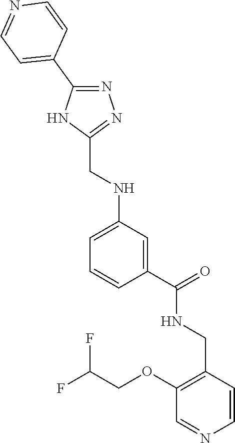 [SMILES: O=C(NCc1ccncc1OCC(F)F)c1cccc(NCc2nnc(-c3ccncc3)[nH]2)c1] | | | >10.0 | 54.4 |
| 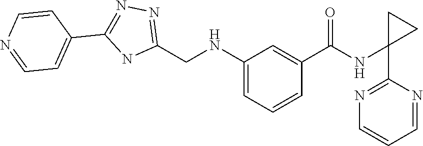 [SMILES: O=C(NC1(c2ncccn2)CC1)c1cccc(NCc2nnc(-c3ccncc3)[nH]2)c1] | | | >10.0 | 134 |
| 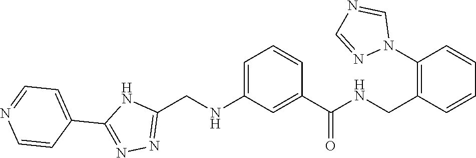 [SMILES: O=C(NCc1ccccc1-n1cncn1)c1cccc(NCc2nnc(-c3ccncc3)[nH]2)c1] | 19 | | >10.0 | 45.3 |
| 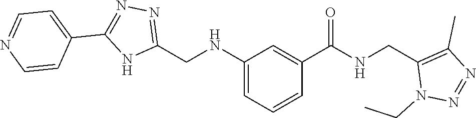 [SMILES: CCn1nnc(C)c1CNC(=O)c1cccc(NCc2nnc(-c3ccncc3)[nH]2)c1] | | | 0.651 | 55.6 |
| 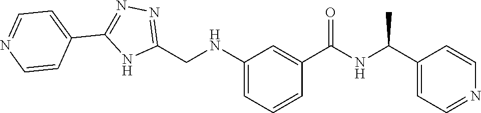 [SMILES: C[C@H](NC(=O)c1cccc(NCc2nnc(-c3ccncc3)[nH]2)c1)c1ccncc1] | | | >10.0 | 75.1 |
| 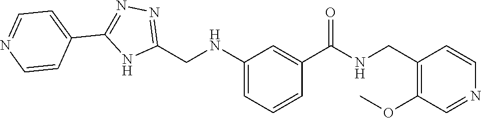 [SMILES: COc1cnccc1CNC(=O)c1cccc(NCc2nnc(-c3ccncc3)[nH]2)c1] | 26 | | >10.0 | 72.7 |
| 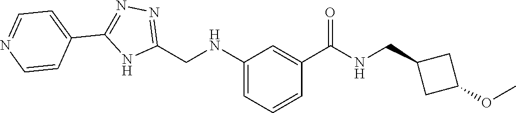 [SMILES: CO[C@H]1C[C@H](CNC(=O)c2cccc(NCc3nnc(-c4ccncc4)[nH]3)c2)C1] | | | >10.0 | 68.6 |
| 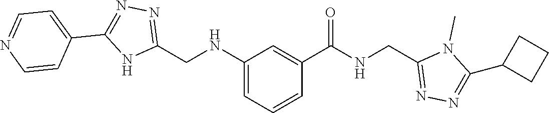 [SMILES: Cn1c(CNC(=O)c2cccc(NCc3nnc(-c4ccncc4)[nH]3)c2)nnc1C1CCC1] | | | >10.0 | 70.1 |
| 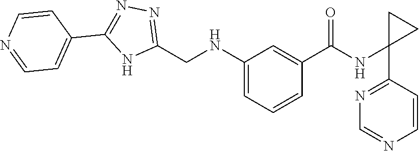 [SMILES: O=C(NC1(c2ccncn2)CC1)c1cccc(NCc2nnc(-c3ccncc3)[nH]2)c1] | 13 | | >10.0 | 61 |
| 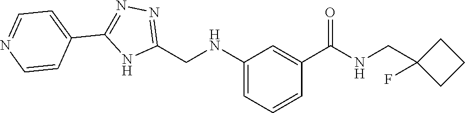 [SMILES: O=C(NCC1(F)CCC1)c1cccc(NCc2nnc(-c3ccncc3)[nH]2)c1] | | | 3.22 | 26.3 |
| 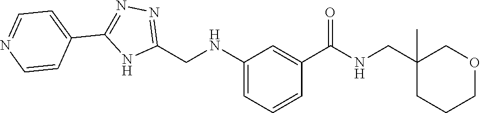 [SMILES: CC1(CNC(=O)c2cccc(NCc3nnc(-c4ccncc4)[nH]3)c2)CCCOC1] | | | >10.0 | 89 |
| 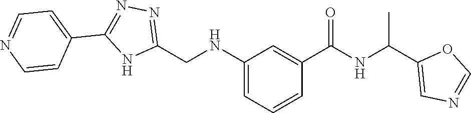 [SMILES: CC(NC(=O)c1cccc(NCc2nnc(-c3ccncc3)[nH]2)c1)c1cnco1] | 24 | | >10.0 | 72.4 |
| 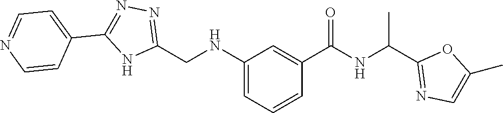 [SMILES: Cc1cnc(C(C)NC(=O)c2cccc(NCc3nnc(-c4ccncc4)[nH]3)c2)o1] | 16 | | >10.0 | 46.6 |
| 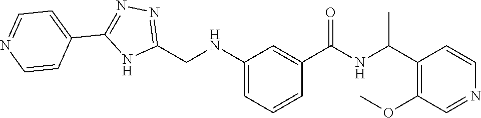 [SMILES: COc1cnccc1C(C)NC(=O)c1cccc(NCc2nnc(-c3ccncc3)[nH]2)c1] | | | >10.0 | 61.7 |
| 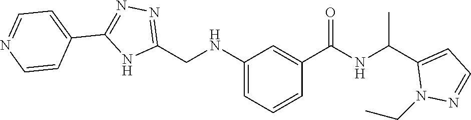 [SMILES: CCn1nccc1C(C)NC(=O)c1cccc(NCc2nnc(-c3ccncc3)[nH]2)c1] | | | >10.0 | 84.6 |
| 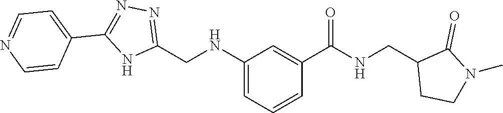 [SMILES: CN1CCC(CNC(=O)c2cccc(NCc3nnc(-c4ccncc4)[nH]3)c2)C1=O] | | | >10.0 | 102 |
| 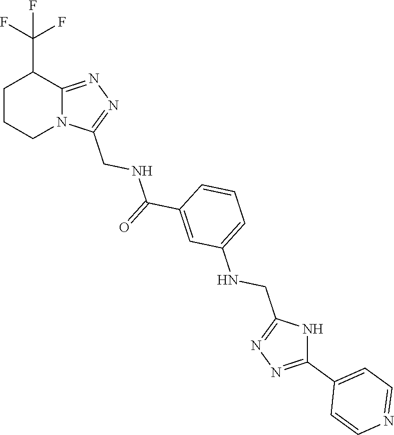 [SMILES: O=C(NCc1nnc2n1CCCC2C(F)(F)F)c1cccc(NCc2nnc(-c3ccncc3)[nH]2)c1] | | | >10.0 | 74.9 |
| 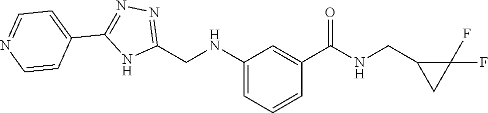 [SMILES: O=C(NCC1CC1(F)F)c1cccc(NCc2nnc(-c3ccncc3)[nH]2)c1] | | | >10.0 | 51.6 |
| 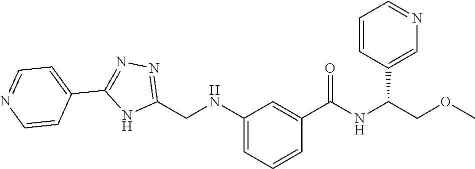 [SMILES: COC[C@H](NC(=O)c1cccc(NCc2nnc(-c3ccncc3)[nH]2)c1)c1cccnc1] | | | >10.0 | 38 |
| 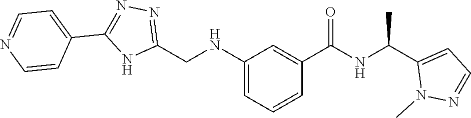 [SMILES: C[C@H](NC(=O)c1cccc(NCc2nnc(-c3ccncc3)[nH]2)c1)c1ccnn1C] | | | 9.81 | 65.9 |
| 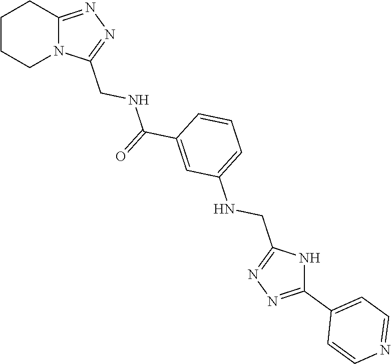 [SMILES: O=C(NCc1nnc2n1CCCC2)c1cccc(NCc2nnc(-c3ccncc3)[nH]2)c1] | 48 | | >10.0 | 74.9 |
| 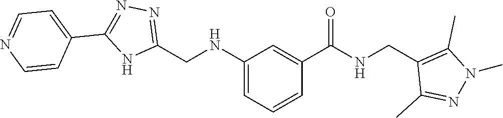 [SMILES: Cc1nn(C)c(C)c1CNC(=O)c1cccc(NCc2nnc(-c3ccncc3)[nH]2)c1] | | | >10.0 | 43.6 |
| 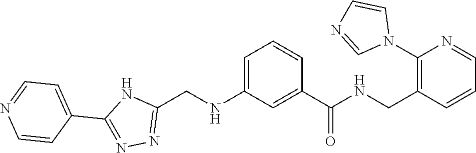 [SMILES: O=C(NCc1cccnc1-n1ccnc1)c1cccc(NCc2nnc(-c3ccncc3)[nH]2)c1] | | | >10.0 | 95.9 |
| 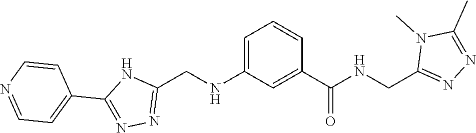 [SMILES: Cc1nnc(CNC(=O)c2cccc(NCc3nnc(-c4ccncc4)[nH]3)c2)n1C] | | | >10.0 | 96.6 |
| 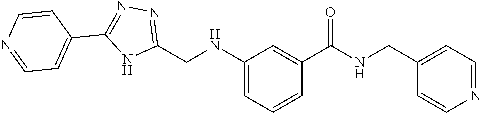 [SMILES: O=C(NCc1ccncc1)c1cccc(NCc2nnc(-c3ccncc3)[nH]2)c1] | 12 | | 5.72 | 50.5 |
| 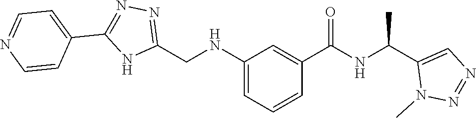 [SMILES: C[C@H](NC(=O)c1cccc(NCc2nnc(-c3ccncc3)[nH]2)c1)c1cnnn1C] | | | >10.0 | 71.4 |
| 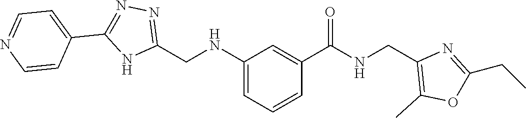 [SMILES: CCc1nc(CNC(=O)c2cccc(NCc3nnc(-c4ccncc4)[nH]3)c2)c(C)o1] | 8.4 | | 0.866 | 39.2 |
| 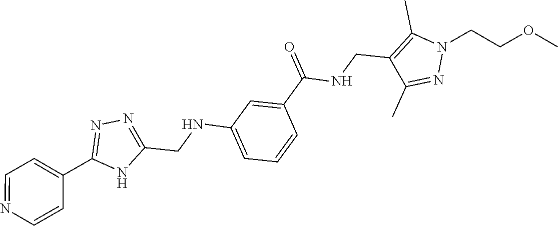 [SMILES: COCCn1nc(C)c(CNC(=O)c2cccc(NCc3nnc(-c4ccncc4)[nH]3)c2)c1C] | | | >10.0 | 71.6 |
| 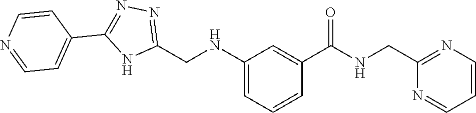 [SMILES: O=C(NCc1ncccn1)c1cccc(NCc2nnc(-c3ccncc3)[nH]2)c1] | 17 | | >10.0 | 64.2 |
| 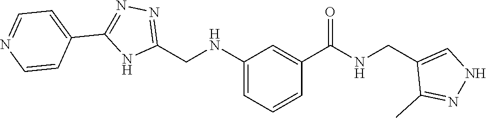 [SMILES: Cc1n[nH]cc1CNC(=O)c1cccc(NCc2nnc(-c3ccncc3)[nH]2)c1] | | | >10.0 | 67.5 |
| 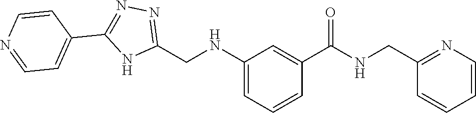 [SMILES: O=C(NCc1ccccn1)c1cccc(NCc2nnc(-c3ccncc3)[nH]2)c1] | 36 | | | |
| 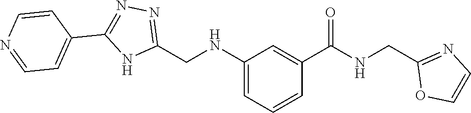 [SMILES: O=C(NCc1ncco1)c1cccc(NCc2nnc(-c3ccncc3)[nH]2)c1] | 25 | | >10.0 | 103 |
| 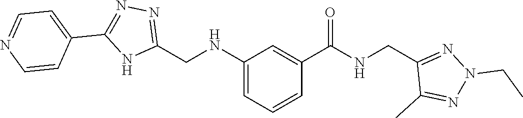 [SMILES: CCn1nc(C)c(CNC(=O)c2cccc(NCc3nnc(-c4ccncc4)[nH]3)c2)n1] | 18 | | 3.11 | 55.7 |
| 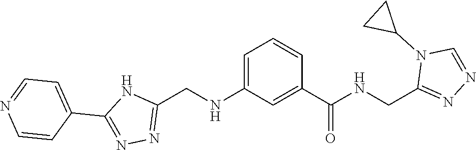 [SMILES: O=C(NCc1nncn1C1CC1)c1cccc(NCc2nnc(-c3ccncc3)[nH]2)c1] | 45 | | >10.0 | 79.1 |
| 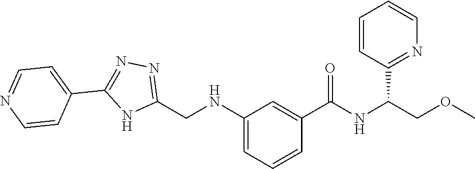 [SMILES: COC[C@H](NC(=O)c1cccc(NCc2nnc(-c3ccncc3)[nH]2)c1)c1ccccn1] | 11 | | 0.405 | 63.4 |
| 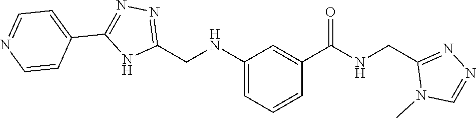 [SMILES: Cn1cnnc1CNC(=O)c1cccc(NCc2nnc(-c3ccncc3)[nH]2)c1] | | | >10.0 | 91.4 |
| 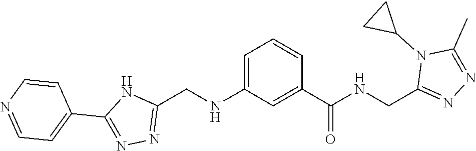 [SMILES: Cc1nnc(CNC(=O)c2cccc(NCc3nnc(-c4ccncc4)[nH]3)c2)n1C1CC1] | 19 | | >10.0 | 94.4 |
| 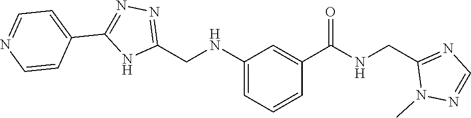 [SMILES: Cn1ncnc1CNC(=O)c1cccc(NCc2nnc(-c3ccncc3)[nH]2)c1] | | | >10.0 | 78.7 |
| 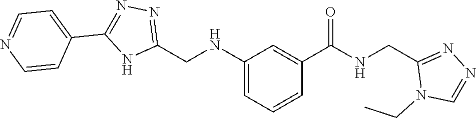 [SMILES: CCn1cnnc1CNC(=O)c1cccc(NCc2nnc(-c3ccncc3)[nH]2)c1] | | | >10.0 | 68.9 |
| 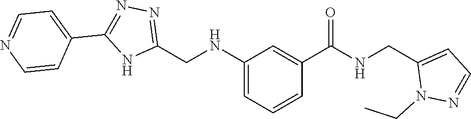 [SMILES: CCn1nccc1CNC(=O)c1cccc(NCc2nnc(-c3ccncc3)[nH]2)c1] | | | 0.739 | 71.1 |
| 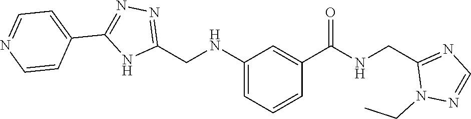 [SMILES: CCn1ncnc1CNC(=O)c1cccc(NCc2nnc(-c3ccncc3)[nH]2)c1] | | | >10.0 | 129 |
| 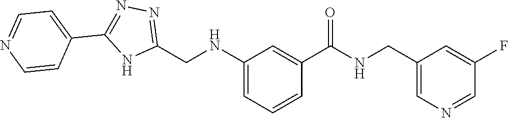 [SMILES: O=C(NCc1cncc(F)c1)c1cccc(NCc2nnc(-c3ccncc3)[nH]2)c1] | 17 | | >10.0 | 98.5 |
| 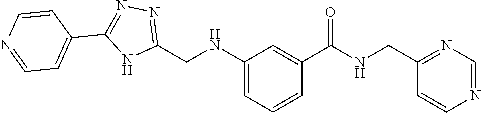 [SMILES: O=C(NCc1ccncn1)c1cccc(NCc2nnc(-c3ccncc3)[nH]2)c1] | | | >10.0 | 129 |
| 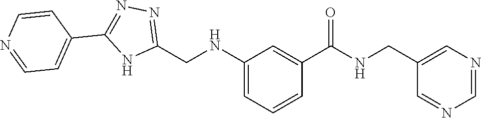 [SMILES: O=C(NCc1cncnc1)c1cccc(NCc2nnc(-c3ccncc3)[nH]2)c1] | 34 | | >10.0 | 120 |
| 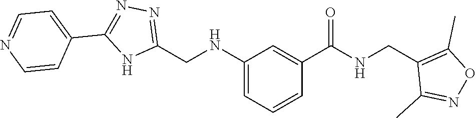 [SMILES: Cc1noc(C)c1CNC(=O)c1cccc(NCc2nnc(-c3ccncc3)[nH]2)c1] | 24 | | >10.0 | 98.9 |
| 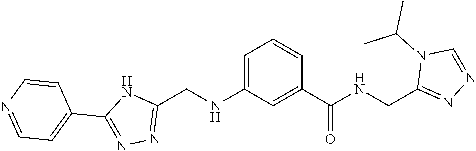 [SMILES: CC(C)n1cnnc1CNC(=O)c1cccc(NCc2nnc(-c3ccncc3)[nH]2)c1] | 210 | | >10.0 | 88.7 |
| 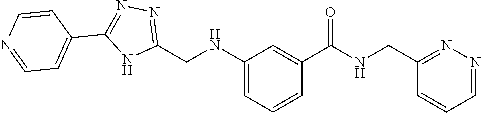 [SMILES: O=C(NCc1cccnn1)c1cccc(NCc2nnc(-c3ccncc3)[nH]2)c1] | | | >10.0 | 116 |
| 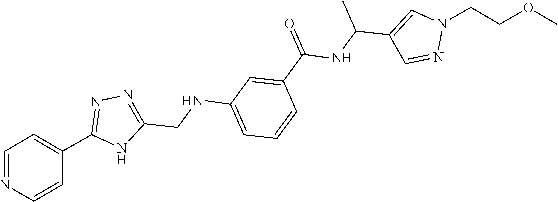 [SMILES: COCCn1cc(C(C)NC(=O)c2cccc(NCc3nnc(-c4ccncc4)[nH]3)c2)cn1] | | | >10.0 | 101 |
| 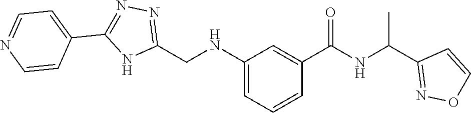 [SMILES: CC(NC(=O)c1cccc(NCc2nnc(-c3ccncc3)[nH]2)c1)c1ccon1] | 20 | | >10.0 | 66.9 |
| 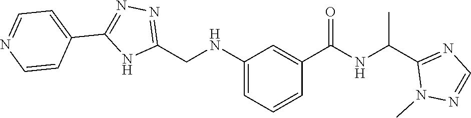 [SMILES: CC(NC(=O)c1cccc(NCc2nnc(-c3ccncc3)[nH]2)c1)c1ncnn1C] | | | >10.0 | 85 |
| 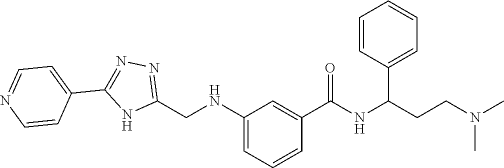 [SMILES: CN(C)CCC(NC(=O)c1cccc(NCc2nnc(-c3ccncc3)[nH]2)c1)c1ccccc1] | | | >10.0 | 83.1 |
| 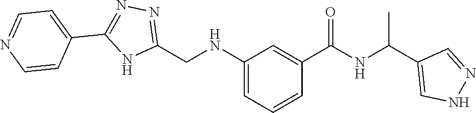 [SMILES: CC(NC(=O)c1cccc(NCc2nnc(-c3ccncc3)[nH]2)c1)c1cn[nH]c1] | | | 0.323 | 71.8 |
| 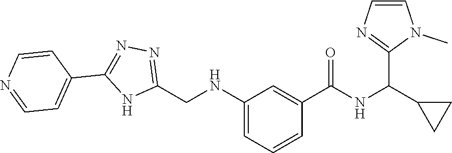 [SMILES: Cn1ccnc1C(NC(=O)c1cccc(NCc2nnc(-c3ccncc3)[nH]2)c1)C1CC1] | 10 | | 5.51 | 21.9 |
| 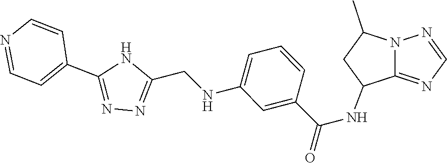 [SMILES: CC1CC(NC(=O)c2cccc(NCc3nnc(-c4ccncc4)[nH]3)c2)c2ncnn21] | 37 | | >10.0 | 105 |
| 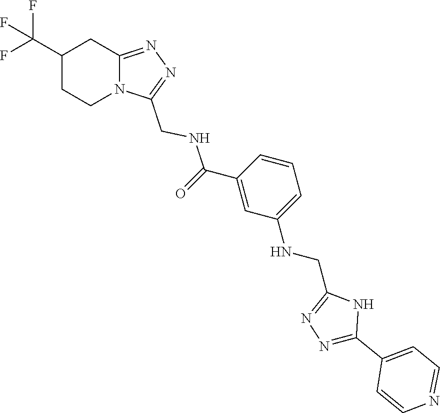 [SMILES: O=C(NCc1nnc2n1CCC(C(F)(F)F)C2)c1cccc(NCc2nnc(-c3ccncc3)[nH]2)c1] | | >10.0 | 89.3 | |
| 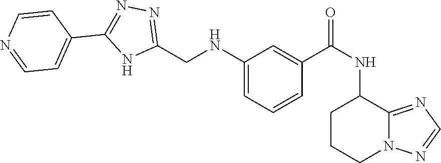 [SMILES: O=C(NC1CCCn2ncnc21)c1cccc(NCc2nnc(-c3ccncc3)[nH]2)c1] | 20 | | >10.0 | 83.1 |
| 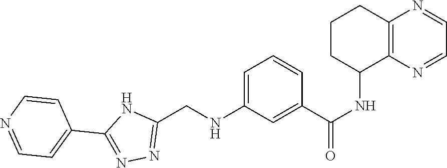 [SMILES: O=C(NC1CCCc2nccnc21)c1cccc(NCc2nnc(-c3ccncc3)[nH]2)c1] | 12 | | 0.736 | 24.3 |
| 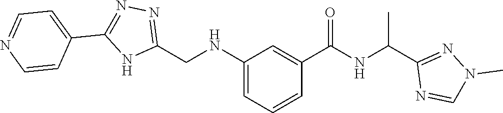 [SMILES: CC(NC(=O)c1cccc(NCc2nnc(-c3ccncc3)[nH]2)c1)c1ncn(C)n1] | | | >10.0 | 86.7 |
| 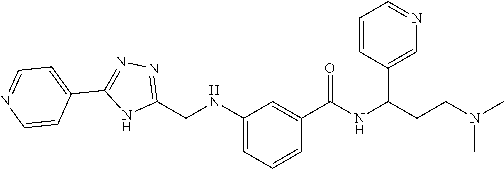 [SMILES: CN(C)CCC(NC(=O)c1cccc(NCc2nnc(-c3ccncc3)[nH]2)c1)c1cccnc1] | | | >10.0 | 82.1 |
| 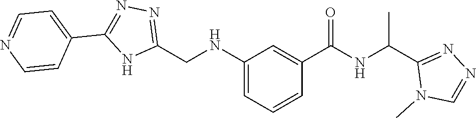 [SMILES: CC(NC(=O)c1cccc(NCc2nnc(-c3ccncc3)[nH]2)c1)c1nncn1C] | | | >10.0 | 78.6 |
| 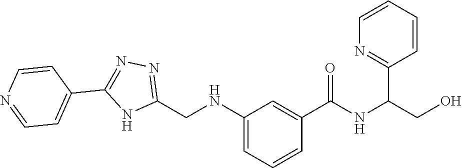 [SMILES: O=C(NC(CO)c1ccccn1)c1cccc(NCc2nnc(-c3ccncc3)[nH]2)c1] | 20 | | >10.0 | 78.5 |
| 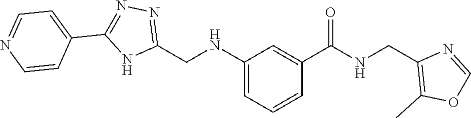 [SMILES: Cc1ocnc1CNC(=O)c1cccc(NCc2nnc(-c3ccncc3)[nH]2)c1] | 8.3 | | >7.50 | 51.9 |
| 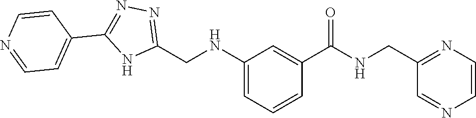 [SMILES: O=C(NCc1cnccn1)c1cccc(NCc2nnc(-c3ccncc3)[nH]2)c1] | 25 | | >10.0 | 100 |
| 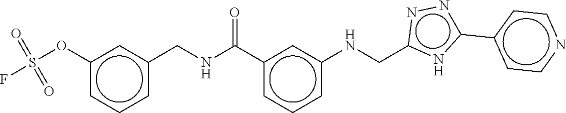 | | 26 | >30.0 | 7.07 |
| 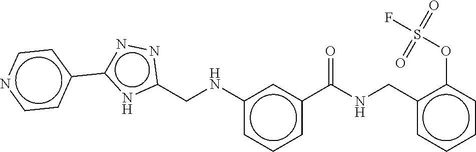 | | 12 | 7.64 | 16.3 |
| 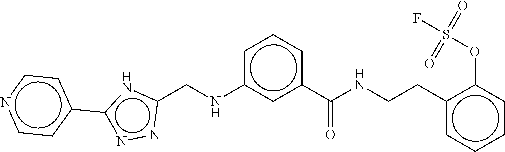 | | 73 | 13.1 | 5.47 |
| 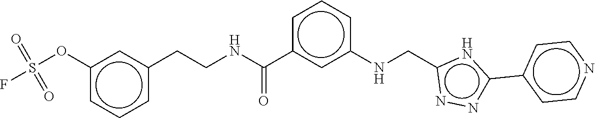 | | 210 | | |
| 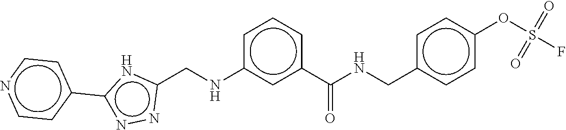 | | 20 | >30.0 | 8.59 |
| 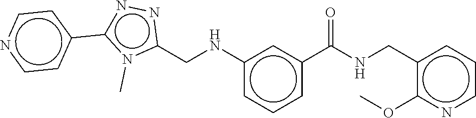 | | 15 | 0.81 | 54.2 |
| 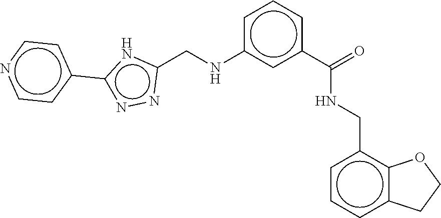 | | 2.9 | >30.0 | 7.94 |
| 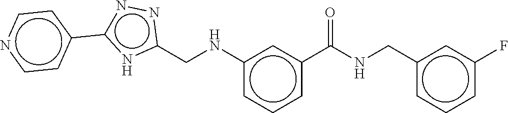 | | 6.1 | 14.5 | 8.49 |
| 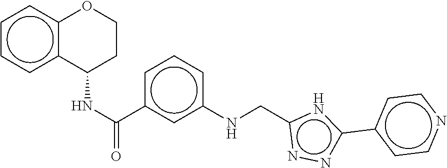 | | 2.1 | >1.76 | 1.5 |
| 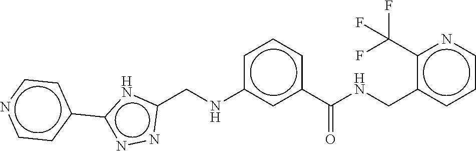 | | 13 | >30.0 | 22.6 |
| 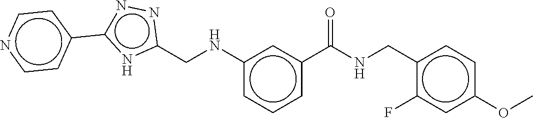 | | 15 | 18.8 | 14.6 |
| 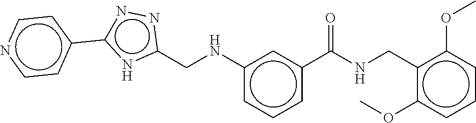 | | 1.4 | 0.981 | 15.3 |
| 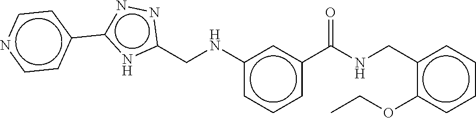 | | 10 | >30.0 | 17.7 |
| 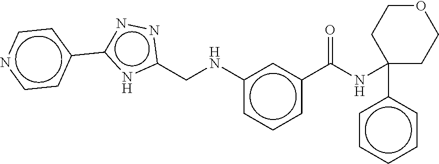 | | 330 | | |
| 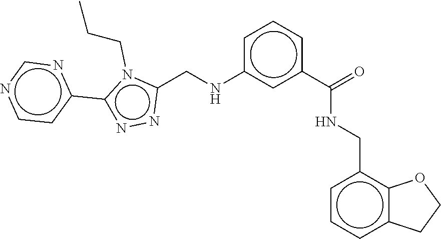 | | 2 | >30.0 | 25 |
| 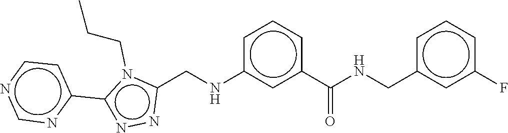 | | 5.3 | 19.2 | 27.9 |
| 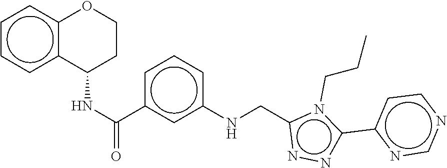 | | 2.3 | >30.0 | 17.5 |
| 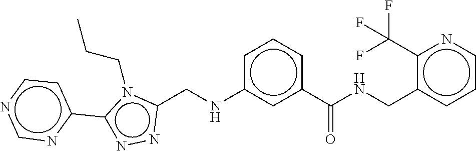 | | 2.6 | 0.667 | 37.8 |
| 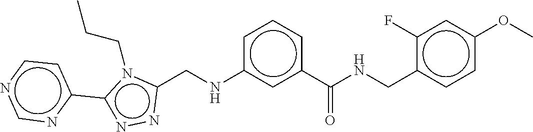 | | 2.1 | >30.0 | 54.1 |
| 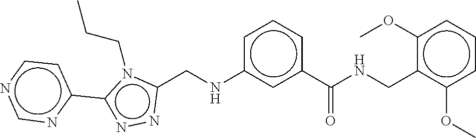 | | 3.1 | >30.0 | 69.8 |
| 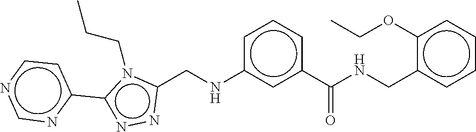 | | 8.2 | >30.0 | 24.2 |
| 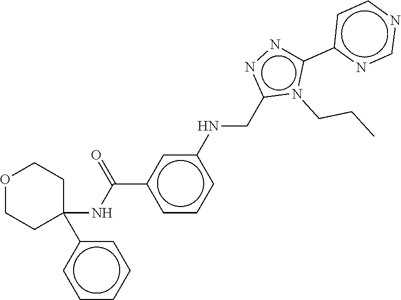 | | 22 | 9.26 | 13.2 |
| 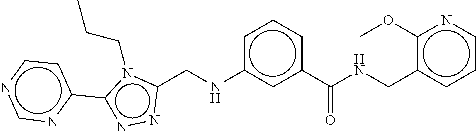 | | 3.8 | >30.0 | 68.7 |
| 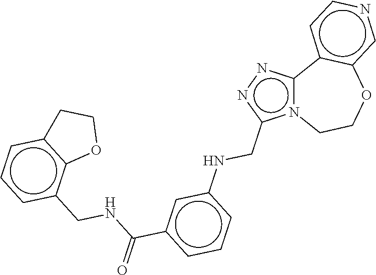 | | 2.4 | 3.01 | 12.3 |
| 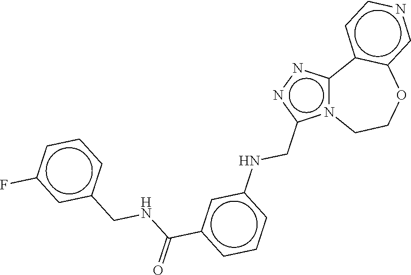 | | 3.7 | 7.02 | 23.8 |
| 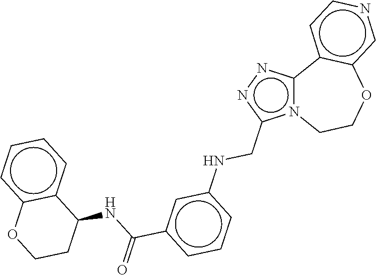 | | 2.6 | >30.0 | 16.4 |
| 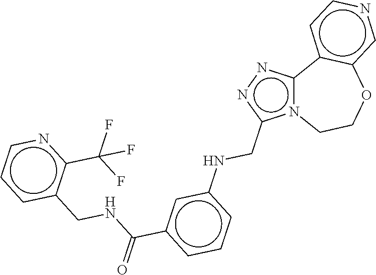 | | 3.5 | 6.57 | 16.3 |
| 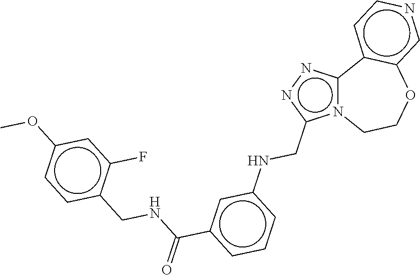 | | 1.6 | >30.0 | 25.9 |
| 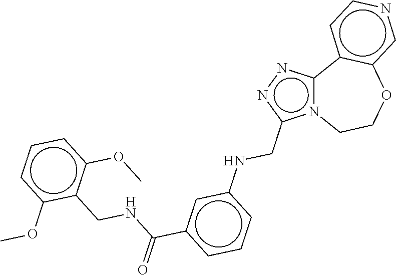 | | 6.7 | >30.0 | 73.7 |
| 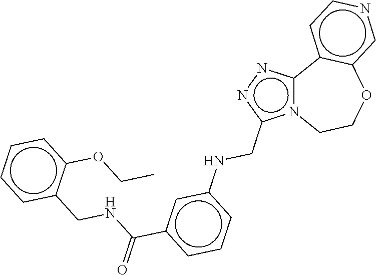 | | 9.1 | 2.32 | 10.7 |
| 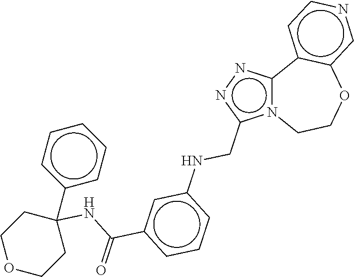 | | 39 | 20.3 | 12.1 |
| 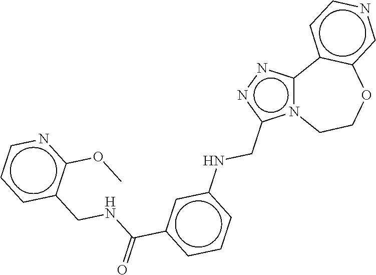 | | 7.5 | >30.0 | 29.4 |
| 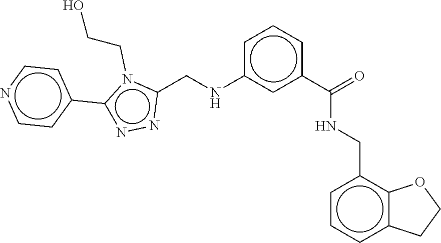 | | 6.3 | 6.08 | 69.5 |
| 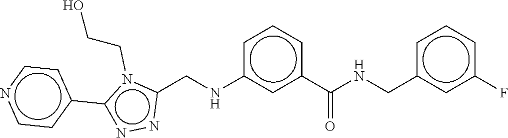 | | 9.2 | >30.0 | 83.2 |
| 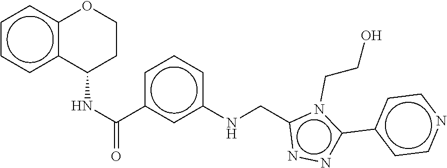 | | 8.2 | 4.58 | 58.8 |
| 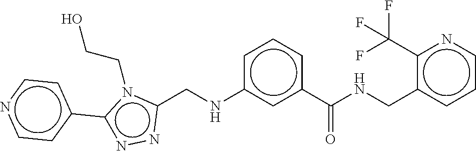 | | 5.2 | >30.0 | 92.1 |
| 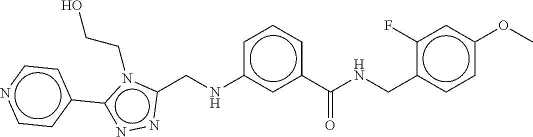 | | 4.9 | >30.0 | 78.9 |
| 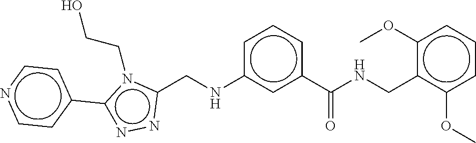 | | 23 | >30.0 | 57.9 |
| 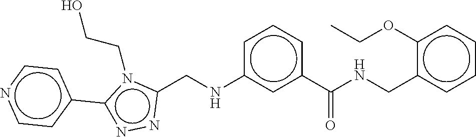 | | 19 | 6.31 | 53.3 |
| 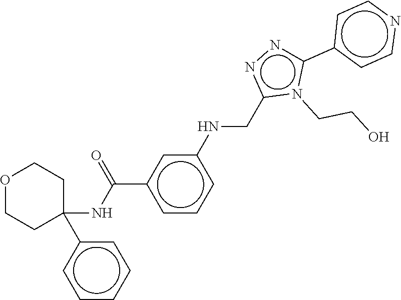 | | 130 | | |
| 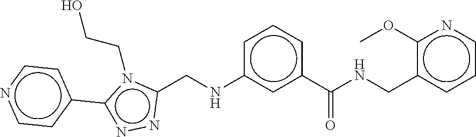 | | 20 | 0.256 | 77 |
| 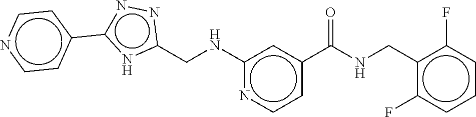 | | 34 | >30.0 | 95.4 |
| 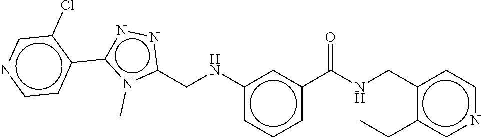 | | 79 | >30.0 | 76.6 |
| 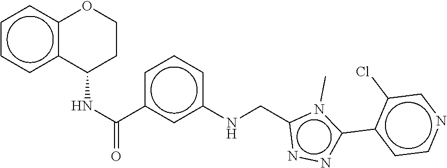 | | 170 | | |
| 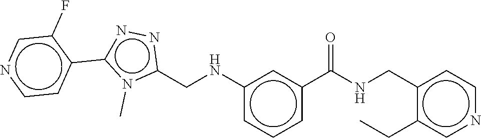 | | 41 | 1.48 | 57.2 |
| 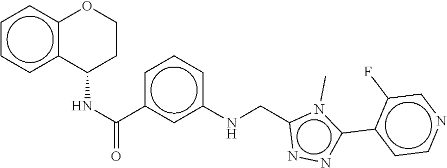 | 44.3 | 59 | 1.49 | 40.4 |
| 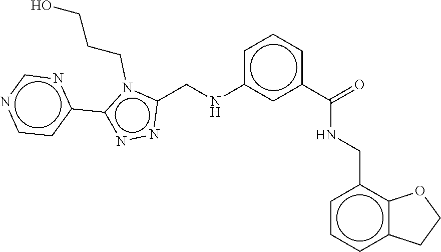 | | 4.2 | 14.5 | 58.7 |
| 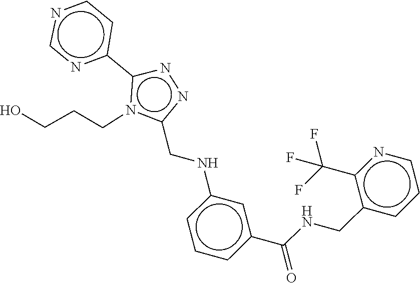 | | 5.8 | >30.0 | 44.6 |
| 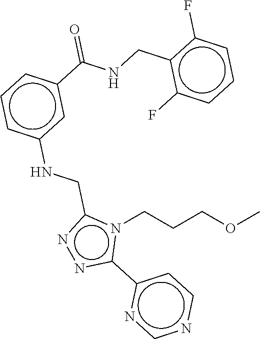 | | | | |
| 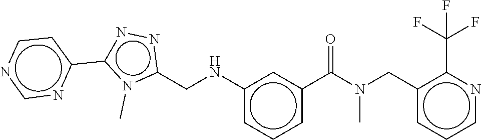 | | >10E + 03 | | |
| 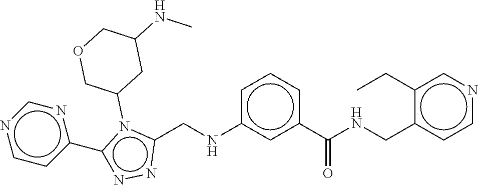 | | | | |
| 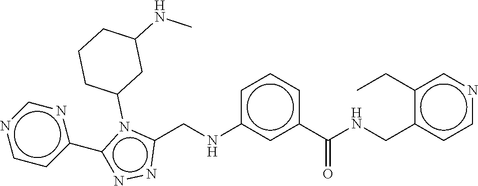 | | | | |
| 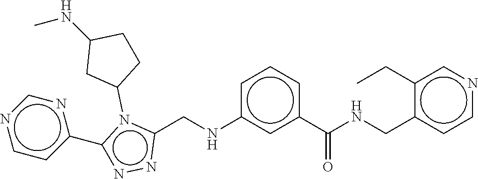 | | | | |
| 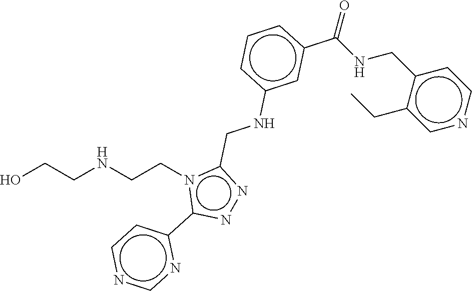 | | | | |
| 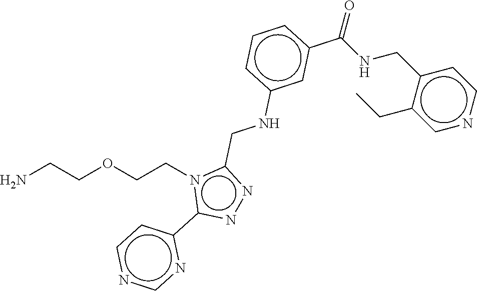 | | | | |
| 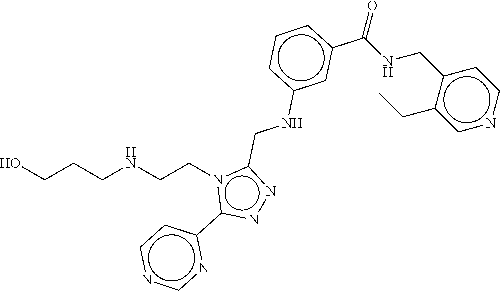 | | | | |
| 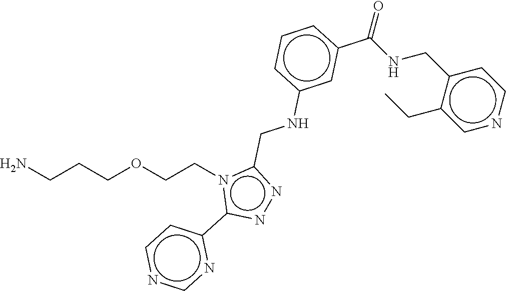 | | | | |
| 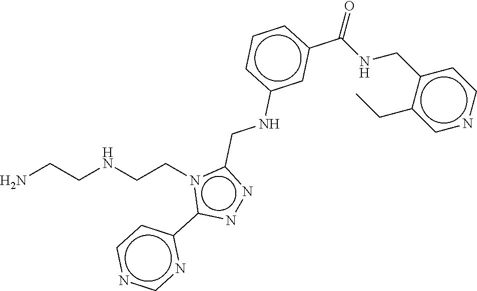 | | | | |
| 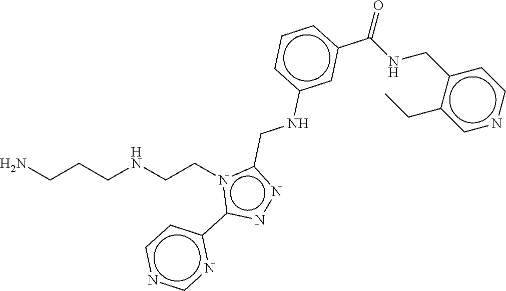 | | | | |
| 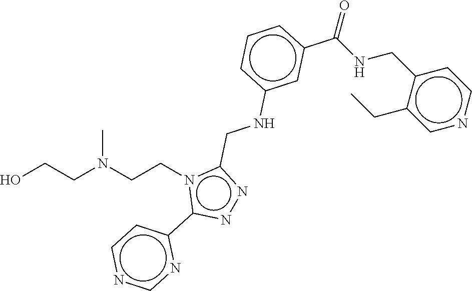 | | | | |
| 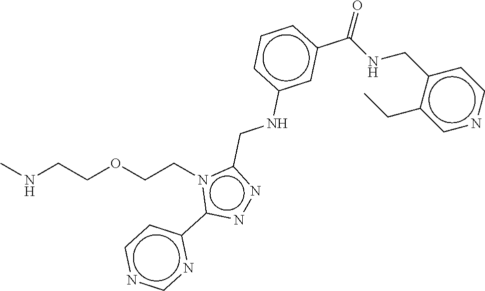 | | | | |
| 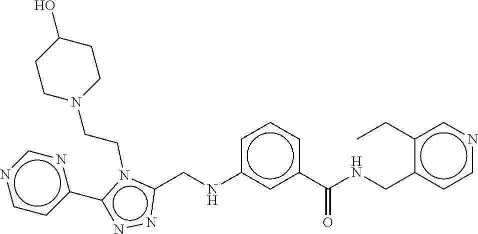 | | | | |
| 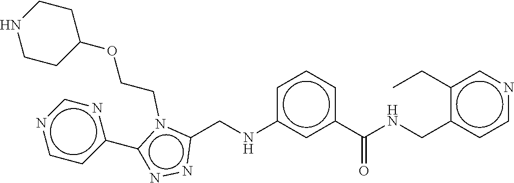 | | | | |
| 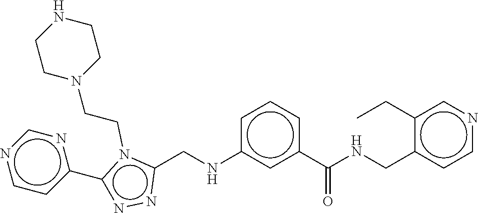 | | | | |
| 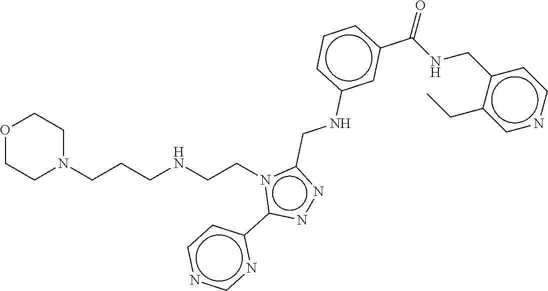 | | | | |
| 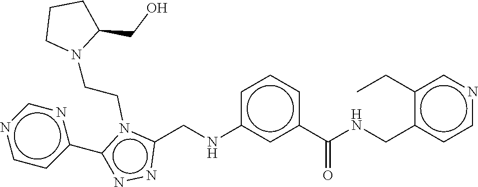 | | | | |
| 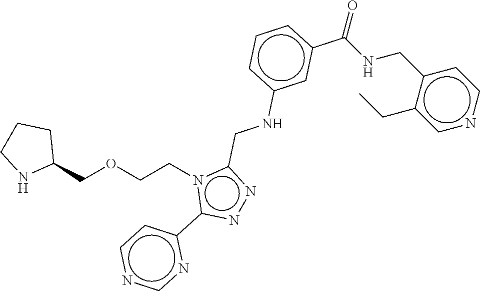 | | | | |
| 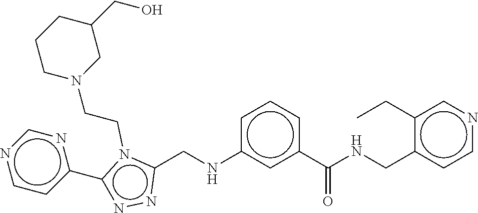 | | | | |
| 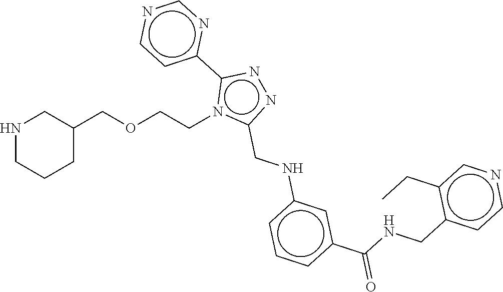 | | | | |
| 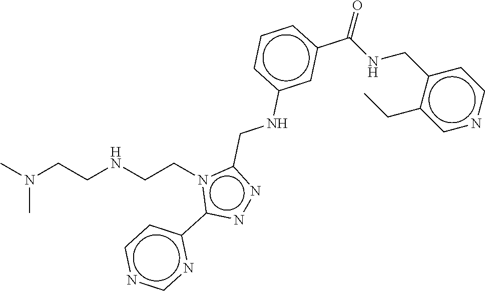 | | | | |
| 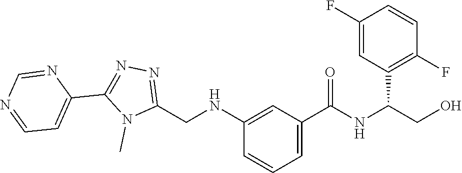 [SMILES: Cn1c(CNc2cccc(C(=O)N[C@@H](CO)c3cc(F)ccc3F)c2)nnc1-c1ccncn1] | 10 | | >30.0 | 68 |
| 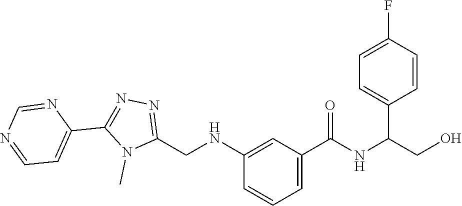 [SMILES: Cn1c(CNc2cccc(C(=O)NC(CO)c3ccc(F)cc3)c2)nnc1-c1ccncn1] | 14 | | >30.0 | 64.3 |
| 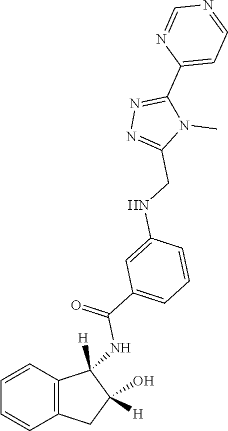 [SMILES: Cn1c(CNc2cccc(C(=O)N[C@H]3c4ccccc4C[C@H]3O)c2)nnc1-c1ccncn1] | 7.3 | 24 | 1.41 | 70 |
| 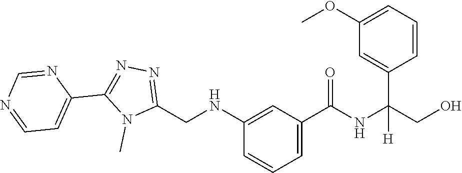 [SMILES: COc1cccc(C(CO)NC(=O)c2cccc(NCc3nnc(-c4ccncn4)n3C)c2)c1] | 5.4 | 13 | >30.0 | 84.6 |
| 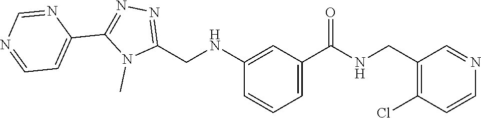 [SMILES: Cn1c(CNc2cccc(C(=O)NCc3cnccc3Cl)c2)nnc1-c1ccncn1] | 4.3 | 17 | 1.19 | 52.2 |
| 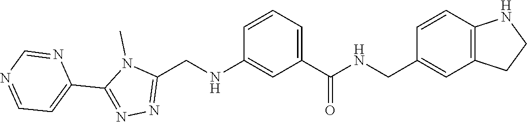 [SMILES: Cn1c(CNc2cccc(C(=O)NCc3ccc4c(c3)CCN4)c2)nnc1-c1ccncn1] | 9.4 | 15 | >30.0 | 75.3 |
| 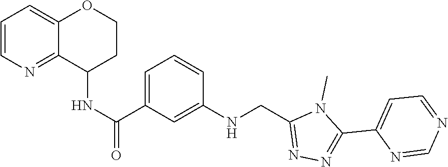 [SMILES: Cn1c(CNc2cccc(C(=O)NC3CCOc4cccnc43)c2)nnc1-c1ccncn1] | 9.6 | 13 | 0.285 | 67.5 |
| 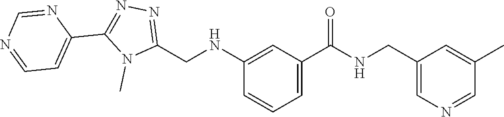 [SMILES: Cc1cncc(CNC(=O)c2cccc(NCc3nnc(-c4ccncn4)n3C)c2)c1] | 15 | | >30.0 | 65.1 |
| 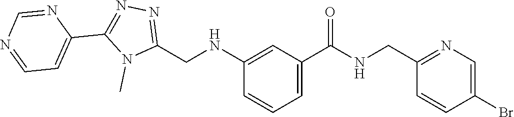 [SMILES: Cn1c(CNc2cccc(C(=O)NCc3ccc(Br)cn3)c2)nnc1-c1ccncn1] | 140 | | | |
| 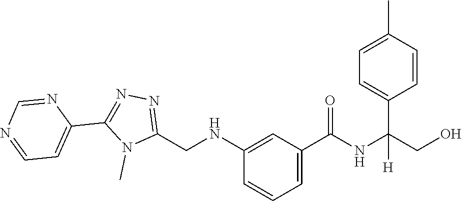 [SMILES: Cc1ccc(C(CO)NC(=O)c2cccc(NCc3nnc(-c4ccncn4)n3C)c2)cc1] | 3.3 | 8.1 | >30.0 | 79.4 |
| 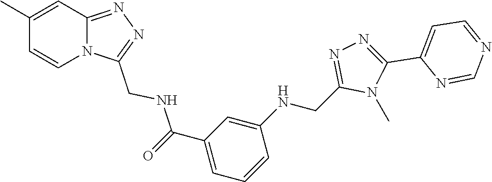 [SMILES: Cc1ccn2c(CNC(=O)c3cccc(NCc4nnc(-c5ccncn5)n4C)c3)nnc2c1] | 14 | | >30.0 | 69.7 |
| 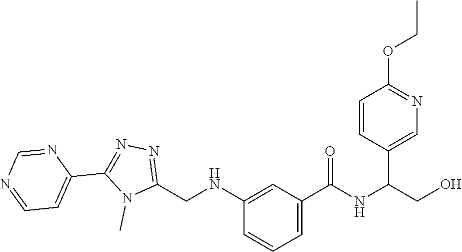 [SMILES: CCOc1ccc(C(CO)NC(=O)c2cccc(NCc3nnc(-c4ccncn4)n3C)c2)cn1] | 1.8 | 12 | >30.0 | 77.2 |
| 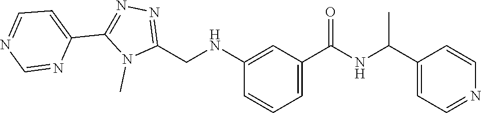 [SMILES: CC(NC(=O)c1cccc(NCc2nnc(-c3ccncn3)n2C)c1)c1ccncc1] | 11 | | 2.15 | 46.2 |
| 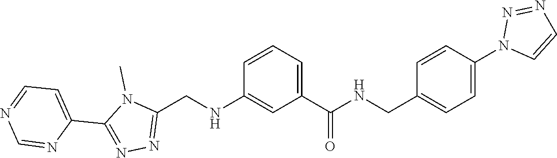 [SMILES: Cn1c(CNc2cccc(C(=O)NCc3ccc(-n4ccnn4)cc3)c2)nnc1-c1ccncn1] | 9.5 | 18 | >30.0 | 94.9 |
| 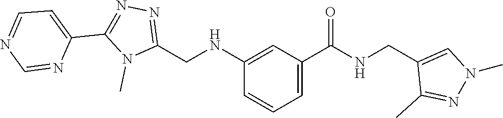 [SMILES: Cc1nn(C)cc1CNC(=O)c1cccc(NCc2nnc(-c3ccncn3)n2C)c1] | 19 | | 6.22 | 64.8 |
| 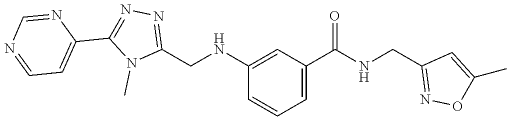 [SMILES: Cc1cc(CNC(=O)c2cccc(NCc3nnc(-c4ccncn4)n3C)c2)no1] | 110 | | | |
| 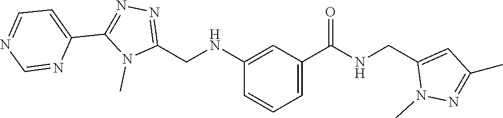 [SMILES: Cc1cc(CNC(=O)c2cccc(NCc3nnc(-c4ccncn4)n3C)c2)n(C)n1] | 17 | | 4.15 | 58.9 |
| 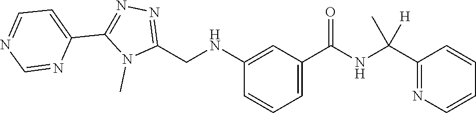 [SMILES: CC(NC(=O)c1cccc(NCc2nnc(-c3ccncn3)n2C)c1)c1ccccn1] | 29 | | 0.522 | 76 |
| 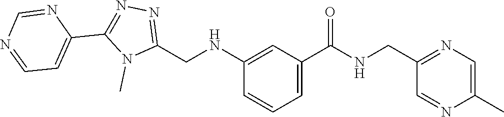 [SMILES: Cc1cnc(CNC(=O)c2cccc(NCc3nnc(-c4ccncn4)n3C)c2)cn1] | 150 | | | |
| 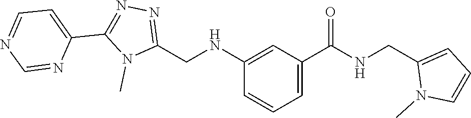 [SMILES: Cn1cccc1CNC(=O)c1cccc(NCc2nnc(-c3ccncn3)n2C)c1] | 9.5 | 6.7 | 0.551 | 70.4 |
| 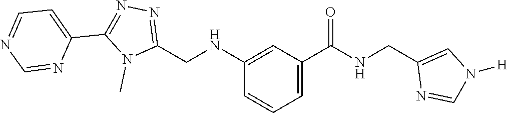 [SMILES: Cn1c(CNc2cccc(C(=O)NCc3c[nH]cn3)c2)nnc1-c1ccncn1] | 110 | | | |
| 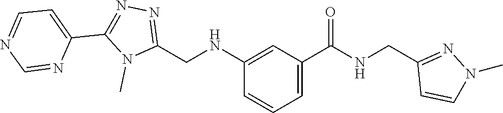 [SMILES: Cn1ccc(CNC(=O)c2cccc(NCc3nnc(-c4ccncn4)n3C)c2)n1] | 60 | | 1.29 | 57.9 |
| 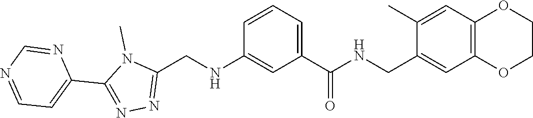 [SMILES: Cc1cc2c(cc1CNC(=O)c1cccc(NCc3nnc(-c4ccncn4)n3C)c1)OCCO2] | 0.5 | 3.3 | 0.996 | 52 |
| 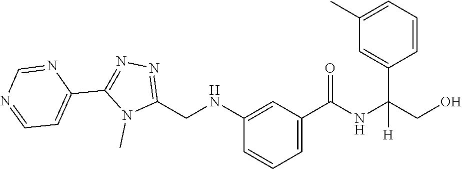 [SMILES: Cc1cccc(C(CO)NC(=O)c2cccc(NCc3nnc(-c4ccncn4)n3C)c2)c1] | 1.1 | 8.6 | 0.407 | 71.9 |
| 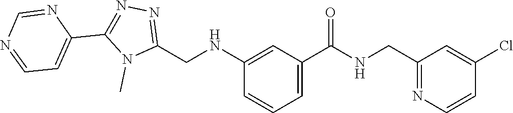 [SMILES: Cn1c(CNc2cccc(C(=O)NCc3cc(Cl)ccn3)c2)nnc1-c1ccncn1] | 45 | | >30.0 | 31.9 |
| 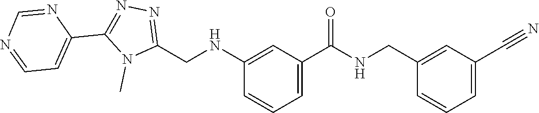 [SMILES: Cn1c(CNc2cccc(C(=O)NCc3cccc(C#N)c3)c2)nnc1-c1ccncn1] | 2.7 | 12 | >30.0 | 66.9 |
| 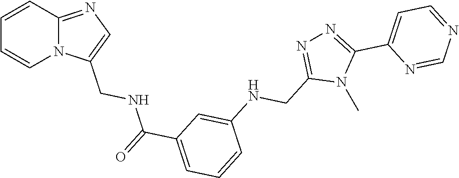 [SMILES: Cn1c(CNc2cccc(C(=O)NCc3cnc4ccccn34)c2)nnc1-c1ccncn1] | 0.96 | 3.2 | 1.49 | 57.1 |
| 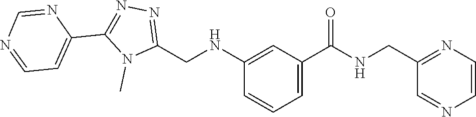 [SMILES: Cn1c(CNc2cccc(C(=O)NCc3cnccn3)c2)nnc1-c1ccncn1] | 37 | | >30.0 | 87.1 |
| 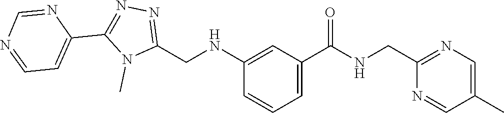 [SMILES: Cc1cnc(CNC(=O)c2cccc(NCc3nnc(-c4ccncn4)n3C)c2)nc1] | 580 | | | |
| 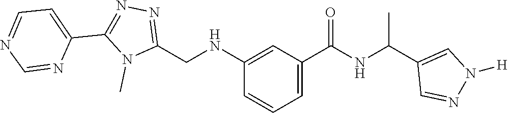 [SMILES: CC(NC(=O)c1cccc(NCc2nnc(-c3ccncn3)n2C)c1)c1cn[nH]c1] | 13 | | 16.8 | 58.8 |
| 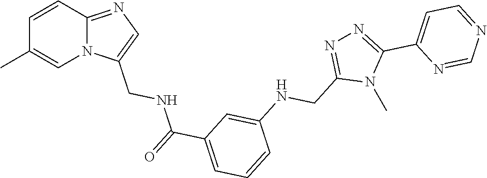 [SMILES: Cc1ccc2ncc(CNC(=O)c3cccc(NCc4nnc(-c5ccncn5)n4C)c3)n2c1] | 2.8 | 7.2 | >30.0 | 59.3 |
| 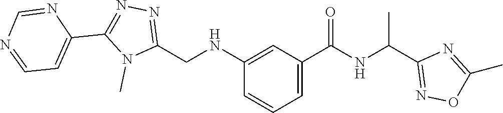 [SMILES: Cc1nc(C(C)NC(=O)c2cccc(NCc3nnc(-c4ccncn4)n3C)c2)no1] | 67 | | >30.0 | 77.2 |
| 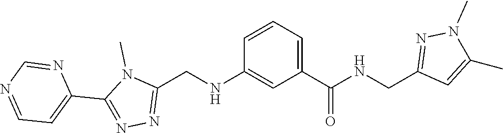 [SMILES: Cc1cc(CNC(=O)c2cccc(NCc3nnc(-c4ccncn4)n3C)c2)nn1C] | 41 | | >30.0 | 38.8 |
| 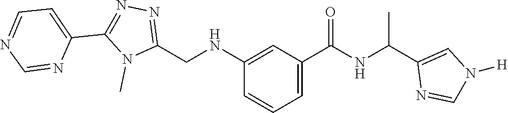 [SMILES: CC(NC(=O)c1cccc(NCc2nnc(-c3ccncn3)n2C)c1)c1c[nH]cn1] | 5.7 | 60 | >30.0 | 87.7 |
| 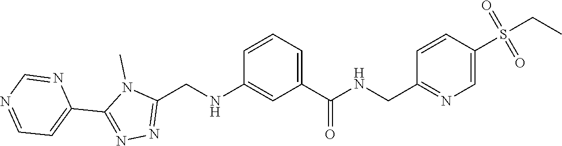 [SMILES: CCS(=O)(=O)c1ccc(CNC(=O)c2cccc(NCc3nnc(-c4ccncn4)n3C)c2)nc1] | 100 | | | |
| 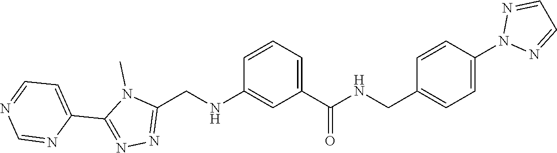 [SMILES: Cn1c(CNc2cccc(C(=O)NCc3ccc(-n4nccn4)cc3)c2)nnc1-c1ccncn1] | 2.1 | 20 | >30.0 | 73.5 |
| 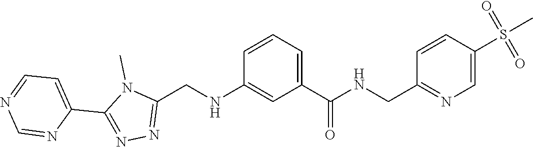 [SMILES: Cn1c(CNc2cccc(C(=O)NCc3ccc(S(C)(=O)=O)cn3)c2)nnc1-c1ccncn1] | 240 | | | |
| 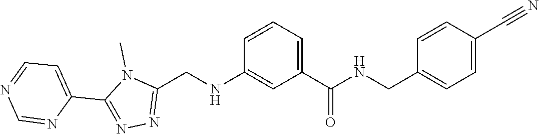 [SMILES: Cn1c(CNc2cccc(C(=O)NCc3ccc(C#N)cc3)c2)nnc1-c1ccncn1] | 6.3 | 20 | >30.0 | 81.8 |
| 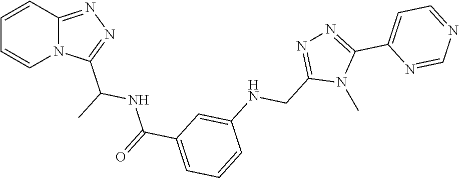 [SMILES: CC(NC(=O)c1cccc(NCc2nnc(-c3ccncn3)n2C)c1)c1nnc2ccccn12] | 260 | | | |
| 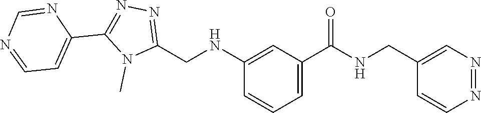 [SMILES: Cn1c(CNc2cccc(C(=O)NCc3ccnnc3)c2)nnc1-c1ccncn1] | 43 | | >30.0 | 62.9 |
| 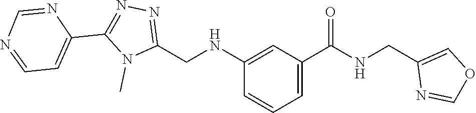 [SMILES: Cn1c(CNc2cccc(C(=O)NCc3cocn3)c2)nnc1-c1ccncn1] | 27 | | >30.0 | 81.9 |
| 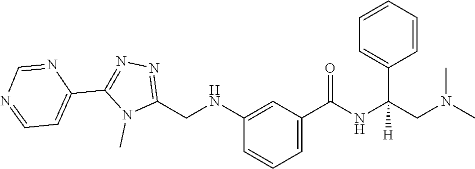 [SMILES: CN(C)C[C@@H](NC(=O)c1cccc(NCc2nnc(-c3ccncn3)n2C)c1)c1ccccc1] | 210 | | | |
| 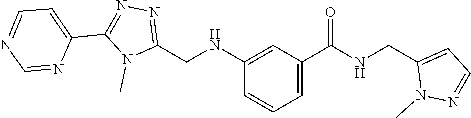 [SMILES: Cn1nccc1CNC(=O)c1cccc(NCc2nnc(-c3ccncn3)n2C)c1] | 5.7 | 29 | >30.0 | 66.9 |
| 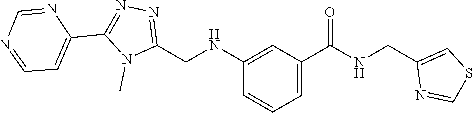 [SMILES: Cn1c(CNc2cccc(C(=O)NCc3cscn3)c2)nnc1-c1ccncn1] | 13 | | >30.0 | 80.8 |
| 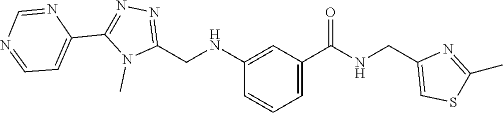 [SMILES: Cc1nc(CNC(=O)c2cccc(NCc3nnc(-c4ccncn4)n3C)c2)cs1] | 9.8 | 13 | >30.0 | 63.7 |
| 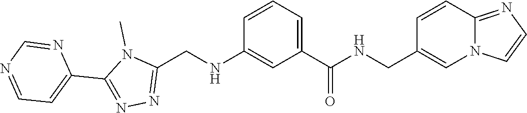 [SMILES: Cn1c(CNc2cccc(C(=O)NCc3ccc4nccn4c3)c2)nnc1-c1ccncn1] | 1.7 | 26 | 2.93 | 70.7 |
| 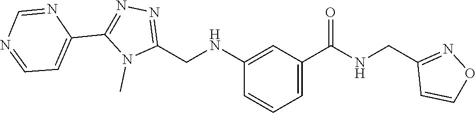 [SMILES: Cn1c(CNc2cccc(C(=O)NCc3ccon3)c2)nnc1-c1ccncn1] | 23 | | >30.0 | 82.9 |
| 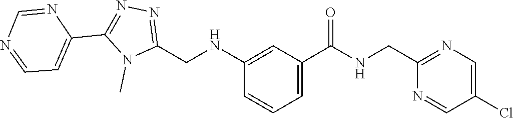 [SMILES: Cn1c(CNc2cccc(C(=O)NCc3ncc(Cl)cn3)c2)nnc1-c1ccncn1] | 240 | | | |
| 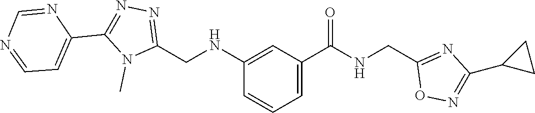 [SMILES: Cn1c(CNc2cccc(C(=O)NCc3nc(C4CC4)no3)c2)nnc1-c1ccncn1] | 27 | | 5.75 | 66.9 |
| 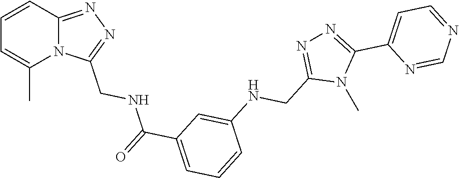 [SMILES: Cc1cccc2nnc(CNC(=O)c3cccc(NCc4nnc(-c5ccncn5)n4C)c3)n12] | 0.79 | 2.1 | >30.0 | 29.3 |
| 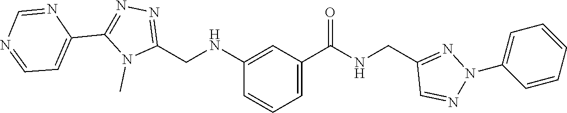 [SMILES: Cn1c(CNc2cccc(C(=O)NCc3cnn(-c4ccccc4)n3)c2)nnc1-c1ccncn1] | 6.5 | 18 | >30.0 | 527 |
| 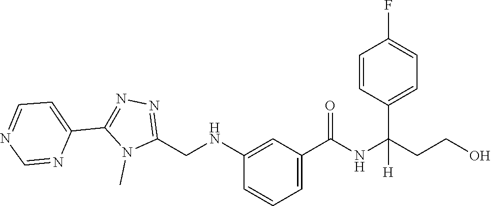 [SMILES: Cn1c(CNc2cccc(C(=O)NC(CCO)c3ccc(F)cc3)c2)nnc1-c1ccncn1] | 52 | | | |
| 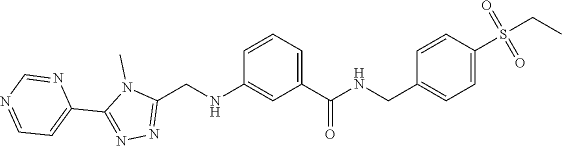 [SMILES: CCS(=O)(=O)c1ccc(CNC(=O)c2cccc(NCc3nnc(-c4ccncn4)n3C)c2)cc1] | 6.2 | 21 | >30.0 | 86.3 |
| 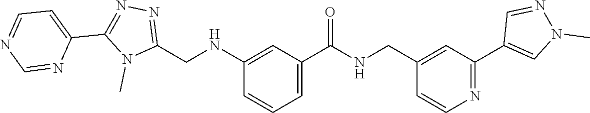 [SMILES: Cn1cc(-c2cc(CNC(=O)c3cccc(NCc4nnc(-c5ccncn5)n4C)c3)ccn2)cn1] | 17 | | >30.0 | 89 |
| 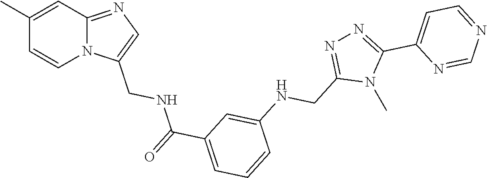 [SMILES: Cc1ccn2c(CNC(=O)c3cccc(NCc4nnc(-c5ccncn5)n4C)c3)cnc2c1] | 3 | 20 | 5.6 | 78.6 |
| 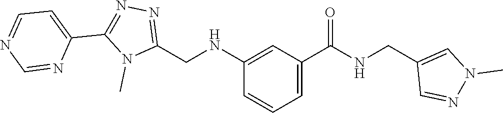 [SMILES: Cn1cc(CNC(=O)c2cccc(NCc3nnc(-c4ccncn4)n3C)c2)cn1] | 36 | | 9.77 | 70 |
| 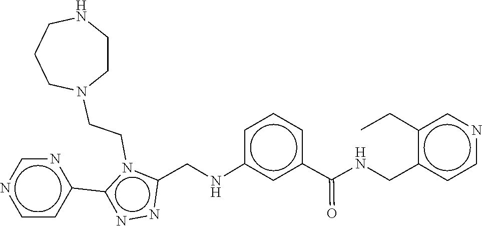 | | | | |
| 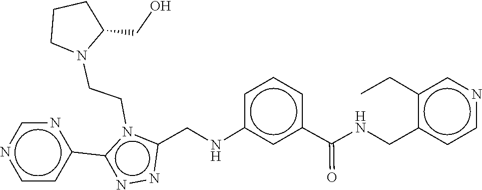 | | | | |
| 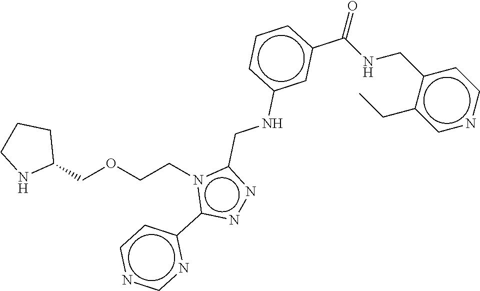 | | | | |
| 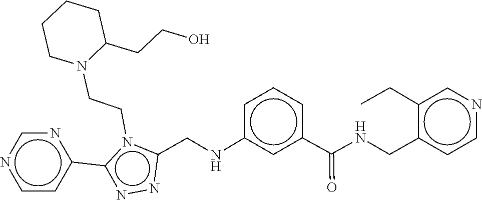 | | | | |
| 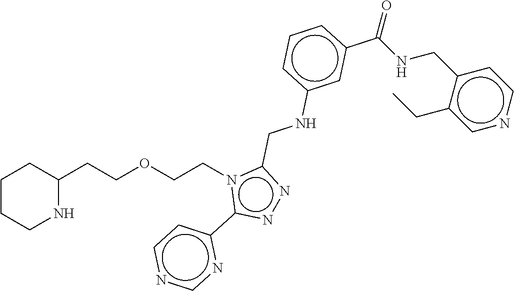 | | | | |
| 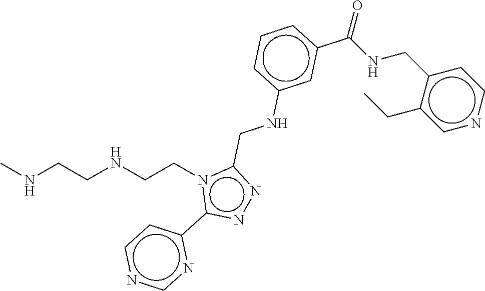 | | | | |
| 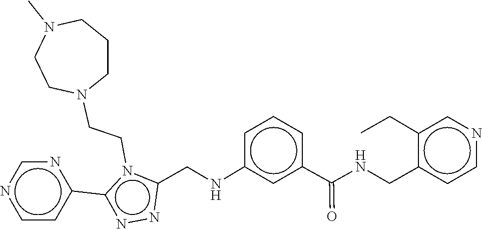 | | | | |
| 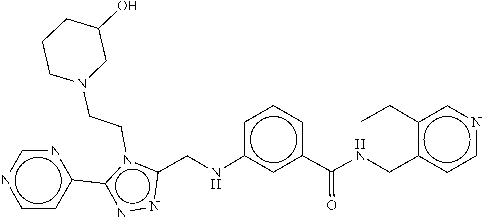 | | | | |
| 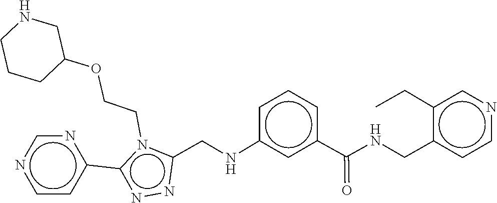 | | | | |
| 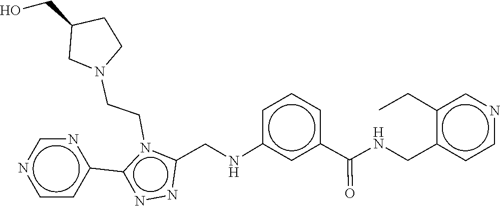 | | | | |
| 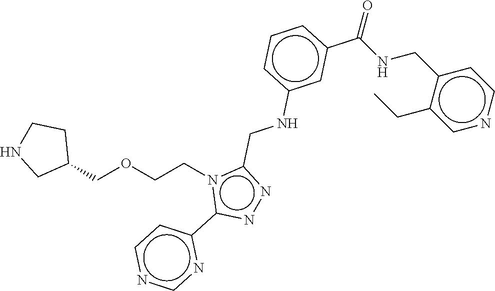 | | | | |
| 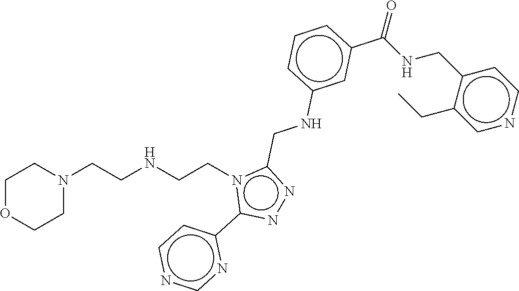 | | | | |
| 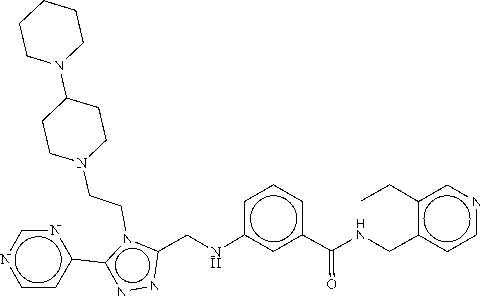 | | | | |
| 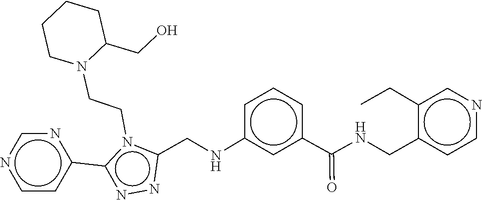 | | | | |
| 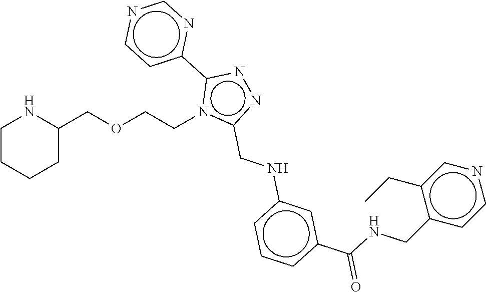 | | | | |
| 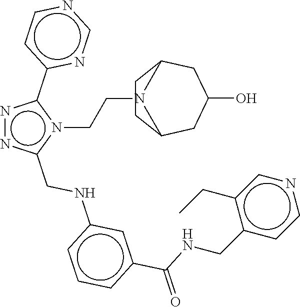 | | | | |
| 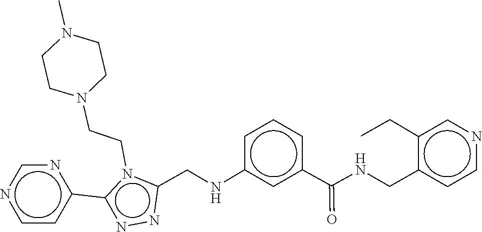 | | | | |
| 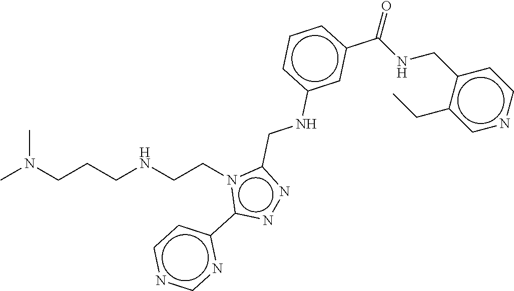 | | | | |
| 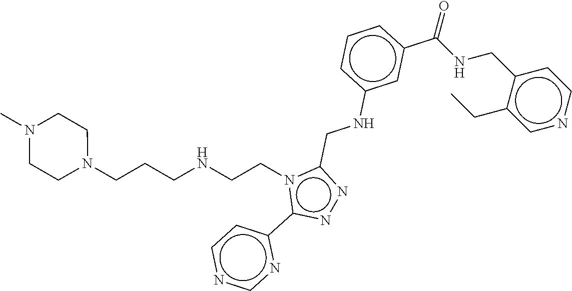 | | | | |
| 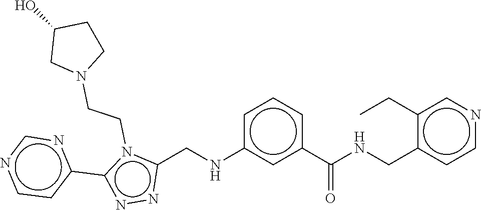 | | | | |
| 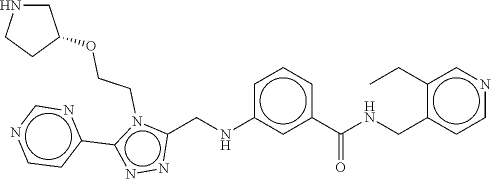 | | | | |
| 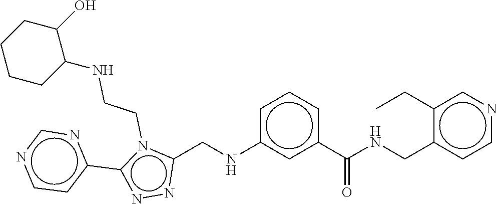 | | | | |
| 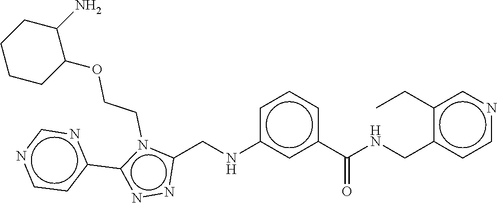 | | | | |
| 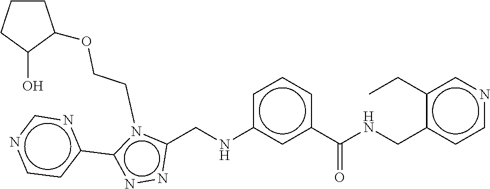 | | | | |
| 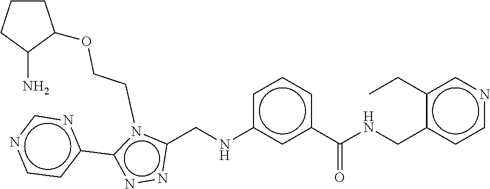 | | | | |
| 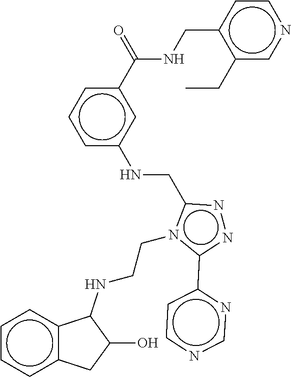 | | | | |
| 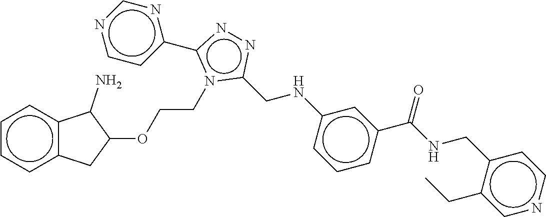 | | | | |
| 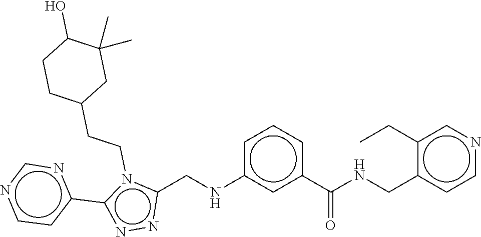 | | | | |
| 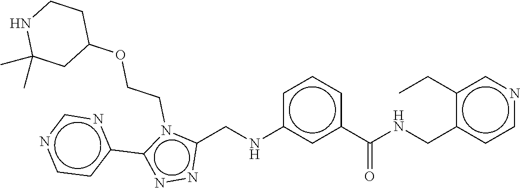 | | | | |
| 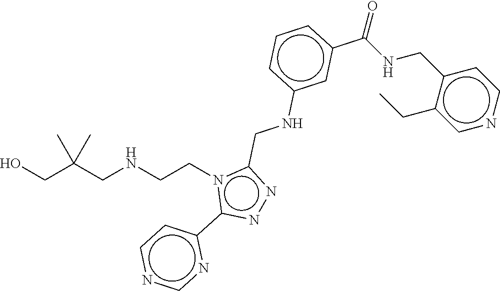 | | | | |
| 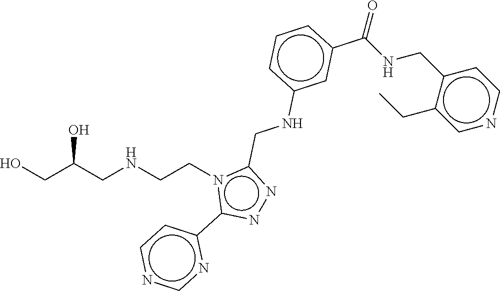 | | | | |
| 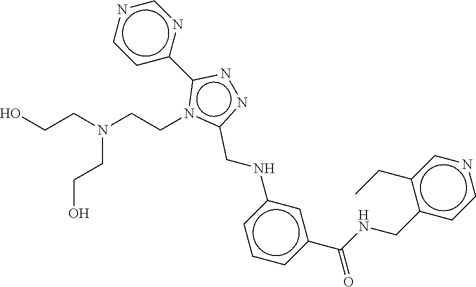 | | | | |
| 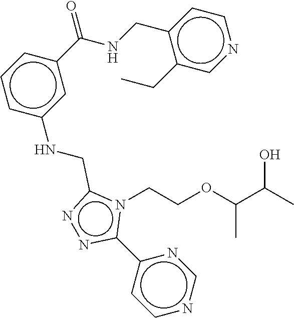 | | | | |
| 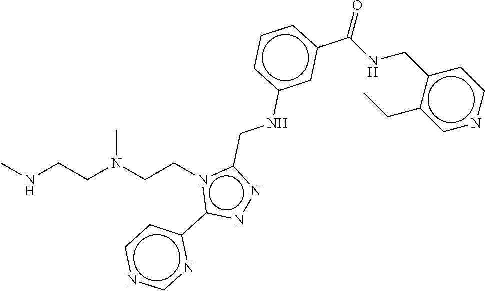 | | | | |
| 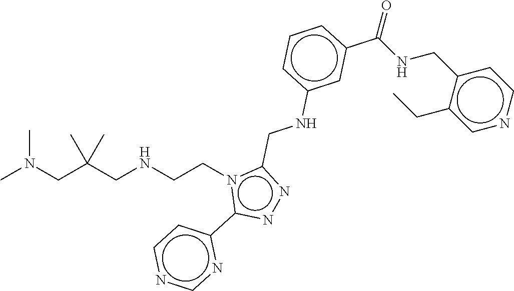 | | | | |
| 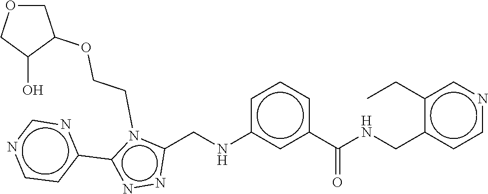 | | | | |
| 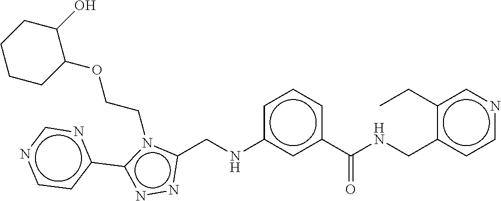 | | | | |
| 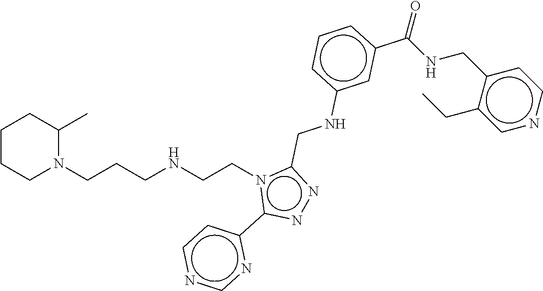 | | | | |
| 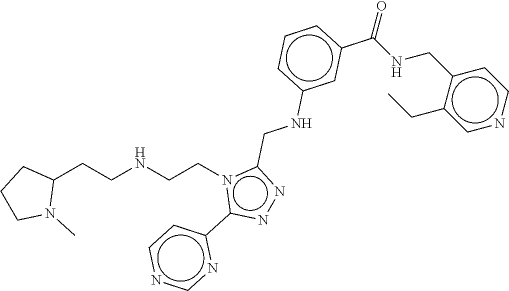 | | | | |
| 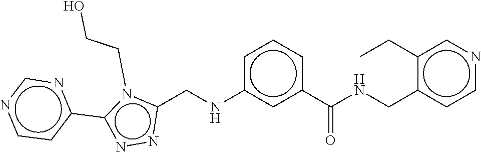 | | 0.82 | | |
| 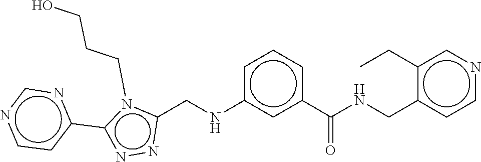 | | 5.5 | | |
| 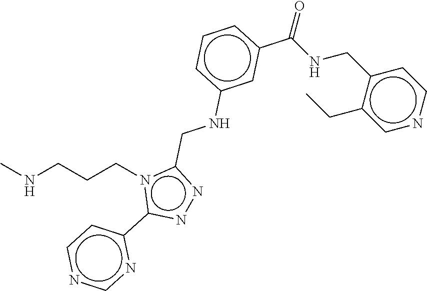 | | 94 | >30.0 | 87.8 |
| 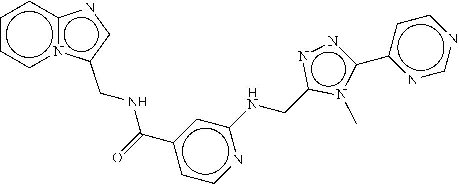 | | 7.9 | 2.75 | 70.2 |
| 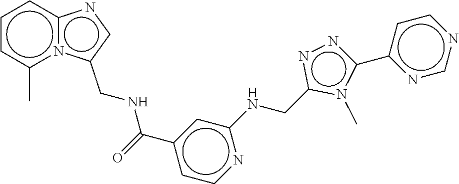 | | 24 | >30.0 | 44 |
| 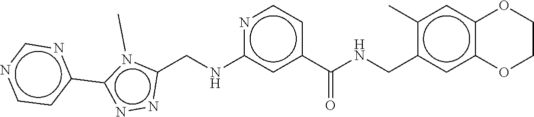 | 22.1 | 11.6 | >0.33 | 45.0 |
| 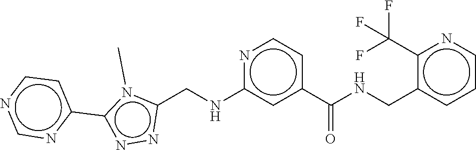 | | 11 | >30.0 | 63.7 |
| 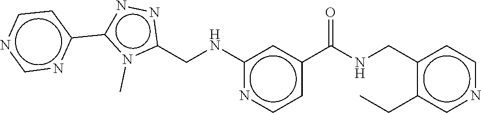 | | 15 | | |
| 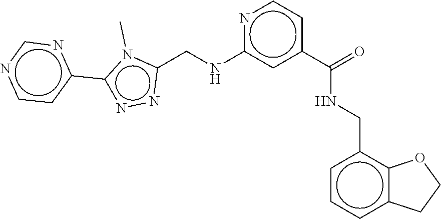 | | 12 | | |
| 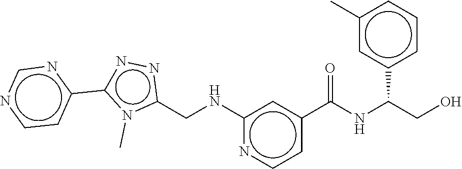 | | 16 | >30.0 | 65.9 |
| 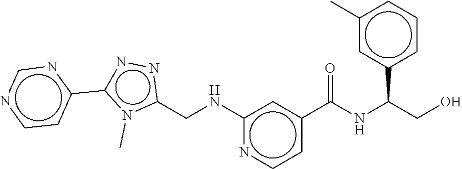 | | 96 | >30.0 | 52.8 |
| 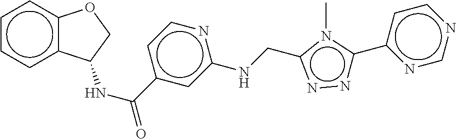 | 12.9 | 15.3 | 0.476 | 49.9 |
| 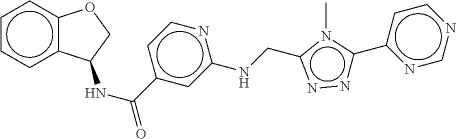 | | 26 | 1.8 | 42.3 |
| 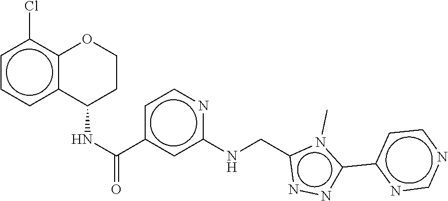 | | 63 | >30.0 | 71.3 |
| 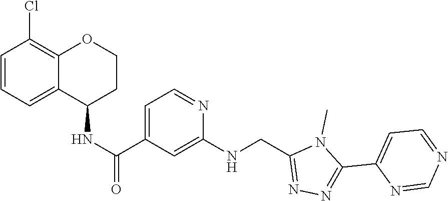 [SMILES: Cn1c(CNc2cc(C(=O)N[C@@H]3CCOc4c(Cl)cccc43)ccn2)nnc1-c1ccncn1] | | 20 | 2.07 | 45.1 |
| 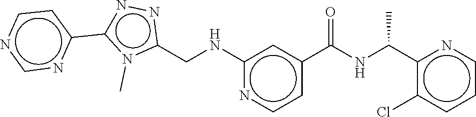 | | 55 | >30.0 | 55.9 |
| 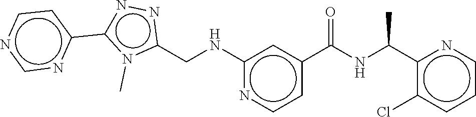 | | 65 | >30.0 | 38.3 |
| 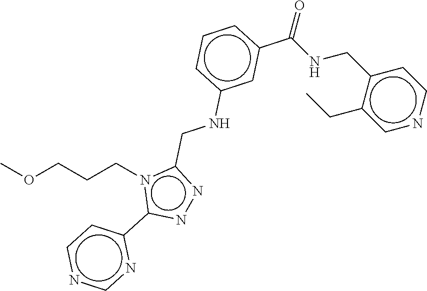 | | 23 | >30.0 | 63.1 |
| 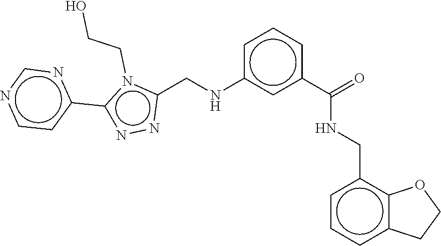 | | 1.7 | >30.0 | 17.4 |
| 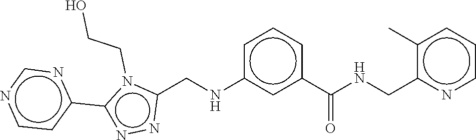 | | 2.5 | 1.16 | 20.9 |
| 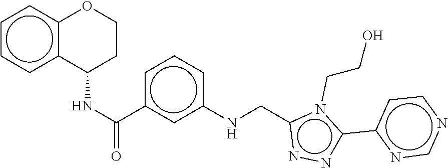 | 4.1 | 2.5 | 0.104 | 49.4 |
| 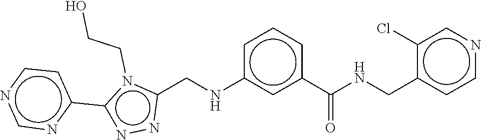 | | <2.9 | >2.97 | 52.6 |
| 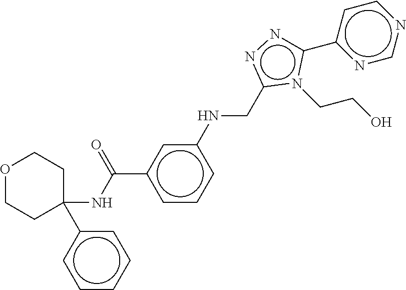 | | 6.3 | | |
| 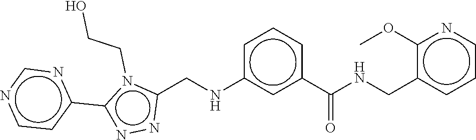 | | 2.4 | | |
| 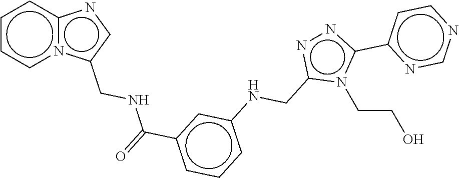 | | <0.51 | 0.58 | 52 |
| 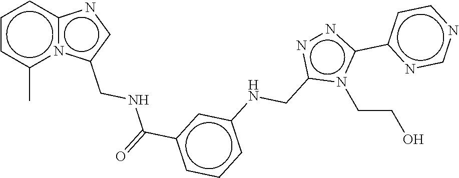 | | 1 | 3.08 | 18.3 |
| 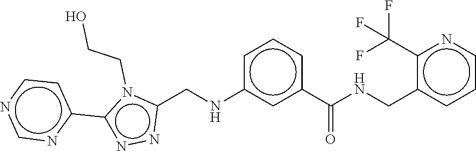 | | 0.98 | >30.0 | 21.4 |
| 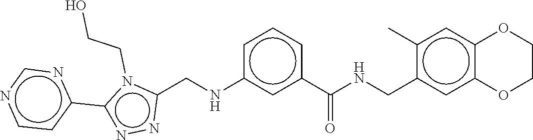 | | 1.4 | >30.0 | 27.8 |
| 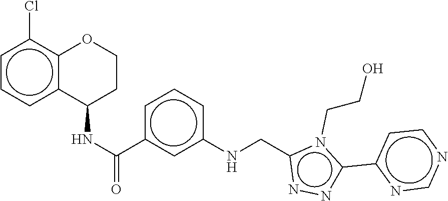 | | 4.6 | 0.123 | 38.7 |
| 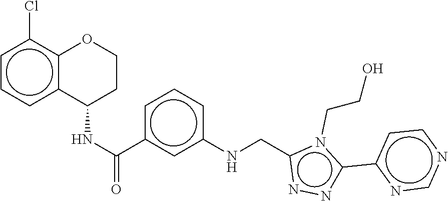 | | 1.5 | 0.163 | 24.1 |
| 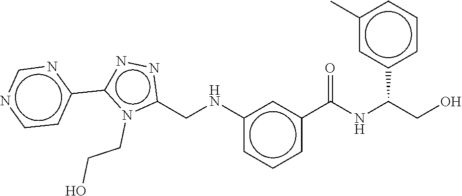 | 2.32 | <0.8 | 0.05 | 56.0 |
| 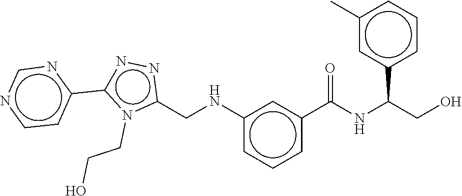 | | 1.1 | 1.51 | 50.2 |
| 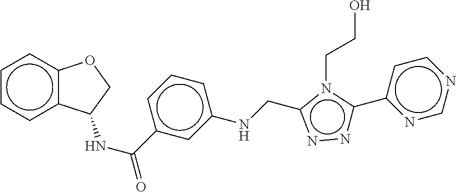 | | <0.51 | 0.107 | 46.8 |
| 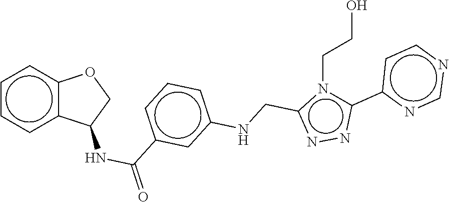 | 1.95 | <3.6 | 0.09 | 47.5 |
| 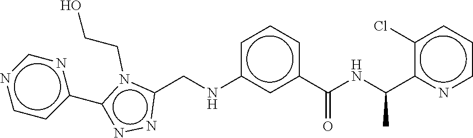 | | <0.51 | 0.803 | 45.2 |
| 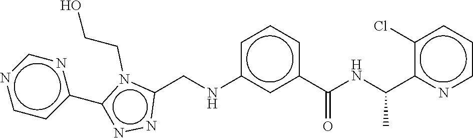 | 4.89 | 0.88 | >0.22 | 35.9 |
| 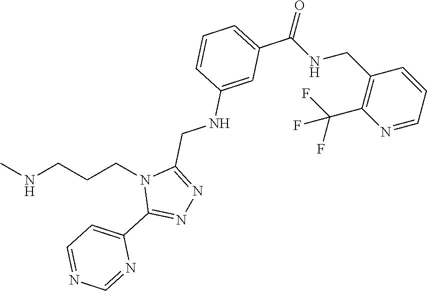 [SMILES: CNCCCn1c(CNc2cccc(C(=O)NCc3cccnc3C(F)(F)F)c2)nnc1-c1ccncn1] | | 280 | | |
| 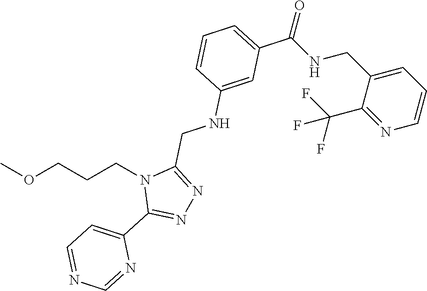 [SMILES: COCCCn1c(CNc2cccc(C(=O)NCc3cccnc3C(F)(F)F)c2)nnc1-c1ccncn1] | | 15 | 1.2 | 25.9 |
| 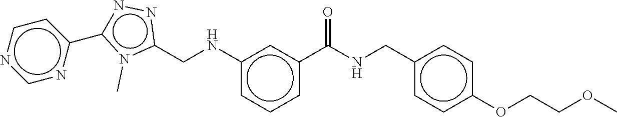 | 20.4 | | >14.2 | 47.1 |
| 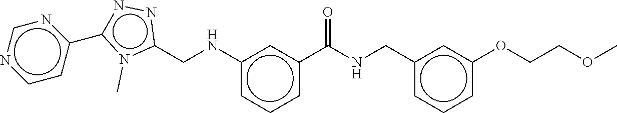 | | 39 | 0.779 | 55.7 |
| 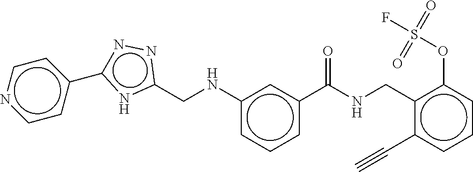 | 8.94 | 4.2 | 1.55 | 6.68 |
| 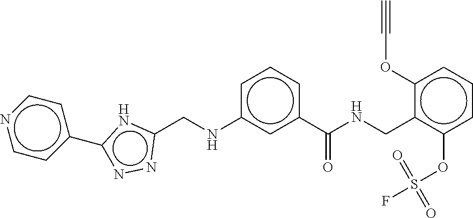 | 15.5 | 7.7 | 13.3 | 52.2 |
| 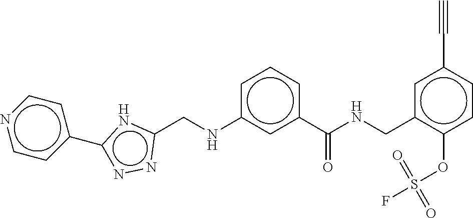 | | 13 | >7.50 | 54 |
| 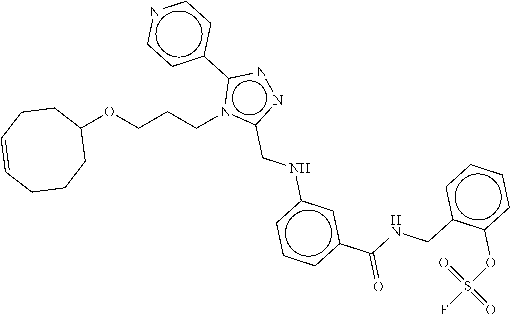 | | | | |
| 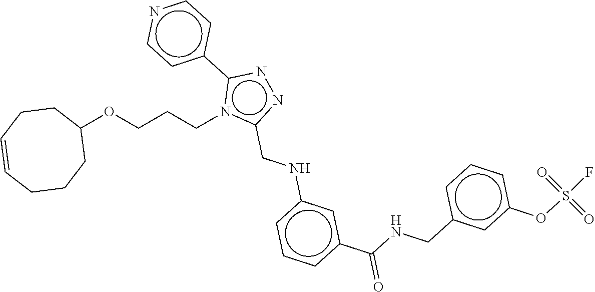 | | | | |
| 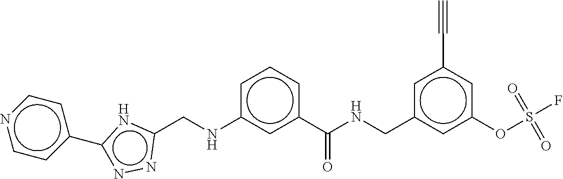 | 34.9 | | 10.8 | 8.62 |
| 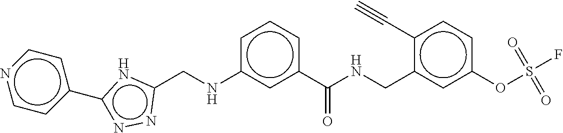 | 28.4 | | 7.44 | 38.1 |
| 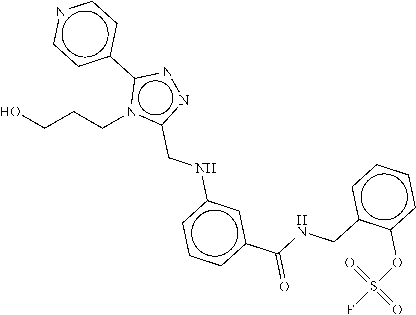 | | 17 | >30.0 | 13.4 |
| 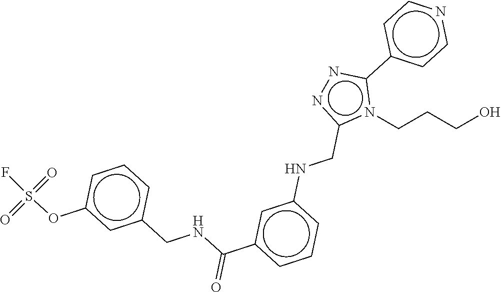 | | 50 | >30.0 | 52.6 |
| 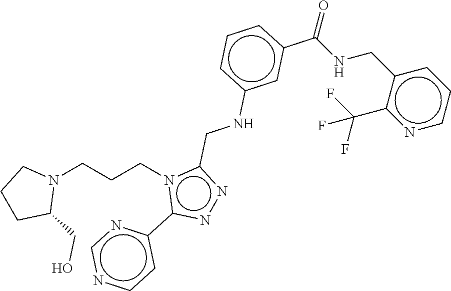 | | 1000 | >30.0 | 76.3 |
| 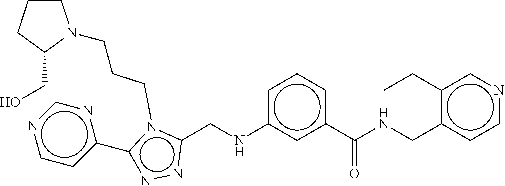 | | | | |
| 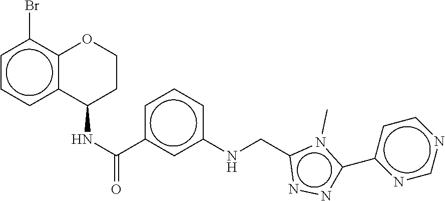 | | 13 | 1.63 | 63.9 |
| 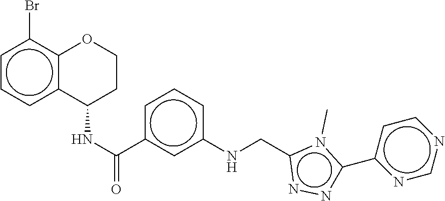 | | 3.6 | >30.0 | 45.6 |
| 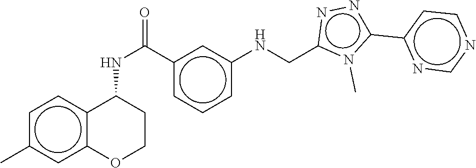 | 9.99 | | 0.648 | 0.644 |
| 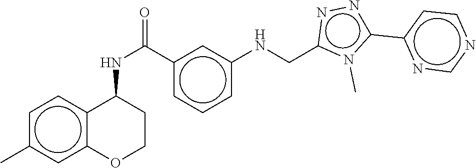 | >1.00E + 03 | | 2.58 | 0.138 |
| 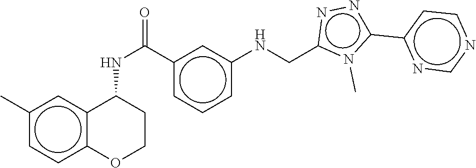 | | 4.6 | 0.196 | 35.6 |
| 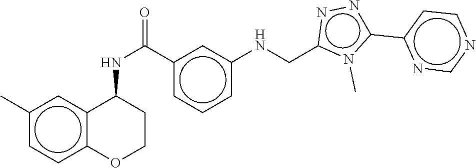 | | 210 | >30.0 | 69.3 |
| 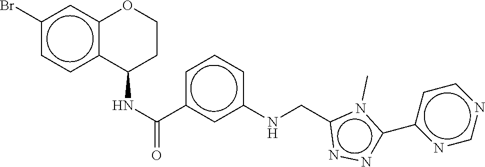 | | 7 | >30.0 | 7.49 |
| 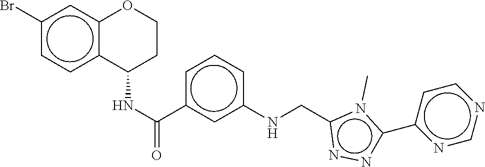 | | 11 | >30.0 | 8.51 |
| 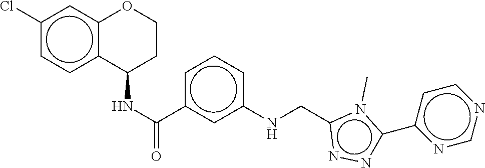 | | 8.4 | 0.24 | 49.9 |
| 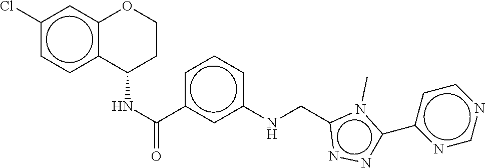 | | 18 | >29.4 | 10.6 |
| 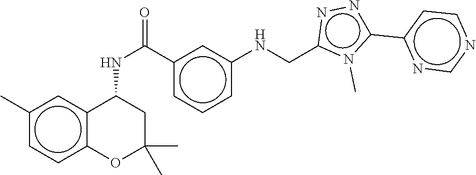 | | 48 | >30.0 | 14.9 |
| 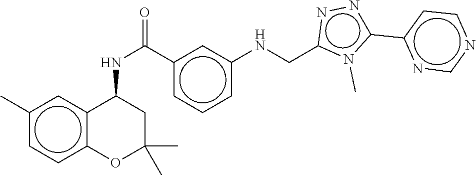 | | 240 | >30.0 | 16.3 |
| 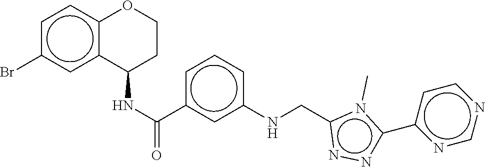 | | 510 | >30.0 | 1.9 |
| 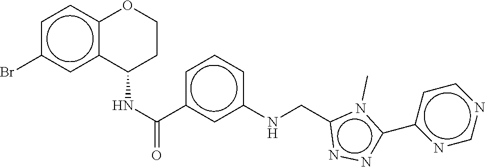 | | 2.6 | 0.0426 | 41.7 |
| 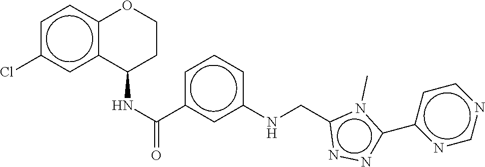 | | 2.9 | 0.0339 | 45.4 |
| 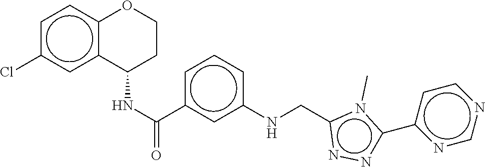 | | 120 | >30.0 | 10.5 |
| 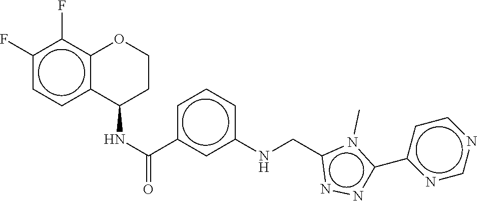 | | 3.3 | 0.178 | 50.5 |
| 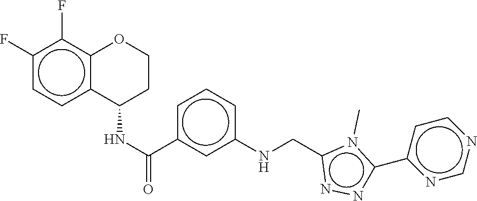 | | 1500 | >30.0 | 50.8 |
| 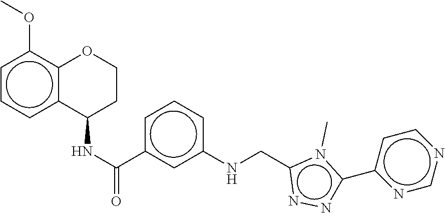 | 26.1 | | 7.74 | 9.11 |
| 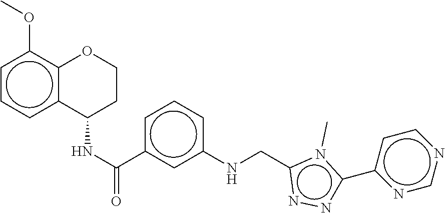 | 87.5 | | 0.47 | 71.2 |
| 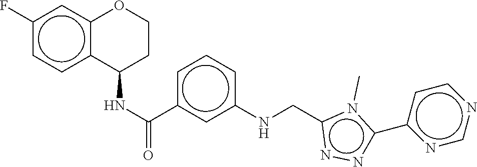 | | 1900 | 0.168 | 46.2 |
| 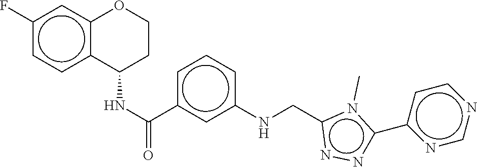 | | 6.4 | >30.0 | 67.9 |
| 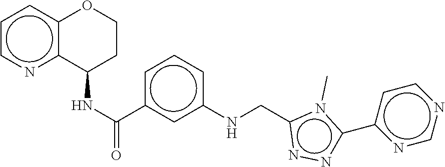 | 1.3 | 7 | 0.192 | 48.7 |
| 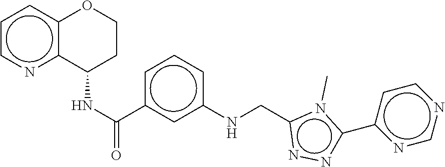 | 144 | 990 | >10.1 | 59.7 |
| 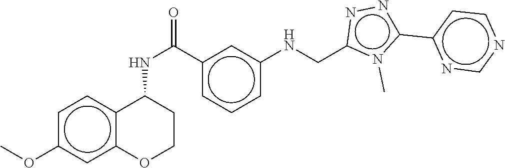 | 60.9 | | 0.485 | 50.7 |
| 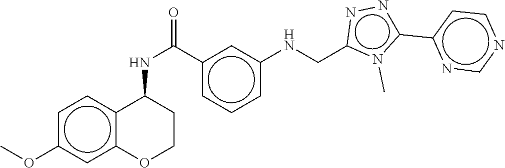 | 1000 | | 10.1 | 18.7 |
| 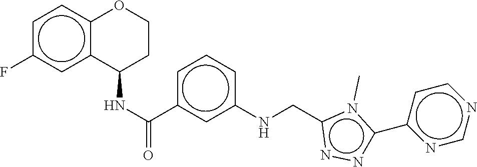 | | 2 | 0.0962 | 47.9 |
| 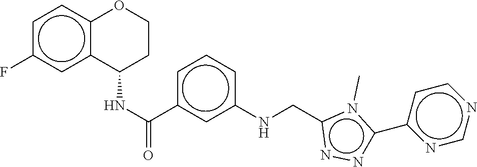 | | 24 | >30.0 | 47.7 |
| 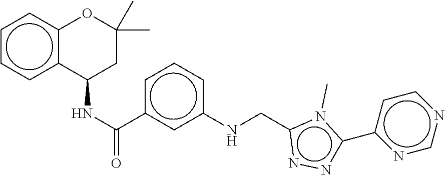 | | 45 | 9.09 | 31.1 |
| 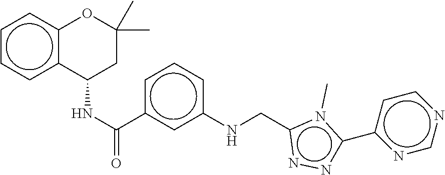 | | 30 | >30.0 | 38.1 |
| 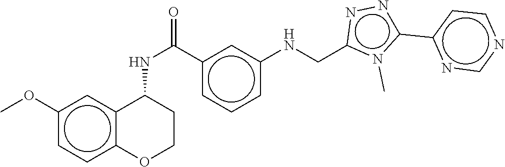 | 15.2 | | >30.0 | 34.7 |
| 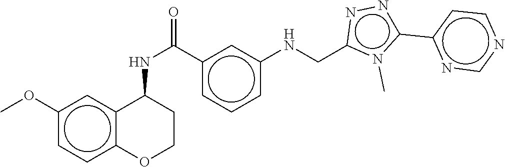 | >1.00E + 03 | | | 43.1 |
| 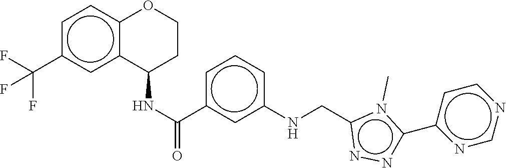 | 8.09 | | 0.594 | 23.7 |
| 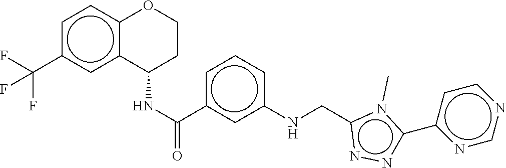 | 834 | | 28.5 | 1.07 |
| 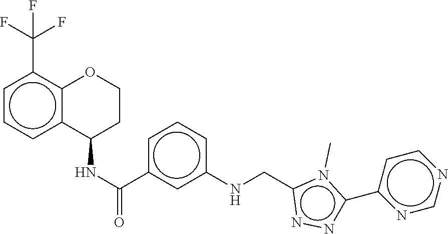 | | 3.8 | >0.972 | 45.9 |
| 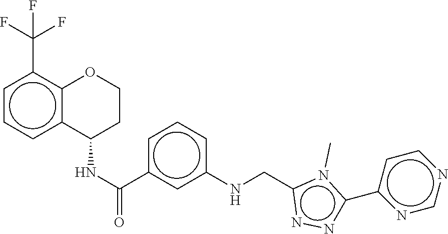 | | 9.7 | >30.0 | 46.5 |
| 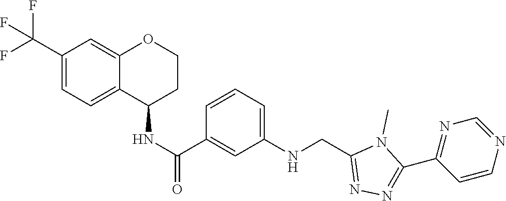 [SMILES: Cn1c(CNc2cccc(C(=O)N[C@@H]3CCOc4cc(C(F)(F)F)ccc43)c2)nnc1-c1ccncn1] | | 56 | 0.0932 | 54 |
| 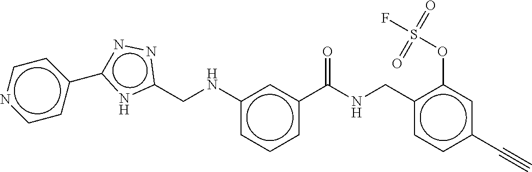 | | | | |
| 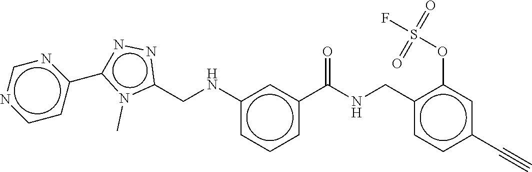 | | | | |
| 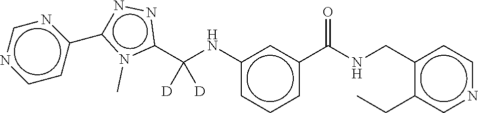 | 20.2 | 9.4 | >30.0 | 27.4 |
| 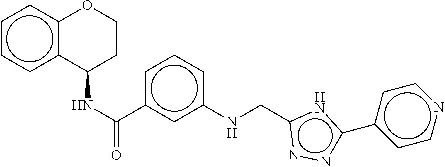 | | | | |
| 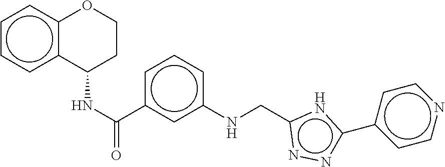 | | | | |
| 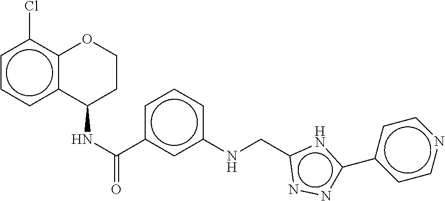 | | | | |
| 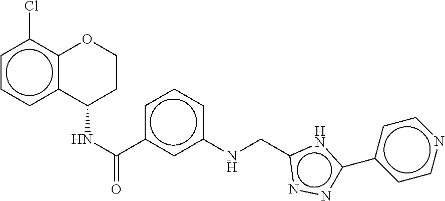 | | | | |
| 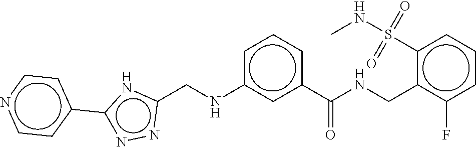 | 5.38 | | 3.62 | 9.33 |
| 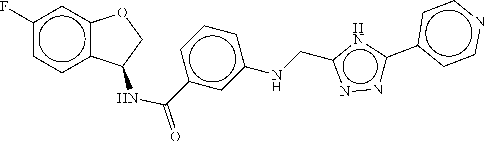 | 7.92 | | 0.607 | 0.911 |
| 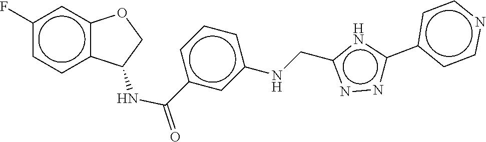 | 27.9 | | 4.07 | 8.79 |
| 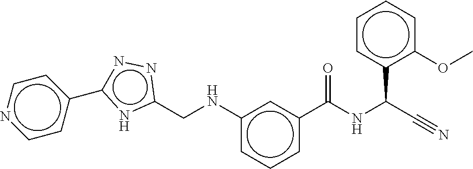 | | | | |
| 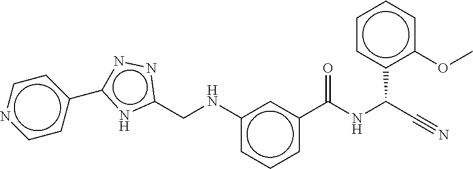 | | | | |
| 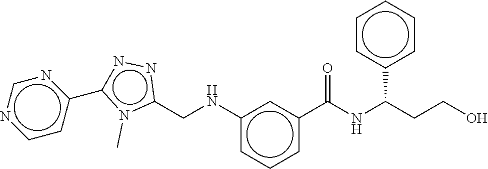 | 65.9 | | 13.1 | 50.5 |
| 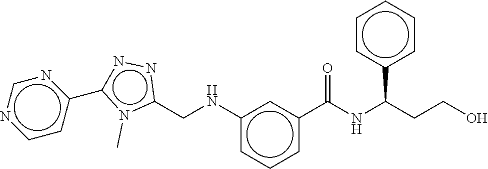 | 9.54 | | 4.19 | 26.3 |
| 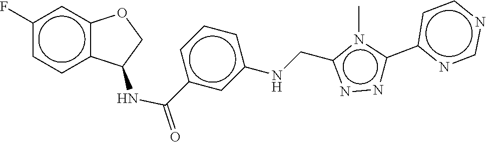 | 25.8 | | 0.117 | 36.9 |
| 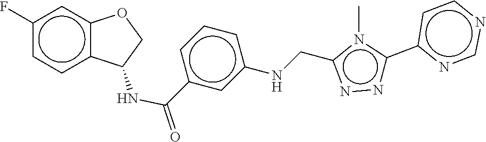 | 9.03 | | >30.0 | 0.766 |
| 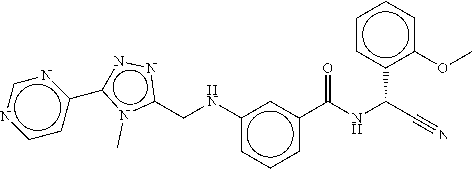 | | | | |
| 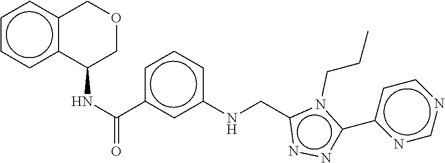 | | | | |
| 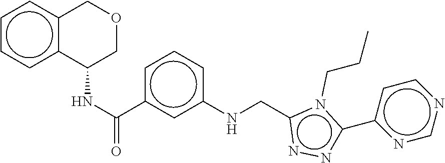 | | | | |
| 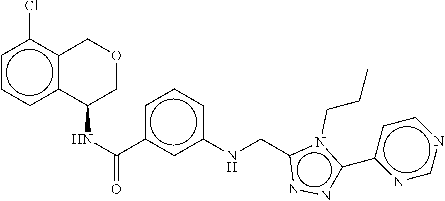 | | | | |
| 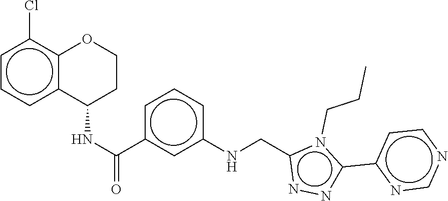 | | | | |
| 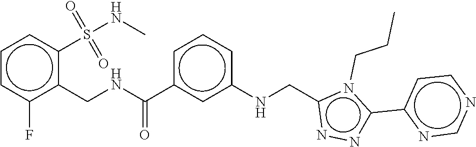 | 22.2 | | >3.20 | 16.4 |
| 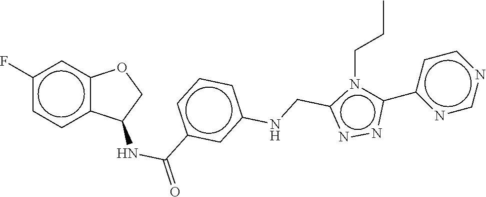 | 3.35 | | >30.0 | 74.3 |
| 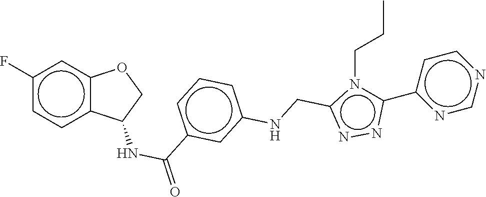 | 10.5 | | >30.0 | 66 |
| 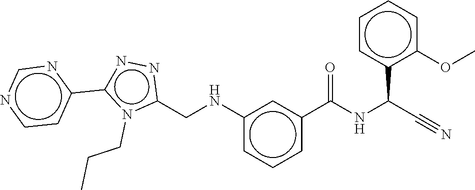 | | | | |
| 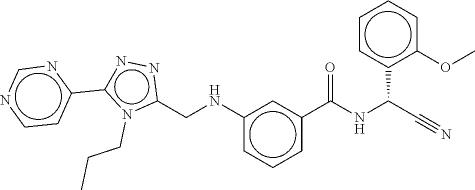 | | | | |
| 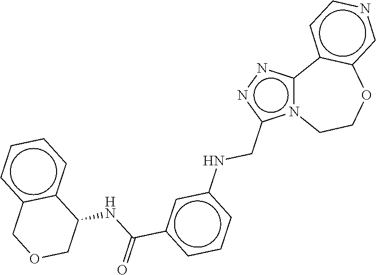 | | | | |
| 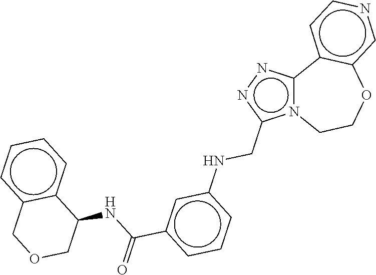 | | | | |
| 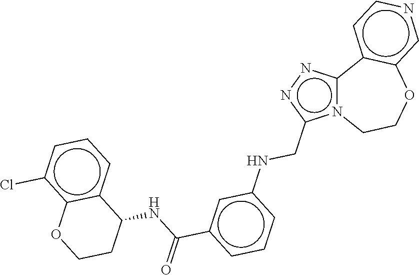 | | | | |
| 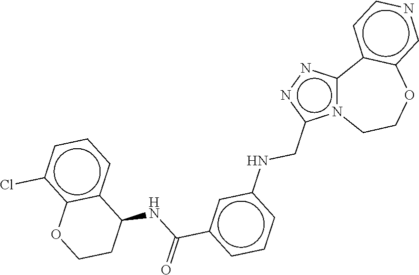 | | | | |
| 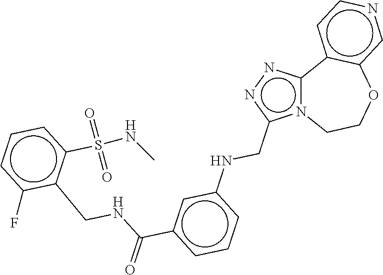 | 85.3 | | 2.84 | 22 |
| 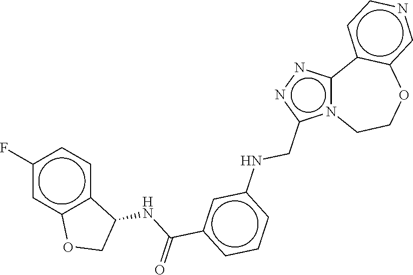 | | | | |
| 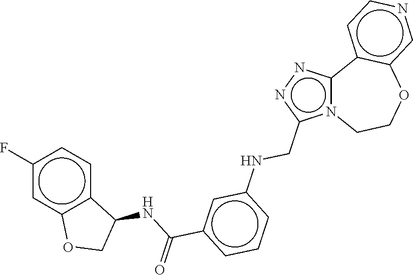 | | | | |
| 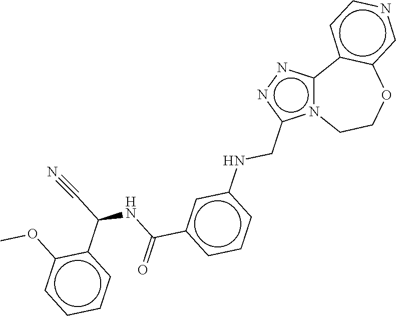 | | | | |
| 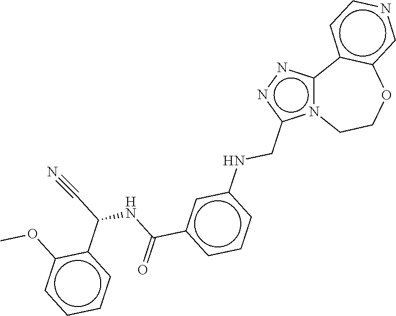 | | | | |
| 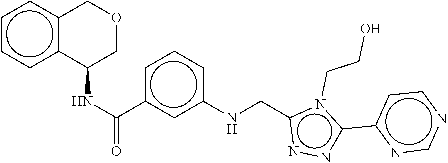 | 1.47 | | 0.592 | 16.5 |
| 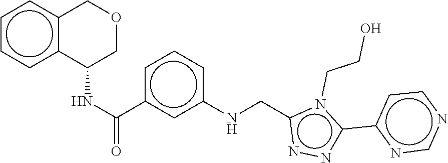 | 38.1 | | >2.11 | 18.5 |
| 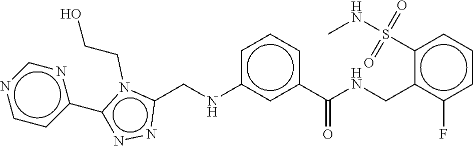 | | | | |
| 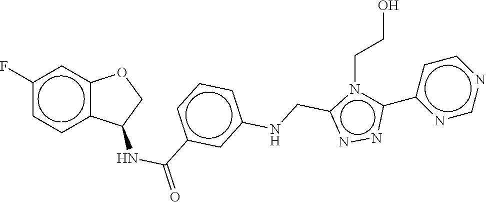 | 22.4 | | 0.0254 | 35.3 |
| 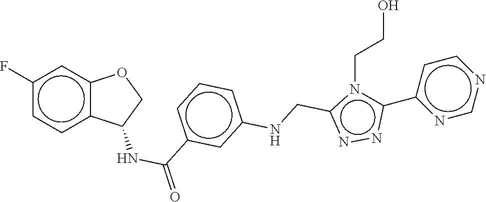 | 3.84 | | 0.256 | 41.6 |
| 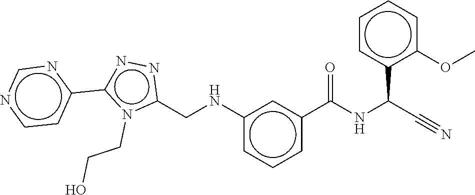 | | | | |
| 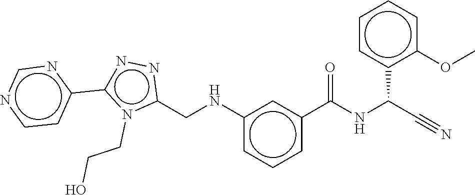 | | | | |
| 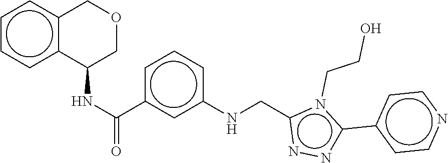 | 9.28 | | >30.0 | 69.2 |
| 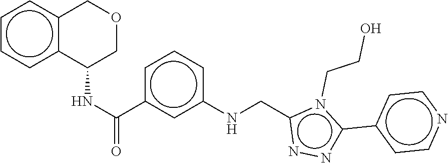 | 10.9 | | 0.0416 | 30.7 |
| 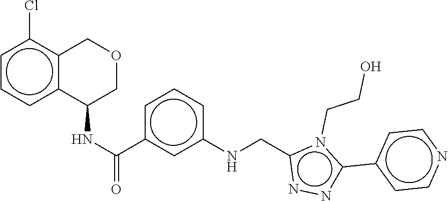 | 9.91 | | 0.0242 | 48.6 |
| 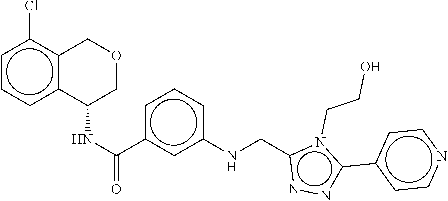 | 7.18 | | >30.0 | 3.17 |
| 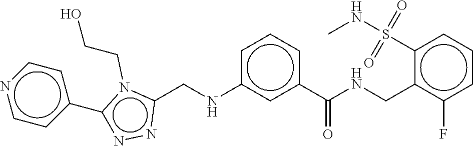 | | | | |
| 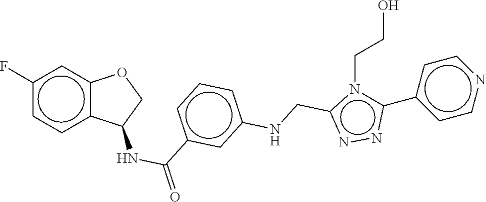 | 4.8 | | 3.13 | 63.3 |
| 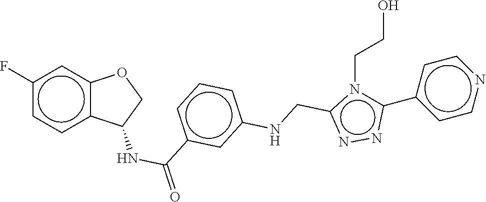 | 23.2 | | >30.0 | 48.1 |
| 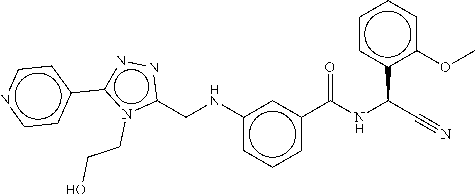 | | | | |
| 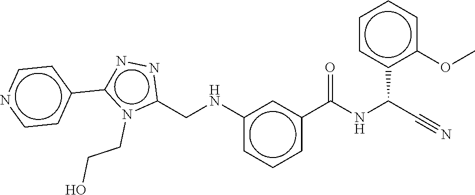 | | | | |
| 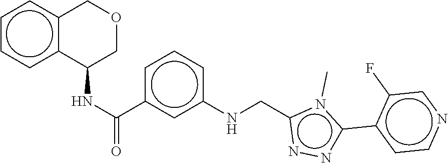 | 227 | | >30.0 | 5.55 |
| 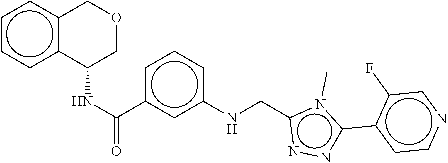 | 32.5 | | 0.0615 | 57.5 |
| 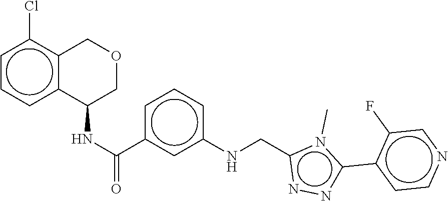 | 60.4 | | >30.0 | 80.1 |
| 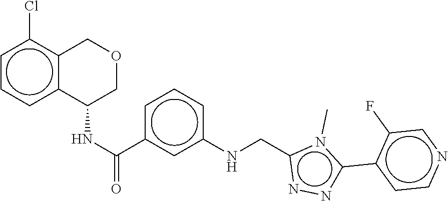 | 21.9 | | 0.245 | 42.1 |
| 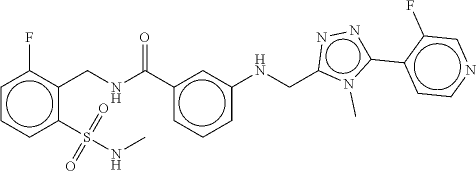 | 33.9 | | 2.7 | 60 |
| 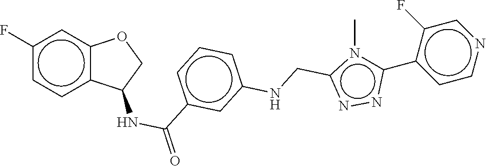 | 22.7 | | 0.201 | 34.6 |
| 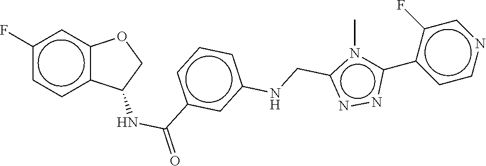 | 111 | | >30.0 | 46.7 |
| 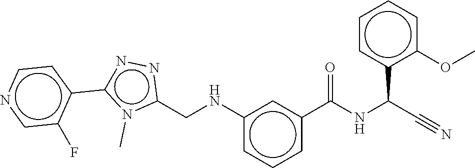 | | | | |
| 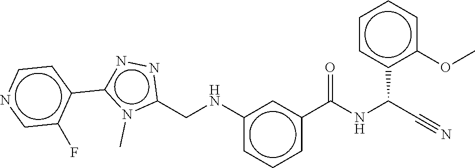 | | | | |
| 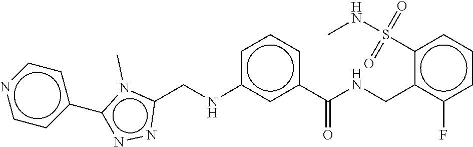 | 15.2 | | 1.24 | 68.6 |
| 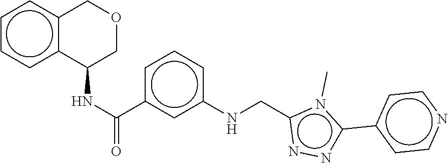 | 201 | | | |
| 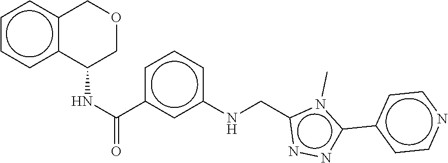 | 100 | | | |
| 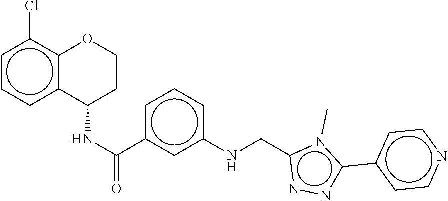 | 32.1 | | >30.0 | 65.3 |
| 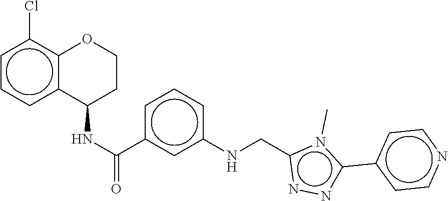 | 27.5 | | >0.865 | 52.7 |
| 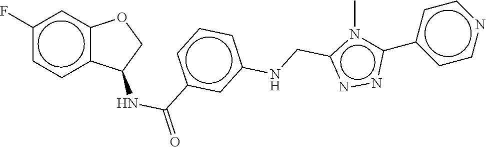 | 143 | | | |
| 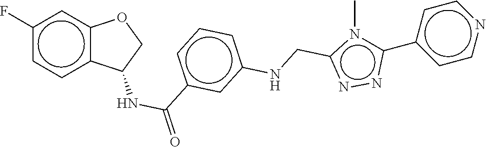 | 6.81 | | >7.49 | 34.7 |
| 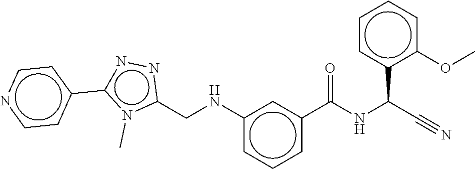 | | | | |
| 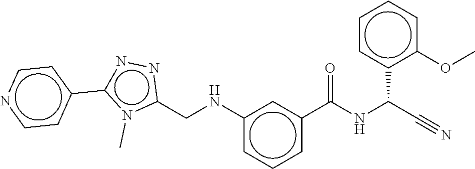 | | | | |
| 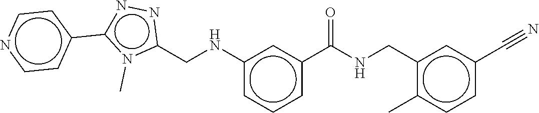 | | | | |
| 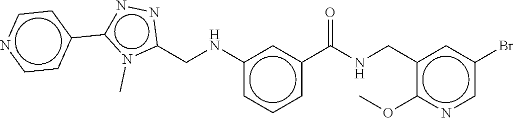 | | | | |
| 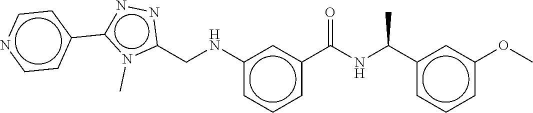 | 117 | | | |
| 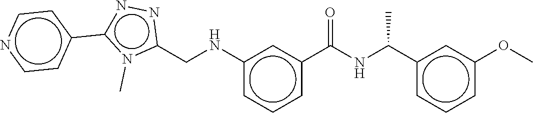 | 7.61 | | >30.0 | 58.6 |
| 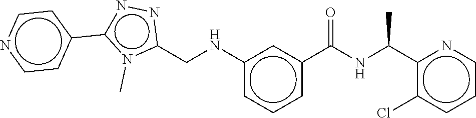 | 2.97 | | >1.85 | 65.4 |
| 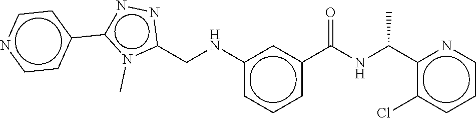 | 11.4 | | >4.78 | 45.5 |
| 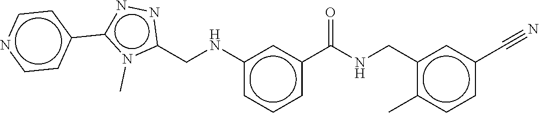 | | | | |
| 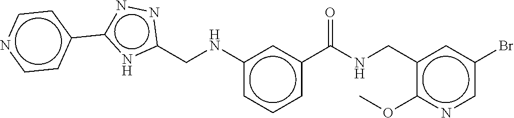 | 10.2 | | 21.7 | 5.28 |
| 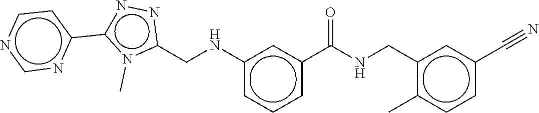 | | | | |
| 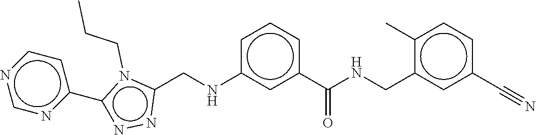 | | | | |
| 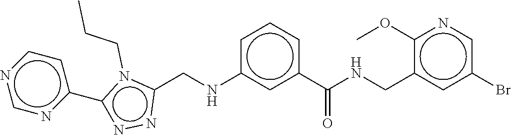 | | | | |
| 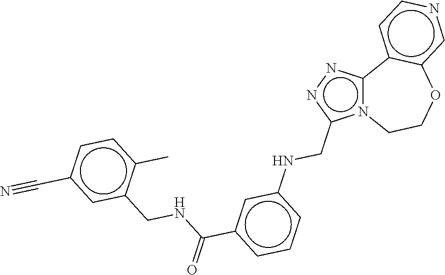 | | | | |
| 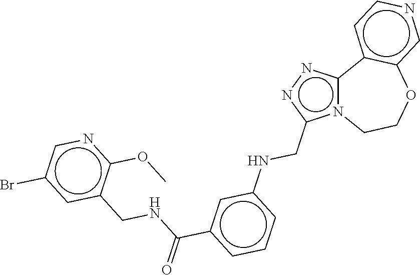 | | | | |
| 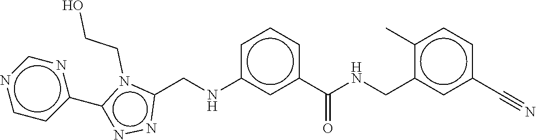 | 3.22 | | >30.0 | 63.6 |
| 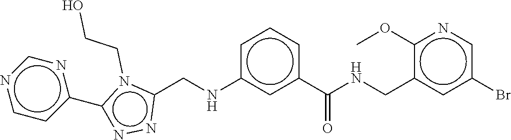 | 4.12 | | >30.0 | 56.7 |
| 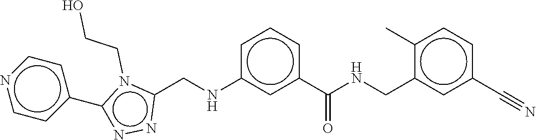 | 4.82 | | >4.60 | 69.5 |
| 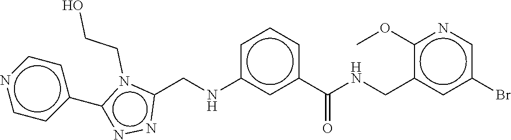 | 7.15 | | 15.1 | 50.5 |
| 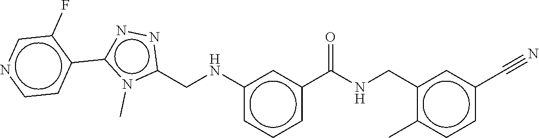 | | | | |
| 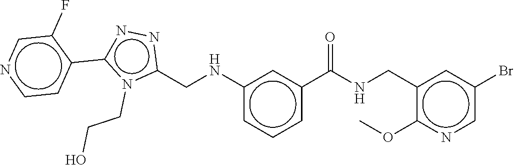 | | | | |
| 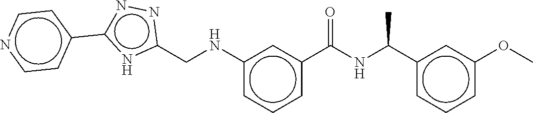 | 17.5 | | >30.0 | 7.94 |
| 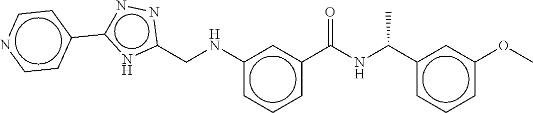 | 81.6 | | | |
| 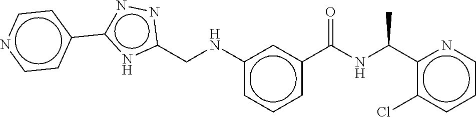 | | | | |
| 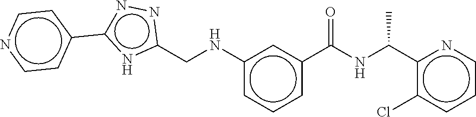 | | | | |
| 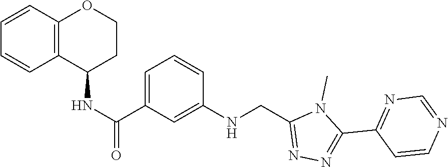 [SMILES: Cn1c(CNc2cccc(C(=O)N[C@@H]3CCOc4ccccc43)c2)nnc1-c1ccncn1] | 23 | | 0.227 | 73.9 |
| 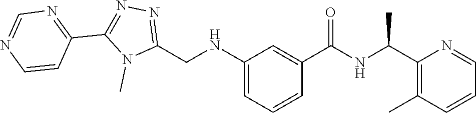 [SMILES: Cc1cccnc1[C@H](C)NC(=O)c1cccc(NCc2nnc(-c3ccncn3)n2C)c1] | 15 | | >30.0 | 51.8 |
| 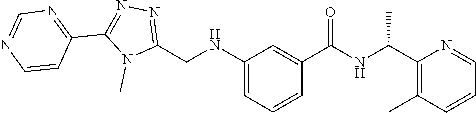 [SMILES: Cc1cccnc1[C@@H](C)NC(=O)c1cccc(NCc2nnc(-c3ccncn3)n2C)c1] | 26 | | 3.79 | 58.9 |
| 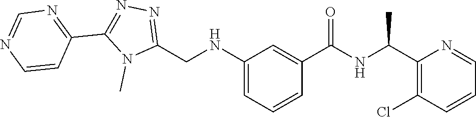 [SMILES: C[C@H](NC(=O)c1cccc(NCc2nnc(-c3ccncn3)n2C)c1)c1ncccc1Cl] | 9.6 | 16 | 0.696 | 61.9 |
| 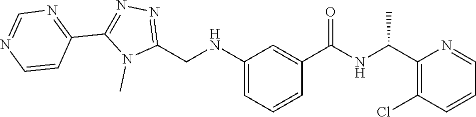 [SMILES: C[C@@H](NC(=O)c1cccc(NCc2nnc(-c3ccncn3)n2C)c1)c1ncccc1Cl] | 12 | 15 | 27.1 | 56.7 |
| 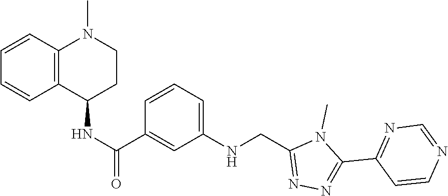 [SMILES: CN1CC[C@@H](NC(=O)c2cccc(NCc3nnc(-c4ccncn4)n3C)c2)c2ccccc21] | 21 | | >30.0 | 16 |
| 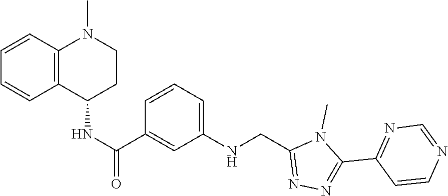 [SMILES: CN1CC[C@H](NC(=O)c2cccc(NCc3nnc(-c4ccncn4)n3C)c2)c2ccccc21] | 85 | | | |
| 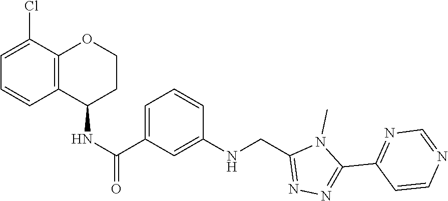 [SMILES: Cn1c(CNc2cccc(C(=O)N[C@@H]3CCOc4c(Cl)cccc43)c2)nnc1-c1ccncn1] | 8.2 | 3.6 | >0.47 | 49.0 |
| 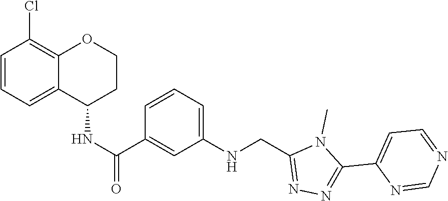 [SMILES: Cn1c(CNc2cccc(C(=O)N[C@H]3CCOc4c(Cl)cccc43)c2)nnc1-c1ccncn1] | 33.6 | 13.2 | >1.01 | 45.9 |
| 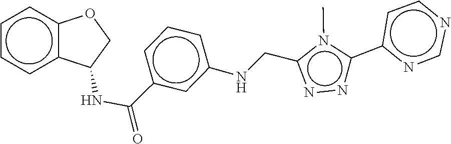 | | 3.4 | | |
| 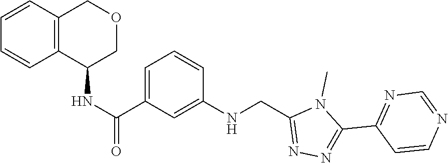 [SMILES: Cn1c(CNc2cccc(C(=O)N[C@@H]3COCc4ccccc43)c2)nnc1-c1ccncn1] | 20.2 | 11.9 | >0.80 | 47.6 |
| 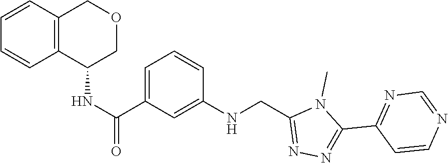 [SMILES: Cn1c(CNc2cccc(C(=O)N[C@H]3COCc4ccccc43)c2)nnc1-c1ccncn1] | 66 | | >11.8 | 38.2 |
| 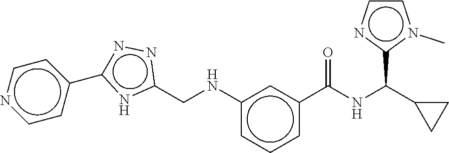 | | | | |
| 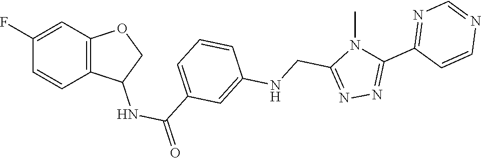 [SMILES: Cn1c(CNc2cccc(C(=O)NC3COc4cc(F)ccc43)c2)nnc1-c1ccncn1] | 35.4 | | 0.547 | 52.1 |
| 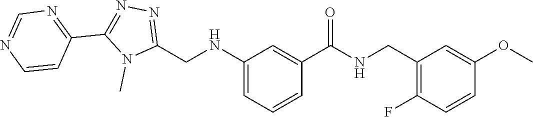 [SMILES: COc1ccc(F)c(CNC(=O)c2cccc(NCc3nnc(-c4ccncn4)n3C)c2)c1] | 20 | | 0.917 | 56.9 |
| 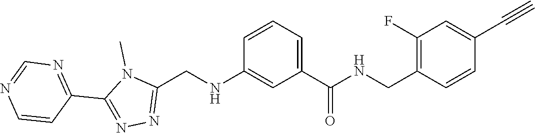 [SMILES: C#Cc1ccc(CNC(=O)c2cccc(NCc3nnc(-c4ccncn4)n3C)c2)c(F)c1] | 12.03 | | 0.413 | 51.3 |
| 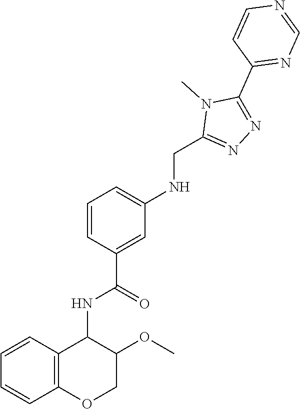 [SMILES: COC1COc2ccccc2C1NC(=O)c1cccc(NCc2nnc(-c3ccncn3)n2C)c1] | 59.1 | | | |
| 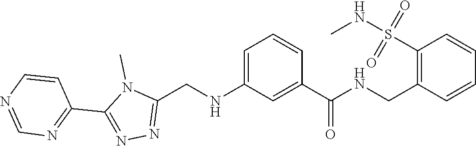 [SMILES: CNS(=O)(=O)c1ccccc1CNC(=O)c1cccc(NCc2nnc(-c3ccncn3)n2C)c1] | 62.5 | | | |
| 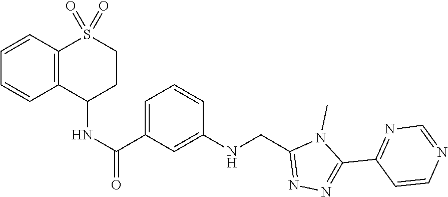 [SMILES: Cn1c(CNc2cccc(C(=O)NC3CCS(=O)(=O)c4ccccc43)c2)nnc1-c1ccncn1] | 18.2 | | 2.79 | 53.6 |
| 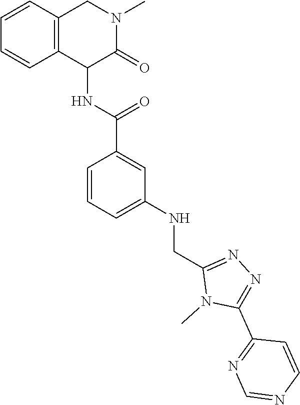 [SMILES: CN1Cc2ccccc2C(NC(=O)c2cccc(NCc3nnc(-c4ccncn4)n3C)c2)C1=O] | 63.5 | | >1.90 | 53.9 |
| 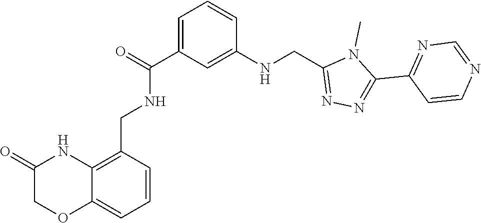 [SMILES: Cn1c(CNc2cccc(C(=O)NCc3cccc4c3NC(=O)CO4)c2)nnc1-c1ccncn1] | 9.78 | | 2.99 | 25.7 |
| 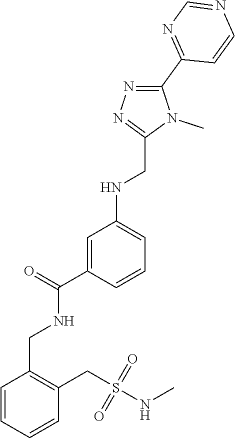 [SMILES: CNS(=O)(=O)Cc1ccccc1CNC(=O)c1cccc(NCc2nnc(-c3ccncn3)n2C)c1] | 68.9 | | | |
| 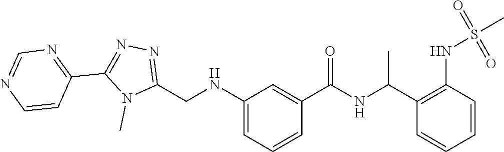 [SMILES: CC(NC(=O)c1cccc(NCc2nnc(-c3ccncn3)n2C)c1)c1ccccc1NS(C)(=O)=O] | 68 | | | |
| 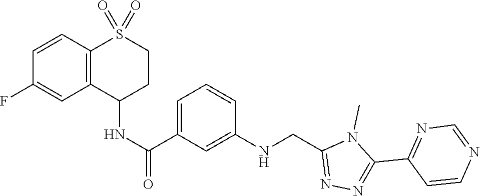 [SMILES: Cn1c(CNc2cccc(C(=O)NC3CCS(=O)(=O)c4ccc(F)cc43)c2)nnc1-c1ccncn1] | 65.4 | | 8.68 | 47.4 |
| 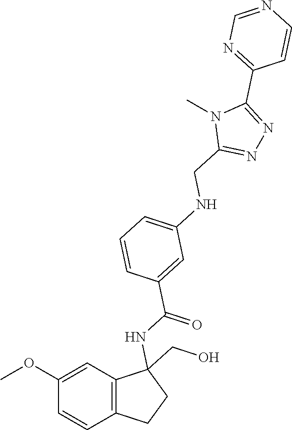 [SMILES: COc1ccc2c(c1)C(CO)(NC(=O)c1cccc(NCc3nnc(-c4ccncn4)n3C)c1)CC2] | 10000 | | | |
| 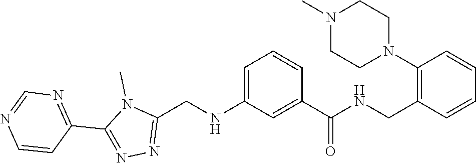 [SMILES: CN1CCN(c2ccccc2CNC(=O)c2cccc(NCc3nnc(-c4ccncn4)n3C)c2)CC1] | 184 | | | |
| 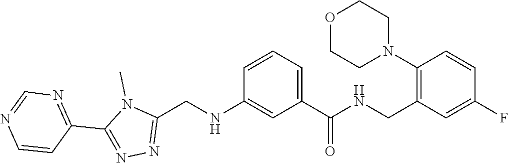 [SMILES: Cn1c(CNc2cccc(C(=O)NCc3cc(F)ccc3N3CCOCC3)c2)nnc1-c1ccncn1] | 104 | | | |
| 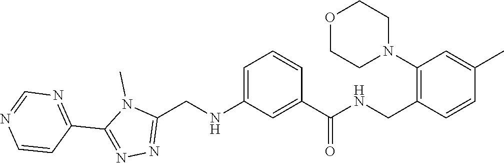 [SMILES: Cc1ccc(CNC(=O)c2cccc(NCc3nnc(-c4ccncn4)n3C)c2)c(N2CCOCC2)c1] | 412 | | | |
| 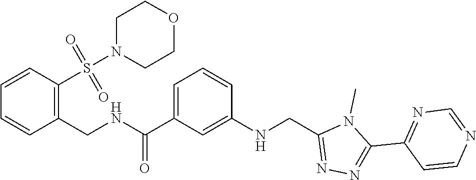 [SMILES: Cn1c(CNc2cccc(C(=O)NCc3ccccc3S(=O)(=O)N3CCOCC3)c2)nnc1-c1ccncn1] | 52.8 | | | |
| 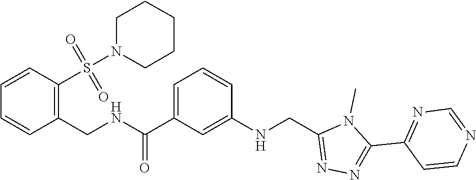 [SMILES: Cn1c(CNc2cccc(C(=O)NCc3ccccc3S(=O)(=O)N3CCCCC3)c2)nnc1-c1ccncn1] | 50.5 | | | |
| 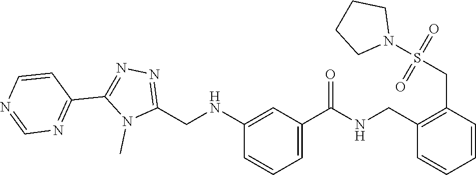 [SMILES: Cn1c(CNc2cccc(C(=O)NCc3ccccc3CS(=O)(=O)N3CCCC3)c2)nnc1-c1ccncn1] | 107 | | | |
| 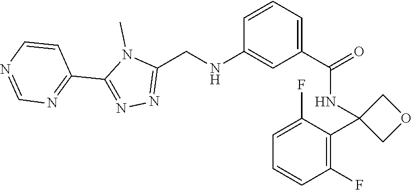 [SMILES: Cn1c(CNc2cccc(C(=O)NC3(c4c(F)cccc4F)COC3)c2)nnc1-c1ccncn1] | 10000 | | | |
| 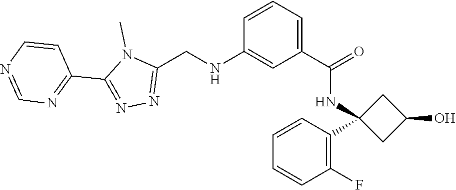 [SMILES: Cn1c(CNc2cccc(C(=O)N[C@]3(c4ccccc4F)C[C@H](O)C3)c2)nnc1-c1ccncn1] | 423 | | | |
| 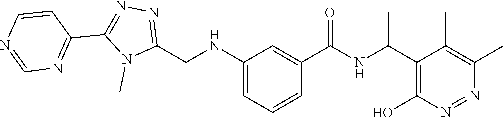 [SMILES: Cc1nnc(O)c(C(C)NC(=O)c2cccc(NCc3nnc(-c4ccncn4)n3C)c2)c1C] | 132 | | | |
| 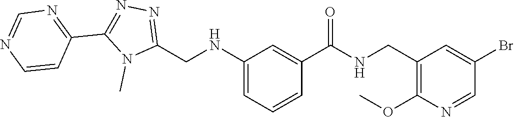 [SMILES: COc1ncc(Br)cc1CNC(=O)c1cccc(NCc2nnc(-c3ccncn3)n2C)c1] | 21.5 | | 0.61 | 52.7 |
| 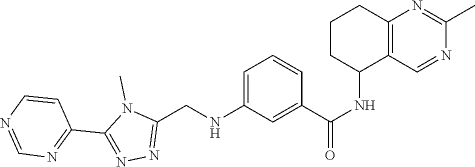 [SMILES: Cc1ncc2c(n1)CCCC2NC(=O)c1cccc(NCc2nnc(-c3ccncn3)n2C)c1] | 108 | | | |
| 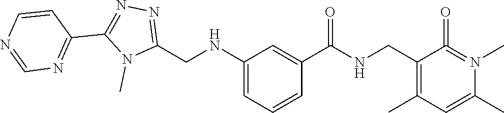 | 9.97 | | 1.7 | 40.1 |
| 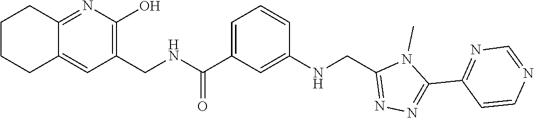 [SMILES: Cn1c(CNc2cccc(C(=O)NCc3cc4c(nc3O)CCCC4)c2)nnc1-c1ccncn1] | 192 | | | |
| 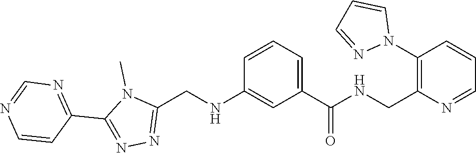 [SMILES: Cn1c(CNc2cccc(C(=O)NCc3ncccc3-n3cccn3)c2)nnc1-c1ccncn1] | 318 | | | |
| 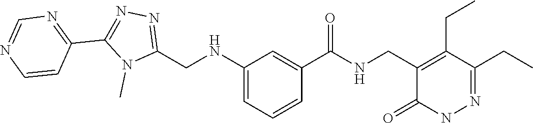 [SMILES: CCc1n[nH]c(=O)c(CNC(=O)c2cccc(NCc3nnc(-c4ccncn4)n3C)c2)c1CC] | 504 | | | |
| 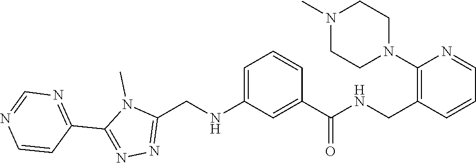 [SMILES: CN1CCN(c2ncccc2CNC(=O)c2cccc(NCc3nnc(-c4ccncn4)n3C)c2)CC1] | 289 | | | |
| 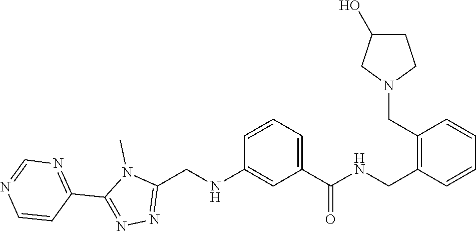 [SMILES: Cn1c(CNc2cccc(C(=O)NCc3ccccc3CN3CCC(O)C3)c2)nnc1-c1ccncn1] | 136 | | | |
| 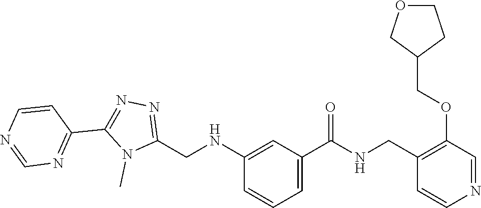 [SMILES: Cn1c(CNc2cccc(C(=O)NCc3ccncc3OCC3CCOC3)c2)nnc1-c1ccncn1] | 871 | | | |
| 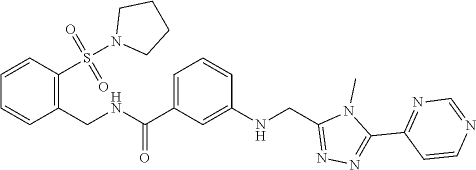 [SMILES: Cn1c(CNc2cccc(C(=O)NCc3ccccc3S(=O)(=O)N3CCCC3)c2)nnc1-c1ccncn1] | 26.9 | | 2.17 | 49.1 |
| 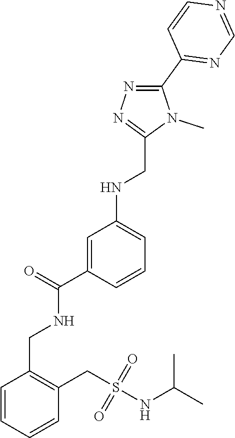 [SMILES: CC(C)NS(=O)(=O)Cc1ccccc1CNC(=O)c1cccc(NCc2nnc(-c3ccncn3)n2C)c1] | 69.8 | | | |
| 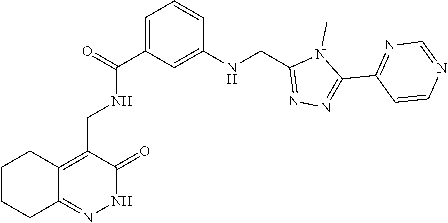 [SMILES: Cn1c(CNc2cccc(C(=O)NCc3c4c(n[nH]c3=O)CCCC4)c2)nnc1-c1ccncn1] | 531 | | | |
| 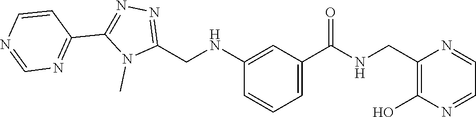 [SMILES: Cn1c(CNc2cccc(C(=O)NCc3nccnc3O)c2)nnc1-c1ccncn1] | 728 | | | |
| 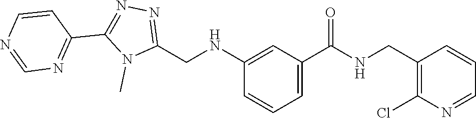 [SMILES: Cn1c(CNc2cccc(C(=O)NCc3cccnc3Cl)c2)nnc1-c1ccncn1] | 27.6 | | 0.506 | 70 |
| 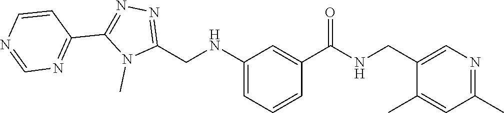 [SMILES: Cc1cc(C)c(CNC(=O)c2cccc(NCc3nnc(-c4ccncn4)n3C)c2)cn1] | 17.8 | | 0.803 | 48.9 |
| 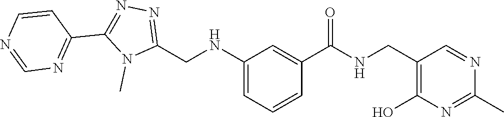 [SMILES: Cc1ncc(CNC(=O)c2cccc(NCc3nnc(-c4ccncn4)n3C)c2)c(O)n1] | 382 | | | |
| 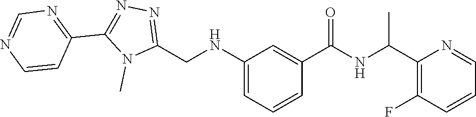 [SMILES: CC(NC(=O)c1cccc(NCc2nnc(-c3ccncn3)n2C)c1)c1ncccc1F] | 69 | | 2.66 | 57.7 |
| 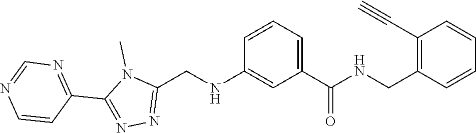 [SMILES: C#Cc1ccccc1CNC(=O)c1cccc(NCc2nnc(-c3ccncn3)n2C)c1] | 46.4 | | 2.32 | 65.6 |
| 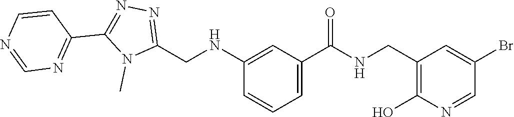 [SMILES: Cn1c(CNc2cccc(C(=O)NCc3cc(Br)cnc3O)c2)nnc1-c1ccncn1] | 103 | | | |
| 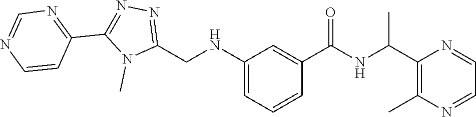 [SMILES: Cc1nccnc1C(C)NC(=O)c1cccc(NCc2nnc(-c3ccncn3)n2C)c1] | 61 | | >12.5 | 48.2 |
| 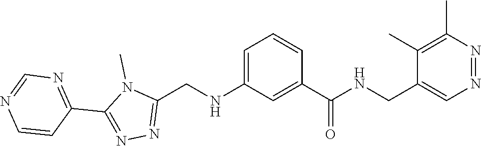 [SMILES: Cc1nncc(CNC(=O)c2cccc(NCc3nnc(-c4ccncn4)n3C)c2)c1C] | 14.3 | | 1.46 | 64.9 |
| 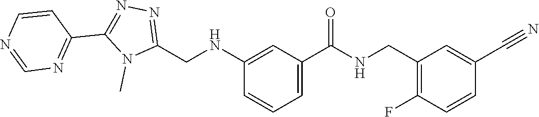 [SMILES: Cn1c(CNc2cccc(C(=O)NCc3cc(C#N)ccc3F)c2)nnc1-c1ccncn1] | 17.6 | | >1.28 | 51.0 |
| 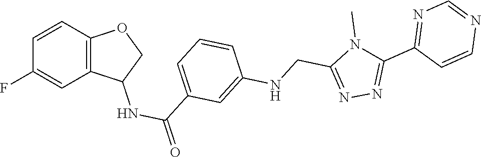 [SMILES: Cn1c(CNc2cccc(C(=O)NC3COc4ccc(F)cc43)c2)nnc1-c1ccncn1] | 26.6 | | 1.26 | 55.1 |
| 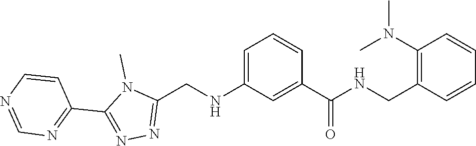 [SMILES: CN(C)c1ccccc1CNC(=O)c1cccc(NCc2nnc(-c3ccncn3)n2C)c1] | 14.8 | | 1.61 | 50.9 |
| 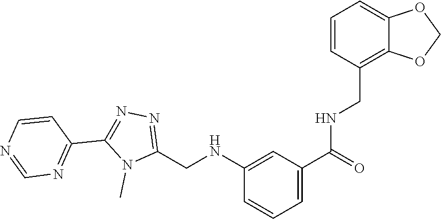 [SMILES: Cn1c(CNc2cccc(C(=O)NCc3cccc4c3OCO4)c2)nnc1-c1ccncn1] | 13.2 | | 0.681 | 65.8 |
| 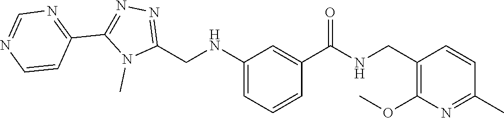 [SMILES: COc1nc(C)ccc1CNC(=O)c1cccc(NCc2nnc(-c3ccncn3)n2C)c1] | 30.1 | | >30.0 | 70 |
| 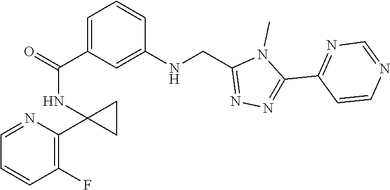 [SMILES: Cn1c(CNc2cccc(C(=O)NC3(c4ncccc4F)CC3)c2)nnc1-c1ccncn1] | 104 | | | |
| 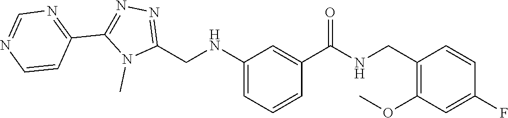 [SMILES: COc1cc(F)ccc1CNC(=O)c1cccc(NCc2nnc(-c3ccncn3)n2C)c1] | 24.7 | | 28 | 17.2 |
| 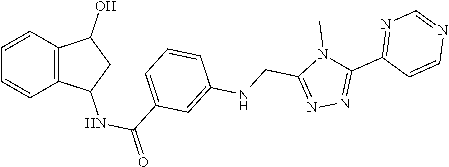 [SMILES: Cn1c(CNc2cccc(C(=O)NC3CC(O)c4ccccc43)c2)nnc1-c1ccncn1] | 65 | | >30.0 | 77.2 |
| 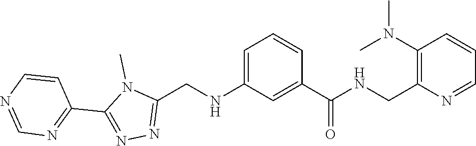 [SMILES: CN(C)c1cccnc1CNC(=O)c1cccc(NCc2nnc(-c3ccncn3)n2C)c1] | 120 | | | |
| 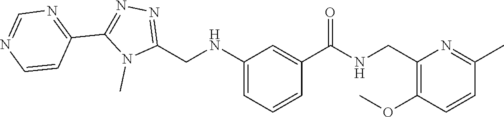 [SMILES: COc1ccc(C)nc1CNC(=O)c1cccc(NCc2nnc(-c3ccncn3)n2C)c1] | 67.5 | | >12.4 | 54.8 |
| 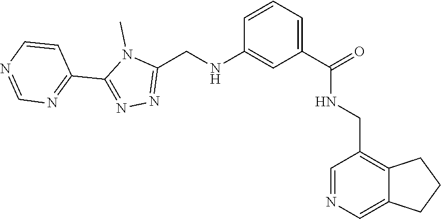 [SMILES: Cn1c(CNc2cccc(C(=O)NCc3cncc4c3CCC4)c2)nnc1-c1ccncn1] | 15.3 | | >11.7 | 10.9 |
| 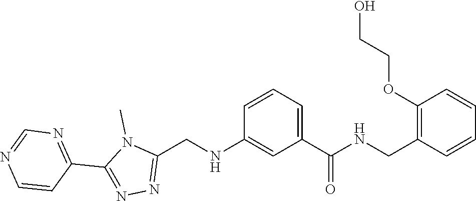 [SMILES: Cn1c(CNc2cccc(C(=O)NCc3ccccc3OCCO)c2)nnc1-c1ccncn1] | 48.9 | | >6.45 | 67.7 |
| 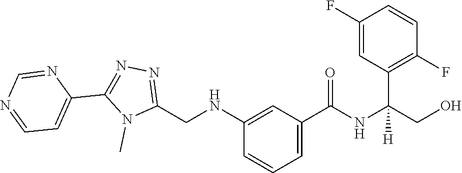 [SMILES: Cn1c(CNc2cccc(C(=O)N[C@H](CO)c3cc(F)ccc3F)c2)nnc1-c1ccncn1] | 43.4 | | >30.0 | 65.8 |
| 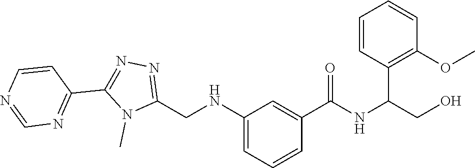 [SMILES: COc1ccccc1C(CO)NC(=O)c1cccc(NCc2nnc(-c3ccncn3)n2C)c1] | 45.4 | | >13.0 | 62.3 |
| 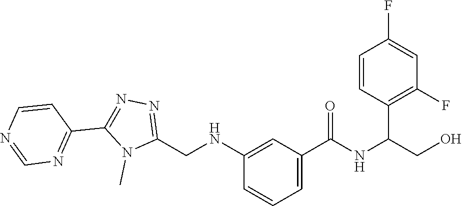 [SMILES: Cn1c(CNc2cccc(C(=O)NC(CO)c3ccc(F)cc3F)c2)nnc1-c1ccncn1] | 83.1 | | 3.07 | 72.9 |
| 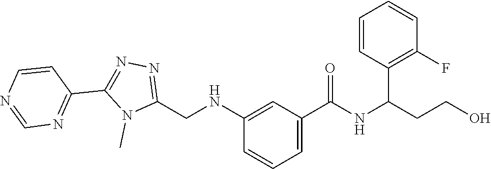 [SMILES: Cn1c(CNc2cccc(C(=O)NC(CCO)c3ccccc3F)c2)nnc1-c1ccncn1] | 35.6 | | 0.536 | 54.4 |
| 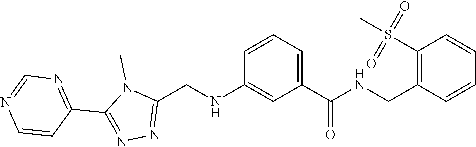 [SMILES: Cn1c(CNc2cccc(C(=O)NCc3ccccc3S(C)(=O)=O)c2)nnc1-c1ccncn1] | 57.1 | | 1.95 | 62.6 |
| 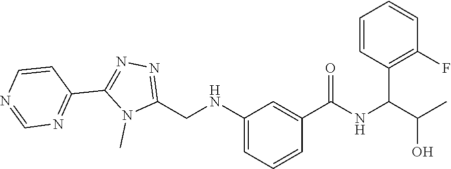 [SMILES: CC(O)C(NC(=O)c1cccc(NCc2nnc(-c3ccncn3)n2C)c1)c1ccccc1F] | 209 | | | |
| 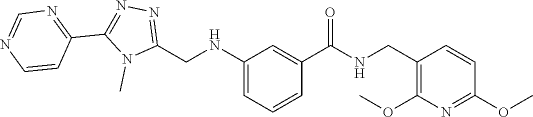 [SMILES: COc1ccc(CNC(=O)c2cccc(NCc3nnc(-c4ccncn4)n3C)c2)c(OC)n1] | 37.2 | | 0.797 | 62.7 |
| 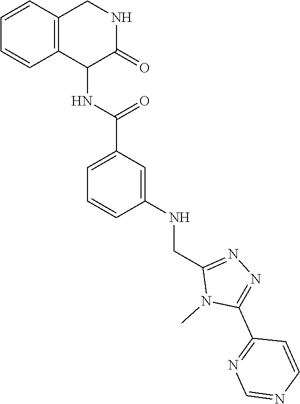 [SMILES: Cn1c(CNc2cccc(C(=O)NC3C(=O)NCc4ccccc43)c2)nnc1-c1ccncn1] | 38.1 | | >30.0 | 73.5 |
| 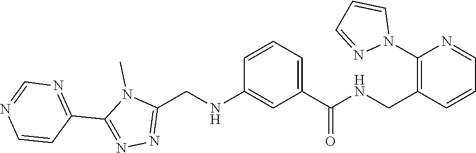 [SMILES: Cn1c(CNc2cccc(C(=O)NCc3cccnc3-n3cccn3)c2)nnc1-c1ccncn1] | 146 | | | |
| 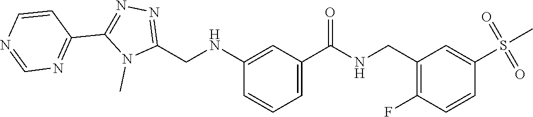 [SMILES: Cn1c(CNc2cccc(C(=O)NCc3cc(S(C)(=O)=O)ccc3F)c2)nnc1-c1ccncn1] | 52 | | >16.0 | 56.3 |
| 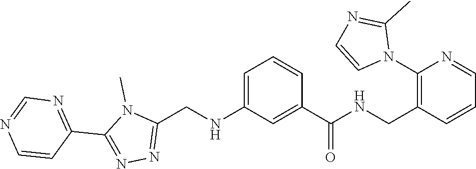 [SMILES: Cc1nccn1-c1ncccc1CNC(=O)c1cccc(NCc2nnc(-c3ccncn3)n2C)c1] | 58.8 | | >25.8 | 72.6 |
| 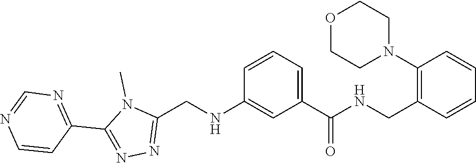 [SMILES: Cn1c(CNc2cccc(C(=O)NCc3ccccc3N3CCOCC3)c2)nnc1-c1ccncn1] | 50 | | 3.25 | 65.1 |
| 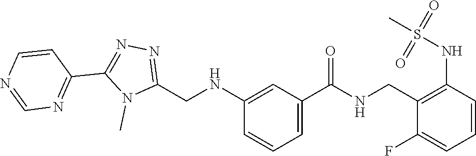 [SMILES: Cn1c(CNc2cccc(C(=O)NCc3c(F)cccc3NS(C)(=O)=O)c2)nnc1-c1ccncn1] | 9.42 | | 3.25 | 59 |
| 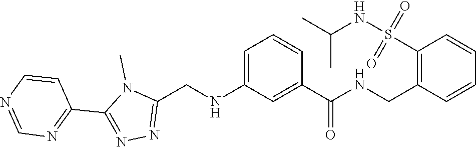 [SMILES: CC(C)NS(=O)(=O)c1ccccc1CNC(=O)c1cccc(NCc2nnc(-c3ccncn3)n2C)c1] | 65.8 | | >15.9 | 57.2 |
| 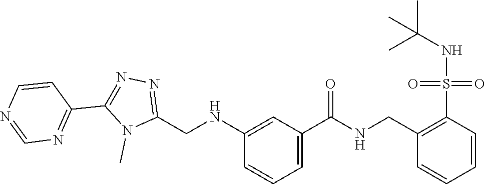 [SMILES: Cn1c(CNc2cccc(C(=O)NCc3ccccc3S(=O)(=O)NC(C)(C)C)c2)nnc1-c1ccncn1] | 54 | | 4.29 | 47.4 |
| 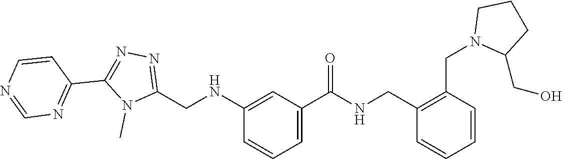 [SMILES: Cn1c(CNc2cccc(C(=O)NCc3ccccc3CN3CCCC3CO)c2)nnc1-c1ccncn1] | 10000 | | | |
| 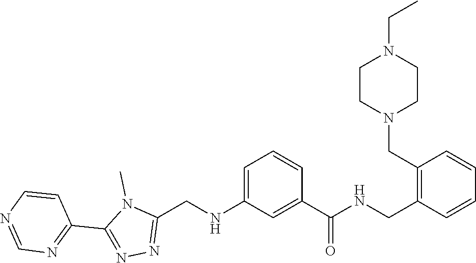 [SMILES: CCN1CCN(Cc2ccccc2CNC(=O)c2cccc(NCc3nnc(-c4ccncn4)n3C)c2)CC1] | 267 | | | |
| 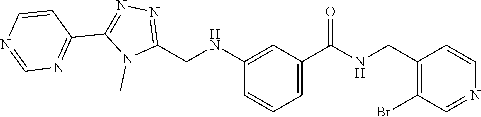 [SMILES: Cn1c(CNc2cccc(C(=O)NCc3ccncc3Br)c2)nnc1-c1ccncn1] | 13.8 | | 0.633 | 56.3 |
| 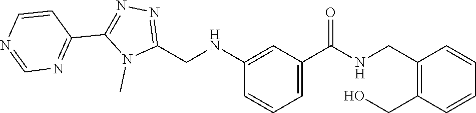 [SMILES: Cn1c(CNc2cccc(C(=O)NCc3ccccc3CO)c2)nnc1-c1ccncn1] | 18.4 | | 1.56 | 51.9 |
| 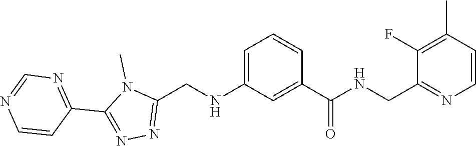 [SMILES: Cc1ccnc(CNC(=O)c2cccc(NCc3nnc(-c4ccncn4)n3C)c2)c1F] | 50.2 | | >30.0 | 33.4 |
| 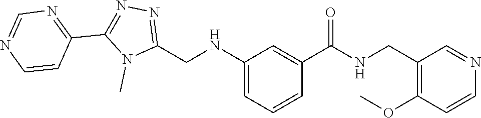 [SMILES: COc1ccncc1CNC(=O)c1cccc(NCc2nnc(-c3ccncn3)n2C)c1] | 28.1 | | 5.09 | 50.5 |
| 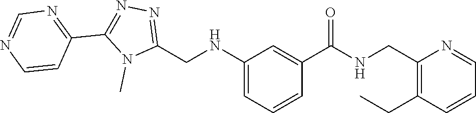 [SMILES: CCc1cccnc1CNC(=O)c1cccc(NCc2nnc(-c3ccncn3)n2C)c1] | 22.6 | | 4.51 | 13.3 |
| 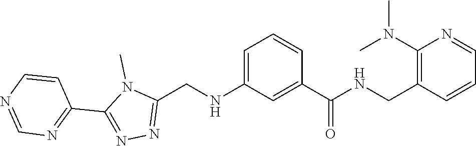 [SMILES: CN(C)c1ncccc1CNC(=O)c1cccc(NCc2nnc(-c3ccncn3)n2C)c1] | 46.1 | | 1.16 | 71.6 |
| 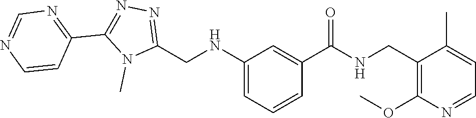 [SMILES: COc1nccc(C)c1CNC(=O)c1cccc(NCc2nnc(-c3ccncn3)n2C)c1] | 37.3 | | 0.93 | 52.7 |
| 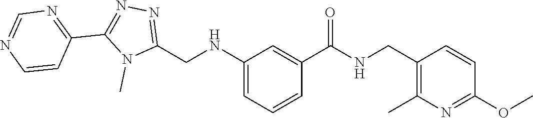 [SMILES: COc1ccc(CNC(=O)c2cccc(NCc3nnc(-c4ccncn4)n3C)c2)c(C)n1] | 28.5 | | >30.0 | 17.2 |
| 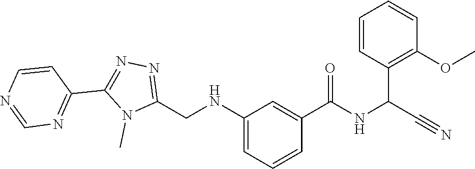 [SMILES: COc1ccccc1C(C#N)NC(=O)c1cccc(NCc2nnc(-c3ccncn3)n2C)c1] | 6.03 | | 0.468 | 37.7 |
| 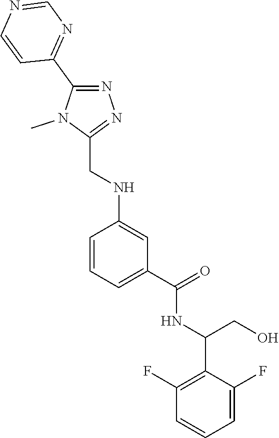 [SMILES: Cn1c(CNc2cccc(C(=O)NC(CO)c3c(F)cccc3F)c2)nnc1-c1ccncn1] | 146 | | | |
| 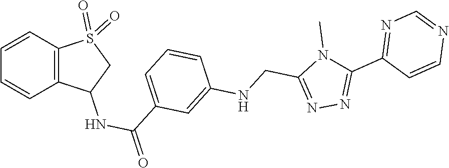 [SMILES: Cn1c(CNc2cccc(C(=O)NC3CS(=O)(=O)c4ccccc43)c2)nnc1-c1ccncn1] | 20.2 | | 1.44 | 56.5 |
| 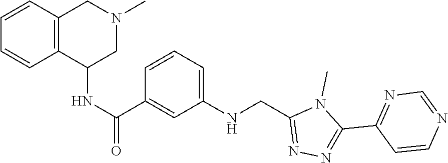 [SMILES: CN1Cc2ccccc2C(NC(=O)c2cccc(NCc3nnc(-c4ccncn4)n3C)c2)C1] | 45.9 | | 7.67 | 52.8 |
| 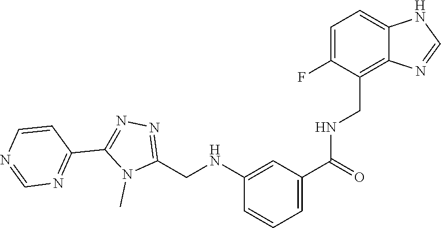 [SMILES: Cn1c(CNc2cccc(C(=O)NCc3c(F)ccc4[nH]cnc34)c2)nnc1-c1ccncn1] | 19.2 | | 22.6 | 55.7 |
| 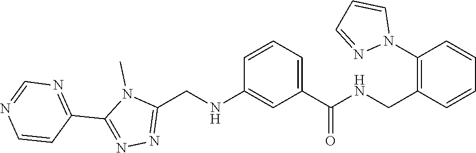 [SMILES: Cn1c(CNc2cccc(C(=O)NCc3ccccc3-n3cccn3)c2)nnc1-c1ccncn1] | 184 | | | |
| 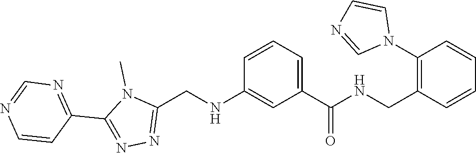 [SMILES: Cn1c(CNc2cccc(C(=O)NCc3ccccc3-n3ccnc3)c2)nnc1-c1ccncn1] | 30 | | 1.15 | 57.6 |
| 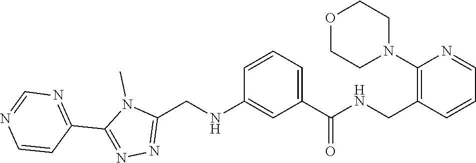 [SMILES: Cn1c(CNc2cccc(C(=O)NCc3cccnc3N3CCOCC3)c2)nnc1-c1ccncn1] | 176 | | | |
| 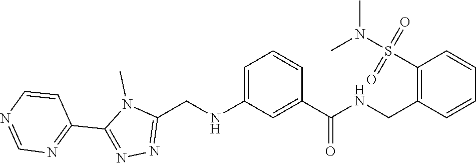 [SMILES: CN(C)S(=O)(=O)c1ccccc1CNC(=O)c1cccc(NCc2nnc(-c3ccncn3)n2C)c1] | 47.2 | | 21.5 | 40.1 |
| 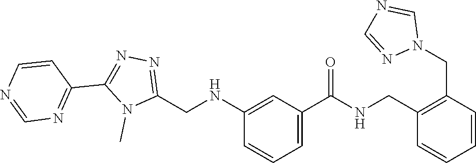 [SMILES: Cn1c(CNc2cccc(C(=O)NCc3ccccc3Cn3cncn3)c2)nnc1-c1ccncn1] | 55.7 | | 7.41 | 74.7 |
| 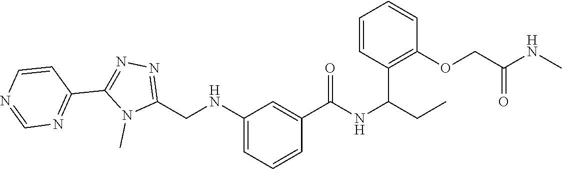 [SMILES: CCC(NC(=O)c1cccc(NCc2nnc(-c3ccncn3)n2C)c1)c1ccccc1OCC(=O)NC] | 67.2 | | >27.8 | 53.5 |
| 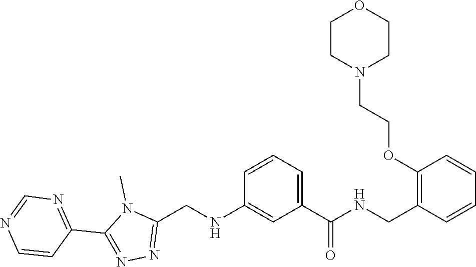 [SMILES: Cn1c(CNc2cccc(C(=O)NCc3ccccc3OCCN3CCOCC3)c2)nnc1-c1ccncn1] | 145 | | | |
| 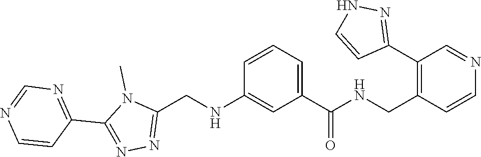 [SMILES: Cn1c(CNc2cccc(C(=O)NCc3ccncc3-c3cc[nH]n3)c2)nnc1-c1ccncn1] | 53.5 | | >23.3 | 68.8 |
| 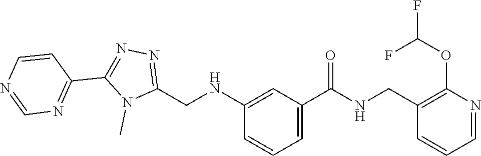 [SMILES: Cn1c(CNc2cccc(C(=O)NCc3cccnc3OC(F)F)c2)nnc1-c1ccncn1] | 16.7 | | 1.44 | 67.1 |
| 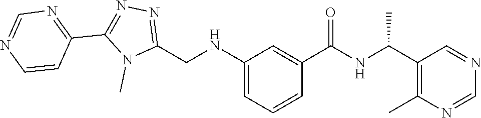 [SMILES: Cc1ncncc1[C@@H](C)NC(=O)c1cccc(NCc2nnc(-c3ccncn3)n2C)c1] | 15.5 | | >30.0 | 66.9 |
| 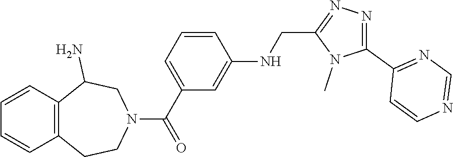 [SMILES: Cn1c(CNc2cccc(C(=O)N3CCc4ccccc4C(N)C3)c2)nnc1-c1ccncn1] | 10000 | | | |
| 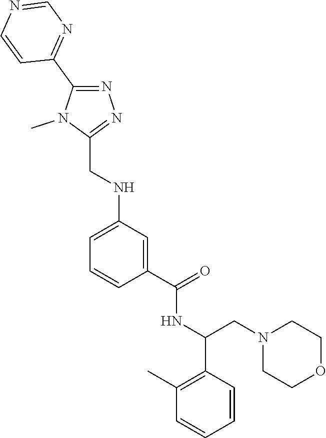 [SMILES: Cc1ccccc1C(CN1CCOCC1)NC(=O)c1cccc(NCc2nnc(-c3ccncn3)n2C)c1] | 505 | | | |
| 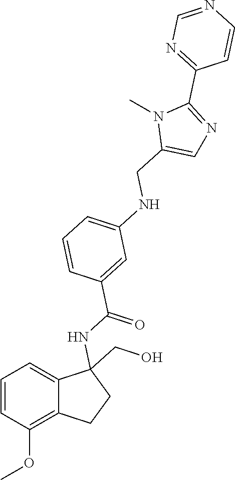 [SMILES: COc1cccc2c1CCC2(CO)NC(=O)c1cccc(NCc2cnc(-c3ccncn3)n2C)c1] | 134 | | | |
| 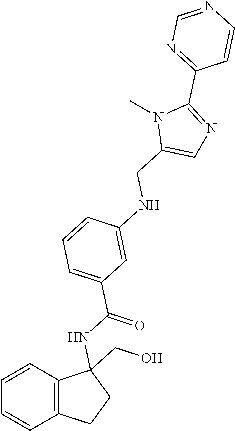 [SMILES: Cn1c(CNc2cccc(C(=O)NC3(CO)CCc4ccccc43)c2)cnc1-c1ccncn1] | 929 | | | |
| 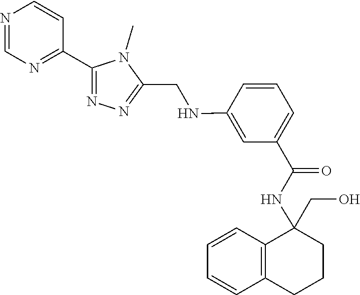 [SMILES: Cn1c(CNc2cccc(C(=O)NC3(CO)CCCc4ccccc43)c2)nnc1-c1ccncn1] | 651 | | | |
| 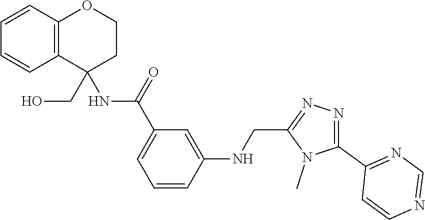 [SMILES: Cn1c(CNc2cccc(C(=O)NC3(CO)CCOc4ccccc43)c2)nnc1-c1ccncn1] | 283 | | | |
| 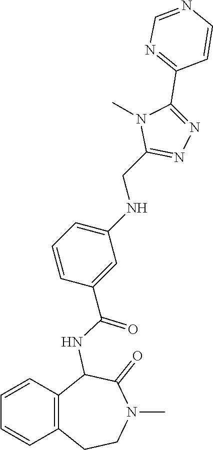 [SMILES: CN1CCc2ccccc2C(NC(=O)c2cccc(NCc3nnc(-c4ccncn4)n3C)c2)C1=O] | 253 | | | |
| 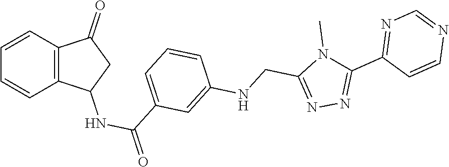 [SMILES: Cn1c(CNc2cccc(C(=O)NC3CC(=O)c4ccccc43)c2)nnc1-c1ccncn1] | 19 | | 0.867 | 61.1 |
| 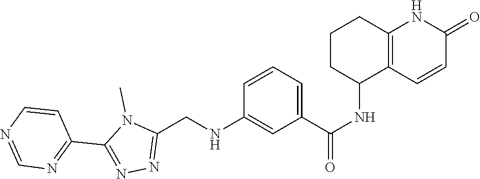 [SMILES: Cn1c(CNc2cccc(C(=O)NC3CCCc4[nH]c(=O)ccc43)c2)nnc1-c1ccncn1] | 392 | | | |
| 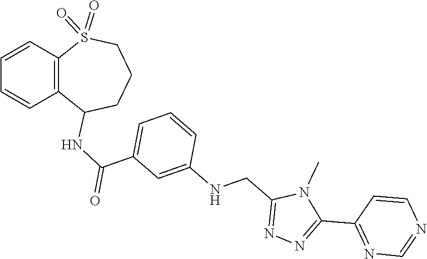 [SMILES: Cn1c(CNc2cccc(C(=O)NC3CCCS(=O)(=O)c4ccccc43)c2)nnc1-c1ccncn1] | 194 | | | |
| 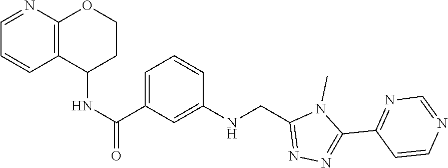 [SMILES: Cn1c(CNc2cccc(C(=O)NC3CCOc4ncccc43)c2)nnc1-c1ccncn1] | 56.7 | | >30.0 | 84.2 |
| 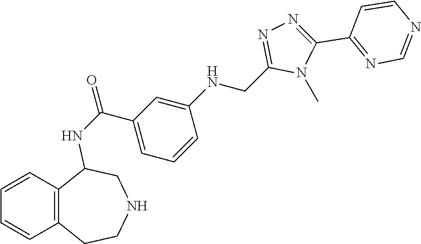 [SMILES: Cn1c(CNc2cccc(C(=O)NC3CNCCc4ccccc43)c2)nnc1-c1ccncn1] | 142 | | | |
| 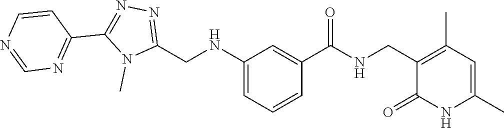 [SMILES: Cc1cc(C)c(CNC(=O)c2cccc(NCc3nnc(-c4ccncn4)n3C)c2)c(=O)[nH]1] | 14.9 | | >30.0 | 78.9 |
| 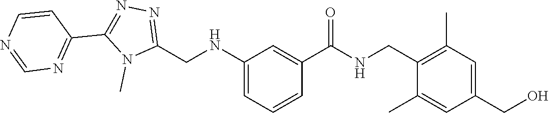 [SMILES: Cc1cc(CO)cc(C)c1CNC(=O)c1cccc(NCc2nnc(-c3ccncn3)n2C)c1] | 10.8 | | 0.738 | 51.3 |
| 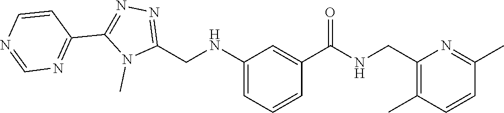 [SMILES: Cc1ccc(C)c(CNC(=O)c2cccc(NCc3nnc(-c4ccncn4)n3C)c2)n1] | 11.2 | | >30.0 | 16.2 |
| 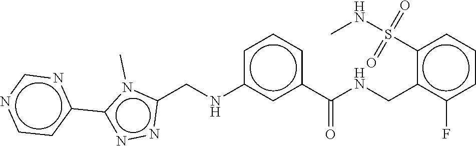 | 7.36 | 12.7 | 0.732 | 47.1 |
| 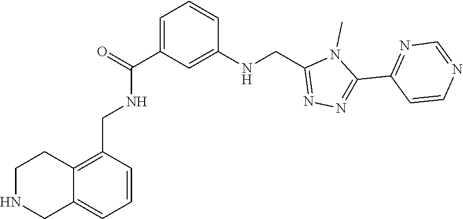 [SMILES: Cn1c(CNc2cccc(C(=O)NCc3cccc4c3CCNC4)c2)nnc1-c1ccncn1] | 18.4 | | >30.00 | 96.1 |
| 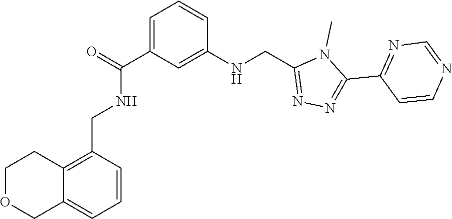 [SMILES: Cn1c(CNc2cccc(C(=O)NCc3cccc4c3CCOC4)c2)nnc1-c1ccncn1] | 8.5 | | >30.0 | 37.8 |
| 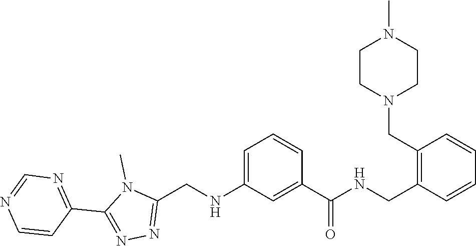 [SMILES: CN1CCN(Cc2ccccc2CNC(=O)c2cccc(NCc3nnc(-c4ccncn4)n3C)c2)CC1] | 159 | | | |
| 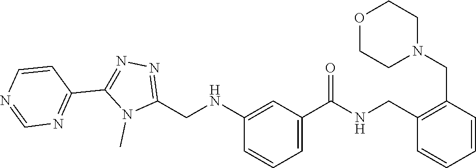 [SMILES: Cn1c(CNc2cccc(C(=O)NCc3ccccc3CN3CCOCC3)c2)nnc1-c1ccncn1] | 71.5 | | 6.71 | 59.8 |
| 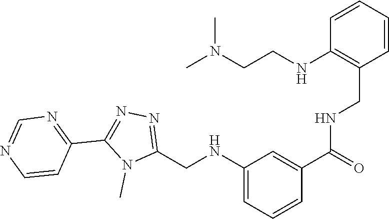 [SMILES: CN(C)CCNc1ccccc1CNC(=O)c1cccc(NCc2nnc(-c3ccncn3)n2C)c1] | 53.3 | | >30.0 | 84.4 |
| 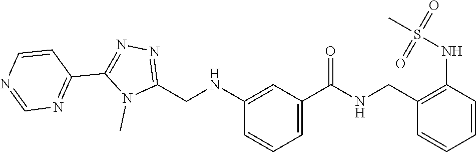 [SMILES: Cn1c(CNc2cccc(C(=O)NCc3ccccc3NS(C)(=O)=O)c2)nnc1-c1ccncn1] | 32.1 | | >30.0 | 65.6 |
| 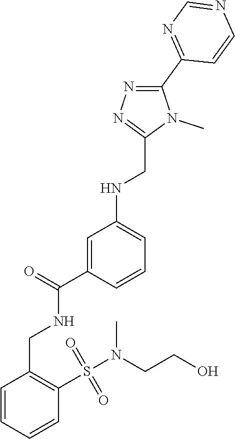 [SMILES: CN(CCO)S(=O)(=O)c1ccccc1CNC(=O)c1cccc(NCc2nnc(-c3ccncn3)n2C)c1] | 42 | | >11.8 | 54.3 |
| 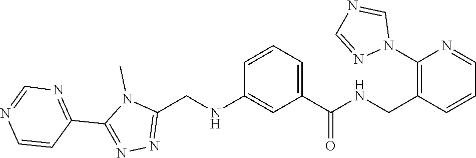 [SMILES: Cn1c(CNc2cccc(C(=O)NCc3cccnc3-n3cncn3)c2)nnc1-c1ccncn1] | 119 | | | |
| 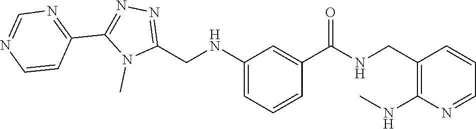 [SMILES: CNc1ncccc1CNC(=O)c1cccc(NCc2nnc(-c3ccncn3)n2C)c1] | 18.3 | | 1.63 | 43.3 |
| 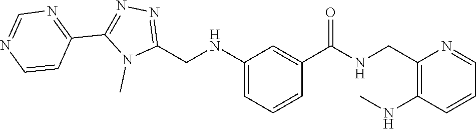 [SMILES: CNc1cccnc1CNC(=O)c1cccc(NCc2nnc(-c3ccncn3)n2C)c1] | 133 | | | |
| 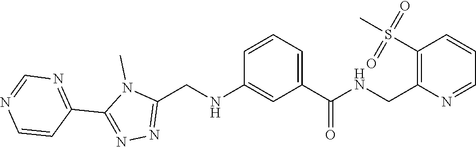 [SMILES: Cn1c(CNc2cccc(C(=O)NCc3ncccc3S(C)(=O)=O)c2)nnc1-c1ccncn1] | 172 | | | |
| 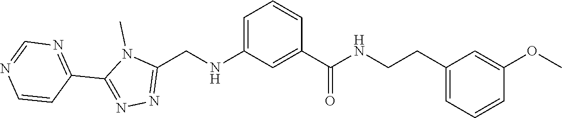 [SMILES: COc1cccc(CCNC(=O)c2cccc(NCc3nnc(-c4ccncn4)n3C)c2)c1] | 340 | | | |
| 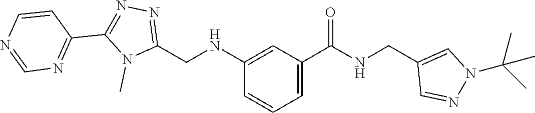 [SMILES: Cn1c(CNc2cccc(C(=O)NCc3cnn(C(C)(C)C)c3)c2)nnc1-c1ccncn1] | 135 | | | |
| 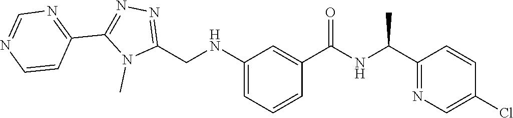 [SMILES: C[C@H](NC(=O)c1cccc(NCc2nnc(-c3ccncn3)n2C)c1)c1ccc(Cl)cn1] | 950 | | | |
| 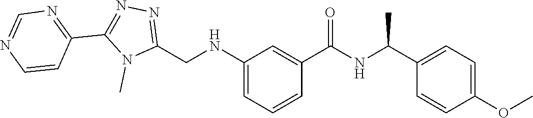 [SMILES: COc1ccc([C@H](C)NC(=O)c2cccc(NCc3nnc(-c4ccncn4)n3C)c2)cc1] | 593 | | | |
| 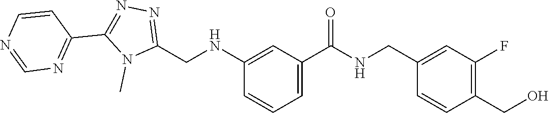 [SMILES: Cn1c(CNc2cccc(C(=O)NCc3ccc(CO)c(F)c3)c2)nnc1-c1ccncn1] | 103 | | | |
| 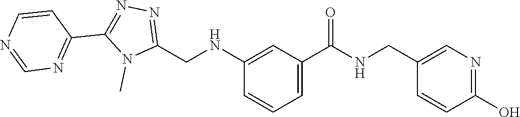 [SMILES: Cn1c(CNc2cccc(C(=O)NCc3ccc(O)nc3)c2)nnc1-c1ccncn1] | 203 | | | |
| 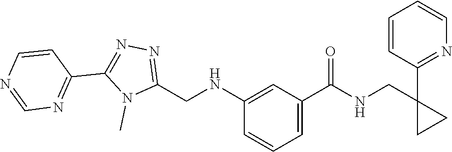 [SMILES: Cn1c(CNc2cccc(C(=O)NCC3(c4ccccn4)CC3)c2)nnc1-c1ccncn1] | 526 | | | |
| 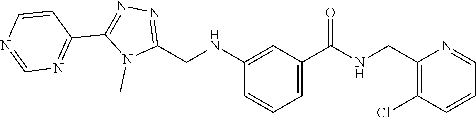 [SMILES: Cn1c(CNc2cccc(C(=O)NCc3ncccc3Cl)c2)nnc1-c1ccncn1] | 48.2 | | >16.4 | 40.6 |
| 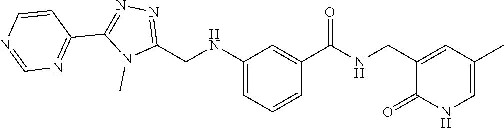 [SMILES: Cc1c[nH]c(=O)c(CNC(=O)c2cccc(NCc3nnc(-c4ccncn4)n3C)c2)c1] | >1.00E + 03 | | | |
| 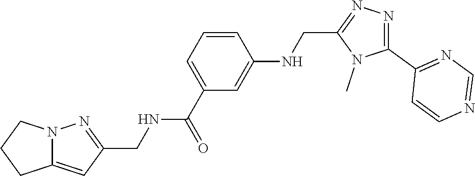 [SMILES: Cn1c(CNc2cccc(C(=O)NCc3cc4n(n3)CCC4)c2)nnc1-c1ccncn1] | >1.00E + 03 | | | |
| 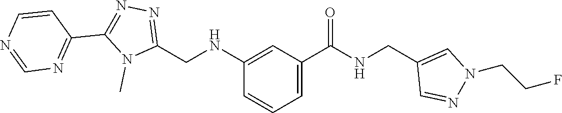 [SMILES: Cn1c(CNc2cccc(C(=O)NCc3cnn(CCF)c3)c2)nnc1-c1ccncn1] | 82.9 | | >5.60 | 67.5 |
| 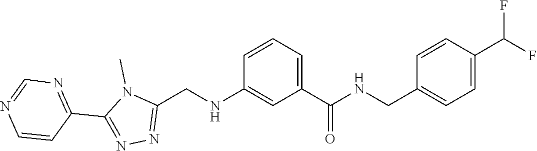 [SMILES: Cn1c(CNc2cccc(C(=O)NCc3ccc(C(F)F)cc3)c2)nnc1-c1ccncn1] | 64.2 | | >13.1 | 72.6 |
| 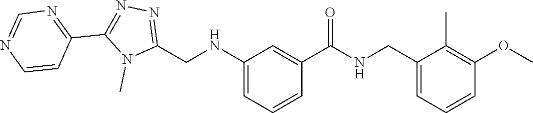 [SMILES: COc1cccc(CNC(=O)c2cccc(NCc3nnc(-c4ccncn4)n3C)c2)c1C] | 12.6 | | >12.6 | 26.4 |
| 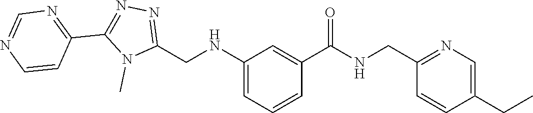 [SMILES: CCc1ccc(CNC(=O)c2cccc(NCc3nnc(-c4ccncn4)n3C)c2)nc1] | 271 | | | |
| 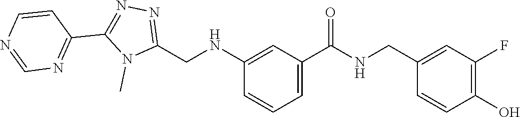 [SMILES: Cn1c(CNc2cccc(C(=O)NCc3ccc(O)c(F)c3)c2)nnc1-c1ccncn1] | 118 | | | |
| 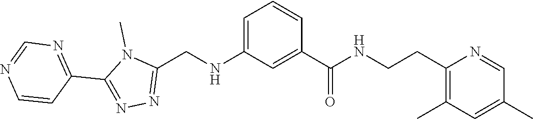 [SMILES: Cc1cnc(CCNC(=O)c2cccc(NCc3nnc(-c4ccncn4)n3C)c2)c(C)c1] | 432 | | | |
| 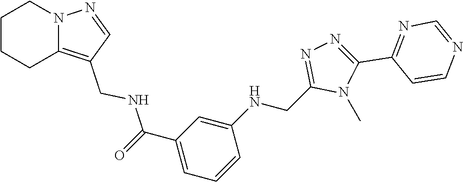 [SMILES: Cn1c(CNc2cccc(C(=O)NCc3cnn4c3CCCC4)c2)nnc1-c1ccncn1] | 71.6 | | >24.8 | 59.3 |
| 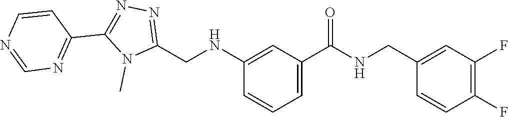 [SMILES: Cn1c(CNc2cccc(C(=O)NCc3ccc(F)c(F)c3)c2)nnc1-c1ccncn1] | 63.7 | | 9.33 | 65.1 |
| 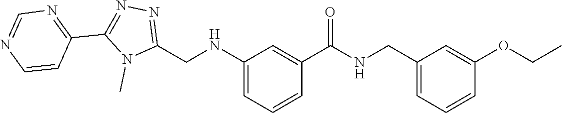 [SMILES: CCOc1cccc(CNC(=O)c2cccc(NCc3nnc(-c4ccncn4)n3C)c2)c1] | 73.6 | | >30.0 | 72 |
| 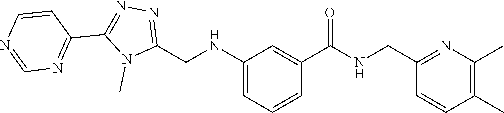 [SMILES: Cc1ccc(CNC(=O)c2cccc(NCc3nnc(-c4ccncn4)n3C)c2)nc1C] | 182 | | | |
| 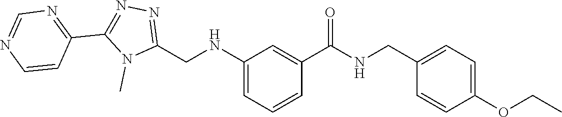 [SMILES: CCOc1ccc(CNC(=O)c2cccc(NCc3nnc(-c4ccncn4)n3C)c2)cc1] | 35.3 | | 1.2 | 66.9 |
| 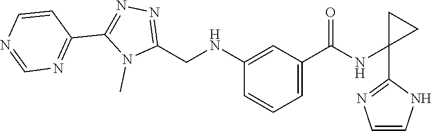 [SMILES: Cn1c(CNc2cccc(C(=O)NC3(c4ncc[nH]4)CC3)c2)nnc1-c1ccncn1] | 748 | | | |
| 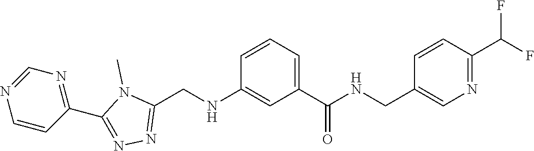 [SMILES: Cn1c(CNc2cccc(C(=O)NCc3ccc(C(F)F)nc3)c2)nnc1-c1ccncn1] | 44.6 | | >4.16 | 71.2 |
| 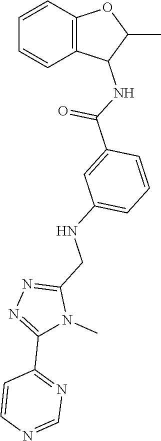 [SMILES: CC1Oc2ccccc2C1NC(=O)c1cccc(NCc2nnc(-c3ccncn3)n2C)c1] | 7.43 | | 1.79 | 62.1 |
| 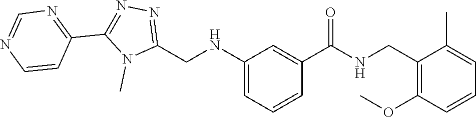 [SMILES: COc1cccc(C)c1CNC(=O)c1cccc(NCc2nnc(-c3ccncn3)n2C)c1] | 8.2 | | 0.246 | 53.2 |
| 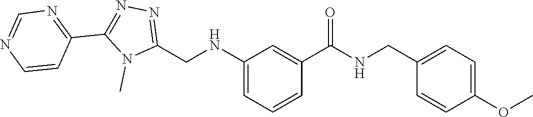 [SMILES: COc1ccc(CNC(=O)c2cccc(NCc3nnc(-c4ccncn4)n3C)c2)cc1] | 913 | | | |
| 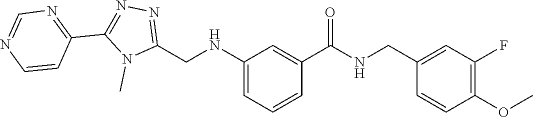 [SMILES: COc1ccc(CNC(=O)c2cccc(NCc3nnc(-c4ccncn4)n3C)c2)cc1F] | 30.1 | | 6.65 | 48.9 |
| 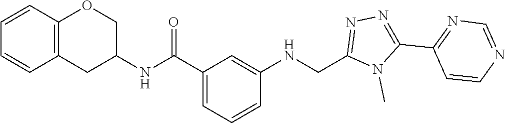 [SMILES: Cn1c(CNc2cccc(C(=O)NC3COc4ccccc4C3)c2)nnc1-c1ccncn1] | 68.4 | | >12.6 | 68.1 |
| 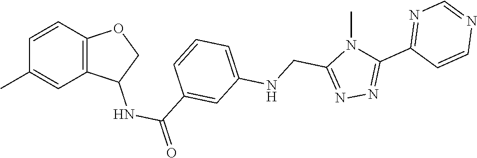 [SMILES: Cc1ccc2c(c1)C(NC(=O)c1cccc(NCc3nnc(-c4ccncn4)n3C)c1)CO2] | 18.8 | | 0.332 | 60.7 |
| 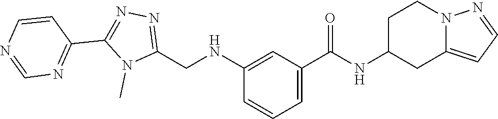 [SMILES: Cn1c(CNc2cccc(C(=O)NC3CCn4nccc4C3)c2)nnc1-c1ccncn1] | 133 | | | |
| 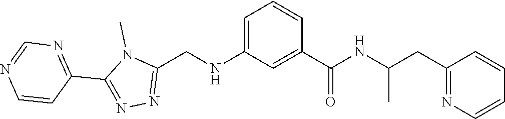 [SMILES: CC(Cc1ccccn1)NC(=O)c1cccc(NCc2nnc(-c3ccncn3)n2C)c1] | 290 | | | |
| 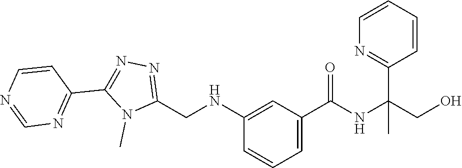 [SMILES: Cn1c(CNc2cccc(C(=O)NC(C)(CO)c3ccccn3)c2)nnc1-c1ccncn1] | 511 | | | |
| 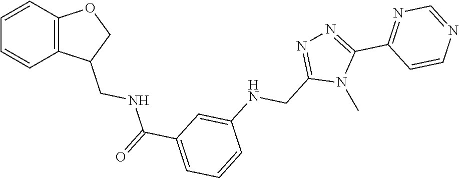 [SMILES: Cn1c(CNc2cccc(C(=O)NCC3COc4ccccc43)c2)nnc1-c1ccncn1] | 54.7 | | >5.21 | 71.9 |
| 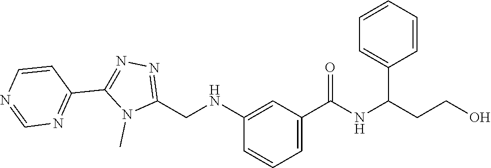 [SMILES: Cn1c(CNc2cccc(C(=O)NC(CCO)c3ccccc3)c2)nnc1-c1ccncn1] | 26.4 | | 1.09 | 60.4 |
| 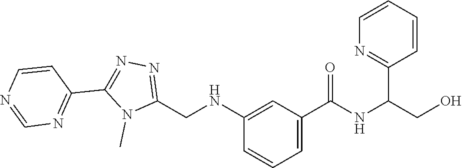 [SMILES: Cn1c(CNc2cccc(C(=O)NC(CO)c3ccccn3)c2)nnc1-c1ccncn1] | 177 | | | |
| 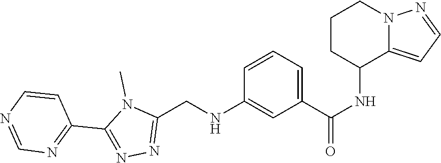 [SMILES: Cn1c(CNc2cccc(C(=O)NC3CCCn4nccc43)c2)nnc1-c1ccncn1] | 171 | | | |
| 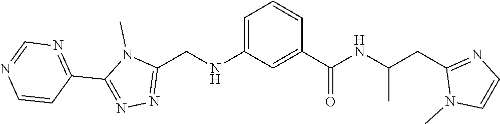 [SMILES: CC(Cc1nccn1C)NC(=O)c1cccc(NCc2nnc(-c3ccncn3)n2C)c1] | 732 | | | |
| 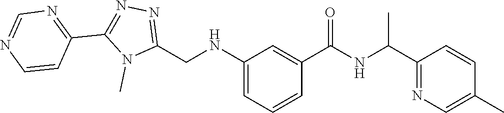 [SMILES: Cc1ccc(C(C)NC(=O)c2cccc(NCc3nnc(-c4ccncn4)n3C)c2)nc1] | 74 | | >5.02 | 25.6 |
| 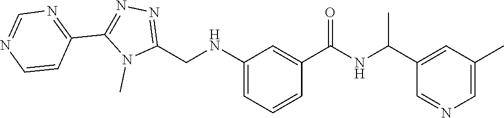 [SMILES: Cc1cncc(C(C)NC(=O)c2cccc(NCc3nnc(-c4ccncn4)n3C)c2)c1] | 16.2 | | 0.784 | 56.8 |
| 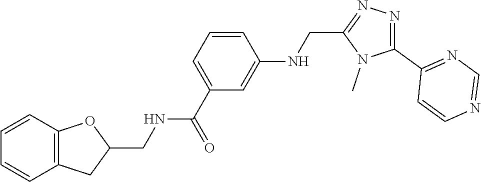 [SMILES: Cn1c(CNc2cccc(C(=O)NCC3Cc4ccccc4O3)c2)nnc1-c1ccncn1] | 208 | | | |
| 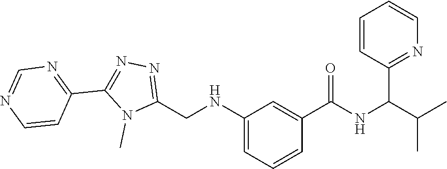 [SMILES: CC(C)C(NC(=O)c1cccc(NCc2nnc(-c3ccncn3)n2C)c1)c1ccccn1] | 52.8 | | 1.9 | 64.5 |
| 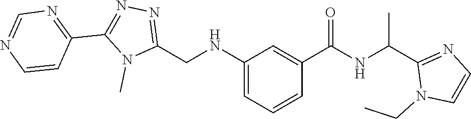 [SMILES: CCn1ccnc1C(C)NC(=O)c1cccc(NCc2nnc(-c3ccncn3)n2C)c1] | 189 | | | |
| 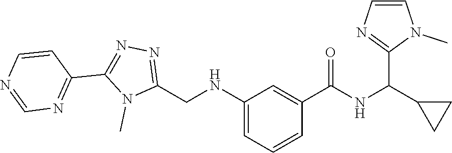 [SMILES: Cn1ccnc1C(NC(=O)c1cccc(NCc2nnc(-c3ccncn3)n2C)c1)C1CC1] | 52.9 | | 3.75 | 47.4 |
| 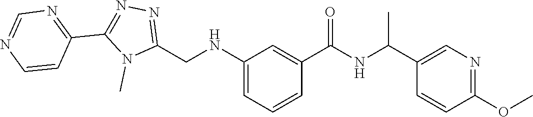 [SMILES: COc1ccc(C(C)NC(=O)c2cccc(NCc3nnc(-c4ccncn4)n3C)c2)cn1] | 22.6 | | 1.71 | 56.3 |
| 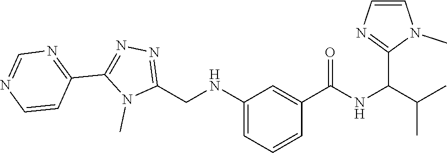 [SMILES: CC(C)C(NC(=O)c1cccc(NCc2nnc(-c3ccncn3)n2C)c1)c1nccn1C] | 152 | | | |
| 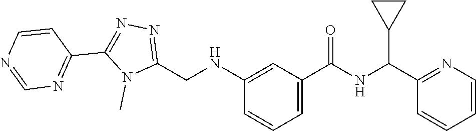 [SMILES: Cn1c(CNc2cccc(C(=O)NC(c3ccccn3)C3CC3)c2)nnc1-c1ccncn1] | 68.9 | | 2.63 | 58.5 |
| 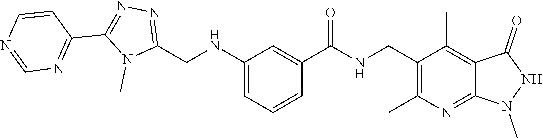 [SMILES: Cc1nc2c(c(C)c1CNC(=O)c1cccc(NCc3nnc(-c4ccncn4)n3C)c1)c(=O)[nH]n2C] | 181 | | | |
| 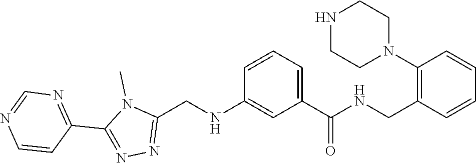 [SMILES: Cn1c(CNc2cccc(C(=O)NCc3ccccc3N3CCNCC3)c2)nnc1-c1ccncn1] | 65.8 | | 7.89 | 71.9 |
| 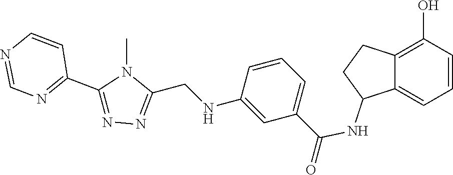 [SMILES: Cn1c(CNc2cccc(C(=O)NC3CCc4c(O)cccc43)c2)nnc1-c1ccncn1] | 47.2 | | 8.84 | 68.9 |
| 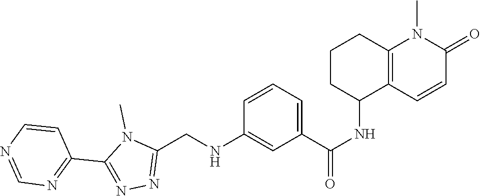 [SMILES: Cn1c(CNc2cccc(C(=O)NC3CCCc4c3ccc(=O)n4C)c2)nnc1-c1ccncn1] | >1.00E + 03 | | | |
| 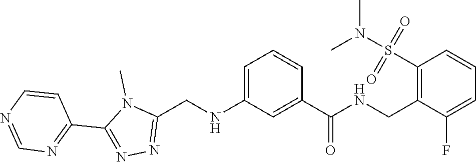 [SMILES: CN(C)S(=O)(=O)c1cccc(F)c1CNC(=O)c1cccc(NCc2nnc(-c3ccncn3)n2C)c1] | 5.78 | | 0.263 | 26.9 |
| 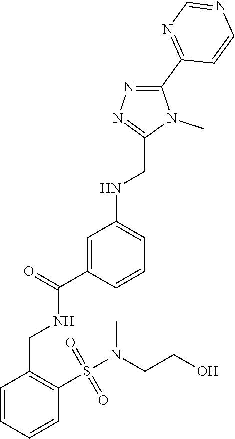 [SMILES: CN(CCO)S(=O)(=O)c1ccccc1CNC(=O)c1cccc(NCc2nnc(-c3ccncn3)n2C)c1] | | | | |
| 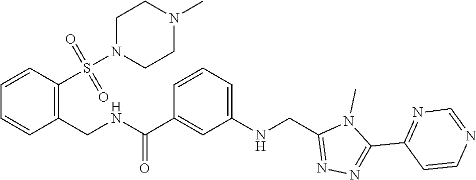 [SMILES: CN1CCN(S(=O)(=O)c2ccccc2CNC(=O)c2cccc(NCc3nnc(-c4ccncn4)n3C)c2)CC1] | 344 | | | |
| 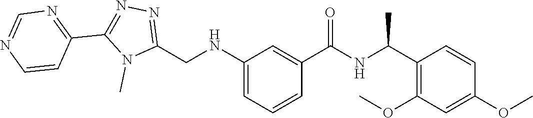 [SMILES: COc1ccc([C@H](C)NC(=O)c2cccc(NCc3nnc(-c4ccncn4)n3C)c2)c(OC)c1] | 310 | | | |
| 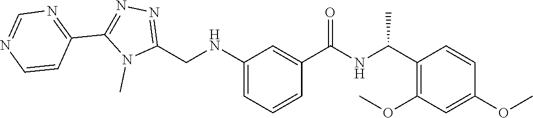 [SMILES: COc1ccc([C@@H](C)NC(=O)c2cccc(NCc3nnc(-c4ccncn4)n3C)c2)c(OC)c1] | 8.56 | | | |
| 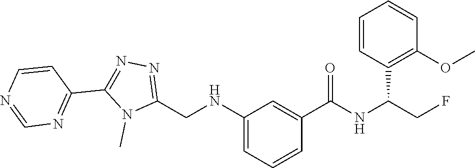 [SMILES: COc1ccccc1[C@H](CF)NC(=O)c1cccc(NCc2nnc(-c3ccncn3)n2C)c1] | | | | |
| 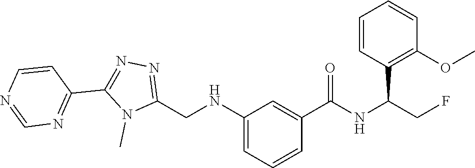 [SMILES: COc1ccccc1[C@@H](CF)NC(=O)c1cccc(NCc2nnc(-c3ccncn3)n2C)c1] | | | | |
| 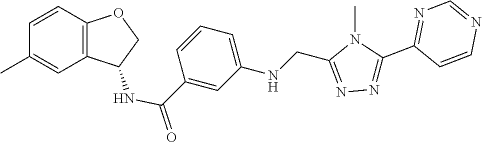 [SMILES: Cc1ccc2c(c1)[C@@H](NC(=O)c1cccc(NCc3nnc(-c4ccncn4)n3C)c1)CO2] | | | | |
| 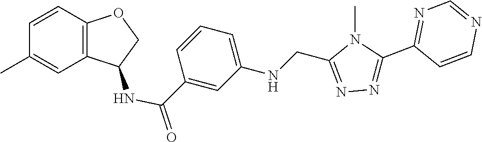 [SMILES: Cc1ccc2c(c1)[C@H](NC(=O)c1cccc(NCc3nnc(-c4ccncn4)n3C)c1)CO2] | | | | |
| 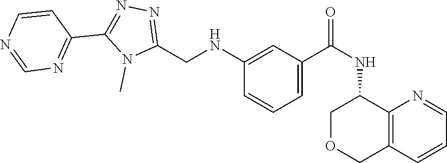 [SMILES: Cn1c(CNc2cccc(C(=O)N[C@H]3COCc4cccnc43)c2)nnc1-c1ccncn1] | | | | |
| 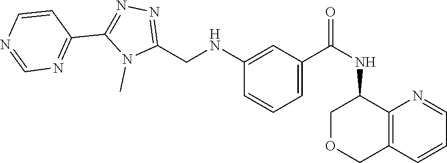 [SMILES: Cn1c(CNc2cccc(C(=O)N[C@@H]3COCc4cccnc43)c2)nnc1-c1ccncn1] | | | | |
| 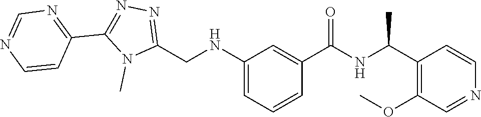 [SMILES: COc1cnccc1[C@H](C)NC(=O)c1cccc(NCc2nnc(-c3ccncn3)n2C)c1] | | | | |
| 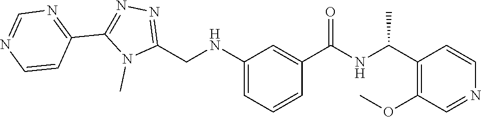 [SMILES: COc1cnccc1[C@@H](C)NC(=O)c1cccc(NCc2nnc(-c3ccncn3)n2C)c1] | | | | |
| 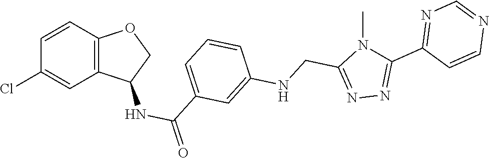 [SMILES: Cn1c(CNc2cccc(C(=O)N[C@@H]3COc4ccc(Cl)cc43)c2)nnc1-c1ccncn1] | | | | |
| 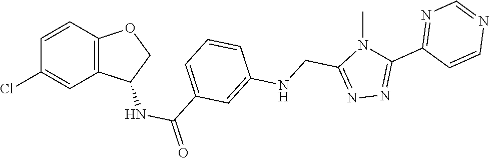 [SMILES: Cn1c(CNc2cccc(C(=O)N[C@H]3COc4ccc(Cl)cc43)c2)nnc1-c1ccncn1] | | | | |
| 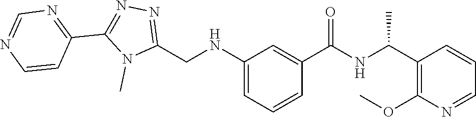 [SMILES: COc1ncccc1[C@@H](C)NC(=O)c1cccc(NCc2nnc(-c3ccncn3)n2C)c1] | | | | |
| 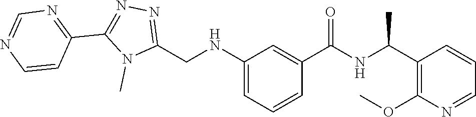 [SMILES: COc1ncccc1[C@H](C)NC(=O)c1cccc(NCc2nnc(-c3ccncn3)n2C)c1] | | | | |
| 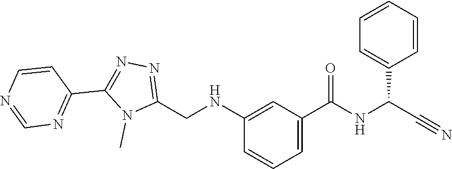 [SMILES: Cn1c(CNc2cccc(C(=O)N[C@@H](C#N)c3ccccc3)c2)nnc1-c1ccncn1] | | | | |
| 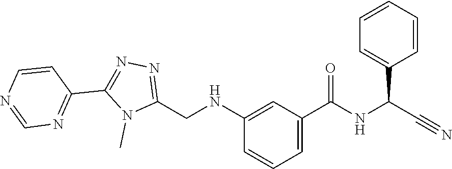 [SMILES: Cn1c(CNc2cccc(C(=O)N[C@H](C#N)c3ccccc3)c2)nnc1-c1ccncn1] | | | | |
| 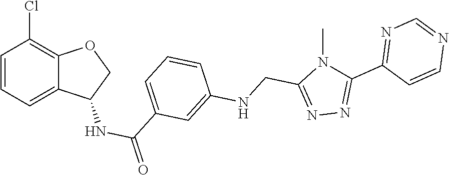 [SMILES: Cn1c(CNc2cccc(C(=O)N[C@H]3COc4c(Cl)cccc43)c2)nnc1-c1ccncn1] | | 7.8 | | |
| 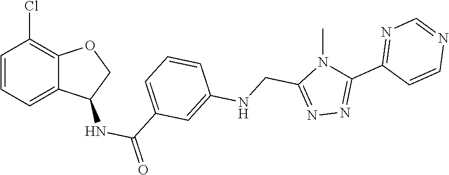 [SMILES: Cn1c(CNc2cccc(C(=O)N[C@@H]3COc4c(Cl)cccc43)c2)nnc1-c1ccncn1] | | 28 | | |
| 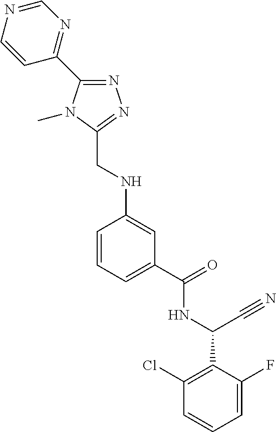 [SMILES: Cn1c(CNc2cccc(C(=O)N[C@H](C#N)c3c(F)cccc3Cl)c2)nnc1-c1ccncn1] | | 5.7 | | |
| 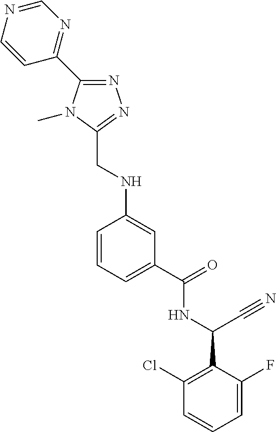 [SMILES: Cn1c(CNc2cccc(C(=O)N[C@@H](C#N)c3c(F)cccc3Cl)c2)nnc1-c1ccncn1] | | 5.1 | | |
| 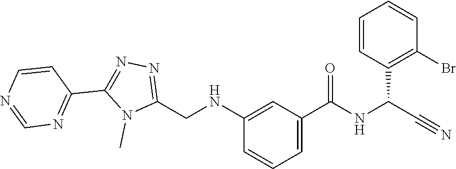 [SMILES: Cn1c(CNc2cccc(C(=O)N[C@@H](C#N)c3ccccc3Br)c2)nnc1-c1ccncn1] | | 4.1 | | |
| 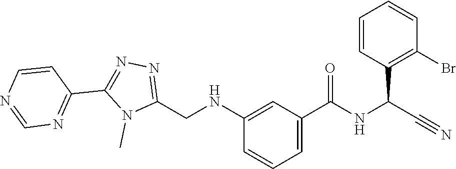 [SMILES: Cn1c(CNc2cccc(C(=O)N[C@H](C#N)c3ccccc3Br)c2)nnc1-c1ccncn1] | | 4.5 | | |
| 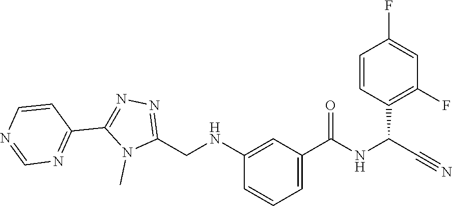 [SMILES: Cn1c(CNc2cccc(C(=O)N[C@@H](C#N)c3ccc(F)cc3F)c2)nnc1-c1ccncn1] | | 10 | | |
| 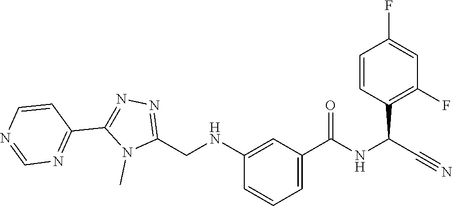 [SMILES: Cn1c(CNc2cccc(C(=O)N[C@H](C#N)c3ccc(F)cc3F)c2)nnc1-c1ccncn1] | | 4.9 | 0.122 | 42.7 |
| 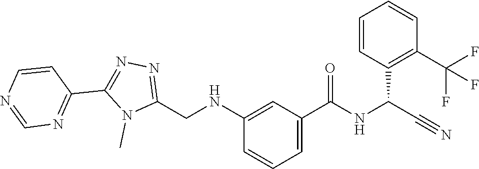 [SMILES: Cn1c(CNc2cccc(C(=O)N[C@@H](C#N)c3ccccc3C(F)(F)F)c2)nnc1-c1ccncn1] | | 3.5 | | |
| 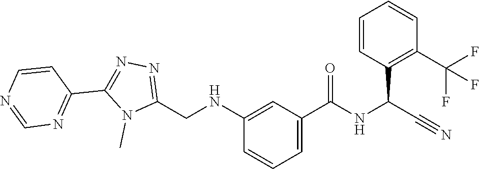 [SMILES: Cn1c(CNc2cccc(C(=O)N[C@H](C#N)c3ccccc3C(F)(F)F)c2)nnc1-c1ccncn1] | | 3.5 | 0.0544 | 25 |
| 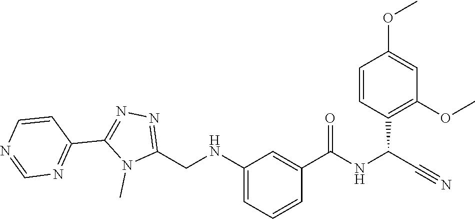 [SMILES: COc1ccc([C@H](C#N)NC(=O)c2cccc(NCc3nnc(-c4ccncn4)n3C)c2)c(OC)c1] | | 68 | | |
| 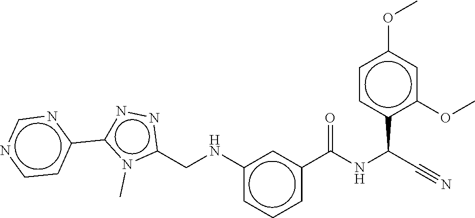 | | 7.8 | | |
| 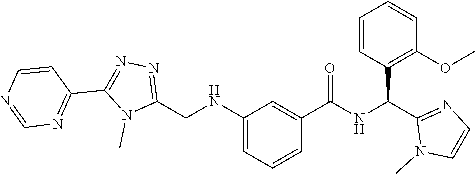 [SMILES: COc1ccccc1[C@H](NC(=O)c1cccc(NCc2nnc(-c3ccncn3)n2C)c1)c1nccn1C] | | 100 | | |
| 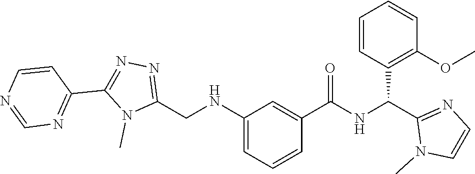 [SMILES: COc1ccccc1[C@@H](NC(=O)c1cccc(NCc2nnc(-c3ccncn3)n2C)c1)c1nccn1C] | 25 | 0.579 | 78.2 | |
| 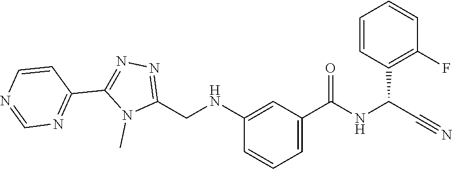 [SMILES: Cn1c(CNc2cccc(C(=O)N[C@@H](C#N)c3ccccc3F)c2)nnc1-c1ccncn1] | | 5.5 | 0.0593 | 29 |
| 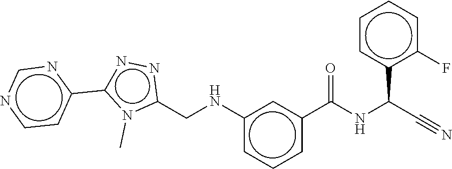 | | 6.8 | 0.0826 | 29.7 |
| 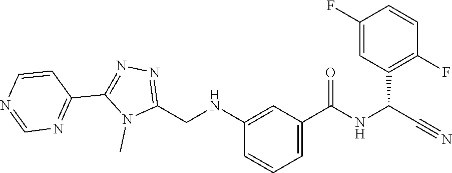 [SMILES: Cn1c(CNc2cccc(C(=O)N[C@@H](C#N)c3cc(F)ccc3F)c2)nnc1-c1ccncn1] | 15.7 | | 0.244 | 35 |
| 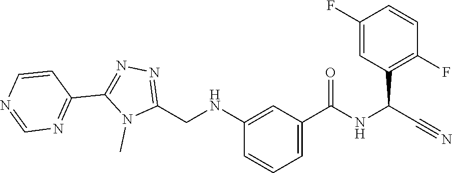 [SMILES: Cn1c(CNc2cccc(C(=O)N[C@H](C#N)c3cc(F)ccc3F)c2)nnc1-c1ccncn1] | 17.7 | | 0.808 | 27.7 |
| 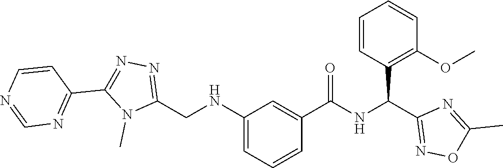 [SMILES: COc1ccccc1[C@H](NC(=O)c1cccc(NCc2nnc(-c3ccncn3)n2C)c1)c1noc(C)n1] | 741 | | | |
| 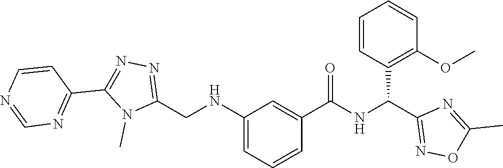 [SMILES: COc1ccccc1[C@@H](NC(=O)c1cccc(NCc2nnc(-c3ccncn3)n2C)c1)c1noc(C)n1] | 333 | | | |
| 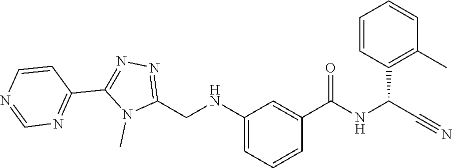 [SMILES: Cc1ccccc1[C@H](C#N)NC(=O)c1cccc(NCc2nnc(-c3ccncn3)n2C)c1] | 4.63 | | 0.0304 | 49 |
| 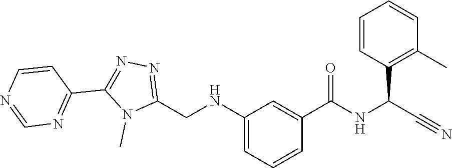 [SMILES: Cc1ccccc1[C@@H](C#N)NC(=O)c1cccc(NCc2nnc(-c3ccncn3)n2C)c1] | 4.97 | | 0.0355 | 48.6 |
| 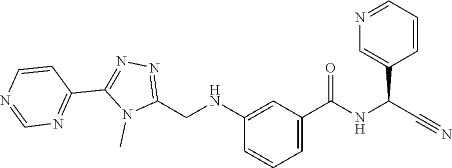 [SMILES: Cn1c(CNc2cccc(C(=O)N[C@H](C#N)c3cccnc3)c2)nnc1-c1ccncn1] | 20.3 | | 3.43 | 4.7 |
| 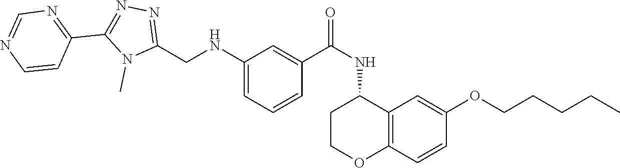 [SMILES: CCCCCOc1ccc2c(c1)[C@@H](NC(=O)c1cccc(NCc3nnc(-c4ccncn4)n3C)c1)CCO2] | 12.6 | | 2.13 | 19.4 |
| 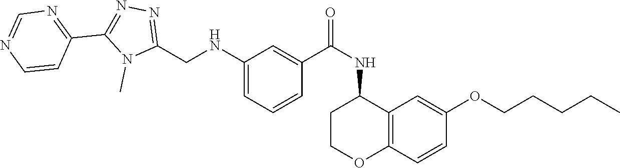 [SMILES: CCCCCOc1ccc2c(c1)[C@H](NC(=O)c1cccc(NCc3nnc(-c4ccncn4)n3C)c1)CCO2] | 732 | | | |
| 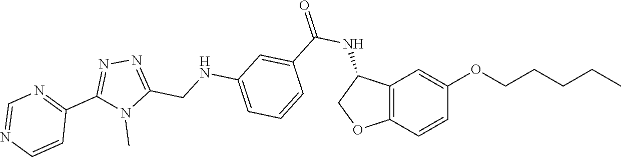 [SMILES: CCCCCOc1ccc2c(c1)[C@@H](NC(=O)c1cccc(NCc3nnc(-c4ccncn4)n3C)c1)CO2] | 8.3 | | 0.236 | 27.8 |
| 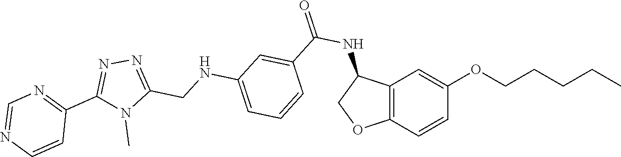 [SMILES: CCCCCOc1ccc2c(c1)[C@H](NC(=O)c1cccc(NCc3nnc(-c4ccncn4)n3C)c1)CO2] | 550 | | | |
| 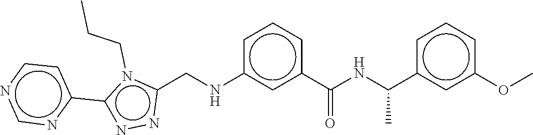 | 14.1 | | >30.0 | 1.79 |
| 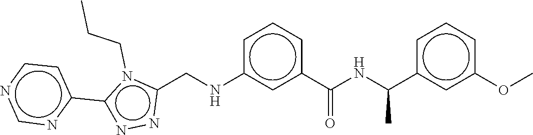 | 40.1 | | >0.219 | 53.5 |
| 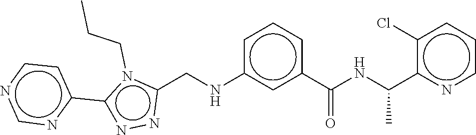 | | | | |
| 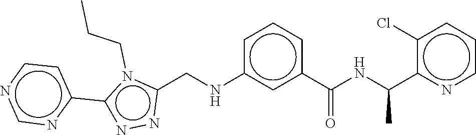 | | | | |
| 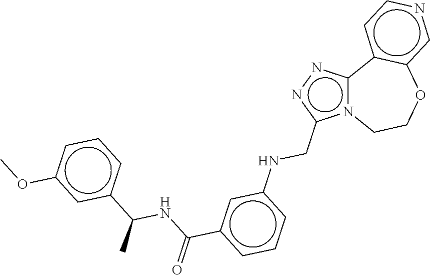 | 6.52 | | 10.1 | 9.01 |
| 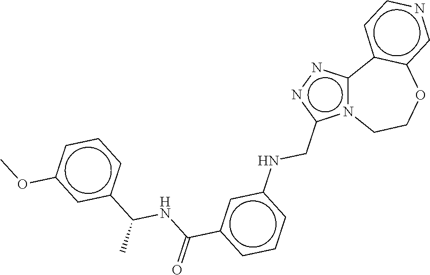 | 15.9 | | >3.65 | 56.5 |
| 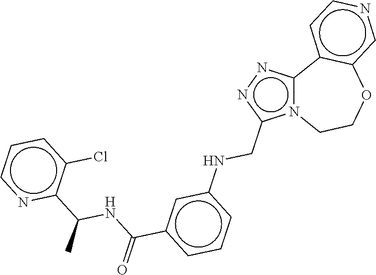 | | | | |
| 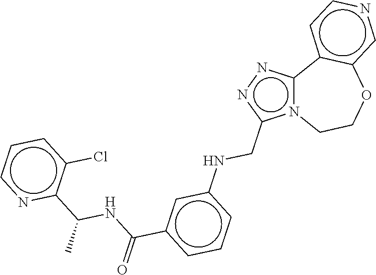 | | | | |
| 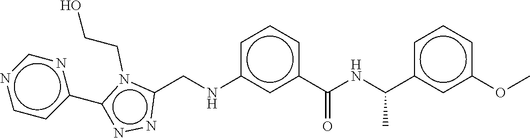 | 2.41 | | 0.0307 | 30.1 |
| 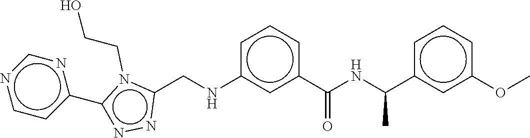 | 4.91 | | 1.23 | 40.5 |
| 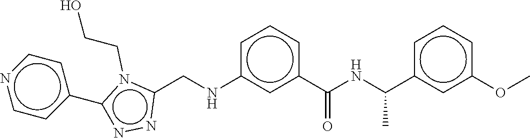 | 2.99 | | >30.0 | 51.2 |
| 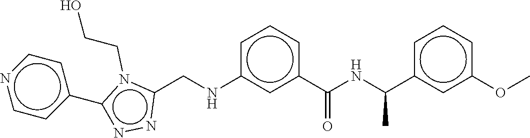 | 99.4 | | >30.0 | 68.8 |
| 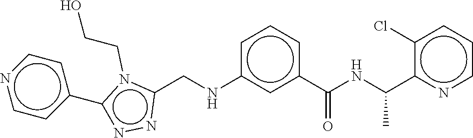 | 28.3 | | 0.0809 | 49.2 |
| 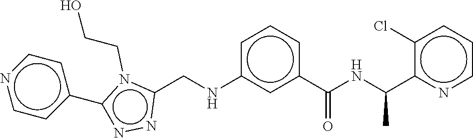 | 11.9 | | | |
| 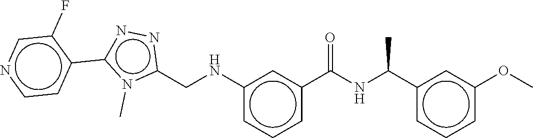 | 11.6 | | >30.0 | 34.1 |
| 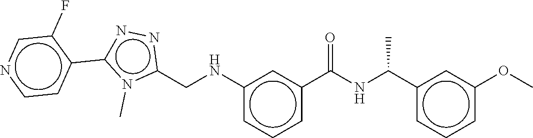 | 181 | | >30.0 | 28.8 |
| 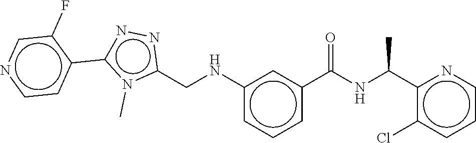 | 52.8 | | 1.03 | 27.6 |
| 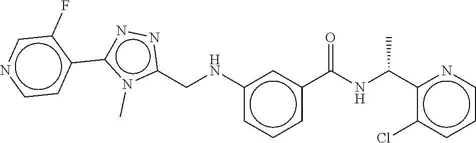 | 54.7 | | 0.135 | 48.3 |
| 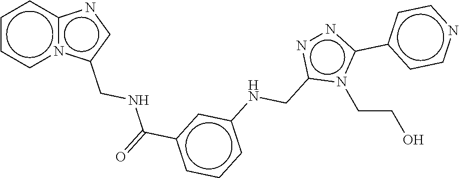 | 5.77 | | >30.0 | 51.6 |
| 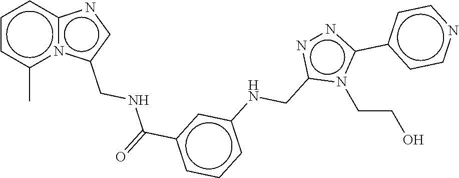 | 5.86 | | >30.0 | 42 |
| 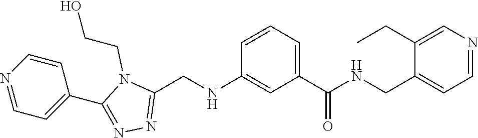 [SMILES: CCc1cnccc1CNC(=O)c1cccc(NCc2nnc(-c3ccncc3)n2CCO)c1] | 14.3 | | >30.0 | 41 |
| 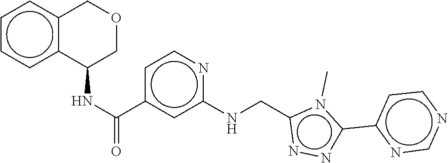 | 18.7 | | | |
| 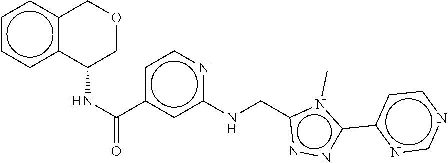 | 71.1 | | 11 | 54.4 |
| 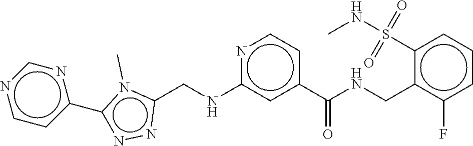 | 15.1 | | | |
| 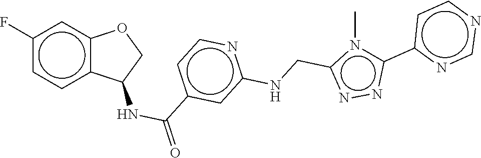 | 37.5 | | | |
| 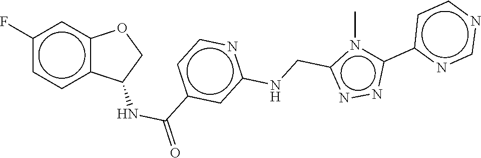 | 118 | | 1.93 | 65 |
| 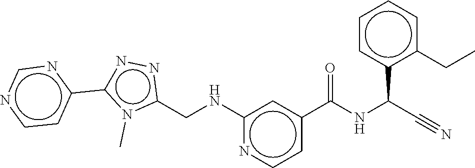 | | | | |
| 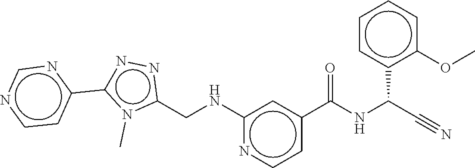 | | | | |
| 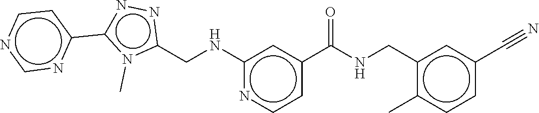 | 7.07 | | 1.41 | 37.1 |
| 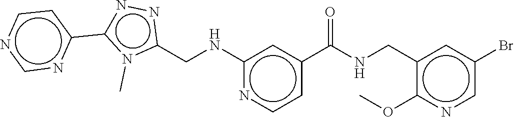 | 28.1 | | >30.0 | 49.7 |
| 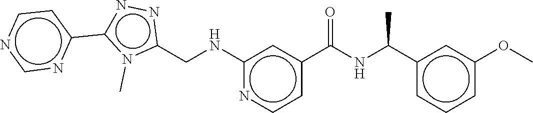 | 125 | | 8 | 52.7 |
| 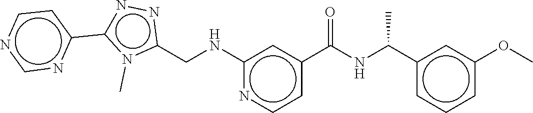 | 9.89 | | 0.468 | 58.6 |
| 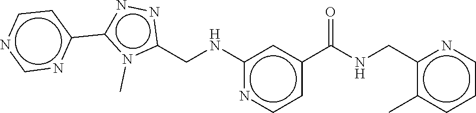 | | | | |
| 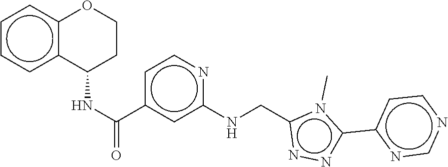 | | | | |
| 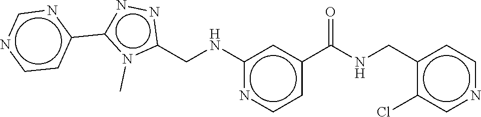 | | | | |
| 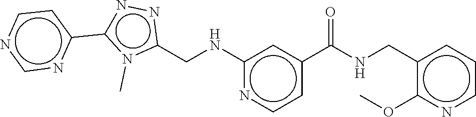 | | | | |
| 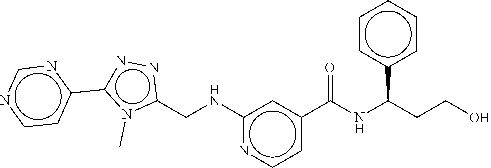 | | | | |
| 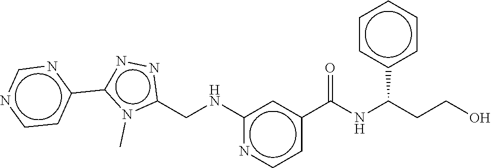 | | | | |
| 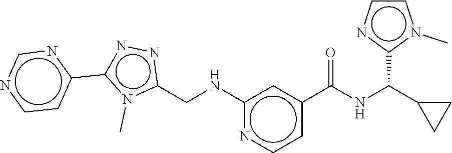 | | | | |
| 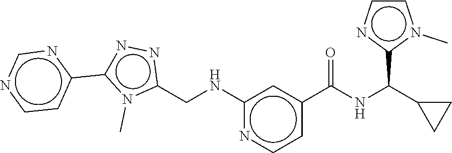 | | | | |
| 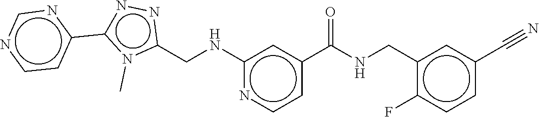 | | | | |
| 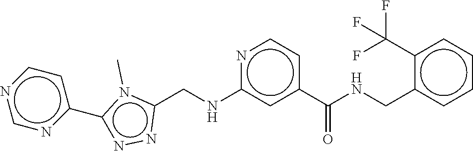 | | | | |
| 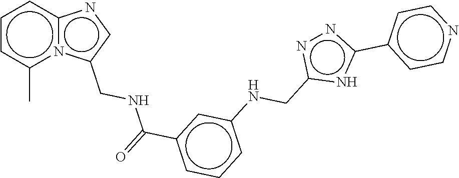 | 8.9 | | >10.2 | 25.6 |
| 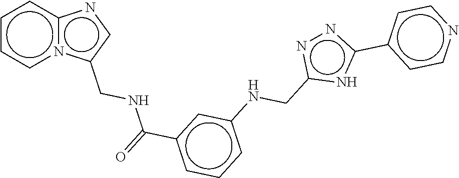 | | | | |
| 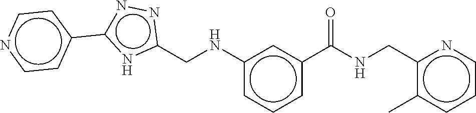 | 12.2 | | >30.0 | 1.37 |
| 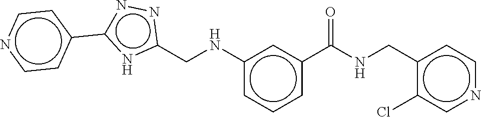 | | | | |
| 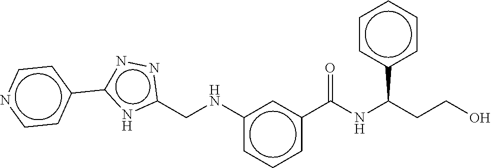 | | | | |
| 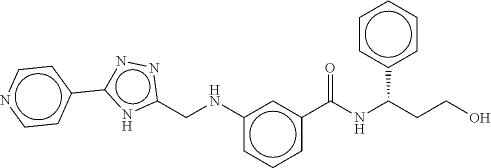 | | | | |
| 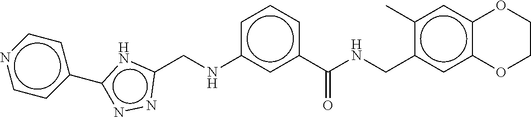 | | | | |
| 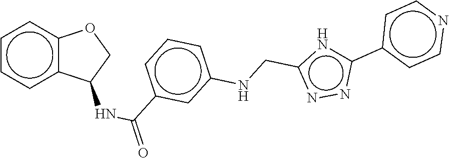 | | | | |
| 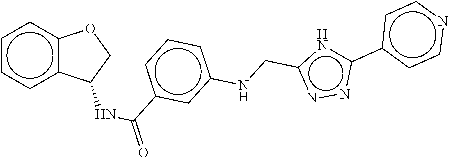 | | | | |
| 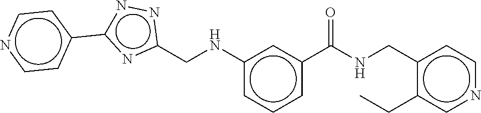 | | | | |
| 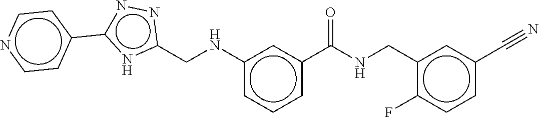 | | | | |
| 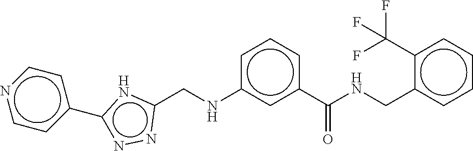 | | | | |
| 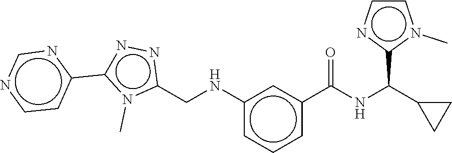 | | | | |
| 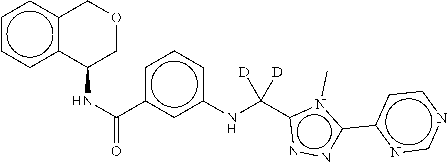 | | | 0.116 | 48.5 |
| 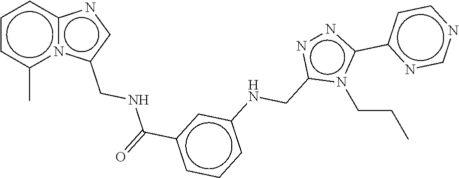 | | | | |
| 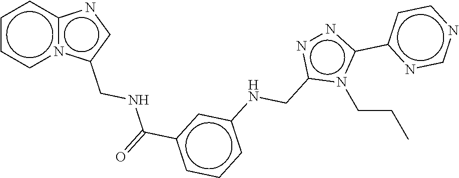 | | | | |
| 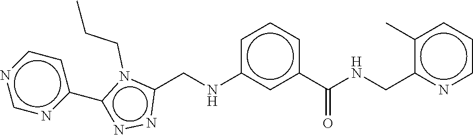 | | | | |
| 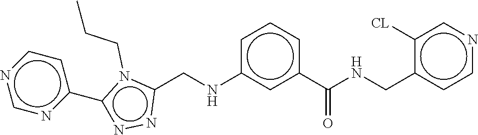 | | | | |
| 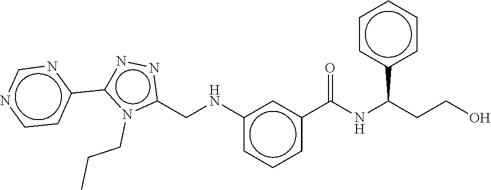 | | | | |
| 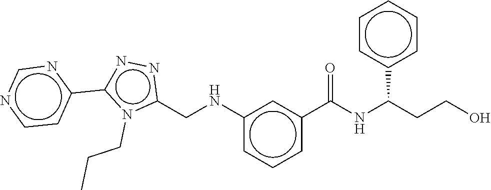 | | | | |
| 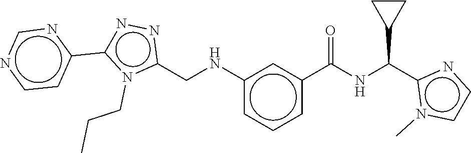 | | | | |
| 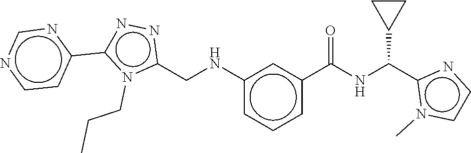 | | | | |
| 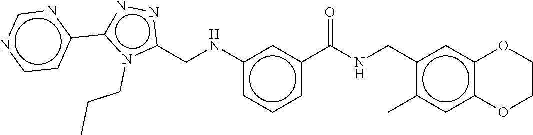 | | | | |
| 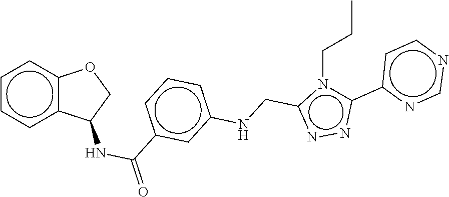 | | | | |
| 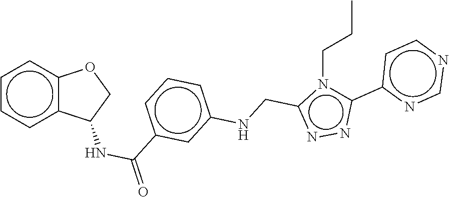 | | | | |
| 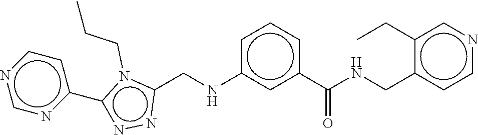 | | | | |
| 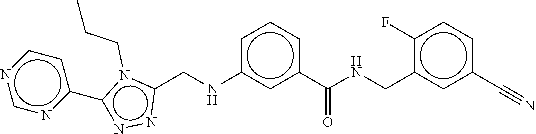 | | | | |
| 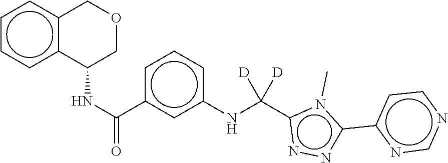 | | | | |
| 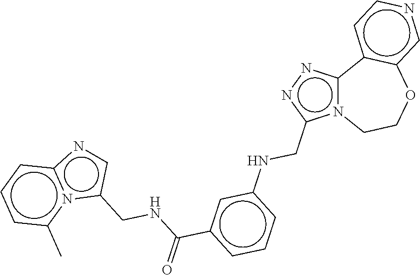 | | | | |
| 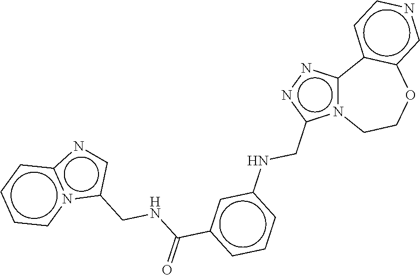 | | | | |
| 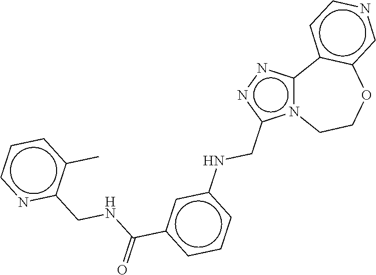 | | | | |
| 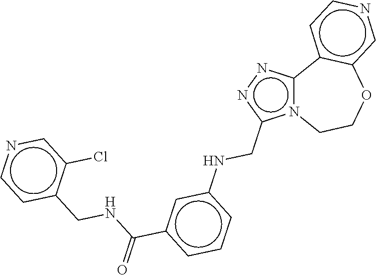 | | | | |
| 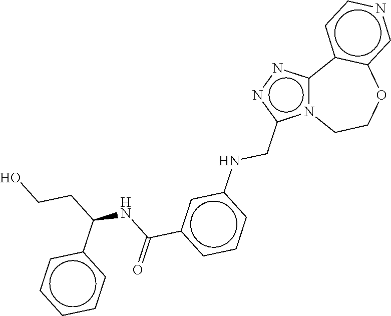 | | | | |
| 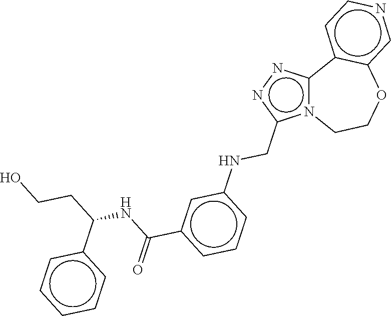 | | | | |
| 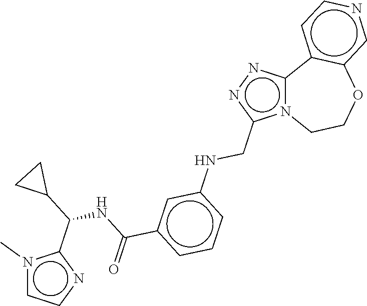 | | | | |
| 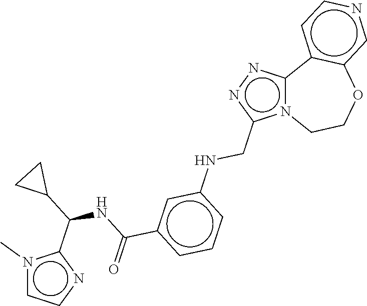 | | | | |
| 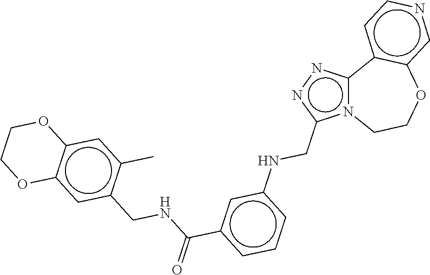 | | | | |
| 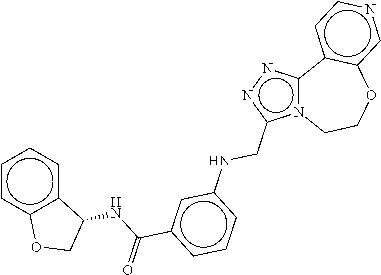 | | | | |
| 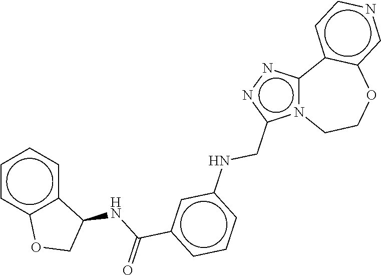 | | | | |
| 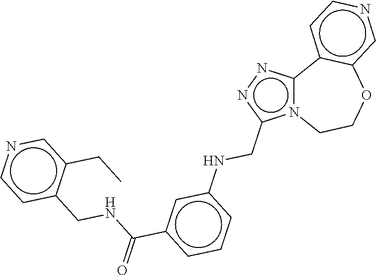 | | | | |
| 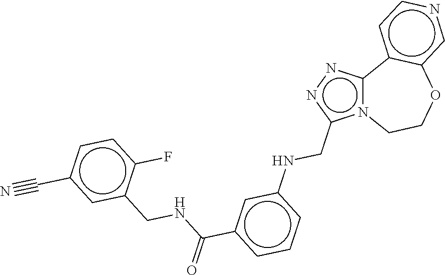 | | | | |
| 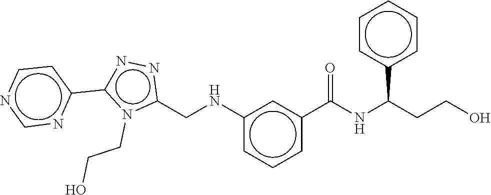 | | | | |
| 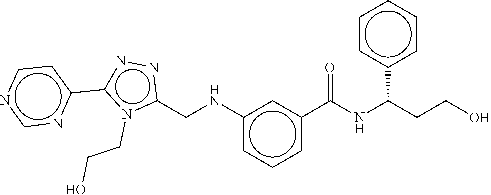 | | | | |
| 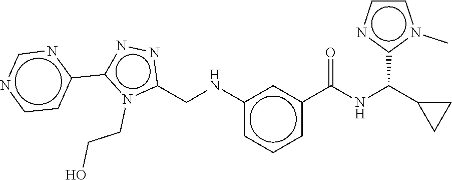 | | | | |
| 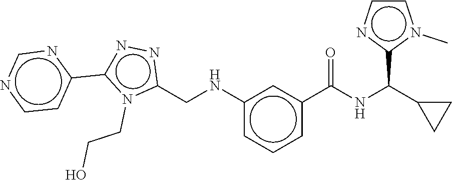 | | | | |
| 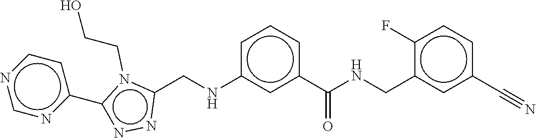 | | | | |
| 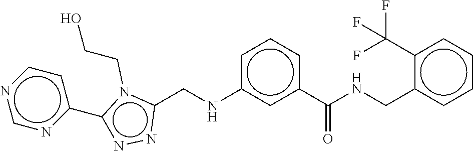 | | | | |
| 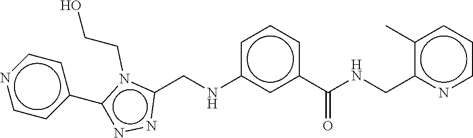 | | | | |
| 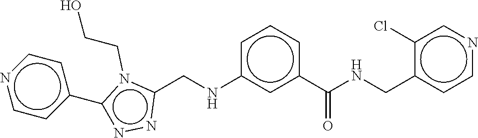 | | | | |
| 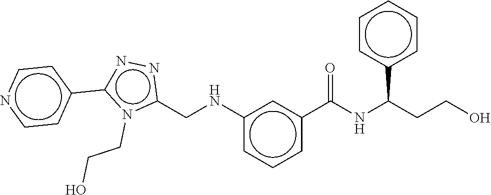 | | | | |
| 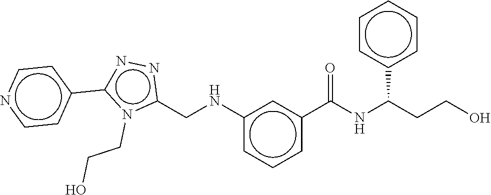 | | | | |
| 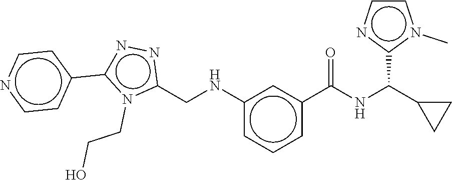 | | | | |
| 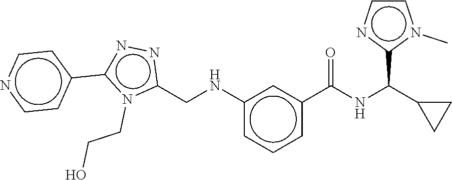 | | | | |
| 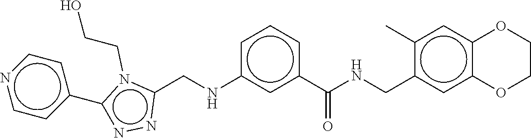 | | | | |
| 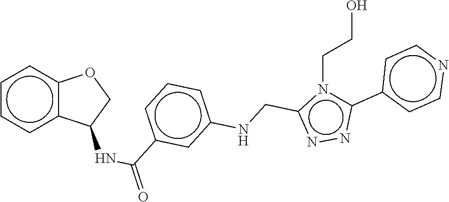 | | | | |
| 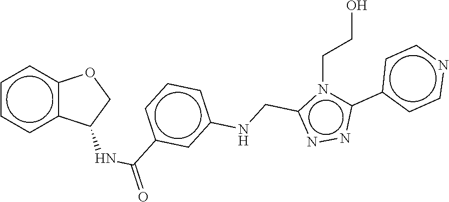 | | | | |
| 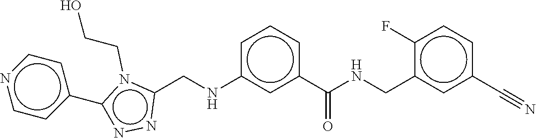 | | | | |
| 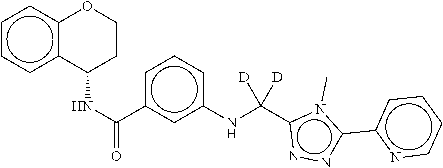 | | 8.3 | 2.65 | 50.6 |
| 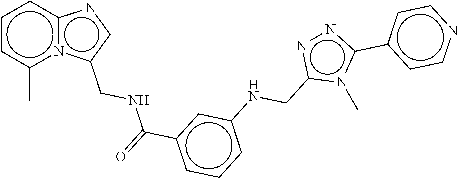 | | | | |
| 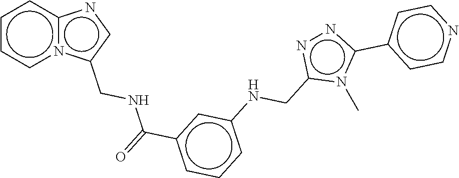 | | | | |
| 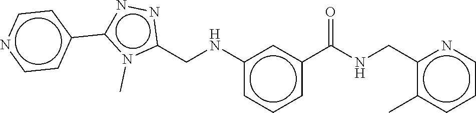 | | | | |
| 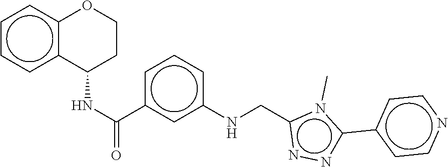 | | | | |
| 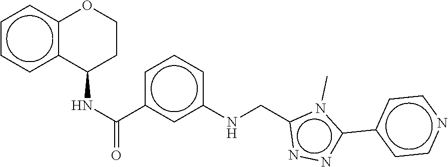 | | | | |
| 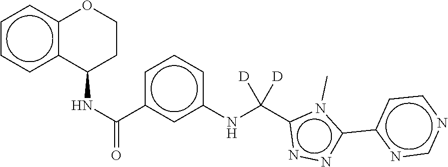 | | | | |
| 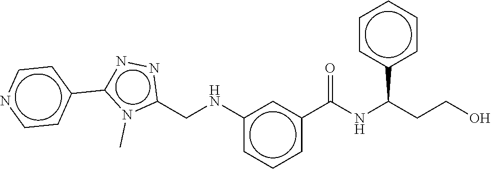 | | | | |
| 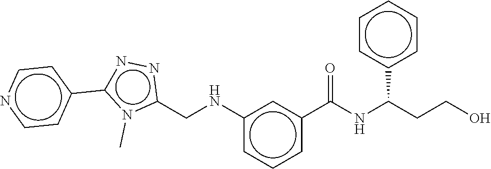 | | | | |
| 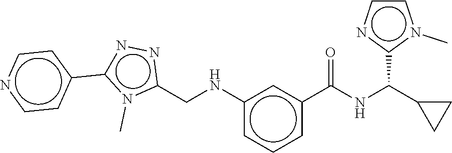 | | | | |
| 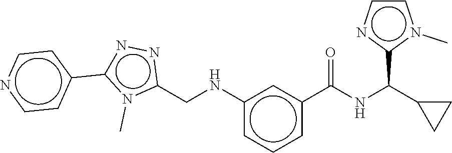 | | | | |
| 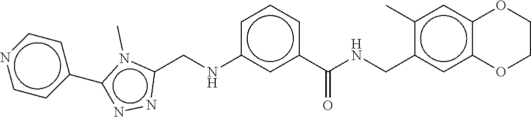 | | | | |
| 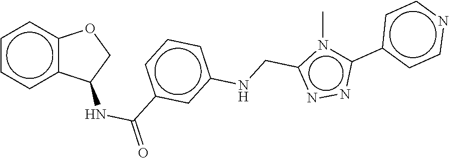 | | | | |
| 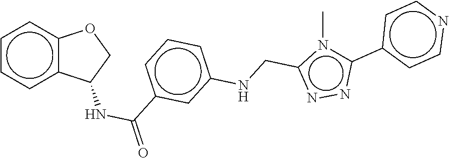 | | | | |
| 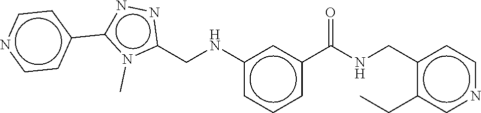 | | | | |
| 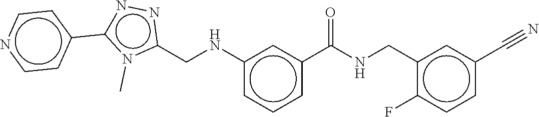 | | | | |
| 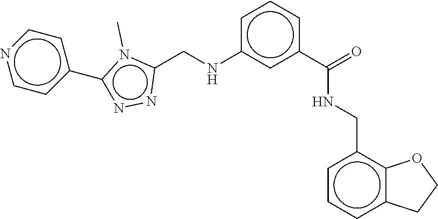 | | | | |
| 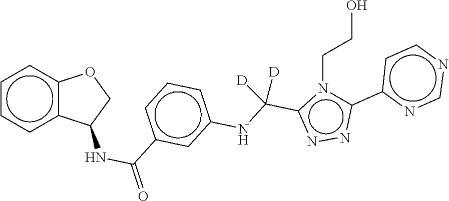 | 3.76 | | 0.096 | 35.4 |
| 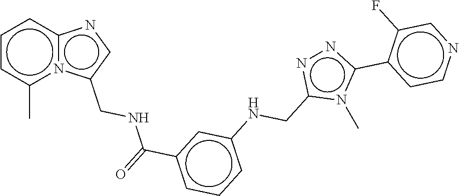 | 3.91 | | >1.36 | 27.7 |
| 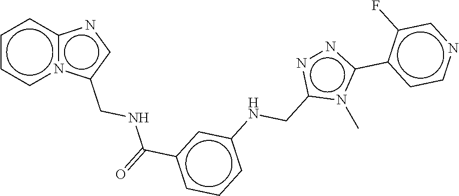 | 39 | | >30.0 | 34.5 |
| 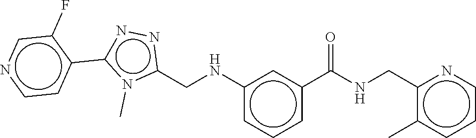 | 312 | | | |
| 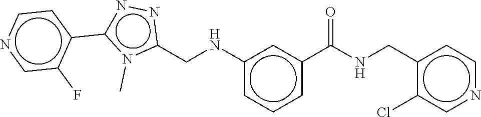 | 31.9 | | | |
| 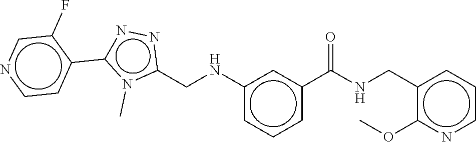 | | | | |
| 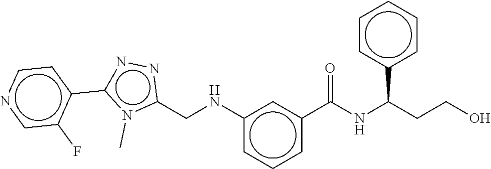 | 18 | | >30.0 | 14.3 |
| 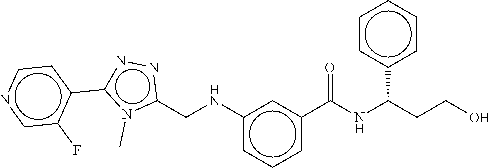 | 28.9 | | >1.44 | 25.3 |
| 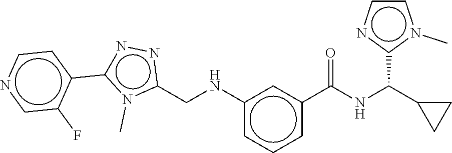 | | | | |
| 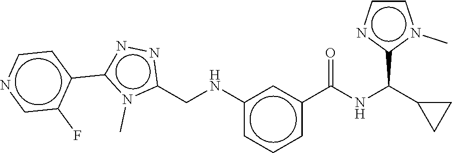 | | | | |
| 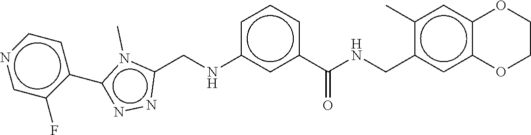 | 8 | | | 30.8 |
| 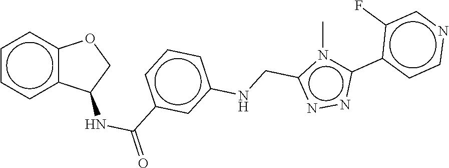 | | | | |
| 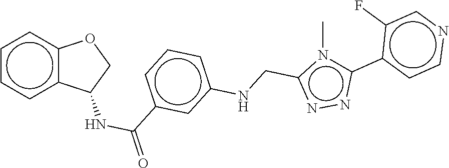 | | | | |
| 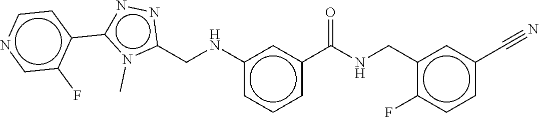 | | | | |
| 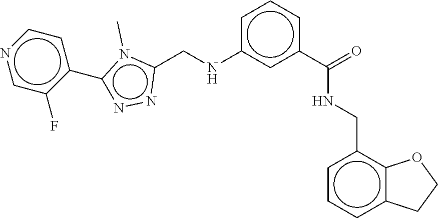 | | | | |
| 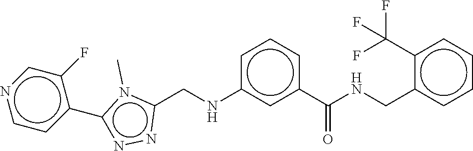 | 39.1 | | >2.32 | 20.7 |
| 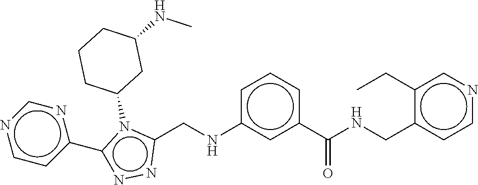 | 456 | 57 | >4.57 | 58.8 |
| 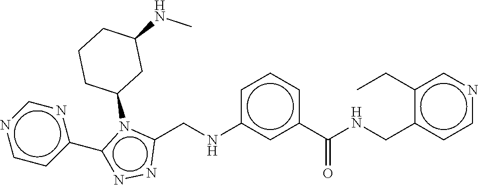 | 10000 | 3900 | | |
| 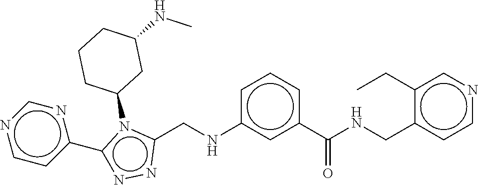 | 529 | 810 | | |
| 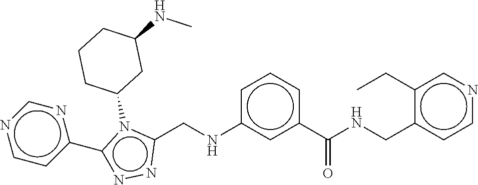 | 10000 | 1000 | | |
| 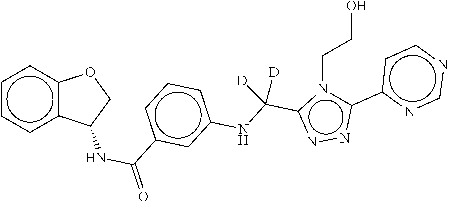 | | | | |
| 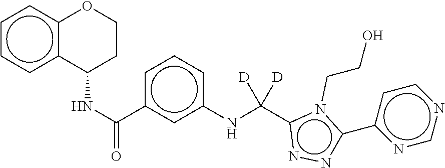 | 3.23 | | 0.094 | 35.3 |
| 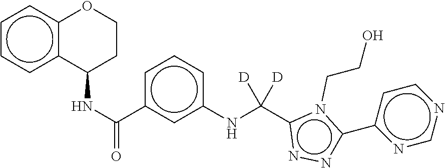 | | | | |
| 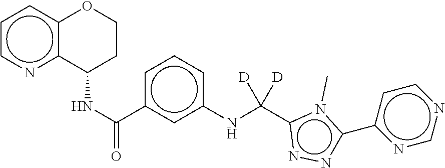 | | | | |
| 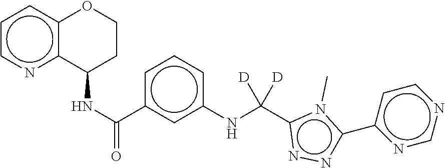 | | | 0.274 | 39.4 |
| 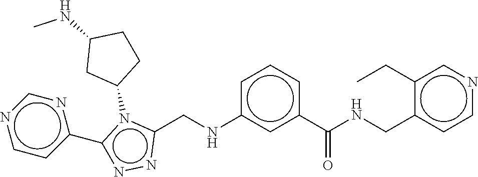 | | 190 | | |
| 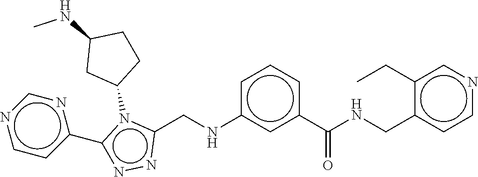 | | 920 | | |
| 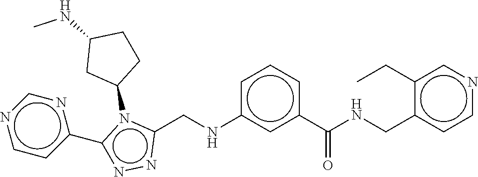 | | 230 | | |
| 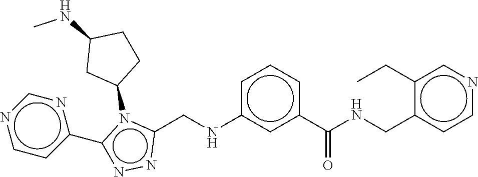 | | 110 | | |
| 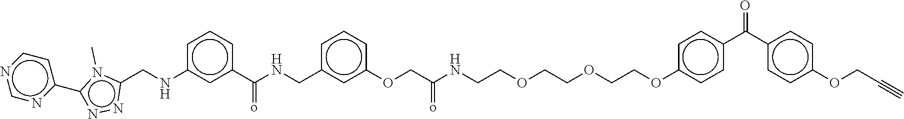 | | | >0.117 | 50.1 |
| 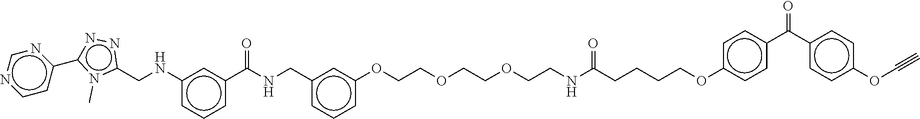 | >1.00E + 03 | | | |
| 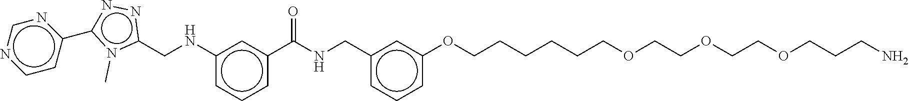 | | | | 67.5 |

### Example 11. In Vivo Efficacy of GRK2 Inhibitor

Compound S1 is effective in vivo in a PAXF1675 (pancreatic cancer) tumor model.

Study Design: Table 8 shows in vivo study design of GRK2 inhibitor Compound S1 in a PAXF1657 tumor model. 5×10⁶ cells per mouse were subcutaneous (s.c.) implanted on right or left flank of the mice in a total volume of 100 μL (50% PBS/50% matrigel). Treatment with Compound S1 started at tumor volume of ˜200 mm³. All compounds were dissolved in 10% PG/50% PEG400/35% Peanut Oil/15% DMSO. Mice were treated for 28 days; treatment was normalized to body weight of the animals. Daily bodyweight measured and body condition score recorded daily. FIG. 3 shows the structure of Compound S1.

TABLE 8
In Vivo Study Design

| | Strain/ | | Inoculation | | | Dose | Dose | | Mice |
|---|---|---|---|---|---|---|---|---|---|
| Group | Sex | Model | Route | Treatment | Vehicle | Concentration | Frequecy | Dosing Route | # |
| G1 | NOD- | PAXF1657 | Subcutaneous | Compound | 10% | 300 mg/kg | QD | subcutaneous | 15 |
| | SCID | | (s.c.) | S1 | PG/50%PEG400/35% | | | (s.c.) | |
| G2 | Female | | | Compound | peanut oil/5%DMSO | 100 mg/kg | BID | | 15 |
| | | | | S1 | | | | | |
| G3 | | | | Vehicle | | | QD | | 15 |
| G4 | | | | Vehicle | | | BID | | 15 |

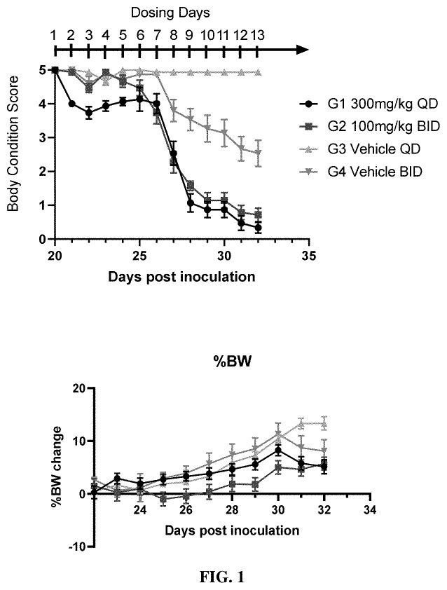

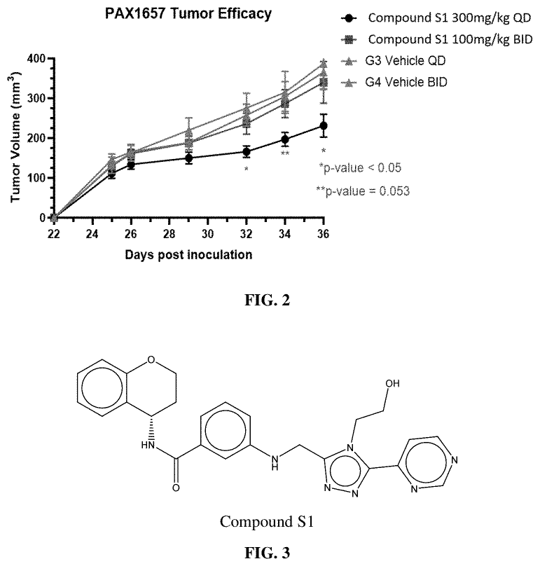

Results: Tolerability of GRK2 inhibitor Compound S1 in mice with PAXF1657 tumors is shown in FIG. 1. In vivo efficacy of Compound S1 in PAXF1657 pancreatic tumor model is shown in FIG. 2. Treatment with Compound S1 led to 35% tumor growth inhibition (TGI) with 300 kg/kg QD dosing in in 10% PG/50% PEG/35% Peanoil/5% DMSO (see Table 9).

TABLE 9
PAXF1657 Tumor Growth Inhibition

| | % TGI | | |
|---|---|---|---|
| Groups | Day 32 | Day 34 | Day 36 |
| Compound S1 300 mg/kg QD | 36 | 36 | 37 |
| Compound S1 100 mg/kg BID | 14 | 9 | 7 |

### Additional Embodiments

Additional embodiments are provided in the following numbered paragraphs:

1. A compound, or a pharmaceutically acceptable salt thereof, having the structure:

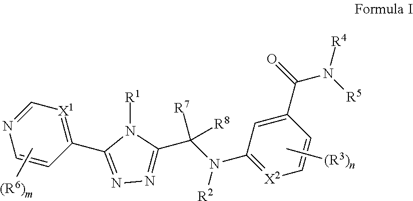

wherein m and n are, independently, 0, 1, 2, or 3;

X¹ is CR⁹ or N;

X² is CR³ or N;

R¹ is hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;

R² and R⁴ are, independently, hydrogen or optionally substituted C₁-C₆ alkyl;

each R³ and R⁶ is, independently, halogen, optionally substituted C₁-C₆ alkyl, optionally substituted C₁-C₆ heteroalkyl, hydroxyl, thiol, or optionally substituted amino;

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl; and

R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl; and

R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl,

wherein the compound is a GRK2-selective compound.

2. The compound of paragraph 1, wherein the compound has the structure:

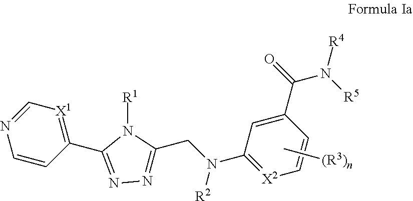

wherein n is 0, 1, 2, or 3;

X¹ and X² are, independently, CR³ or N;

R¹, R², and R⁴ are, independently, hydrogen or optionally substituted C₁-C₆ alkyl;

each R³ is, independently, hydrogen, halogen, optionally substituted C₁-C₆ alkyl, optionally substituted C₁-C₆ heteroalkyl, hydroxyl, thiol, or optionally substituted amino; and

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl, wherein if R⁵ is optionally substituted C₁-C₆ alkyl C₆-C₁₀ aryl then X² is N.

3. The compound, or a pharmaceutically acceptable salt thereof, of paragraph 1 or 2, wherein X¹ is N.

4. The compound, or a pharmaceutically acceptable salt thereof, of paragraph 1 or 2, wherein X¹ is CH.

5. The compound, or a pharmaceutically acceptable salt thereof, of any one of paragraphs 1 to 4, wherein R² is hydrogen.

6. The compound, or a pharmaceutically acceptable salt thereof, of any one of paragraphs 1 to 5, wherein R⁴ is hydrogen.

7. The compound, or a pharmaceutically acceptable salt thereof, of any one of paragraphs 1 to 6, wherein R¹ is hydrogen.

8. The compound, or a pharmaceutically acceptable salt thereof, of any one of paragraphs 1 to 6, wherein R¹ is optionally substituted C₁-C₆ alkyl.

9. The compound, or a pharmaceutically acceptable salt thereof, of paragraph 8, wherein the optionally substituted C₁-C₆ alkyl is methyl or ethyl.

10. The compound, or a pharmaceutically acceptable salt thereof, of paragraph 9, wherein the optionally substituted C₁-C₆ alkyl is methyl.

11. The compound, or a pharmaceutically acceptable salt thereof, of any one of paragraphs 1 to 10, wherein n is 0.

12. The compound, or a pharmaceutically acceptable salt thereof, of any one of paragraphs 1 to 11, wherein R⁵ is optionally substituted C₁-C₆ alkyl C₆-C₁₀ aryl.

13. The compound, or a pharmaceutically acceptable salt thereof of paragraph 12, wherein the optionally substituted C₁-C₆ alkyl C₆-C₁₀ aryl is optionally substituted C₂ alkyl C₆-C₁₀ aryl.

14. The compound, or a pharmaceutically acceptable salt thereof, of paragraph 13, wherein the optionally substituted C₂ alkyl C₆-C₁₀ aryl is:

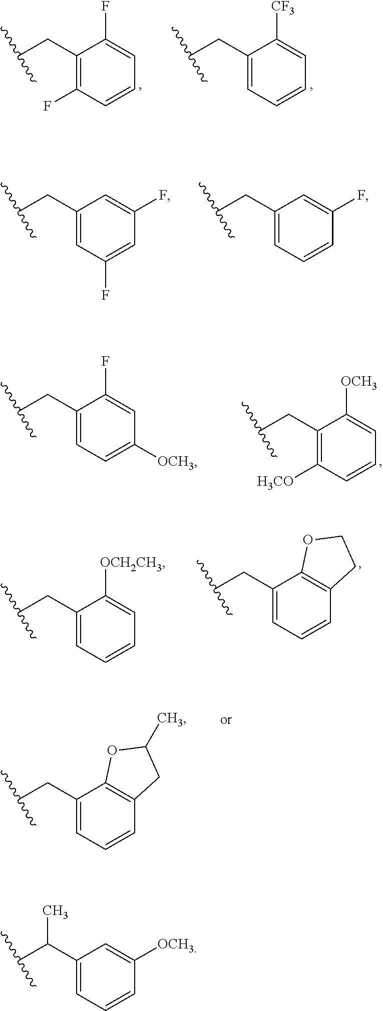

15. The compound, or a pharmaceutically acceptable salt thereof, of paragraph 14, wherein the optionally substituted C₂ alkyl C₆-C₁₀ aryl is:

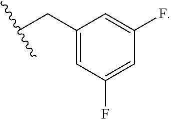 [SMILES: *Cc1cc(F)cc(F)c1]

16. The compound, or a pharmaceutically acceptable salt thereof, of any one of paragraphs 1 to 11, wherein R⁵ is optionally substituted C₂-C₉ heterocyclyl.

17. The compound, or a pharmaceutically acceptable salt thereof, of paragraph 16, wherein the optionally substituted C₂-C₉ heterocyclyl is:

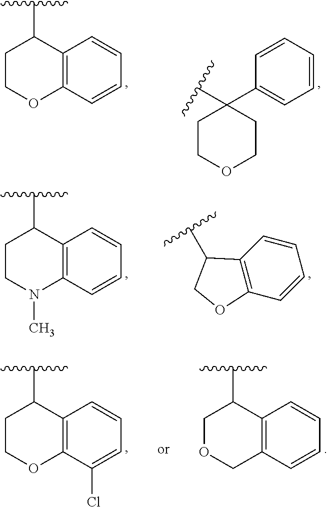

18. The compound, or a pharmaceutically acceptable salt thereof, of any one of paragraphs 1 to 11, wherein R⁵ is optionally substituted C₃-C₈ cycloalkyl.

19. The compound, or a pharmaceutically acceptable salt thereof, of paragraph 18, wherein the optionally substituted C₃-C₈ cycloalkyl is:

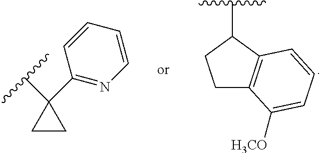 [SMILES: *C1(c2ccccn2)CC1 ; *C1CCc2c(OC)cccc21]

20. The compound, or a pharmaceutically acceptable salt thereof, of any one of paragraphs 1 to 11, wherein R⁵ is optionally substituted C₁-C₆ alkyl C₂-C₉ heteroaryl.

21. The compound, or a pharmaceutically acceptable salt thereof, of paragraph 20, wherein the optionally substituted C₁-C₆ alkyl C₂-C₉ heteroaryl is:

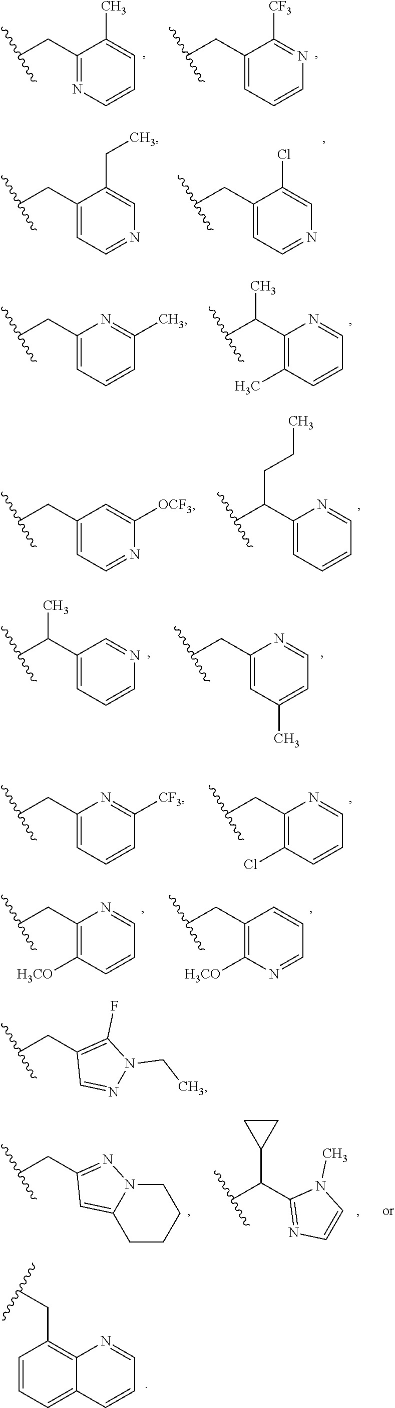

22. A compound, or a pharmaceutically acceptable salt thereof, having the structure of Formula II:

A-L-B Formula II,

wherein

L is a linker;

B is a degradation moiety; and

A has the structure of Formula III:

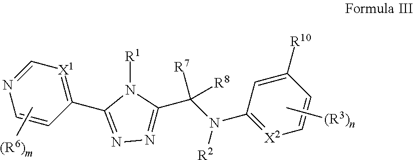

wherein m and n are, independently, 0, 1, 2, or 3;

X¹ is CR⁹ or N;

X² is CR³ or N;

R¹ is A¹, hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;

R² is hydrogen or optionally substituted C₁-C₆ alkyl;

each R³ and R⁶ is, independently, hydrogen, halogen, optionally substituted C₁-C₆ alkyl, optionally substituted C₁-C₆ heteroalkyl, hydroxyl, thiol, or optionally substituted amino;

R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl;

R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl;

R¹⁰ is —C(O)NR¹¹R¹² or —NHC(O)—R¹¹;

R¹¹ is A¹, optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl;

R¹² is hydrogen or optionally substituted C₁-C₆ alkyl; and

A¹ is a bond between A and the linker, wherein at least one, and only one of R¹ and R⁸ is A¹.

23. The compound of paragraph 22, wherein A has the structure:

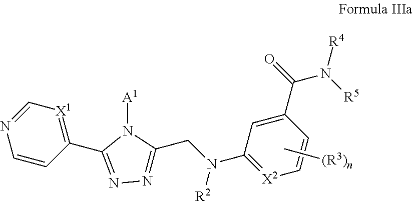

wherein n is 0, 1, 2, or 3;

X¹ and X² are, independently, CR³ or N;

R¹, R², and R⁴ are, independently, hydrogen or optionally substituted C₁-C₆ alkyl;

each R³ is, independently, hydrogen, halogen, optionally substituted C₁-C₆ alkyl, optionally substituted C₁-C₆ heteroalkyl, hydroxyl, thiol, or optionally substituted amino; and

R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl; and

A¹ is a bond between A and the linker.

24. The compound, or a pharmaceutically acceptable salt thereof, of paragraph 22 or 23, wherein X¹ is N.

25. The compound, or a pharmaceutically acceptable salt thereof, of paragraph 22 or 23, wherein X¹ is CH.

26. The compound, or a pharmaceutically acceptable salt thereof, of any one of paragraphs 22 to 25, wherein R² is hydrogen.

27. The compound, or a pharmaceutically acceptable salt thereof, of any one of paragraphs 22 to 26, wherein R⁴ is hydrogen.

28. The compound, or a pharmaceutically acceptable salt thereof, of any one of paragraphs 22 to 27, wherein n is 0.

29. The compound, or a pharmaceutically acceptable salt thereof, of any one of paragraphs 22 to 28, wherein R⁵ is optionally substituted C₁-C₆ alkyl C₆-C₁₀ aryl.

30. The compound, or a pharmaceutically acceptable salt thereof of paragraph 29, wherein the optionally substituted C₁-C₆ alkyl C₆-C₁₀ aryl is optionally substituted C₂ alkyl C₆-C₁₀ aryl.

31. The compound, or a pharmaceutically acceptable salt thereof, of paragraph 30, wherein the optionally substituted C₂ alkyl C₆-C₁₀ aryl is:

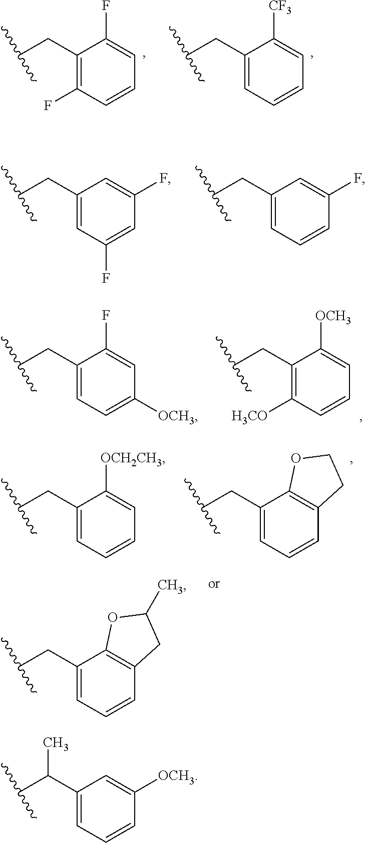

32. The compound, or a pharmaceutically acceptable salt thereof, of paragraph 31, wherein the optionally substituted C₂ alkyl C₆-C₁₀ aryl is:

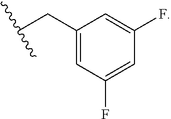 [SMILES: *Cc1cc(F)cc(F)c1]

33. The compound, or a pharmaceutically acceptable salt thereof, of any one of paragraphs 22 to 28, wherein R⁵ is optionally substituted C₂-C₉ heterocyclyl.

34. The compound, or a pharmaceutically acceptable salt thereof, of paragraph 33, wherein the optionally substituted C₂-C₉ heterocyclyl is:

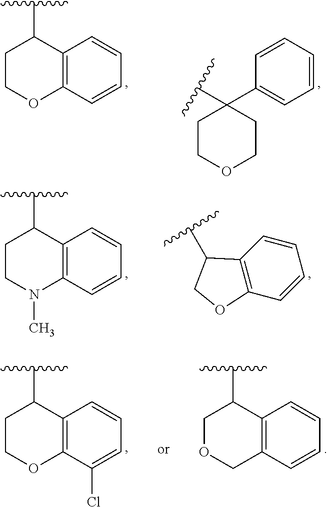

35. The compound, or a pharmaceutically acceptable salt thereof, of any one of paragraphs 22 to 28, wherein R⁵ is optionally substituted C₃-C₈ cycloalkyl.

36. The compound, or a pharmaceutically acceptable salt thereof, of paragraph 35, wherein the optionally substituted C₃-C₈ cycloalkyl is:

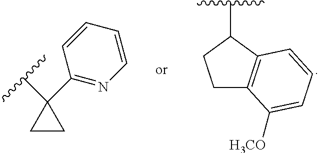 [SMILES: *C1(c2ccccn2)CC1 ; *C1CCc2c(OC)cccc21]

37. The compound, or a pharmaceutically acceptable salt thereof, of any one of paragraphs 22 to 28, wherein R⁵ is optionally substituted C₁-C₆ alkyl C₂-C₉ heteroaryl.

38. The compound, or a pharmaceutically acceptable salt thereof, of paragraph 37, wherein the optionally substituted C₁-C₆ alkyl C₂-C₉ heteroaryl is:

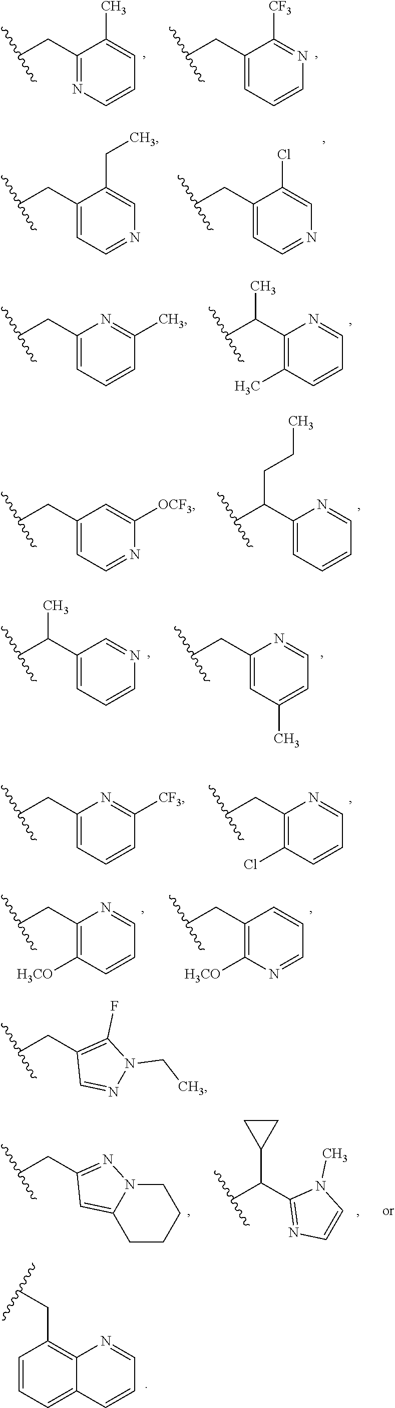

39. The compound, or a pharmaceutically acceptable salt thereof, of any one of paragraphs 22 to 38, wherein the degradation moiety is a ubiquitin ligase binding moiety.

40. The compound, or pharmaceutically acceptable salt thereof, of paragraph 39, wherein the ubiquitin ligase binding moiety comprises Cereblon ligands, IAP (Inhibitors of Apoptosis) ligands, mouse double minute 2 homolog (MDM2), or von Hippel-Lindau (VHL) ligands, or derivatives or analogs thereof.

41. The compound, or pharmaceutically acceptable salt thereof, of any one of paragraphs 22 to 40, wherein the linker has the structure of Formula IV:

A¹-(B¹)_{f}—(C¹)_{g}—(B²)ₕ-(D)-(B³)ᵢ—(C²)ⱼ—(B⁴)ᵢ-A² Formula IV

wherein

A¹ is a bond between A and the linker;

A² is a bond between the linker and B;

each of B¹, B², B³, and B⁴ is, independently, optionally substituted C₁-C₆ alkyl, optionally substituted C₁-C₆ heteroalkyl, O, S, S(O)₂, or NRN;

each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;

each of C¹ and C² is, independently, carbonyl, thiocarbonyl, sulphonyl, or phosphoryl;

each of f, g, h, i, j, and k is, independently, 0 or 1; and

D is optionally substituted C₁₋₁₂ alkyl, optionally substituted C₂₋₁₂ alkenyl, optionally substituted C₂₋₁₂ alkynyl, optionally substituted C₂-C₁₂ polyethylene glycol, or optionally substituted C₁₋₁₂ heteroalkyl, or a chemical bond linking A¹-(B¹)_{f}—(C¹)_{g}—(B²)ₕ— to —(B³)ᵢ—(C²)ⱼ—(B⁴)ₖ-A².

42. A compound, or a pharmaceutically acceptable salt thereof, having the structure of any one of compounds 1-65 in Table 1, compounds D1-D51 in Table 2, or any compound in Table 7.

43. A pharmaceutical composition comprising the compound, or pharmaceutically acceptable salt thereof, of any one of paragraphs 1 to 42 and a pharmaceutically acceptable excipient.

44. A method of decreasing the activity of GRK2 in a cell, the method comprising contacting the cell with an effective amount of the compound of any one of paragraphs 1 to 42 or a pharmaceutical composition of paragraph 43.

45. A method of treating a GRK2-related disorder in a subject in need thereof, the method comprising administering to the subject an effective amount of a GRK2-selective compound, or pharmaceutically acceptable salt thereof, or a composition thereof.

46. The method of paragraph 45, wherein the GRK2-selective compound, or pharmaceutically acceptable salt thereof, or a composition thereof is the compound of any one of paragraphs 1 to 42 or a pharmaceutical composition of paragraph 43.

47. A method of treating cancer in a subject in need thereof, the method comprising administering to the subject an effective amount of the compound, or pharmaceutically acceptable salt thereof, of any one of paragraphs 1 to 42 or a pharmaceutical composition of paragraph 43.

48. The method of paragraph 47, wherein the cancer is pancreatic cancer.

49. A method of identifying a GRK2-selective compound, the method comprising:

a. contacting a first cell line that expresses GRK2 with a test compound;

b. contacting a second cell line that has been engineered to overexpress GRK2 with the test compound;

c. assessing whether the proliferation of the first cell line is decreased in step a relative to the proliferation of the second cell line in step b,

wherein a decrease in proliferation of the first cell line in step a of at least 2-fold indicates that the test compound is a GRK2-selective compound.

### Other Embodiments

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the methods have been described in conjunction with various embodiments and examples, it is not intended that the methods be limited to such embodiments or examples. On the contrary, the present disclosure encompasses various alternatives, modifications, and equivalents, as will be appreciated by those of skill in the art.

While the methods have been particularly shown and described with reference to specific illustrative embodiments, it should be understood that various changes in form and detail may be made without departing from the spirit and scope of the present disclosure. Therefore, all embodiments that come within the scope and spirit of the present disclosure, and equivalents thereto, are intended to be claimed. The claims, descriptions and diagrams of the methods, systems, and assays of the present disclosure should not be read as limited to the described order of elements unless stated to that effect.

1. A compound having the structure: 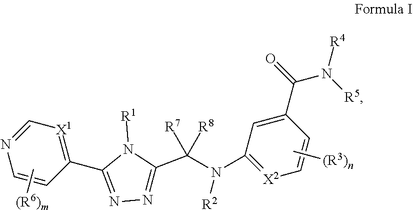
or a pharmaceutically acceptable salt thereof:
wherein m and n are, independently, 0, 1, 2, or 3;
X¹ is CR⁹ or N;
X² is CR³ or N;
R¹ is hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;
R² and R⁴ are, independently, hydrogen or optionally substituted C₁-C₆ alkyl;
each R³ and R⁶ is, independently, halogen, optionally substituted C₁-C₆ alkyl, optionally substituted C₁-C₆ heteroalkyl, hydroxyl, thiol, or optionally substituted amino;
R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆alkyl C₂-C₉ heterocyclyl;
R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl; and
R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl,
wherein the compound or pharmaceutically acceptable salt thereof is a GRK2-selective compound.

2. The compound of claim 1, wherein the compound has the structure: 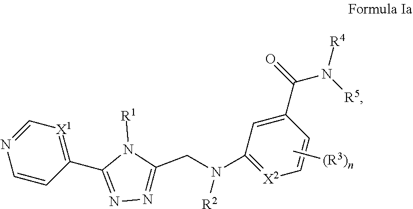
or a pharmaceutically acceptable salt thereof:
wherein n is 0, 1, 2, or 3;
X¹ and X² are, independently, CR³ or N;
R¹, R², and R⁴ are, independently, hydrogen or optionally substituted C₁-C₆ alkyl;
each R³ is, independently, hydrogen, halogen, optionally substituted C₁-C₆ alkyl, optionally substituted C₁-C₆ heteroalkyl, hydroxyl, thiol, or optionally substituted amino; and
R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆alkyl C₂-C₉ heterocyclyl, wherein if R⁵ is optionally substituted C₁-C₆ alkyl C₆-C₁₀ aryl then X² is N.

3-21. (canceled)

22. A compound, having the structure of Formula II:
A-L-B Formula II,
or a pharmaceutically acceptable salt thereof:
wherein L is a linker;
B is a degradation moiety; and
A has the structure of Formula III: 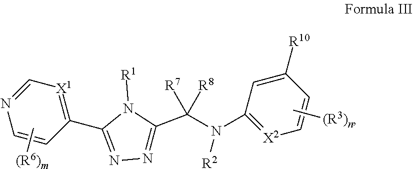
wherein m and n are, independently, 0, 1, 2, or 3;
X¹ is CR⁹ or N;
X² is CR³ or N;
R¹ is A¹, hydrogen, optionally substituted C₁-C₆ alkyl, optionally substituted C₁-C₆ heteroalkyl, optionally substituted C₃-C₈ cycloalkyl, optionally substituted C₂-C₉ heterocyclyl, optionally substituted C₁-C₆ alkyl C₃-C₈ cycloalkyl, optionally substituted C₁-C₆ heteroalkyl C₃-C₈ cycloalkyl, optionally substituted C₁-C₆ alkyl C₂-C₉ heterocyclyl, or optionally substituted C₁-C₆ heteroalkyl C₂-C₉ heterocyclyl;
R² is hydrogen or optionally substituted C₁-C₆ alkyl;
each R³ and R⁶ is, independently, hydrogen, halogen, optionally substituted C₁-C₆ alkyl, optionally substituted C₁-C₆ heteroalkyl, hydroxyl, thiol, or optionally substituted amino;
R⁷ and R⁸ are, independently, hydrogen, deuterium, optionally substituted C₁-C₆ alkyl, or R⁷ and R⁸ combine with the atoms to which they are attached to form a optionally substituted C₃-C₈ cycloalkyl or C₂-C₉ heterocyclyl;
R⁹ is hydrogen, halogen, optionally substituted C₁-C₆ alkyl, optionally substituted C₁-C₆ heteroalkyl, hydroxyl, thiol, or optionally substituted amino, or R⁹ combines with R¹ and the atoms to which they are attached to form a C₂-C₉ heterocyclyl;
R¹⁰ is —C(O)NR¹¹R¹² or —NHC(O)—R¹¹;
R¹¹ is A¹, optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆ alkyl C₂-C₉ heterocyclyl;
R¹² is hydrogen or optionally substituted C₁-C₆ alkyl; and
A¹ is a bond between A and the linker, wherein at least one, and only one of R¹ and R⁸ is A¹.

23. The compound of claim 22, or a pharmaceutically acceptable salt thereof, wherein A has the structure: 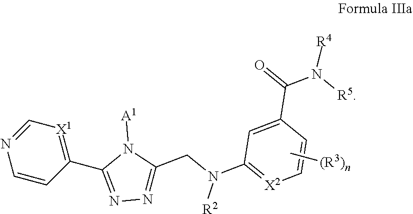
wherein n is 0, 1, 2, or 3;
X¹ and X² are, independently, CR³ or N;
R¹, R², and R⁴ are, independently, hydrogen or optionally substituted C₁-C₆ alkyl;
each R³ is, independently, hydrogen, halogen, optionally substituted C₁-C₆ alkyl, optionally substituted C₁-C₆ heteroalkyl, hydroxyl, thiol, or optionally substituted amino;
R⁵ is optionally substituted C₆-C₁₀ aryl, optionally substituted C₂-C₉ heteroaryl, optionally substituted C₂-C₉ heterocyclyl, optionally substituted C₃-C₈ cycloalkyl, optionally substituted C₁-C₆ alkyl C₆-C₁₀ aryl, optionally substituted C₁-C₆ alkyl C₂-C₉ heteroaryl, or optionally substituted C₁-C₆alkyl C₂-C₉ heterocyclyl; and
A¹ is a bond between A and the linker.

24-38. (canceled)

39. The compound of claim 22, or a pharmaceutically acceptable salt thereof, wherein the degradation moiety is a ubiquitin ligase binding moiety.

40. (canceled)

41. The compound of claim 22, or a pharmaceutically acceptable salt thereof, wherein the linker has the structure of Formula IV:
A¹-(B¹)_{f}—(C¹)_{g}—(B²)ₕ-(D)-(B³)ᵢ—(C²)ⱼ—(B⁴)ₖ-A² Formula IV,
wherein A¹ is a bond between A and the linker;
A² is a bond between the linker and B;
each of B¹, B², B³, and B⁴ is, independently, optionally substituted C₁-C₆ alkyl, optionally substituted C₁-C₆ heteroalkyl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₂ alkyl, optionally substituted C₂₋₁₂ alkenyl, optionally substituted C₂₋₁₂ alkynyl, optionally substituted C₂-C₁₂ polyethylene glycol, or optionally substituted C₁₋₁₂ heteroalkyl, or a chemical bond linking A¹-(B¹)_{f}—(C¹)_{g}—(B²)ₕ— to —(B³)ᵢ—(C²)ⱼ—(B⁴)ₖ-A².

42. The compound of claim 1, wherein the compound is selected from any one of the compounds in Table 1 and Table 7, and pharmaceutically acceptable salts thereof.

43. A pharmaceutical composition comprising the compound of claim 1, or a pharmaceutically acceptable salt thereof, and a pharmaceutically acceptable excipient.

44. A method of decreasing the activity of GRK2 in a cell, the method comprising contacting the cell with an effective amount of the compound of claim 1, or a pharmaceutically acceptable salt thereof, or a pharmaceutical composition thereof.

45. A method of treating a GRK2-related disorder in a subject in need thereof, the method comprising administering to the subject an effective amount of the compound of claim 1, or pharmaceutically acceptable salt thereof, or a pharmaceutical composition thereof.

46. (canceled)

47. A method of treating cancer in a subject in need thereof, the method comprising administering to the subject an effective amount of the compound of claim 1, or a pharmaceutically acceptable salt thereof, or a pharmaceutical composition thereof.

48. The method of claim 47, wherein the cancer is pancreatic cancer.

49. (canceled)

50. The compound of claim 22, wherein the compound is selected from any one of the compounds in Table 2, and pharmaceutically acceptable salts thereof.

51. A pharmaceutical composition comprising the compound of claim 22, or a pharmaceutically acceptable salt thereof, and a pharmaceutically acceptable excipient.

52. The compound of claim 1, wherein the compound has the structure: 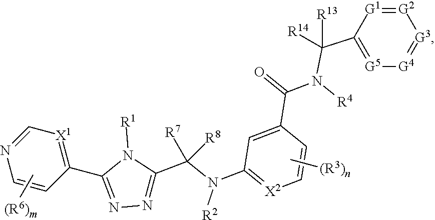
or a pharmaceutically acceptable salt thereof;
wherein G¹ is CR¹⁵ or N;
G², G³, G⁴, and G⁵ are each independently CR¹⁶, CH, or N;
each instance of R¹⁵ and R¹⁶ is independently hydrogen, halogen, —CN, —N3, —NO₂, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₈ carbocyclyl, optionally substituted 3-8 membered heterocyclyl, optionally substituted C₆-C₁₀ aryl, optionally substituted 5-10 membered heteroaryl, optionally substituted C₁-C₆ acyl, optionally substituted hydroxyl, optionally substituted amino, or optionally substituted thiol;
R¹³ and R¹⁴ are independently hydrogen or optionally substituted C₁₋₆ alkyl, or R¹³ and R¹⁴ are joined together with the intervening atoms to form optionally substituted C₃-C₈ carbocyclyl or 3-8 membered heterocyclyl; and
optionally wherein R¹³ and R¹⁵ are joined together with the intervening atoms to form optionally substituted C₄-C₈ carbocyclyl or optionally substituted 4-8 membered heterocyclyl.

53. The compound of claim 52, wherein the compound has the structure: 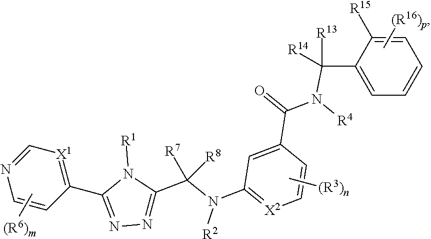
or a pharmaceutically acceptable salt thereof;
wherein p is 0, 1, 2, 3, or 4.

54. The compound of claim 53, wherein the compound has the structure: 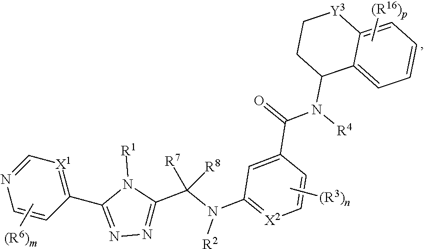
or a pharmaceutically acceptable salt thereof;
wherein Y³ is O, S, or optionally substituted N.

55. The compound of claim 54, wherein the compound has the structure: 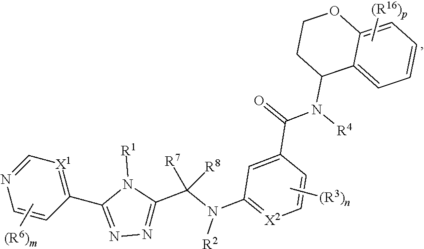
or a pharmaceutically acceptable salt thereof.

56. The compound of claim 1, wherein the compound has the structure: 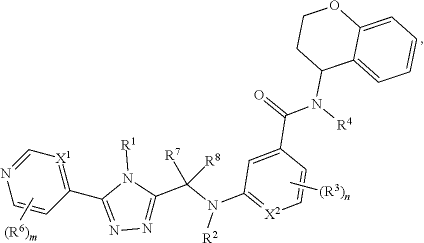
or a pharmaceutically acceptable salt thereof.

57. The compound of claim 1, wherein the compound has the structure: 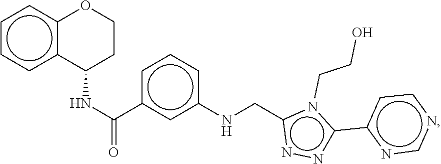
or a pharmaceutically acceptable salt thereof.


SEQUENCE LISTING (2 sequences)

SEQ ID NO: 1 - DNA, 2070 bp, Unknown
  Naturally Occurring
atggcggacctggaggcggtgctggccgacgtgagctacctgatggccatggagaagagcaaggccacgccggccgcgcgcgccagcaagaagatcctgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaaggctgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacgctccctgctggaggggttgctgcagagggatgtcaaccggagattgggctgcctgggccgaggggctcaggaggtgaaagagagcccctttttccgctccctggactggcagatggtcttcttgcagaagtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaaccccttcctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctctga

SEQ ID NO: 2 - protein, 689 aa, Unknown
  Naturally Occurring
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKADTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELRSLLEGLLQRDVNRRLGCLGRGAQEVKESPFFRSLDWQMVFLQKYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGL